(19) United States
(12) Patent Application Publication
(10) Pub. No.: US 2023/0024108 A1
(43) Pub. Date: Jan. 26, 2023

(54) ADENOSINE A2A RECEPTOR ANTAGONISTS

(71) Applicant: Teon Therapeutics, Inc., Redwood City, CA (US)

(72) Inventors: Elfatih Elzein, Redwood City, CA (US); Jiwen Liu, Redwood City, CA (US)

(21) Appl. No.: 17/626,075

(22) PCT Filed: Jul. 15, 2020
(86) PCT No.: PCT/US2020/042158

§ 371 Date: Jan. 10, 2022

Related U.S. Application Data
(60) Provisional application No. 62/875,251, filed on Jul. 17, 2019.

(51) Int. Cl. A61K 31/519; C07D 487/14; C07D 519/00; A61K 31/551; A61K 9/08; A61K 9/20; A61K 9/48; A61K 9/00

(52) U.S. Cl. CPC A61K 31/519; C07D 487/14; C07D 519/00; A61K 31/551; A61K 9/08; A61K 9/2054; A61K 9/4825; A61K 9/0019; A61K 9/0053

(57) ABSTRACT

Disclosed herein are compounds, compositions, and methods for modulating the A_{2A} adenosine receptor with the compounds and compositions disclosed herein. Also described are methods of treating diseases or disorders that are mediated by the A_{2A} adenosine receptor, such as cancer, with A_{2A} adenosine receptor antagonists.

### CROSS-REFERENCE

This application claims benefit of U.S. Provisional Application No. 62/875,251, filed on Jul. 17, 2019, which is herein incorporated by reference in its entirety.

### FIELD OF THE INVENTION

Described herein are compounds, methods of making such compounds, pharmaceutical compositions and medicaments comprising such compounds, and methods of using such compounds in the treatment of conditions, diseases, or disorders that would benefit from modulation of A_{2A} adenosine receptor activity.

### BACKGROUND OF THE INVENTION

Adenosine, an endogenous nucleoside, ubiquitously exists inside and outside of living cells. It plays multiple physiological roles to maintain the homeostasis of cells, tissues, and organs. Four distinct adenosine receptor subtypes have been identified to date, A1, A2A, A2B, and A3, all of which belong to the family of G-protein-coupled receptors characterized by 7-transmembrane-spanning helical domains. Interaction of adenosine with its receptors initiates signal transduction pathways, including the classical adenylate cyclase effector system that utilizes cyclic adenosine monophosphate (cAMP) as a second messenger. Activation of the A1 and A3 adenosine receptors (A1-AdoR and A3-AdoR) inhibits adenylate cyclase activity through activation of pertussis-sensitive Gi proteins and results in a decrease in intracellular levels of cAMP. Activation of the A2A and A2B adenosine receptors (A2A-AdoR and A2B-AdoR) stimulates adenylate cyclase via activation of Gs proteins and leads to intracellular accumulation of cAMP. Coupling of adenosine receptors to other second messenger systems can also occur, such as, stimulation of phospholipase C (A1-, A2B-, and A3-AdoR's), activation of potassium and inhibition of calcium channels in cardiac muscles and neurons (A1-AdoR), mobilization of intracellular calcium (A3-AdoR), and coupling to mitogen activated protein kinase (all four receptors).

### SUMMARY OF THE INVENTION

In one aspect, described herein is a compound of Formula (X), or a pharmaceutically acceptable salt or solvate thereof:

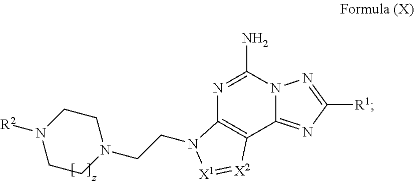

wherein:
X¹═X² is —C(R³)═N—, —N═C(R⁴)—, —C(R⁵)═C(R⁶)—, or —N═N—;
R¹ is

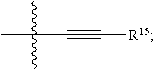

R¹ is a 6-membered heteroaryl ring optionally substituted with m R^{7a} groups;
   m is 0, 1, 2, 3, or 4;
R² is phenyl or a monocyclic or bicyclic heteroaryl ring, wherein the phenyl or monocyclic or bicyclic heteroaryl ring is optionally substituted with n R^{7b};
   n is 0, 1, 2, 3, 4, or 5;
R³ is H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, or —C(═O)N(R⁹)S(═O)₂R¹⁰;
R⁴ is H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, or —C(═O)N(R⁹)S(═O)₂R¹⁰;
R⁵ and R⁶ are each independently selected from H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, and —C(═O)N(R⁹)S(═O)₂R¹⁰;
   wherein at least one of R⁵ and R⁶ is not hydrogen;
each R^{7a} is independently selected from hydrogen, halogen, —CN, C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁹, —SR⁹, —N(R⁹)₂, —C(O)OR⁹, —C(O)N(R⁹)₂, —OC(O)N(R⁹)₂, —N(R¹¹)C(O)N(R⁹)₂, —N(R¹¹)C(O)OR¹⁰, —N(R¹¹)C(O)R¹⁰, —N(R¹¹)S(O)₂R¹⁰, —C(O)R¹⁰, —S(O)R¹⁰, —S(O)₂R¹⁰, —S(O)₂N(R⁹)₂, and —OC(O)R¹⁰, wherein C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R⁸;
each R^{7b} is independently selected from hydrogen, halogen, —CN, C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁹, —SR⁹, —N(R⁹)₂, —C(O)OR⁹, —C(O)N(R⁹)₂, —OC(O)N(R⁹)₂, —N(R¹¹)C(O)N(R⁹)₂, —N(R¹¹)C(O)OR¹⁰, —N(R¹¹)C(O)R¹⁰, —N(R¹¹)S(O)₂R¹⁰, —C(O)R¹⁰, —S(O)R¹⁰, —S(O)₂R¹⁰, —S(O)₂N(R⁹)₂, and —OC(O)R¹⁰, wherein C₁₋₆alkyl, C₁₋₆alkoxy, C₂₋₆alkenyl, C₂₋₆alkynyl, C₃₋₆cycloalkyl, C₂₋₉ heterocycloalkyl, C₆₋₁₀aryl, and C₁₋₉heteroaryl are optionally substituted with one, two, or three R⁸;
or two R^{7b} on adjacent atoms of R² are joined together with the intervening atoms connecting the adjacent R^{7b} groups to form a phenyl, a 5-membered heteroaryl or a 6-membered heteroaryl, wherein the phenyl, the 5-membered heteroaryl or the 6-membered heteroaryl are optionally substituted with one, two, or three R⁸;
each R⁸ is independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR¹², —SR¹², —N(R¹²)₂, —C(O)OR¹², —C(O)N(R¹²)₂, —C(O)C(O)N(R¹²)₂, —OC(O)N(R¹²)₂, —N(R¹⁴)C(O)N(R¹²)₂, —N(R¹⁴)C(O)OR¹³, —N(R¹⁴)C(O)R¹³, —N(R¹⁴)S(O)₂R¹³, —C(O)R¹³, —S(O)₂R¹³, —S(O)₂N(R¹²)₂, and —OC(O)R¹³, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹², —SR¹², —N(R¹²)₂, —C(O)OR¹², —C(O)N(R¹²)₂, —C(O)C(O)N(R¹²)₂, —OC(O)N(R¹²)₂, —N(R¹⁴)C(O)N(R¹²)₂, —N(R¹⁴)C(O)OR¹³, —N(R¹⁴)C(O)R¹³, —N(R¹⁴)S(O)₂R¹³, —C(O)R¹³, —S(O)₂R¹³, —S(O)₂N(R¹²)₂, and —OC(O)R¹³;
each R⁹ is independently selected from H, C₁₋₆alkyl, and C₃₋₆cycloalkyl;
or two R⁹ attached to the same N atom are taken together with the N atom to which they are attached to form an optionally substituted C₂₋₆heterocycloalkyl
each R¹⁰ is independently selected from H, C₁₋₆alkyl and C₃₋₆cycloalkyl;
each R¹¹ is independently selected from H and C₁₋₆alkyl;
each R¹² is independently selected from H, C₁₋₆alkyl, and C₃₋₆cycloalkyl;
each R¹³ is independently selected from H, C₁₋₆alkyl and C₃₋₆cycloalkyl;
each R¹⁴ is independently selected from H and C₁₋₆alkyl;
R¹⁵ is H, C₁-C₆alkyl, or C₃₋₆cycloalkyl; and
z is 1 or 2.

In another aspect, described herein is a compound of Formula (I), or a pharmaceutically acceptable salt or solvate thereof:

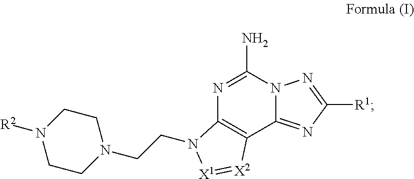

wherein:
X¹═X² is —C(R³)═N—, —N═C(R⁴)—, —C(R⁵)═C(R⁶)—, or —N═N—;
R¹ is

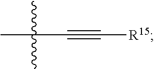

or
R¹ is a 6-membered heteroaryl ring optionally substituted with m R^{7a} groups;
   m is 0, 1, 2, 3, or 4;
R² is phenyl or a monocyclic or bicyclic heteroaryl ring, wherein the phenyl or monocyclic or bicyclic heteroaryl ring is optionally substituted with n R^{7b};
   n is 0, 1, 2, 3, 4, or 5;
R³ is H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, or —C(═O)N(R⁹)S(═O)₂R¹⁰;
R⁴ is H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, or —C(═O)N(R⁹)S(═O)₂R¹⁰;
R⁵ and R⁶ are each independently selected from H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, and —C(═O)N(R⁹)S(═O)₂R¹⁰;
   wherein at least one of R⁵ and R⁶ is not hydrogen;
each R^{7a} is independently selected from hydrogen, halogen, —CN, C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁹, —SR⁹, —N(R⁹)₂, —C(O)OR⁹, —C(O)N(R⁹)₂, —OC(O)N(R⁹)₂, —N(R¹¹)C(O)N(R⁹)₂, —N(R¹¹)C(O)OR¹⁰, —N(R¹¹)C(O)R¹⁰, —N(R¹¹)S(O)₂R¹⁰, —C(O)R¹⁰, —S(O)R¹⁰, —S(O)₂R¹⁰, —S(O)₂N(R⁹)₂, and —OC(O)R¹⁰, wherein C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R⁸;
each R^{7b} is independently selected from hydrogen, halogen, —CN, C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁹, —SR⁹, —N(R⁹)₂, —C(O)OR⁹, —C(O)N(R⁹)₂, —OC(O)N(R⁹)₂, —N(R¹¹)C(O)N(R⁹)₂, —N(R¹¹)C(O)OR¹⁰, —N(R¹¹)C(O)R¹⁰, —N(R¹¹)S(O)₂R¹⁰, —C(O)R¹⁰, —S(O)R¹⁰, —S(O)₂R¹⁰, —S(O)₂N(R⁹)₂ and —OC(O)R¹⁰, wherein C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R⁸;
or two R^{7b} on adjacent atoms of R² are joined together with the intervening atoms connecting the adjacent R^{7b} groups to form a phenyl, a 5-membered heteroaryl or a 6-membered heteroaryl, wherein the phenyl, the 5-membered heteroaryl or the 6-membered heteroaryl are optionally substituted with one, two, or three R⁸;
each R⁸ is independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR¹², —SR¹², —N(R¹²)₂, —C(O)OR¹², —C(O)N(R¹²)₂, —C(O)C(O)N(R¹²)₂, —OC(O)N(R¹²)₂, —N(R¹⁴)C(O)N(R¹²)₂, —N(R¹⁴)C(O)OR¹³, —N(R¹⁴)C(O)R¹³, —N(R¹⁴)S(O)₂R¹³, —C(O)R¹³, —S(O)₂R¹³, —S(O)₂N(R¹²)₂, and —OC(O)R¹³, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹², —SR¹², —N(R¹²)₂, —C(O)OR¹², —C(O)N(R¹²)₂, —C(O)C(O)N(R¹²)₂, —OC(O)N(R¹²)₂, —N(R¹⁴)C(O)N(R¹²)₂, —N(R¹⁴)C(O)OR¹³, —N(R¹⁴)C(O)R¹³, —N(R¹⁴)S(O)₂R¹³—C(O)R¹³, —S(O)₂R¹³, —S(O)₂N(R¹²)₂, and —OC(O)R¹³;
each R⁹ is independently selected from H, C₁₋₆alkyl, and C₃₋₆cycloalkyl;
or two R⁹ attached to the same N atom are taken together with the N atom to which they are attached to form an optionally substituted C₂₋₆heterocycloalkyl;
each R¹⁰ is independently selected from H, C₁₋₆alkyl and C₃₋₆cycloalkyl;
each R¹¹ is independently selected from H and C₁₋₆alkyl;
each R¹² is independently selected from H, C₁₋₆alkyl, and C₃₋₆cycloalkyl;
each R¹³ is independently selected from H, C₁₋₆alkyl and C₃₋₆cycloalkyl;
each R¹⁴ is independently selected from H and C₁₋₆alkyl; and
R¹⁵ is H, C₁-C₆alkyl, or C₃₋₆cycloalkyl.

In certain embodiments, provided herein is a compound of Formula (XI), or a pharmaceutically acceptable salt or solvate thereof:

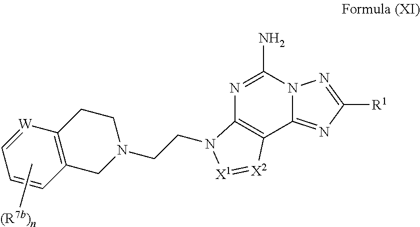

wherein:
X¹═X² is —C(R³)═N—, —N═C(R⁴)—, —C(R⁵)═C(R⁶)—, or —N═N—;
R¹ is a 6-membered heteroaryl ring optionally substituted with m R^{7a} groups; or
R¹ is

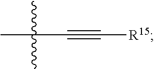

m is 0, 1, 2, 3, or 4;
R³ is H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, or —C(═O)N(R⁹)S(═O)₂R¹⁰;
R⁴ is H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, or —C(═O)N(R⁹)S(═O)₂R¹⁰;
R⁵ and R⁶ are each independently selected from H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, and —C(═O)N(R⁹)S(═O)₂R¹⁰;
   wherein at least one of R⁵ and R⁶ is not hydrogen;
each R^{7a} is independently selected from halogen, —CN, C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁹, —SR⁹, —N(R⁹)₂, —C(O)OR⁹, —C(O)N(R⁹)₂, —OC(O)N(R⁹)₂, —N(R¹¹)C(O)N(R⁹)₂, —N(R¹¹)C(O)OR¹⁰, —N(R¹¹)C(O)R¹⁰, —N(R¹¹)S(O)₂R¹⁰, —C(O)R¹⁰, —S(O)R¹⁰, —S(O)₂R¹⁰, —S(O)₂N(R⁹)₂, and —OC(O)R¹⁰, wherein C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R⁸;
each R^{7b} is independently selected from halogen, —CN, C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁹, —SR⁹, —N(R⁹)₂, —C(O)OR⁹, —C(O)N(R⁹)₂, —OC(O)N(R⁹)₂, —N(R¹¹)C(O)N(R⁹)₂, —N(R¹¹)C(O)OR¹⁰, —N(R¹¹)C(O)R¹⁰, —N(R¹¹)S(O)₂R¹⁰, —C(O)R¹⁰, —S(O)R¹⁰, —S(O)₂R¹⁰, —S(O)₂N(R⁹)₂, and —OC(O)R¹⁰, wherein C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R⁸;
n is 0, 1, 2, or 3;
or two R^{7b} on adjacent atoms of R² are joined together with the intervening atoms connecting the adjacent R^{7b} groups to form a phenyl, a 5-membered heteroaryl or a 6-membered heteroaryl, wherein the phenyl, the 5-membered heteroaryl or the 6-membered heteroaryl are optionally substituted with one, two, or three R⁸;
W is CR^{7c} or N;
R^{7c} is selected from hydrogen, halogen, —CN, C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁹, —SR⁹, —N(R⁹)₂, —C(O)OR⁹, —C(O)N(R⁹)₂, —OC(O)N(R⁹)₂, —N(R¹¹)C(O)N(R⁹)₂, —N(R¹¹)C(O)OR¹⁰, —N(R¹¹)C(O)R¹⁰, —N(R¹¹)S(O)₂R¹⁰, —C(O)R¹⁰, —S(O)R¹⁰, —S(O)₂R¹⁰, —S(O)₂N(R⁹)₂, and —OC(O)R¹⁰, wherein C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R⁸;
each R⁸ is independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR¹², —SR¹², —N(R¹²)₂, —C(O)OR¹², —C(O)N(R¹²)₂, —C(O)C(O)N(R¹²)₂, —OC(O)N(R¹²)₂, —N(R¹⁴)C(O)N(R¹²)₂, —N(R¹⁴)C(O)OR¹³, —N(R¹⁴)C(O)R¹³, —N(R¹⁴)S(O)₂R¹³, —C(O)R¹³, —S(O)₂R¹³, —S(O)₂N(R¹²)₂, and —OC(O)R¹³, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹², —SR¹², —N(R¹²)₂, —C(O)OR¹², —C(O)N(R¹²)₂, —C(O)C(O)N(R¹²)₂, —OC(O)N(R¹²)₂, —N(R¹⁴)C(O)N(R¹²)₂, —N(R¹⁴)C(O)OR¹³, —N(R¹⁴)C(O)R¹³, —N(R¹⁴)S(O)₂R¹³, —C(O)R¹³, —S(O)₂R¹³, —S(O)₂N(R¹²)₂, and —OC(O)R¹³;
each R⁹ is independently selected from H, C₁₋₆alkyl, and C₃₋₆cycloalkyl;
or two R⁹ attached to the same N atom are taken together with the N atom to which they are attached to form an optionally substituted C₂₋₆heterocycloalkyl
each R¹⁰ is independently selected from H, C₁₋₆alkyl and C₃₋₆cycloalkyl;
each R¹¹ is independently selected from H and C₁₋₆alkyl;
each R¹² is independently selected from H, C₁₋₆alkyl, and C₃₋₆cycloalkyl;
each R¹³ is independently selected from H, C₁₋₆alkyl and C₃₋₆cycloalkyl;
each R¹⁴ is independently selected from H and C₁₋₆alkyl; and
R¹⁵ is H, C₁-C₆alkyl, or C₃₋₆cycloalkyl.

In one aspect, provided herein is a pharmaceutical composition comprising a compound disclosed herein, or a pharmaceutically acceptable salt, or solvate thereof, and at least one pharmaceutically acceptable excipient.

In some embodiments, the compounds disclosed herein, or a pharmaceutically acceptable salt thereof, are formulated for administration to a mammal by intravenous administration, subcutaneous administration, oral administration, inhalation, nasal administration, dermal administration, or ophthalmic administration. In some embodiments, the compound disclosed herein, or a pharmaceutically acceptable salt thereof, is in the form of a tablet, a pill, a capsule, a liquid, a suspension, a gel, a dispersion, a solution, an emulsion, an ointment, or a lotion.

In one aspect, described herein is a method of modulating the activity of the A_{2A} adenosine receptor in a mammal comprising administering to the mammal a compound described herein, or any pharmaceutically acceptable salt or solvate thereof.

In another aspect, described herein is a method of modulating the A_{2A} adenosine receptor in a mammal comprising administering to the mammal a compound described herein, or any pharmaceutically acceptable salt or solvate thereof.

In another aspect, described herein is a method of treating a disease or disorder that is mediated by the A_{2A} adenosine receptor in a mammal comprising administering to the mammal in need thereof a therapeutically effective amount of a compound of Formula (I), Formula (X), Formula (XI), or a pharmaceutically acceptable salt or solvate thereof.

In another aspect, described herein is a method for treating cancer in a mammal, the method comprising administering to the mammal a compound of Formula (I), Formula (X), Formula (XI), or a pharmaceutically acceptable salt or solvate thereof.

In some embodiments, the cancer is a solid tumor. In some embodiments, the cancer is bladder cancer, colon cancer, brain cancer, breast cancer, endometrial cancer, heart cancer, kidney cancer, lung cancer, liver cancer, uterine cancer, blood and lymphatic cancer, ovarian cancer, pancreatic cancer, prostate cancer, thyroid cancer, or skin cancer. In some embodiments, the cancer is prostate cancer, breast cancer, colon cancer, or lung cancer. In some embodiments, the cancer is a sarcoma, carcinoma, or lymphoma.

In some embodiments of the methods of treatment described herein are further embodiments that include the co-administration of at least one additional therapy to the mammal in addition to the compound of Formula (I), Formula (X), Formula (XI), or a pharmaceutically acceptable salt or solvate thereof.

In some embodiments, the mammal is a human.

In any of the aforementioned aspects are further embodiments in which an effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal.

In any of the aforementioned aspects are further embodiments comprising single administrations of an effective amount of the compound, including further embodiments in which the compound is administered once a day to the mammal, or the compound is administered to the mammal multiple times over the span of one day. In some embodiments, the compound is administered on a continuous dosing schedule. In some embodiments, the compound is administered on a continuous daily dosing schedule.

Articles of manufacture, which include packaging material, a formulation within the packaging material (e.g. a formulation suitable for topical administration), and a label that indicates that the compound or composition, or pharmaceutically acceptable salt, or solvate thereof, is used for reducing or inhibiting A_{2A} adenosine receptor activity, or for the treatment, prevention or amelioration of one or more symptoms of a disease or disorder that is associated with A_{2A} adenosine receptor activity, are provided.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

The tumor microenvironment (TME) includes a host of cells (mesenchymal, immune, vascular), cytokines, and other signaling molecules that serve to abrogate the innate and adaptive immune responses against the tumor. This is appropriate to maintain tissue homeostasis, and to prevent autoimmunity and tumor evasion of the immune system. Cancer cells adapt many of these pathways to terminate anti-tumor immune response and create an immunosuppressive TME that allows the tumor to proliferate, invade and metastasize. The most amount of attention in clinical trials is being directed toward the two immune checkpoints PD-L-1 and CTLA4. Anti-PD(L)1 and anti-CTLA4 strategies, have demonstrated clinical efficacy in different types of cancer and have revolutionized the treatment of cancer. Antibodies against CTLA4 and PD-1/PD-L1 have been approved as anticancer therapies for a variety of malignancies including Metastatic Melanoma, Non-Small Cell Lung Cancer, Renal Cell Carcinoma, Hodgkin's Lymphoma, Head and Neck Cancer and Urothelial Carcinoma.

Adenosine is another immune checkpoint molecule that is present in the TME that modulates the anti-tumor immune response. It is generated in response to hypoxia-induced ATP release into the extracellular space. ATP is then converted to AMP by the ectonucleotidase CD39 and dephosphorylation of AMP by a second ectonucleotidase CD73 leads to adenosine. Among the four known subtypes of adenosine receptors (A₁, A_{2A}, A₃, A_{2B}), A_{2A} adenosine receptor (A_{2A}AdoR) is the predominantly expressed subtype in most immune cells. Chronic high levels of adenosine in the TME transmits immunosuppressive signals through activation of A_{2A}AdoR on various immune cells. (Allard et ah, Curr. Opin. Pharmacol., 2016, 29, 7-16; Otta A., Frontiers in Immunology, 2016, 7: 109). The A_{2A}AdoR adenosine receptors are cell surface receptors found to be upregulated in various tumor cells. A_{2A}AdoR stimulation in effector T cells (Teff) blocks T cell receptor signaling and impairs effector functions including IFN-γ production and cytotoxicity. In antigen-presenting cells (APC), signal through A_{2A}AdoR reduces Th1-type cytokine milieu and induce tolerogenic APC. Interaction of Teff with these APC will impair activation of cellular immune response against cancer cells. A_{2A}AdoR stimulation enhances immunoregulatory activity of regulatory T cells (Treg). The qualitative and quantitative increase of Treg results in stronger inactivation of antitumor immune response. In addition, adenosine can promote proliferation, survival and metastatic activity of cancer cells.

It has been shown that A_{2A}AdoR-deficient mice spontaneously regress the inoculated tumor, while wild-type mice showed no tumor regression.

Moreover, adenosine receptor A_{2A}AdoR blockade has been shown to increase the efficacy of anti-PD-1 through enhanced anti-tumor T cell responses (P. Beavis, et al., Cancer Immunol Res DOT 10.1158/2326-6066. CIR-14-0211 Published 11 Feb. 2015).

Moreover, adenosine signaling through the A_{2A}AdoR receptor has been found to be a promising negative feedback loop, and preclinical studies have confirmed that blockade of A_{2A}AdoR activation can markedly enhance anti-tumor immunity (Sitkovsky, M V, et al. (2014) Cancer Immun Res 2:598-605).

Thus, adenosine-induced immunosuppressive tumor microenvironment allows the tumor to escape the immune system and promotes tumor metastasis and progression. This immunosuppressive effect is functionally mediated by increased cyclic adenosine 5′-monophosphate (AMP) levels and phosphorylation of cyclic AMP response element binding protein (CREB).

Therefore, inhibition of hypoxia-induced adenosine A_{2A}AdoR activation represents a new class of promising oncology therapeutics.

### Cancer

In some embodiments, disclosed herein are methods of treating cancer with a compound of Formula (I), Formula (X), Formula (XI), or a pharmaceutically acceptable salt or solvate thereof.

The term “cancer” as used herein, refers to an abnormal growth of cells that tend to proliferate in an uncontrolled way and, in some cases, to metastasize (spread). Types of cancer include, but are not limited to, solid tumors (such as those of the bladder, bowel, brain, breast, endometrium, heart, kidney, lung, liver, uterus, lymphatic tissue (lymphoma), ovary, pancreas or other endocrine organ (thyroid), prostate, skin (melanoma or basal cell cancer) or hematological tumors (such as the leukemias and lymphomas) at any stage of the disease with or without metastases.

In some embodiments, a mammal treated with a compound described herein has a disease or disorder that is or is associated with a cancer or tumor. Thus, in some embodiments, the mammal is a human that is an oncology patient. Such diseases and disorders and cancers include carcinomas, sarcomas, benign tumors, primary tumors, tumor metastases, solid tumors, non-solid tumors, blood tumors, leukemias and lymphomas, and primary and metastatic tumors.

In some embodiments, the adenosine A2A receptor antagonists described herein are used in the treatment of solid tumors. A solid tumor is a n abnormal mass of tissue that usually does not contain cysts or liquid areas. Solid tumors may be benign (not cancer), or malignant (cancer). Different types of solid tumors are named for the type of cells that form them. Examples of solid tumors are carcinomas, sarcomas, and lymphomas.

Carcinomas include, but are not limited to, esophageal carcinoma, hepatocellular carcinoma, basal cell carcinoma, squamous cell carcinoma, bladder carcinoma, bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, renal cell carcinoma, Wilm's tumor, cervical carcinoma, uterine carcinoma, testicular carcinoma, osteogenic carcinoma, epithelial carcinoma, and nasopharyngeal carcinoma.

Sarcomas include, but are not limited to, fibrosarcoma, myosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Leukemias include, but are not limited to, a) chronic myeloproliferative syndromes (neoplastic disorders of multipotential hematopoietic stem cells); b) acute myelogenous leukemias; c) chronic lymphocytic leukemias (CLL), including B-cell CLL, T-cell CLL prolymphocyte leukemia, and hairy cell leukemia; and d) acute lymphoblastic leukemias (characterized by accumulation of lymphoblasts). Lymphomas include, but are not limited to, B-cell lymphomas (e.g., Burkitt's lymphoma); Hodgkin's lymphoma; and the like.

Benign tumors include, e.g., hemangiomas, hepatocellular adenoma, cavernous hemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

Primary and metastatic tumors include, e.g., lung cancer; breast cancer; colorectal cancer; anal cancer; pancreatic cancer; prostate cancer; ovarian carcinoma; liver and bile duct carcinoma; esophageal carcinoma; bladder carcinoma; carcinoma of the uterus; glioma, glioblastoma, medulloblastoma, and other tumors of the brain; kidney cancers; cancer of the head and neck; cancer of the stomach; multiple myeloma; testicular cancer; germ cell tumor; neuroendocrine tumor; cervical cancer; carcinoids of the gastrointestinal tract, breast, and other organs.

In one aspect, a compound of Formula (I), Formula (X), Formula (XI), or a pharmaceutically acceptable salt or solvate thereof, reduces, ameliorates or inhibits cell proliferation associated with cancers.

### Compounds

Compounds described herein, including pharmaceutically acceptable salts, prodrugs, active metabolites and solvates thereof, are adenosine A2A receptor modulators. In some embodiments, the adenosine A2A receptor modulators are adenosine A2A receptor antagonists.

In one aspect, described herein is a compound of Formula (X), or a pharmaceutically acceptable salt or solvate thereof:

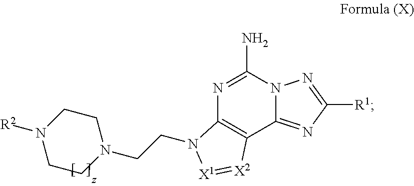

wherein:
X¹═X² is —C(R³)═N—, —N═C(R⁴)—, —C(R⁵)═C(R⁶)—, or —N═N—;
R¹ is

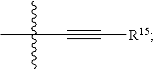

or
R¹ is a 6-membered heteroaryl ring optionally substituted with m R^{7a} groups;
m is 0, 1, 2, 3, or 4;
R² is phenyl or a monocyclic or bicyclic heteroaryl ring, wherein the phenyl or monocyclic or bicyclic heteroaryl ring is optionally substituted with n R^{7b};
   n is 0, 1, 2, 3, 4, or 5;
R³ is H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, or —C(═O)N(R⁹)S(═O)₂R¹⁰;
R⁴ is H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, or —C(═O)N(R⁹)S(═O)₂R¹⁰;
R⁵ and R⁶ are each independently selected from H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, and —C(═O)N(R⁹)S(═O)₂R¹⁰; wherein at least one of R⁵ and R⁶ is not hydrogen;
each R^{7a} is independently selected from hydrogen, halogen, —CN, C₁₋₆alkyl, C₁₋₆alkoxy, C₂-6alkenyl, C₂₋₆alkynyl, C₃₋₆cycloalkyl, C₂₋₉heterocycloalkyl, C₆₋₁₀aryl, C₁₋₉heteroaryl, —OR⁹, —SR⁹, —N(R⁹)₂, —C(O)OR⁹, —C(O)N(R⁹)₂, —OC(O)N(R⁹)₂, —N(R¹¹)C(O)N(R⁹)₂, —N(R¹¹)C(O)OR¹⁰, —N(R¹¹)C(O)R¹⁰, —N(R¹¹)S(O)₂R¹⁰, —C(O)R¹⁰, —S(O)R¹⁰, —S(O)₂R¹⁰, —S(O)₂N(R⁹)₂, and —OC(O)R¹⁰, wherein C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R⁸;
each R^{7b} is independently selected from hydrogen, halogen, —CN, C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁹, —SR⁹, —N(R⁹)₂, —C(O)OR⁹, —C(O)N(R⁹)₂, —OC(O)N(R⁹)₂, —N(R¹¹)C(O)N(R⁹)₂, —N(R¹¹)C(O)OR¹⁰, —N(R¹¹)C(O)R¹⁰, —N(R¹¹)S(O)₂R¹⁰, —C(O)R¹⁰, —S(O)R¹⁰, —S(O)₂R¹⁰, —S(O)₂N(R⁹)₂, and —OC(O)R¹⁰, wherein C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R⁸;
or two R^{7b} on adjacent atoms of R² are joined together with the intervening atoms connecting the adjacent R^{7b} groups to form a phenyl, a 5-membered heteroaryl or a 6-membered heteroaryl, wherein the phenyl, the 5-membered heteroaryl or the 6-membered heteroaryl are optionally substituted with one, two, or three R⁸;
each R⁸ is independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR¹², —SR¹², —N(R¹²)₂, —C(O)OR¹², —C(O)N(R¹²)₂, —C(O)C(O)N(R¹²)₂, —OC(O)N(R¹²)₂, —N(R¹⁴)C(O)N(R¹²)₂,—N(R¹⁴)C(O)OR¹³, —N(R¹⁴)C(O)R¹³, —N(R¹⁴)S(O)₂R¹³, —C(O)R¹³, —S(O)₂R¹³, —S(O)₂N(R¹²)₂, and —OC(O)R¹³, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹², —SR¹², —N(R¹²)₂, —C(O)OR¹², —C(O)N(R¹²)₂, —C(O)C(O)N(R¹²)₂, —OC(O)N(R¹²)₂, —N(R¹⁴)C(O)N(R¹²)₂, —N(R¹⁴)C(O)OR¹³, —N(R¹⁴)C(O)R¹³, —N(R¹⁴)S(O)₂R¹³, —C(O)R¹³, —S(O)₂R¹³, —S(O)₂N(R¹²)₂, and —OC(O)R¹³;
each R⁹ is independently selected from H, C₁₋₆alkyl, and C₃₋₆cycloalkyl;
or two R⁹ attached to the same N atom are taken together with the N atom to which they are attached to form an optionally substituted C₂₋₆heterocycloalkyl
each R¹⁰ is independently selected from H, C₁₋₆alkyl and C₃₋₆cycloalkyl;
each R¹¹ is independently selected from H and C₁₋₆alkyl;
each R¹² is independently selected from H, C₁₋₆alkyl, and C₃₋₆cycloalkyl;
each R¹³ is independently selected from H, C₁₋₆alkyl and C₃₋₆cycloalkyl;
each R¹⁴ is independently selected from H and C₁₋₆alkyl;
R¹⁵ is H, C₁-C₆alkyl, or C₃₋₆cycloalkyl; and
z is 1 or 2.

In some embodiments, the compound of Formula (X) has the structure of Formula (I), or a pharmaceutically acceptable salt or solvate thereof:

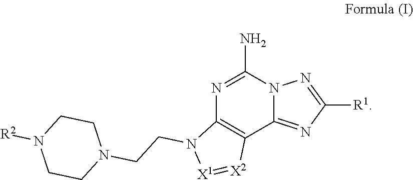

In another aspect, described herein is a compound of Formula (I), or a pharmaceutically acceptable salt or solvate thereof:

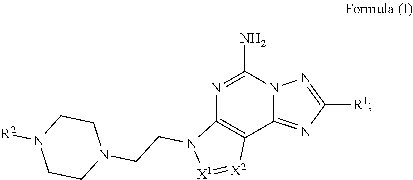

wherein:
X¹═X² is —C(R³)═N—, —N═C(R⁴)—, —C(R⁵)═C(R⁶)—, or —N═N—;
R¹ is a 6-membered heteroaryl ring optionally substituted with m R^{7a} groups; or
R¹ is

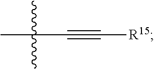

m is 0, 1, 2, 3, or 4;
R² is phenyl or a monocyclic or bicyclic heteroaryl ring, wherein the phenyl or monocyclic or bicyclic heteroaryl ring is optionally substituted with n R^{7b};
   n is 0, 1, 2, 3, 4, or 5;
R³ is H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, or —C(═O)N(R⁹)S(═O)₂R¹⁰;
R⁴ is H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, or —C(═O)N(R⁹)S(═O)₂R¹⁰;
R⁵ and R⁶ are each independently selected from H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, and —C(═O)N(R⁹)S(═O)₂R¹⁰; wherein at least one of R⁵ and R⁶ is not hydrogen;
each R^{7a} is independently selected from halogen, —CN, C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁹, —SR⁹, —N(R⁹)₂, —C(O)OR⁹, —C(O)N(R⁹)₂, —OC(O)N(R⁹)₂, —N(R¹¹)C(O)N(R⁹)₂, —N(R¹¹)C(O)OR¹⁰, —N(R¹¹)C(O)R¹⁰, —N(R¹¹)S(O)₂R¹⁰, —C(O)R¹⁰, —S(O)R¹⁰, —S(O)₂R¹⁰, —S(O)₂N(R⁹)₂, and —OC(O)R¹⁰, wherein C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R⁸;
each R^{7b} is independently selected from halogen, —CN, C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁹, —SR⁹, —N(R⁹)₂, —C(O)OR⁹, —C(O)N(R⁹)₂, —OC(O)N(R⁹)₂, —N(R¹¹)C(O)N(R⁹)₂, —N(R¹¹)C(O)OR¹⁰, —N(R¹¹)C(O)R¹⁰, —N(R¹¹)S(O)₂R¹⁰, —C(O)R¹⁰, —S(O)R¹⁰, —S(O)₂R¹⁰, —S(O)₂N(R⁹)₂, and —OC(O)R¹⁰, wherein C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R⁸;
or two R^{7b} on adjacent atoms of R² are joined together with the intervening atoms connecting the adjacent R^{7b} groups to form a phenyl, a 5-membered heteroaryl or a 6-membered heteroaryl, wherein the phenyl, the 5-membered heteroaryl or the 6-membered heteroaryl are optionally substituted with one, two, or three R⁸;
each R⁸ is independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR¹², —SR¹², —N(R¹²)₂, —C(O)OR¹², —C(O)N(R¹²)₂, —C(O)C(O)N(R¹²)₂, —OC(O)N(R¹²)₂, —N(R¹⁴)C(O)N(R¹²)₂, —N(R¹⁴)C(O)OR¹³, —N(R¹⁴)C(O)R¹³, —N(R¹⁴)S(O)₂R¹³, —C(O)R¹³, —S(O)₂R¹³, —S(O)₂N(R¹²)₂, and —OC(O)R¹³, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹², —SR¹², —N(R¹²)₂, —C(O)OR¹², —C(O)N(R¹²)₂, —C(O)C(O)N(R¹²)₂, —OC(O)N(R¹²)₂, —N(R¹⁴)C(O)N(R¹²)₂, —N(R¹⁴)C(O)OR¹³, —N(R¹⁴)C(O)R¹³, —N(R¹⁴)S(O)₂R¹³, —C(O)R¹³, —S(O)₂R¹³, —S(O)₂N(R¹²)₂, and —OC(O)R¹³;
each R⁹ is independently selected from H, C₁₋₆alkyl, and C₃₋₆cycloalkyl;
or two R⁹ attached to the same N atom are taken together with the N atom to which they are attached to form an optionally substituted C₂₋₆heterocycloalkyl;
each R¹⁰ is independently selected from H, C₁₋₆alkyl and C₃₋₆cycloalkyl;
each R¹¹ is independently selected from H and C₁₋₆alkyl;
each R¹² is independently selected from H, C₁₋₆alkyl, and C₃₋₆cycloalkyl;
each R¹³ is independently selected from H, C₁₋₆alkyl and C₃₋₆cycloalkyl;
each R¹⁴ is independently selected from H and C₁₋₆alkyl; and
R¹⁵ is H, C₁-C₆alkyl, or C₃₋₆cycloalkyl.

In another aspect, described herein is a compound of Formula (I), or a pharmaceutically acceptable salt or solvate thereof:

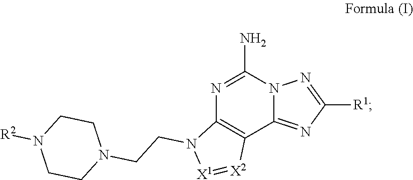

wherein:
X¹═X² is —C(R³)═N—, —N═C(R⁴)—, —C(R⁵)═C(R⁶)—, or —N═N—;
R¹ is a 6-membered or 5-membered heteroaryl ring optionally substituted with m R^{7a}; or
R¹ is

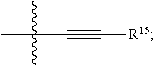

m is 0, 1, 2, 3, or 4;
R² is phenyl or a monocyclic or bicyclic heteroaryl ring, wherein the phenyl or monocyclic or bicyclic heteroaryl ring is optionally substituted with n R^{7b};
   n is 0, 1, 2, 3, 4, or 5;
R³ is H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, or —C(═O)N(R⁹)S(═O)₂R¹⁰;
R⁴ is halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, or —C(═O)N(R⁹)S(═O)₂R¹⁰;
R⁵ and R⁶ are independently selected from H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, and —C(═O)N(R⁹)S(═O)₂R¹⁰; wherein at least one of R⁵ and R⁶ is not hydrogen;
each R^{7a} is independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁹, —SR⁹, —N(R⁹)₂, —C(O)OR⁹, —C(O)N(R⁹)₂, —OC(O)N(R⁹)₂, —N(R¹¹)C(O)N(R⁹)₂, —N(R¹¹)C(O)OR¹⁰, —N(R¹¹)C(O)R¹⁰, —N(R¹¹)S(O)₂R¹⁰, —C(O)R¹⁰, —S(O)R¹⁰, —S(O)₂R¹⁰, —S(O)₂N(R⁹)₂, and —OC(O)R¹⁰, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R⁸;
each R^{7b} is independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁹, —SR⁹, —N(R⁹)₂, —C(O)OR⁹, —C(O)N(R⁹)₂, —OC(O)N(R⁹)₂, —N(R¹¹)C(O)N(R⁹)₂, —N(R¹¹)C(O)OR¹⁰, —N(R¹¹)C(O)R¹⁰, —N(R¹¹)S(O)₂R¹⁰, —C(O)R¹⁰, —S(O)R¹⁰, —S(O)₂R¹⁰, —S(O)₂N(R⁹)₂, and —OC(O)R¹⁰, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R⁸;
or two R^{7b} on adjacent atoms of R² are joined together with the intervening atoms connecting the adjacent R^{7b} groups to form a phenyl, a 5-membered heteroaryl or a 6-membered heteroaryl;
each R⁸ is independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR¹², —SR¹², —N(R¹²)₂, —C(O)OR¹², —C(O)N(R¹²)₂, —C(O)C(O)N(R¹²)₂, —OC(O)N(R¹²)₂, —N(R¹⁴)C(O)N(R¹²)₂, —N(R¹⁴)C(O)OR¹³, —N(R¹⁴)C(O)R¹³, —N(R¹⁴)S(O)₂R¹³, —C(O)R¹³, —S(O)₂R¹³, —S(O)₂N(R¹²)₂, and —OC(O)R¹³, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀ aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹², —SR¹², —N(R¹²)₂, —C(O)OR¹², —C(O)N(R¹²)₂, —C(O)C(O)N(R¹²)₂, —OC(O)N(R¹²)₂, —N(R¹⁴)C(O)N(R¹²)₂, —N(R¹⁴)C(O)OR¹³, —N(R¹⁴)C(O)R¹³, —N(R¹⁴)S(O)₂R¹³, —C(O)R¹³, —S(O)₂R¹³, —S(O)₂N(R¹²)₂, and —OC(O)R¹³;
each R⁹ is independently selected from H, C₁₋₆alkyl, and C₃₋₆cycloalkyl;
each R¹⁰ is independently selected from C₁₋₆alkyl and C₃₋₆cycloalkyl;
each R¹¹ is independently selected from H and C₁₋₆alkyl;
each R¹² is independently selected from H, C₁₋₆alkyl, and C₃₋₆cycloalkyl;
each R¹³ is independently selected from C₁₋₆alkyl and C₃₋₆cycloalkyl;
each R¹⁴ is independently selected from H and C₁₋₆alkyl; and
R¹⁵ is H or C₁-C₆alkyl.

For any and all of the embodiments, substituents are selected from among a subset of the listed alternatives. For example, in some embodiments, R¹ is a 6-membered or a 5-membered heteroaryl ring optionally substituted with m R^{7a}; or R¹ is

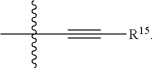

In some embodiments, R¹ is a 6-membered heteroaryl ring optionally substituted with m R^{7a}; or R¹ is

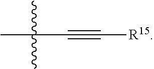

In some embodiments, R¹ is a 5-membered heteroaryl ring optionally substituted with m R^{7a}; or R¹ is

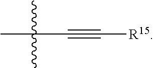

In some embodiments, R¹ is a 6-membered or 5-membered heteroaryl ring optionally substituted with m R^{7a}. In some embodiments, R¹ is a 5-membered heteroaryl ring optionally substituted with m R^{7a}. In some embodiments, R¹ is a 6-membered heteroaryl ring optionally substituted with m R^{7a}.

In some embodiments, R¹ is a 6-membered heteroaryl ring optionally substituted with m R^{7a}.

In some embodiments, R¹ is a pyridinyl optionally substituted with m R^{7a}, pyrimidinyl optionally substituted with m R^{7a}, pyrazinyl optionally substituted with m R^{7a}, pyridazinyl optionally substituted with m R^{7a}, or triazinyl optionally substituted with m R^{7a}.

In some embodiments, R¹ is a pyridinyl optionally substituted with m R^{7a}.

In some embodiments, R¹ is

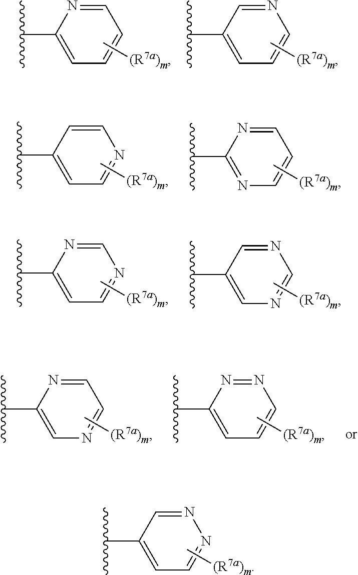

In some embodiments, the compound has the structure of Formula (Ia), or a pharmaceutically acceptable salt or solvate thereof:

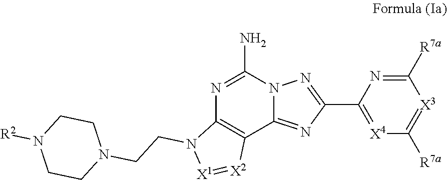

wherein,
X³ is CR^{7a} or N;
X⁴ is CR^{7a} or N.

In some embodiments, m is 0. In some embodiments, m is 1. In some embodiments, m is 2.

In some embodiments, R¹ is

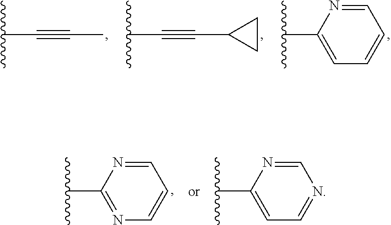

In some embodiments, R¹ is

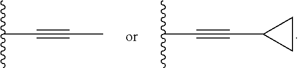

In some embodiments, R¹ is

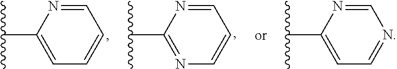

In some embodiments, R¹ is

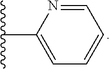 [SMILES: *c1ccccn1]

In some embodiments, the compound has the structure of Formula (Ib), or a pharmaceutically acceptable salt or solvate thereof:

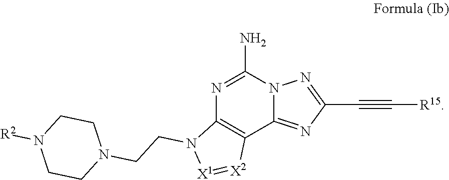

In some embodiments, R¹ is

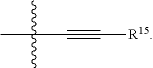

In some embodiments, R¹⁵ is C₁-C₆alkyl or C₃₋₆cycloalkyl. In some embodiments, R¹⁵ is C₁-C₆alkyl. In some embodiments, R¹⁵ is —CH₃. In some embodiments, R¹⁵ is cyclopropyl.

In some embodiments, X¹═X² is —C(R³)═N—.

In some embodiments, the compound has the structure of Formula (IIa), or a pharmaceutically acceptable salt or solvate thereof:

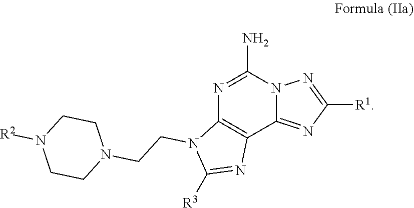

In some embodiments, R is H, halogen, C₁-C₆alkyl, C₃₋₆cycloalkyl, —CN, —C(═O)N(R⁹)₂, or —C(═O)N(R⁹)S(═O)₂R¹⁰.

In some embodiments, R³ is H, C₁-C₆alkyl, C₃₋₆cycloalkyl, —CN, —C(═O)N(R⁹)₂, or —C(═O)N(R⁹)S(═O)₂R¹⁰.

In some embodiments, X¹═X² is —N═C(R⁴)—.

In some embodiments, the compound has the structure of Formula (IIb), or a pharmaceutically acceptable salt or solvate thereof:

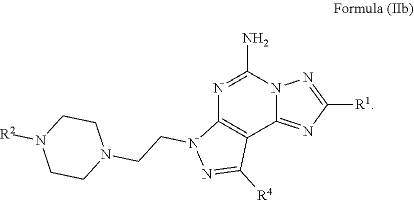

In some embodiments, R⁴ is H, halogen, C₁-C₆alkyl, C₃₋₆cycloalkyl, —CN, —CO₂R⁹, —C(═O)N(R⁹)₂, or —C(═O)N(R⁹)S(═O)₂R¹⁰.

In some embodiments, R⁴ is halogen, C₁-C₆alkyl, C₃₋₆cycloalkyl, —CN, —CO₂R⁹, —C(═O)N(R⁹)₂, or —C(═O)N(R⁹)S(═O)₂R¹⁰.

In some embodiments, R⁴ is halogen, C₁-C₆alkyl, or C₃₋₆cycloalkyl.

In some embodiments, X¹═X² is —C(R⁵)═C(R⁶)—.

In some embodiments, the compound has the structure of Formula (IIc), or a pharmaceutically acceptable salt or solvate thereof:

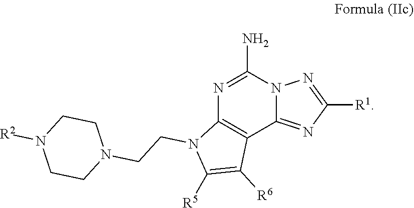

In some embodiments, R⁵ and R⁶ are each independently selected from H, halogen, C₁-C₆alkyl, C₃-6cycloalkyl, —CN, —CO₂R⁹, —C(═O)N(R⁹)₂, and —C(═O)N(R⁹)S(═O)₂R¹⁰.

In some embodiments, R⁵ and R⁶ are independently selected from H, halogen, C₁-C₆alkyl, C₃-6cycloalkyl, —CN, —C(═O)N(R⁹)₂, and —C(═O)N(R⁹)S(═O)₂R¹⁰.

In some embodiments, R⁵ is halogen, C₁-C₆alkyl, C₃₋₆cycloalkyl, —CN, —CO₂R⁹, —C(═O)N(R⁹)₂, or —C(═O)N(R⁹)S(═O)₂R¹⁰; and R⁶ is H.

In some embodiments, R⁵ is halogen, C₁-C₆alkyl, C₃₋₆cycloalkyl, —CN, —C(═O)N(R⁹)₂, or —C(═O)N(R⁹)S(═O)₂R¹⁰; and R⁶ is H.

In some embodiments, R⁵ is —CN, —CO₂H, —CO₂CH₃, or —C(═O)NH₂; and R⁶ is H.

In some embodiments, R⁵ is —CN or —C(═O)NH₂; and R⁶ is H.

In some embodiments, R⁵ is —CN, —CO₂H, —CO₂CH₃, —C(═O)NHCH₃,

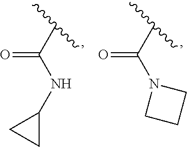 [SMILES: *C(=O)NC1CC1 ; *C(=O)N1CCC1]

or —C(═O)NH₂; and R⁶ is H, Cl, or CH₃.

In some embodiments, R⁵ is —CN, —CO₂H, —CO₂CH₃, or —C(═O)NH₂; and R⁶ is Cl or CH₃.

In some embodiments, R⁹ is H. In some embodiments, R⁹ is C₁₋₆alkyl. In some embodiments, R⁹ is C₃₋₆cycloalkyl. In some embodiments, two R⁹ attached to the same N atom are taken together with the N atom to which they are attached to form an optionally substituted C₂₋₆heterocycloalkyl. In some embodiments, two R⁹ attached to the same N atom are taken together with the N atom to which they are attached to form an optionally substituted 3-membered, optionally substituted 4-membered, optionally substituted 5-membered, or optionally substituted 6-membered C₂₋₆heterocycloalkyl. In some embodiments, two R⁹ attached to the same N atom are taken together with the N atom to which they are attached to form an azetidinyl.

In some embodiments, X¹═X² is —N═N—.

In some embodiments, the compound has the structure of Formula (IIIa), or a pharmaceutically acceptable salt or solvate thereof:

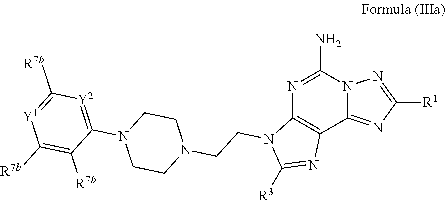

wherein:
Y¹ is CH, CR^{7b} or N;
Y² is CH, CR^{7b} or N.

In some embodiments, the compound has the structure of Formula (IIIb), or a pharmaceutically acceptable salt or solvate thereof:

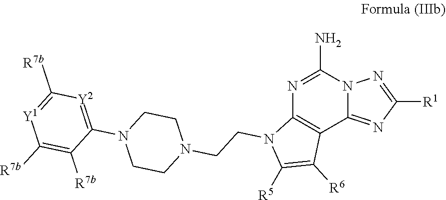

wherein:
Y¹ is CH, CR^{7b} or N;
Y² is CH, CR^{7b} or N.

In some embodiments, the compound has the structure of Formula (IIIc), or a pharmaceutically acceptable salt or solvate thereof:

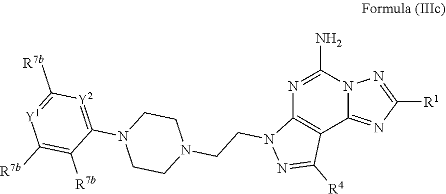

wherein:
Y¹ is CH, CR^{7b} or N;
Y² is CH, CR^{7b} or N.

In some embodiments, n is 0. In some embodiments, n is 1. In some embodiments, n is 2.

In some embodiments, n is 3.

In some embodiments, each R^{7b} is independently selected from halogen, —CN, C₁₋₆alkyl, C₁₋₆alkoxy, C₁₋₉heteroaryl, and —OR⁹, wherein C₁₋₆alkyl, C₁₋₆alkoxy, and C₁₋₉heteroaryl are optionally substituted with one, two, or three R⁸.

In some embodiments, n is one or two; and each R^{7b} is independently selected from C₁₋₆ alkyl, halogen, —CN, and C₁₋₉heteroaryl. In some embodiments, n is one or two; and each R^{7b} is independently selected from halogen and a 5-membered heteroaryl. In some embodiments, n is one or two; and each R^{7b} is independently selected from C₁₋₆alkyl and C₁₋₉heteroaryl. In some embodiments, n is one or two; and each R^{7b} is independently selected from halogen and C₁₋₆alkyl. In some embodiments, n is one or two; and each R^{7b} is independently selected from F and CH₃. In some embodiments, n is one or two; and each R^{7b} is independently selected from halogen and CN. In some embodiments, n is two; and each R^{7b} is independently selected from F and CN. In some embodiments, n is two; and each R^{7b} is halogen. In some embodiments, n is two; and each R^{7b} is F.

In some embodiments, n is one or two; and each R^{7b} is independently selected from halogen and C₁₋₆alkoxy substituted with one, two, or three R⁸. In some embodiments, n is one or two; and each R^{7b} is independently selected from F and C₁₋₆alkoxy substituted with one, two, or three R⁸. In some embodiments, n is one; and R^{7b} C₁₋₆alkoxy substituted with one, two, or three R⁸. In some embodiments, n is one; and R^{7b} is C₁₋₆alkoxy substituted with one, two, or three R⁸, wherein each R⁸ is independently selected from C₁₋₆alkyl, —OR¹², —C(O)OR¹², and —C(O)N(R¹⁴)S(O)₂R¹³. In some embodiments, n is one; and R^{7b} is C₁₋₆alkoxy substituted with one, two, or three R⁸, wherein each R⁸ is independently selected from CH₃, —OCH₃, —C(O)OH, and —C(O)N(H)S(O)₂H. In some embodiments, R^{7b} is C₁₋₆alkoxy substituted with one, two, or three R⁸, wherein each R⁸ is independently selected from CH₃, —OCH₃, —C(O)OH, and —C(O)N(H)S(O)₂H.

In some embodiments, R^{7b} is C₁alkoxy, C₂alkoxy, C₃alkoxy, or C₄alkoxy substituted with one, two, or three R⁸. In some embodiments, R^{7b} is methoxy substituted with one, two, or three R⁸. In some embodiments, R^{7b} is ethoxy substituted with one, two, or three R⁸. In some embodiments, R^{7b} is isopropoxy substituted with one, two, or three R⁸.

In some embodiments, two R^{7b} on adjacent atoms of R² are joined together with the intervening atoms connecting the adjacent R^{7b} groups to a 5-membered heteroaryl optionally substituted with one, two, or three R⁸.

In some embodiments, R⁸ is C₁₋₆alkyl, —OR¹², —C(O)OR¹², or —N(R¹⁴)S(O)₂R¹³, wherein C₁₋₆alkyl is optionally substituted with one, two, or three groups independently selected from oxo, C₁₋₆alkyl, C₁₋₆alkoxy, —OR¹², —C(O)OR¹², and —N(R¹⁴)S(O)₂R¹³. In some embodiments, R⁸ is C₁₋₆ alkyl substituted with oxo and —N(R¹⁴)S(O)₂R¹³. In some embodiments, each R⁸ is independently selected from CH₃, —OCH₃, —C(O)OH, and —C(O)N(H)S(O)₂H.

In some embodiments, R² is phenyl optionally substituted with one, two, or three R⁷.

In some embodiments, R² is

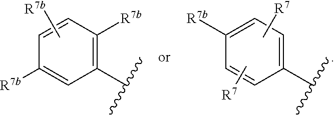

In some embodiments, R² is phenyl substituted with one R^{7b}; R^{7b} is C₁₋₆alkoxy substituted with one, two, or three R⁸; and each R⁸ is independently selected from —CH₃, —OCH₃, —C(O)OH, and —C(O)NHSO₂CH₃.

In some embodiments, R² is

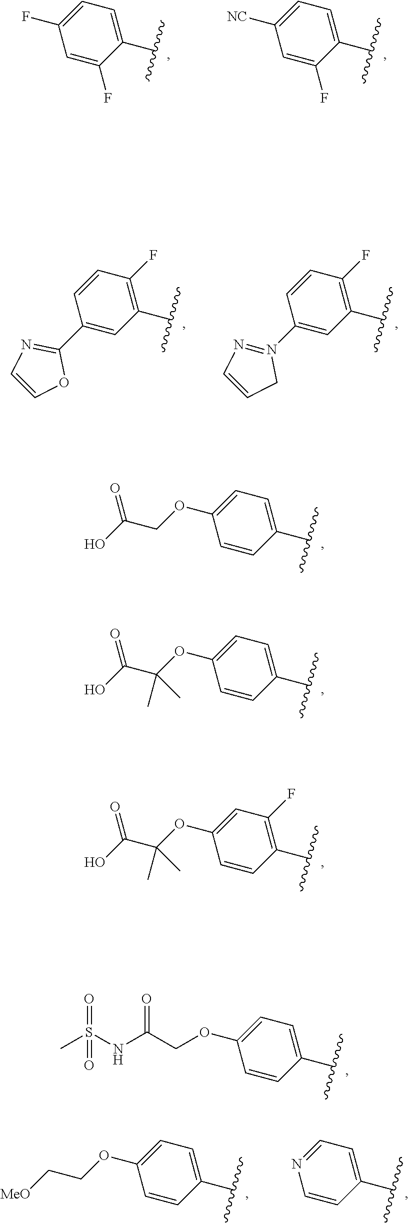

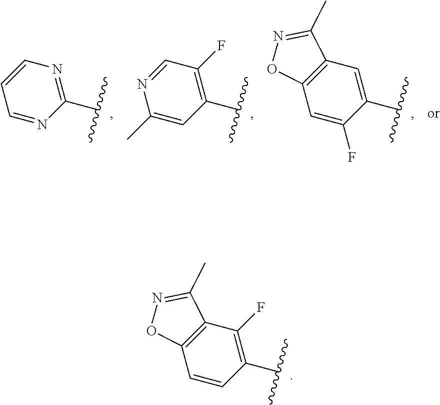

In some embodiments, R² is

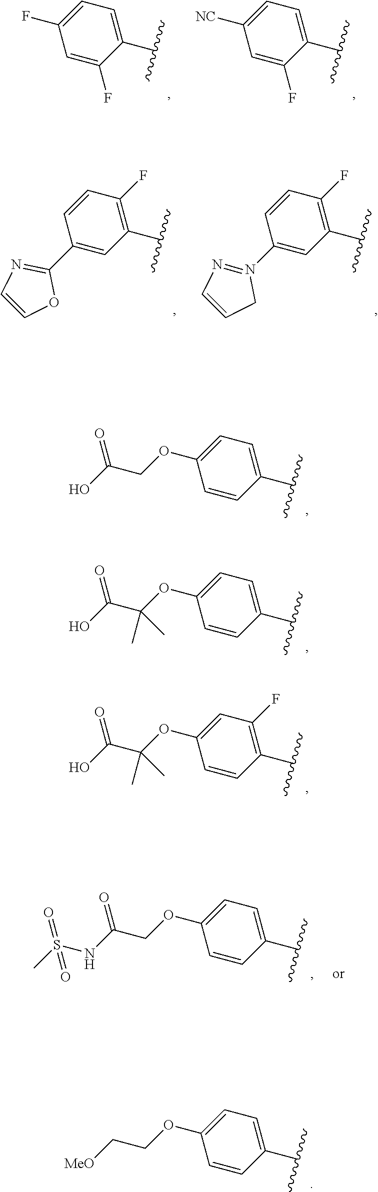

In some embodiments, R² is a monocyclic or bicyclic heteroaryl ring optionally substituted with one, two, or three R^{7b}.

In some embodiments, R² is a monocyclic heteroaryl ring selected from oxazolyl, thiazolyl, pyrazolyl, furanyl, thienyl, pyrrolyl, imidazolyl, triazolyl, tetrazolyl, isoxazolyl, isothiazolyl, oxadiazolyl, thiadiazolyl, pyridinyl, pyrimidinyl, pyrazinyl, and pyridazinyl.

In some embodiments, R² is

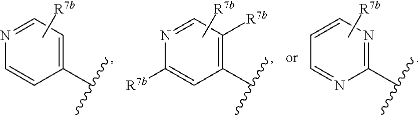

In some embodiments, each R^{7b} is independently selected from halogen and C₁₋₆alkyl.

In some embodiments, R² is

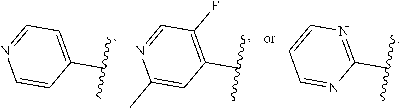

In some embodiments, the compound has the structure of Formula (IVa), or a pharmaceutically acceptable salt or solvate thereof:

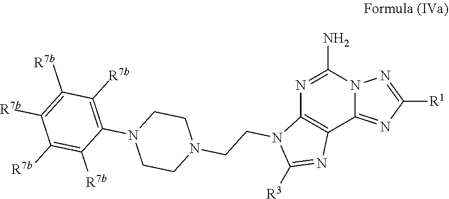

In some embodiments, the compound has the structure Formula (IVb), or a pharmaceutically acceptable salt or solvate thereof:

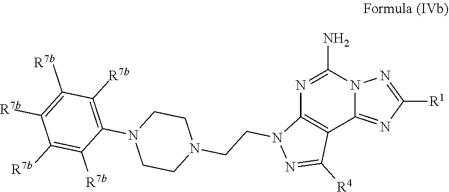

In some embodiments, the compound has the structure of Formula (IVc), or a pharmaceutically acceptable salt or solvate thereof:

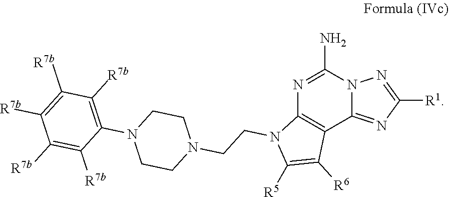

In some embodiments, R² is a bicyclic heteroaryl ring selected from indolyl, benzofuranyl, benzothienyl, benzoxazolyl, benzisoxazolyl, benzimidazolyl, imidazopyrdinyl, imidazopyridazinyl, purinyl, quinolinyl, quinazolinyl, and pyridopyrimidinyl.

In some embodiments, R² is

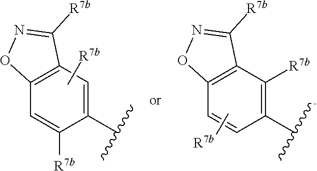

In some embodiments, each R^{7b} is independently selected from halogen and C₁₋₆alkyl.

In some embodiments, R² is

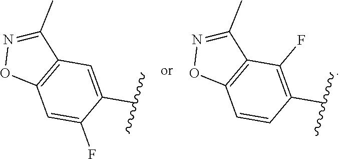 [SMILES: *c1cc2c(C)noc2cc1F ; *c1ccc2onc(C)c2c1F]

In some embodiments, z is 2. In some embodiments, the compound has the structure of Formula (Xa), or a pharmaceutically acceptable salt or solvate thereof:

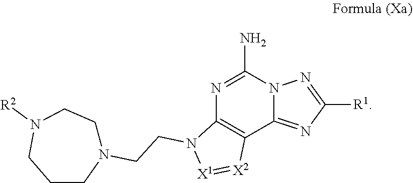

In some embodiments, z is 2; and R¹ is pyridyl.

In some embodiments, the compound has the structure of Formula (Xb), or a pharmaceutically acceptable salt or solvate thereof:

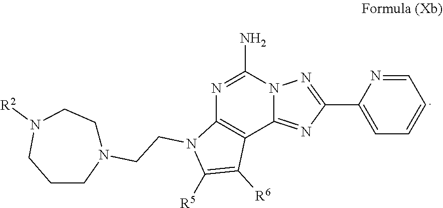

In certain embodiments, provided herein is a compound of Formula (XI), or a pharmaceutically acceptable salt or solvate thereof:

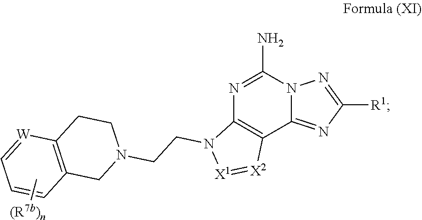

wherein:
X¹═X² is —C(R³)═N—, —N═C(R⁴)—, —C(R⁵)═C(R⁶)—, or —N═N—;
R¹ is a 6-membered heteroaryl ring optionally substituted with m R^{7a} groups; or
R¹ is

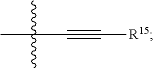

m is 0, 1, 2, 3, or 4;
R³ is H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, or —C(═O)N(R⁹)S(═O)₂R¹⁰;
R⁴ is H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, or —C(═O)N(R⁹)S(═O)₂R¹⁰;
R⁵ and R⁶ are each independently selected from H, halogen, C₁-C₆alkyl, C₁-C₆haloalkyl, C₁-C₆heteroalkyl, C₃-6cycloalkyl, —CN, —CO₂R⁹, —C(═O)N(R⁹)₂, and —C(═O)N(R⁹)S(═O)₂R¹⁰; wherein at least one of R⁵ and R⁶ is not hydrogen;
each R^{7a} is independently selected from halogen, —CN, C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁹, —SR⁹, —N(R⁹)₂, —C(O)OR⁹, —C(O)N(R⁹)₂, —OC(O)N(R⁹)₂, —N(R¹¹)C(O)N(R⁹)₂, —N(R¹¹)C(O)OR¹⁰, —N(R¹¹)C(O)R¹⁰, —N(R¹¹)S(O)₂R¹⁰, —C(O)R¹⁰, —S(O)R¹⁰, —S(O)₂R¹⁰, —S(O)₂N(R⁹)₂, and —OC(O)R¹⁰, wherein C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R⁸;
each R^{7b} is independently selected from halogen, —CN, C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁹, —SR⁹, —N(R⁹)₂, —C(O)OR⁹, —C(O)N(R⁹)₂, —OC(O)N(R⁹)₂, —N(R¹¹)C(O)N(R⁹)₂, —N(R¹¹)C(O)OR¹⁰, —N(R¹¹)C(O)R¹⁰, —N(R¹¹)S(O)₂R¹⁰, —C(O)R¹⁰, —S(O)R¹⁰, —S(O)₂R¹⁰, —S(O)₂N(R⁹)₂, and —OC(O)R¹⁰, wherein C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R⁸;
n is 0, 1, 2, or 3;
or two R^{7b} on adjacent atoms of R² are joined together with the intervening atoms connecting the adjacent R^{7b} groups to form a phenyl, a 5-membered heteroaryl or a 6-membered heteroaryl, wherein the phenyl, the 5-membered heteroaryl or the 6-membered heteroaryl are optionally substituted with one, two, or three R⁸;
W is CR^{7c} or N;
R^{7c} is selected from hydrogen, halogen, —CN, C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁹, —SR⁹, —N(R⁹)₂, —C(O)OR⁹, —C(O)N(R⁹)₂, —OC(O)N(R⁹)₂, —N(R¹¹)C(O)N(R⁹)₂, —N(R¹¹)C(O)OR¹⁰, —N(R¹¹)C(O)R¹⁰, —N(R¹¹)S(O)₂R¹⁰, —C(O)R¹⁰, —S(O)R¹⁰, —S(O)₂R¹⁰, —S(O)₂N(R⁹)₂, and —OC(O)R¹⁰, wherein C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R⁸;
each R⁸ is independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀ aryl, C₁₋₉heteroaryl, —OR¹², —SR¹², —N(R¹²)₂, —C(O)OR¹², —C(O)N(R¹²)₂, —C(O)C(O)N(R¹²)₂, —OC(O)N(R¹²)₂, —N(R¹⁴)C(O)N(R¹²)₂, —N(R¹⁴)C(O)OR¹³, —N(R¹⁴)C(O)R¹³, —N(R¹⁴)S(O)₂R¹³, —C(O)R¹³, —S(O)₂R¹³, —S(O)₂N(R¹²)₂, and —OC(O)R¹³, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀ aryl, and C₁₋₉heteroaryl are optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR¹², —SR¹², —N(R¹²)₂, —C(O)OR¹², —C(O)N(R¹²)₂, —C(O)C(O)N(R¹²)₂, —OC(O)N(R¹²)₂, —N(R¹⁴)C(O)N(R¹²)₂, —N(R¹⁴)C(O)OR¹³, —N(R¹⁴)C(O)R¹³, —N(R¹⁴)S(O)₂R¹³, —C(O)R¹³, —S(O)₂R¹³, —S(O)₂N(R¹²)₂, and —OC(O)R¹³;
each R⁹ is independently selected from H, C₁₋₆alkyl, and C₃₋₆cycloalkyl;
or two R⁹ attached to the same N atom are taken together with the N atom to which they are attached to form an optionally substituted C₂₋₆heterocycloalkyl
each R¹⁰ is independently selected from H, C₁₋₆alkyl and C₃₋₆cycloalkyl;
each R¹¹ is independently selected from H and C₁₋₆alkyl;
each R¹² is independently selected from H, C₁₋₆alkyl, and C₃₋₆cycloalkyl;
each R¹³ is independently selected from H, C₁₋₆alkyl and C₃₋₆cycloalkyl;
each R¹⁴ is independently selected from H and C₁₋₆alkyl; and
R¹⁵ is H, C₁-C₆alkyl, or C₃₋₆cycloalkyl.

In some embodiments, W is N.

In some embodiments, n is 0. In some embodiments, n is 1. In some embodiments, n is 2.

In some embodiments, n is 1; and R^{7b} is C₁₋₆alkyl, C₂₋₉heterocycloalkyl, or C₁₋₉heteroaryl.

In some embodiments, R^{7b} is CH₃. In some embodiments, R^{7b} is a 5-membered heterocycloalkyl.

In some embodiments, R^{7b} is oxetanyl. In some embodiments, R^{7b} is a 5-membered heteroaryl. In some embodiments, R^{7b} is thiazole.

In some embodiments,

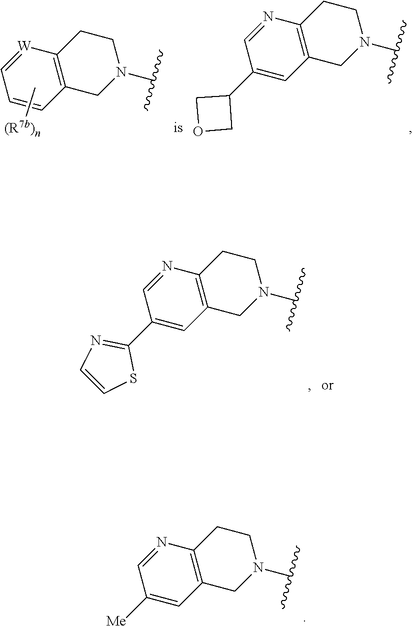

In some embodiments, R¹, R², X¹, and X² are described above.

In some embodiments, the compound has the structure of Formula (Xb), or a pharmaceutically acceptable salt or solvate thereof:

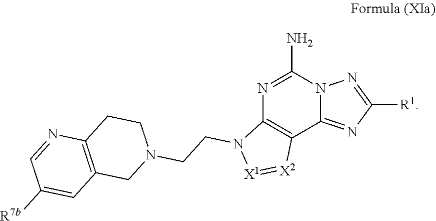

In some embodiments, the compound has the following structure, or a pharmaceutically acceptable salt or solvate thereof:

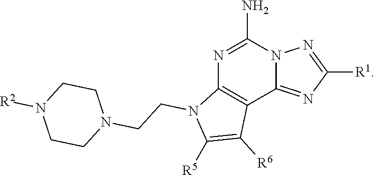

In some embodiments, R¹ is as described in Table 1, Table 2, Table 3 or Table 5. In some embodiments, R² is as described in Table 1, Table 2, Table 3 or Table 4. In some embodiments, R⁵ and R⁶ are as described in Table 1 or Table 4. In some embodiments, R¹ is as described in Table 1, Table 2, Table 3 or Table 5; R² is as described in Table 1, Table 2, Table 3 or Table 4; R⁵ and R⁶ are as described in Table 1 or Table 4. In some embodiments, R¹ and R² are as described in Table 1, Table 2, or Table 3. In some embodiments, R¹, R², R⁵, and R⁶ are as described in Table 1.

In some embodiments, the compound has the following structure, or a pharmaceutically acceptable salt or solvate thereof:

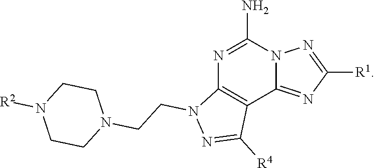

In some embodiments, R¹ is as described in Table 1, Table 2, Table 3 or Table 5. In some embodiments, R² is as described in Table 1, Table 2, Table 3 or Table 4. In some embodiments, R⁴ is as described in Table 2. In some embodiments, R¹ and R² are as described in Table 1, Table 2, or Table 3. In some embodiments, R¹, R², and R⁴ are as described in Table 2.

In some embodiments, the compound has the following structure, or a pharmaceutically acceptable salt or solvate thereof:

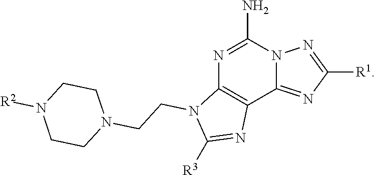

In some embodiments, R¹ is as described in Table 1, Table 2, Table 3 or Table 5. In some embodiments, R² is as described in Table 1, Table 2, Table 3 or Table 4. In some embodiments, R³ is as described in Table 3. In some embodiments, R¹ and R² are as described in Table 1, Table 2, or Table 3. In some embodiments, R¹, R², R³ are as described in Table 3.

In some embodiments, the compound has the following structure, or a pharmaceutically acceptable salt or solvate thereof:

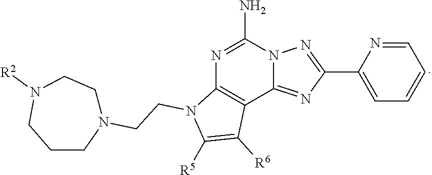

In some embodiments, R² is as described in Table 1, Table 2, Table 3 or Table 4. In some embodiments, R⁵ is as described in Table 1 or Table 4. In some embodiments, R⁶ is as described in Table 1 or Table 4. In some embodiments, R², R⁵, R⁶ are as described in Table 4.

In some embodiments, the compound has the following structure, or a pharmaceutically acceptable salt or solvate thereof:

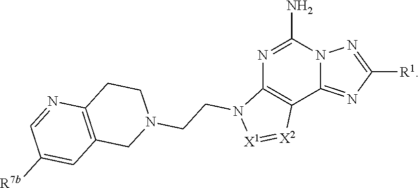

In some embodiments, R is as described in Table 1, Table 2, Table 3 or Table 5. In some embodiments, X¹ is as described in Table 5. In some embodiments, X² is as described in Table 5.

In some embodiments, R¹ is as described in Table 1, Table 2, Table 3 or Table 5; X¹ is as described in Table 5; X² is as described in Table 5; and R^{7b} is as described in Table 5. In some embodiments, X¹, X², R¹, and R^{7b} are described herein. In some embodiments, X¹, X², R¹, and R^{7b}are as described in Table 5.

Any combination of the groups described above for the various variables is contemplated herein. Throughout the specification, groups and substituents thereof are chosen by one skilled in the field to provide stable moieties and compounds.

Representative compounds of Formula (I) include, but are not limited to, to the compounds disclosed in Table 1, 2, 3, 4 and 5.

TABLE 1

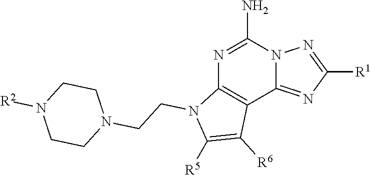

| R² | R⁵ | R⁶ | R¹ |
|---|---|---|---|
| 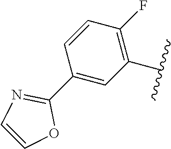 [SMILES: *c1cc(-c2ncco2)ccc1F] | 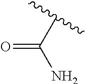 [SMILES: *C(N)=O] | H | 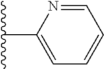 [SMILES: *c1ccccn1] |
| 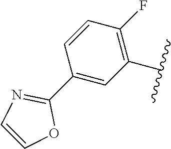 [SMILES: *c1cc(-c2ncco2)ccc1F] | —CN | H | 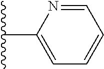 [SMILES: *c1ccccn1] |
| 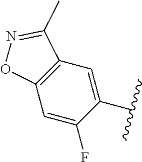 [SMILES: *c1cc2c(C)noc2cc1F] | —CN | H | 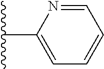 [SMILES: *c1ccccn1] |
| 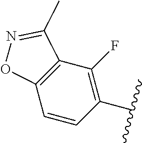 [SMILES: *c1ccc2onc(C)c2c1F] | —CN | H | 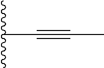 [SMILES: *C#CC] |
| 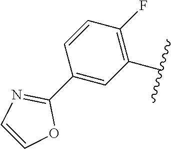 [SMILES: *c1cc(-c2ncco2)ccc1F] | —CN | H | 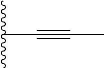 [SMILES: *C#CC] |
| 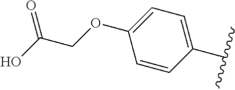 [SMILES: *c1ccc(OCC(=O)O)cc1] | —CN | H | 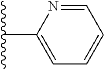 [SMILES: *c1ccccn1] |
| 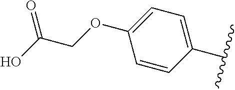 [SMILES: *c1ccc(OCC(=O)O)cc1] | 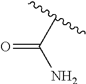 [SMILES: *C(N)=O] | H | 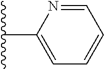 [SMILES: *c1ccccn1] |
| 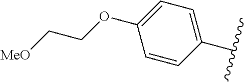 [SMILES: *c1ccc(OCCOC)cc1] | —CN | H | 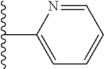 [SMILES: *c1ccccn1] |
| 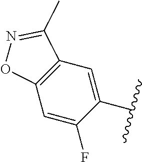 [SMILES: *c1cc2c(C)noc2cc1F] | —CN | —Cl | 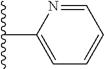 [SMILES: *c1ccccn1] |
| 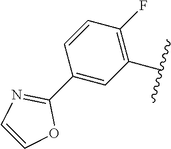 [SMILES: *c1cc(-c2ncco2)ccc1F] | —CN | —Cl | 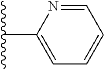 [SMILES: *c1ccccn1] |
| 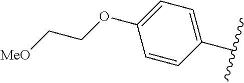 [SMILES: *c1ccc(OCCOC)cc1] | —CN | H | 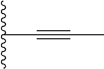 [SMILES: *C#CC] |
| 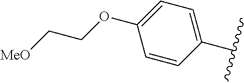 [SMILES: *c1ccc(OCCOC)cc1] | 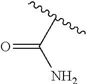 [SMILES: *C(N)=O] | H | 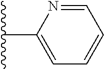 [SMILES: *c1ccccn1] |
| 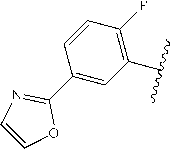 [SMILES: *c1cc(-c2ncco2)ccc1F] | 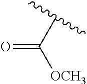 [SMILES: *C(=O)OC] | —Cl | 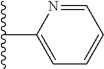 [SMILES: *c1ccccn1] |
| 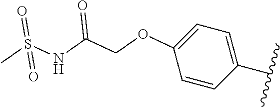 [SMILES: *c1ccc(OCC(=O)NS(C)(=O)=O)cc1] | —CN | —Cl | 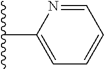 [SMILES: *c1ccccn1] |
| 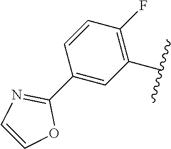 [SMILES: *c1cc(-c2ncco2)ccc1F] | 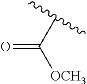 [SMILES: *C(=O)OC] | —CH₃ | 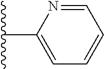 [SMILES: *c1ccccn1] |
| 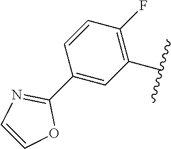 [SMILES: *c1cc(-c2ncco2)ccc1F] | 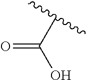 [SMILES: *C(=O)O] | —CH₃ | 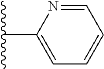 [SMILES: *c1ccccn1] |
| 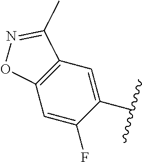 [SMILES: *c1cc2c(C)noc2cc1F] | 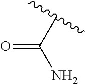 [SMILES: *C(N)=O] | Cl | 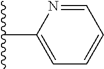 [SMILES: *c1ccccn1] |
| 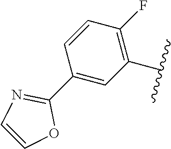 [SMILES: *c1cc(-c2ncco2)ccc1F] | 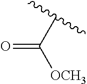 [SMILES: *C(=O)OC] | H | 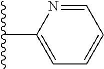 [SMILES: *c1ccccn1] |
| 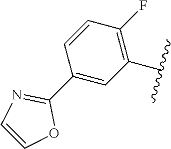 [SMILES: *c1cc(-c2ncco2)ccc1F] | 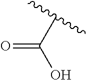 [SMILES: *C(=O)O] | H | 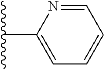 [SMILES: *c1ccccn1] |
| 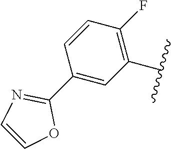 [SMILES: *c1cc(-c2ncco2)ccc1F] | 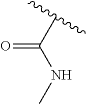 [SMILES: *C(=O)NC] | H | 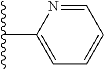 [SMILES: *c1ccccn1] |
| 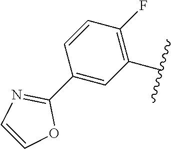 [SMILES: *c1cc(-c2ncco2)ccc1F] | 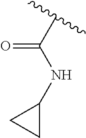 [SMILES: *C(=O)NC1CC1] | —CH₃ | 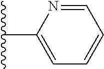 [SMILES: *c1ccccn1] |
| 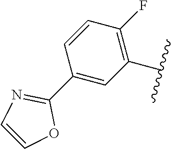 [SMILES: *c1cc(-c2ncco2)ccc1F] | 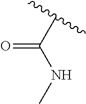 [SMILES: *C(=O)NC] | —CH₃ | 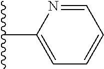 [SMILES: *c1ccccn1] |
| 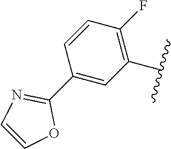 [SMILES: *c1cc(-c2ncco2)ccc1F] | 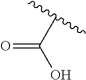 [SMILES: *C(=O)O] | —Cl | 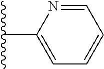 [SMILES: *c1ccccn1] |
| 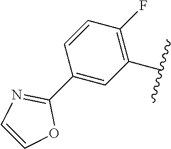 [SMILES: *c1cc(-c2ncco2)ccc1F] | 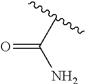 [SMILES: *C(N)=O] | —CH₃ | 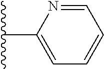 [SMILES: *c1ccccn1] |
| 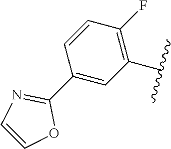 [SMILES: *c1cc(-c2ncco2)ccc1F] | 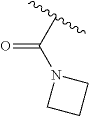 [SMILES: *C(=O)N1CCC1] | —CH₃ | 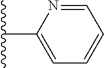 [SMILES: *c1ccccn1] |
| 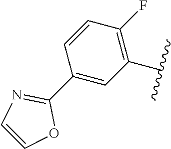 [SMILES: *c1cc(-c2ncco2)ccc1F] | 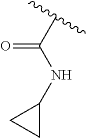 [SMILES: *C(=O)NC1CC1] | —Cl | 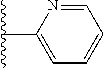 [SMILES: *c1ccccn1] |
| 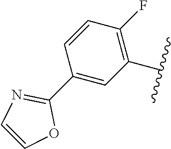 [SMILES: *c1cc(-c2ncco2)ccc1F] | 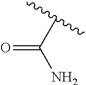 [SMILES: *C(N)=O] | Cl | 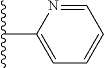 [SMILES: *c1ccccn1] |
| 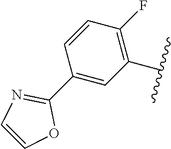 [SMILES: *c1cc(-c2ncco2)ccc1F] | 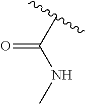 [SMILES: *C(=O)NC] | —Cl | 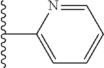 [SMILES: *c1ccccn1] |
| 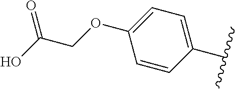 [SMILES: *c1ccc(OCC(=O)O)cc1] | 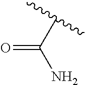 [SMILES: *C(N)=O] | —CH₃ | 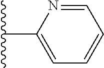 [SMILES: *c1ccccn1] |
| 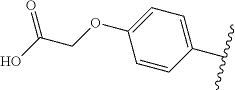 [SMILES: *c1ccc(OCC(=O)O)cc1] | 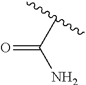 [SMILES: *C(N)=O] | H | 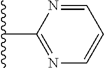 [SMILES: *c1ncccn1] |
| 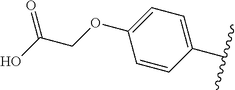 [SMILES: *c1ccc(OCC(=O)O)cc1] | 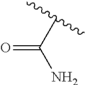 [SMILES: *C(N)=O] | H | 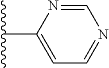 [SMILES: *c1ccncn1] |
| 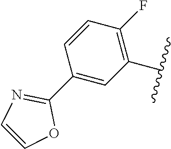 [SMILES: *c1cc(-c2ncco2)ccc1F] | H | —CH₃ | 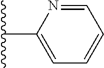 [SMILES: *c1ccccn1] |
| 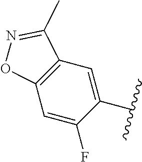 [SMILES: *c1cc2c(C)noc2cc1F] | 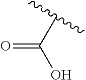 [SMILES: *C(=O)O] | —CH₃ | 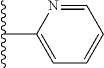 [SMILES: *c1ccccn1] |
| 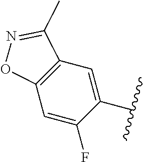 [SMILES: *c1cc2c(C)noc2cc1F] | 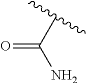 [SMILES: *C(N)=O] | —CH₃ | 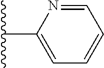 [SMILES: *c1ccccn1] |
| 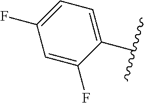 [SMILES: *c1ccc(F)cc1F] | 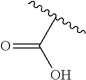 [SMILES: *C(=O)O] | —CH₃ | 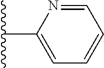 [SMILES: *c1ccccn1] |
| 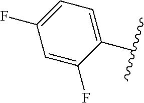 [SMILES: *c1ccc(F)cc1F] | 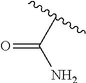 [SMILES: *C(N)=O] | —CH₃ | 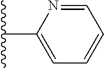 [SMILES: *c1ccccn1] |
| 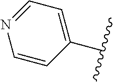 [SMILES: *c1ccncc1] | 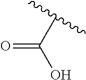 [SMILES: *C(=O)O] | —CH₃ | 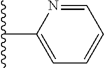 [SMILES: *c1ccccn1] |
| 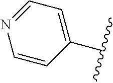 [SMILES: *c1ccncc1] | 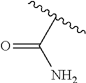 [SMILES: *C(N)=O] | —CH₃ | 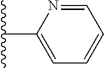 [SMILES: *c1ccccn1] |
| 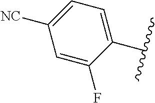 [SMILES: *c1ccc(C#N)cc1F] | 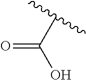 [SMILES: *C(=O)O] | —CH₃ | 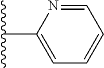 [SMILES: *c1ccccn1] |
| 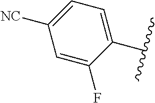 [SMILES: *c1ccc(C#N)cc1F] | 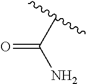 [SMILES: *C(N)=O] | —CH₃ | 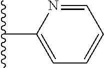 [SMILES: *c1ccccn1] |
| 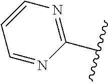 [SMILES: *c1ncccn1] | 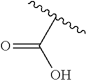 [SMILES: *C(=O)O] | —CH₃ | 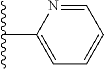 [SMILES: *c1ccccn1] |
| 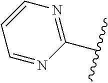 [SMILES: *c1ncccn1] | 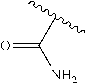 [SMILES: *C(N)=O] | —CH₃ | 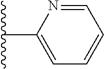 [SMILES: *c1ccccn1] |
| 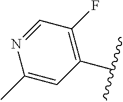 [SMILES: *c1cc(C)ncc1F] | 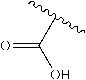 [SMILES: *C(=O)O] | —CH₃ | 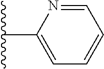 [SMILES: *c1ccccn1] |
| 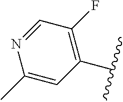 [SMILES: *c1cc(C)ncc1F] | 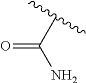 [SMILES: *C(N)=O] | —CH₃ | 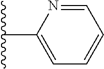 [SMILES: *c1ccccn1] |
| 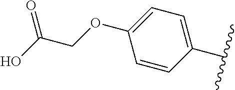 [SMILES: *c1ccc(OCC(=O)O)cc1] | 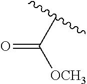 [SMILES: *C(=O)OC] | —CH₃ | 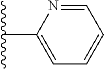 [SMILES: *c1ccccn1] |
| 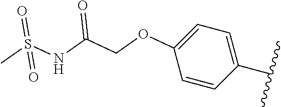 [SMILES: *c1ccc(OCC(=O)NS(C)(=O)=O)cc1] | —CN | H | 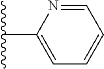 [SMILES: *c1ccccn1] |
| 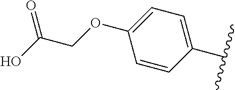 [SMILES: *c1ccc(OCC(=O)O)cc1] | 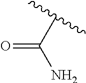 [SMILES: *C(N)=O] | —Cl | 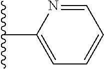 [SMILES: *c1ccccn1] |
| 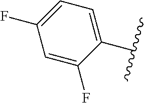 [SMILES: *c1ccc(F)cc1F] | 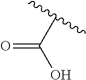 [SMILES: *C(=O)O] | —Cl | 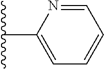 [SMILES: *c1ccccn1] |
| 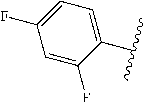 [SMILES: *c1ccc(F)cc1F] | 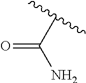 [SMILES: *C(N)=O] | —Cl | 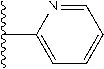 [SMILES: *c1ccccn1] |
| 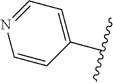 [SMILES: *c1ccncc1] | 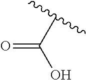 [SMILES: *C(=O)O] | —Cl | 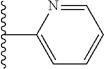 [SMILES: *c1ccccn1] |
| 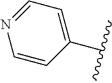 [SMILES: *c1ccncc1] | 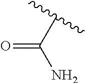 [SMILES: *C(N)=O] | —Cl | 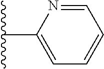 [SMILES: *c1ccccn1] |
| 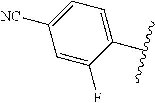 [SMILES: *c1ccc(C#N)cc1F] | 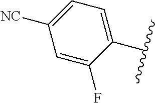 [SMILES: *c1ccc(C#N)cc1F] | —Cl | 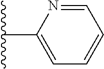 [SMILES: *c1ccccn1] |
| 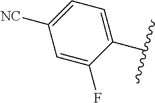 [SMILES: *c1ccc(C#N)cc1F] | 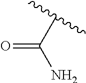 [SMILES: *C(N)=O] | —Cl | 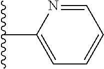 [SMILES: *c1ccccn1] |
| 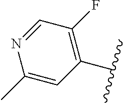 [SMILES: *c1cc(C)ncc1F] | 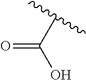 [SMILES: *C(=O)O] | —Cl | 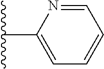 [SMILES: *c1ccccn1] |
| 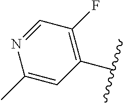 [SMILES: *c1cc(C)ncc1F] | 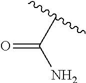 [SMILES: *C(N)=O] | —Cl | 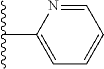 [SMILES: *c1ccccn1] |
| 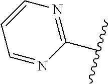 [SMILES: *c1ncccn1] | 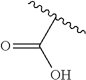 [SMILES: *C(=O)O] | —Cl | 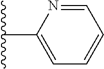 [SMILES: *c1ccccn1] |
| 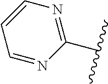 [SMILES: *c1ncccn1] | 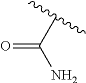 [SMILES: *C(N)=O] | —Cl | 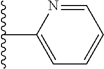 [SMILES: *c1ccccn1] |

TABLE 2

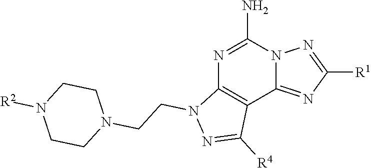

| R² | R⁴ | R¹ |
|---|---|---|
| 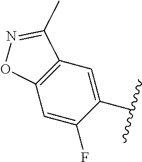 [SMILES: *c1cc2c(C)noc2cc1F] | —Cl | 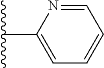 [SMILES: *c1ccccn1] |
| 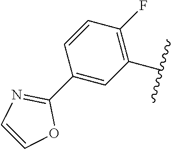 [SMILES: *c1cc(-c2ncco2)ccc1F] | —Cl | 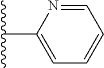 [SMILES: *c1ccccn1] |

TABLE 3

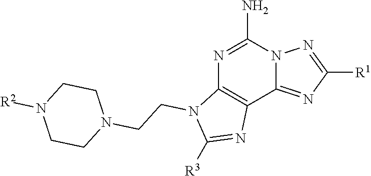

| R² | R³ | R¹ |
|---|---|---|
| 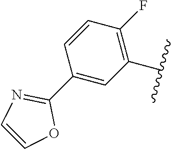 [SMILES: *c1cc(-c2ncco2)ccc1F] | H | 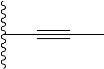 [SMILES: *C#CC] |
| 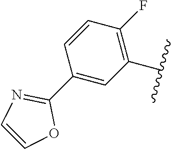 [SMILES: *c1cc(-c2ncco2)ccc1F] | H | 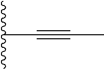 [SMILES: *C#CC] |
| 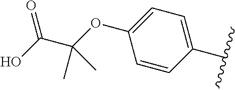 [SMILES: *c1ccc(OC(C)(C)C(=O)O)cc1] | H | 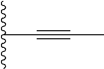 [SMILES: *C#CC] |
| 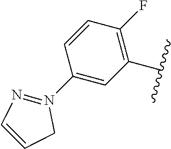 | H | 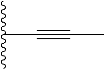 [SMILES: *C#CC] |
| 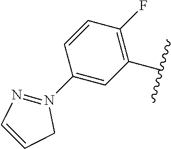 | H | 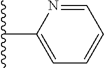 [SMILES: *c1ccccn1] |
| 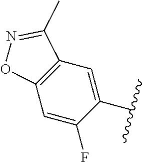 [SMILES: *c1cc2c(C)noc2cc1F] | H | 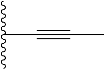 [SMILES: *C#CC] |
| 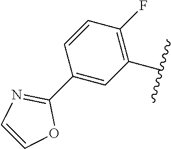 [SMILES: *c1cc(-c2ncco2)ccc1F] | H | 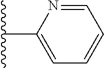 [SMILES: *c1ccccn1] |
| 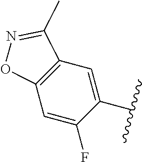 [SMILES: *c1cc2c(C)noc2cc1F] | H | 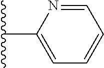 [SMILES: *c1ccccn1] |
| 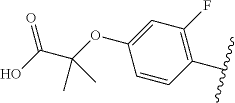 [SMILES: *c1ccc(OC(C)(C)C(=O)O)cc1F] | H | 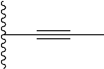 [SMILES: *C#CC] |
| 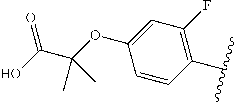 [SMILES: *c1ccc(OC(C)(C)C(=O)O)cc1F] | H | 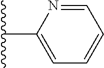 [SMILES: *c1ccccn1] |
| 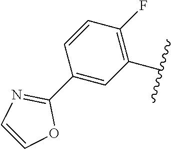 [SMILES: *c1cc(-c2ncco2)ccc1F] | H | 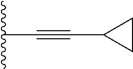 [SMILES: *C#CC1CC1] |

TABLE 4

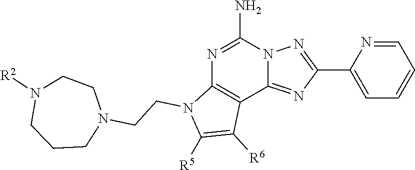

| R² | R⁵ | R⁶ |
|---|---|---|
| 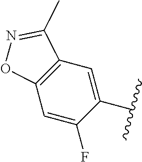 [SMILES: *c1cc2c(C)noc2cc1F] | 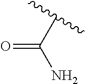 [SMILES: *C(N)=O] | —CH₃ |
| 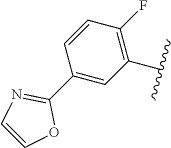 [SMILES: *c1cc(-c2ncco2)ccc1F] | 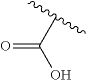 [SMILES: *C(=O)O] | —CH₃ |
| 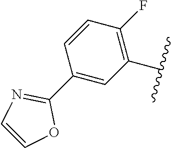 [SMILES: *c1cc(-c2ncco2)ccc1F] | H | —CH₃ |
| 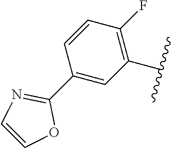 [SMILES: *c1cc(-c2ncco2)ccc1F] | 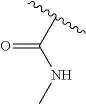 [SMILES: *C(=O)NC] | —CH₃ |
| 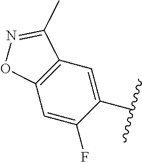 [SMILES: *c1cc2c(C)noc2cc1F] | 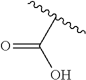 [SMILES: *C(=O)O] | —Cl |
| 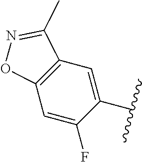 [SMILES: *c1cc2c(C)noc2cc1F] | 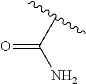 [SMILES: *C(N)=O] | —Cl |

TABLE 5

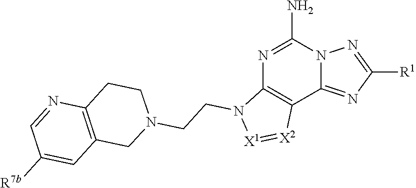

| R^{7b} | X¹ | X2 | R¹ |
|---|---|---|---|
| —CH₃ | N | C(Cl) | 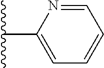 [SMILES: *c1ccccn1] |
| —CH₃ | 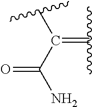 [SMILES: *C(=*)C(N)=O] | C(Cl) | 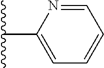 [SMILES: *c1ccccn1] |
| 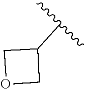 [SMILES: *C1COC1] | C(H) | N | 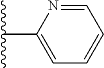 [SMILES: *c1ccccn1] |
| 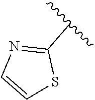 [SMILES: *c1nccs1] | C(H) | N | 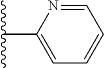 [SMILES: *c1ccccn1] |
| 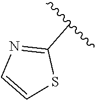 [SMILES: *c1nccs1] | C(H) | N | 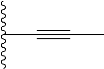 [SMILES: *C#CC] |
| 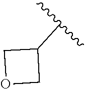 [SMILES: *C1COC1] | C(H) | N | 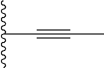 [SMILES: *C#CC] |
| 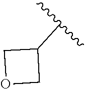 [SMILES: *C1COC1] | C(H) | N | 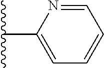 [SMILES: *c1ccccn1] |

### Further Forms of Compounds

In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich: Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound of Formula (I) Formula (X), or Formula (XI) with an acid. In some embodiments, the compound of Formula (I) Formula (X), or Formula (XI) (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, pharmaceutically acceptable salts are obtained by reacting a compound of Formula (I), Formula (X), or Formula (XI) with a base. In some embodiments, the compound of Formula (I), Formula (X), or Formula (XI) is acidic and is reacted with a base. In such situations, an acidic proton of the compound of Formula (I), Formula (X), or Formula (XI) is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), or pharmaceutically acceptable salts of compounds having the structure of Formula (I), Formula (X), or Formula (XI), as well as active metabolites of these compounds having the same type of activity.

In some embodiments, sites on the organic radicals (e.g. alkyl groups, aromatic rings) of compounds of Formula (I), Formula (X), or Formula (XI) are susceptible to various metabolic reactions. Incorporation of appropriate substituents on the organic radicals will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine chlorine, iodine, phosphorus, such as, for example, ²H, ³H, ¹³C, ¹⁴C, ¹⁵N, ¹⁸O, ¹⁷O, ³⁵S, ¹⁸F, ³⁶Cl, ¹²³I, ¹²⁴I, ¹²⁵I, ¹³¹I, ³²P and ³³P. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements. In some embodiments, one or more hydrogens of the compounds of Formula (I), Formula (X), or Formula (XI) are replaced with deuterium.

In some embodiments, the compounds of Formula (I), Formula (X), or Formula (XI) possess one or more stereocenters and each stereocenter exists independently in either the R or S configuration. In some embodiments, the compound of Formula (I), Formula (X), or Formula (XI) exists in the R configuration. In some embodiments, the compound of Formula (I), Formula (X), or Formula (XI) exists in the S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents. In certain embodiments, compounds of Formula (I), Formula (X), or Formula (XI) are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of steroisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrugs of the compounds described herein include, but are not limited to, esters, ethers, carbonates, thiocarbonates, N-acyl derivatives, N-acyloxyalkyl derivatives, N-alkyloxyacyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (i.e. the prodrug), which is then metabolized in vivo to provide a carboxylic acid group. In some embodiments, compounds described herein are prepared as alkyl ester prodrugs.

Prodrug forms of the herein described compounds, wherein the prodrug is metabolized in vivo to produce a compound of Formula (I), Formula (X), or Formula (XI) as set forth herein are included within the scope of the claims. In some cases, some of the herein-described compounds is a prodrug for another derivative or active compound.

In some embodiments, any one of the hydroxyl group(s), amino group(s) and/or carboxylic acid group(s) are functionalized in a suitable manner to provide a prodrug moiety. In some embodiments, the prodrug moiety is as described above.

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

### Synthesis of Compounds

Compounds described herein are synthesized using standard synthetic techniques or using methods known in the art in combination with methods described herein. Alternative reaction conditions for the synthetic transformations described herein may be employed such as variation of solvent, reaction temperature, reaction time, as well as different chemical reagents and other reaction conditions. The starting materials are available from commercial sources or are readily prepared.

Unless otherwise indicated, conventional methods of mass spectroscopy, NMR, HPLC, protein chemistry, biochemistry, recombinant DNA techniques and pharmacology are employed.

In some embodiments, compounds described herein are prepared as outlined in Scheme 1.

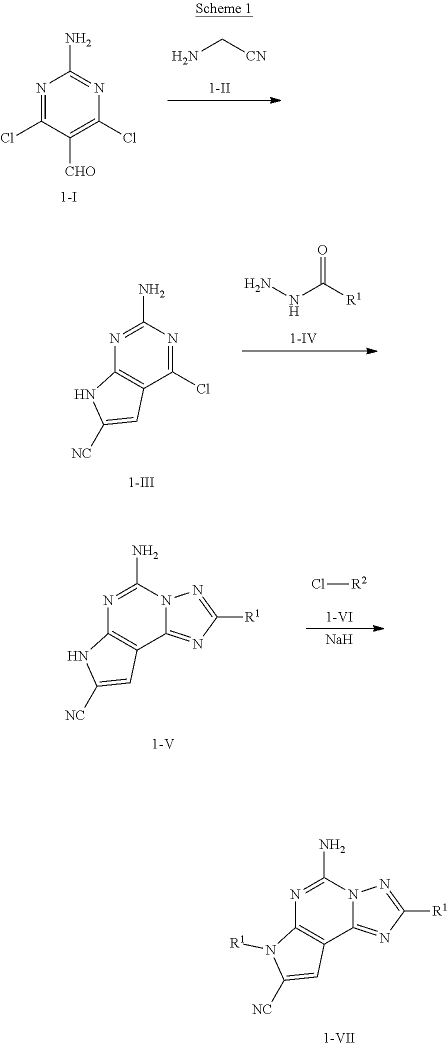

In some embodiments, the preparation of compounds described herein begins with appropriately substituted aldehyde 1-I. In some embodiments, the pyrrolopyrimidine analog 1-III is prepared by an intramolecular cyclization of 1-I with aminoacetonitrile 1-II) in a solvent, such as ethanol, and base, such as triethylamine. In some embodiments, the halide, such as chlorine, of 1-III is displaced with a hydrazide, such as 1-IV (where R¹ is 6-membered heteroaryl ring or alkyne as described herein), using a solvent, such as acetonitrile, and a base, such as N, N-diisopropylethylamine (DIPEA). Subsequently, a dehydrative rearrangement takes place upon subjecting the intermediate to hexamethyldisilazine (HMDS) and N, O-bis(trimethylsilyl)acetamide (BSA) followed by heating overnight, yielding 1-V. In some embodiments, the indole NH is alkylated by treatment of 1-V with, for example, NaH and an alkylating agent (1-VI, where R² is heteroalkyl substituted with phenyl or a monocyclic or bicyclic heteroaryl ring as described herein) in a solvent, such as DMF. In some further embodiments, the resulting N-alkylated analog (1-VII) is further modified using standard chemical transformations.

An alternative route to preparing compounds described herein is shown in Scheme 2.

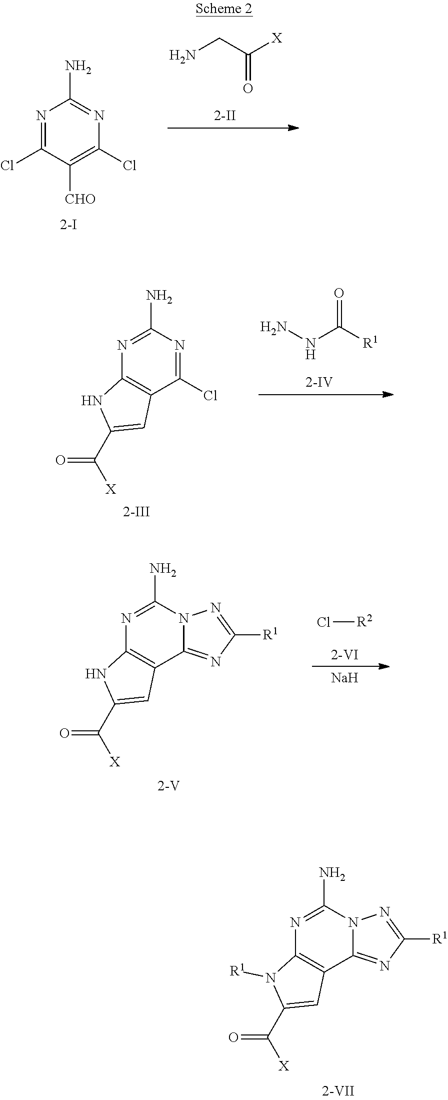

In some embodiments, the preparation of compounds described herein begins with appropriately substituted aldehyde 2-I. In some embodiments, the pyrrolopyrimidine analog 2-III is prepared by an intramolecular cyclization of 2-I with amino compounds, such as 2-II (where X may be OR, NRR′, or R″, where R and R′ may be H or C¹-C⁶ alkyl and R″ may be C¹-C⁶ alkyl), in a solvent, such as ethanol, and base, such as triethylamine. In some embodiments, the halide, such as chlorine, of 2-III is displaced with a hydrazide, such as 2-IV (where R¹ is 6-membered heteroaryl ring or alkyne as described herein), using a solvent, such as acetonitrile, and a base, such as N, N-diisopropylethylamine (DIPEA). Subsequently, a dehydrative rearrangement takes place upon subjecting the intermediate to hexamethyldisilazine (HMDS) and N, O-bis(trimethylsilyl)acetamide (BSA) followed by heating overnight, yielding compounds like 2-V. In some embodiments, the indole NH is alkylated by treatment of 2-V with, for example, NaH and an alkylating agent (2-VI, where R² is heteroalkyl substituted with phenyl or a monocyclic or bicyclic heteroaryl ring as described herein) in a solvent, such as DMF. In some further embodiments, the resulting N-alkylated analog (2-VII) is further modified using standard chemical transformations.

An alternative route to preparing compounds described herein is shown in Scheme 3.

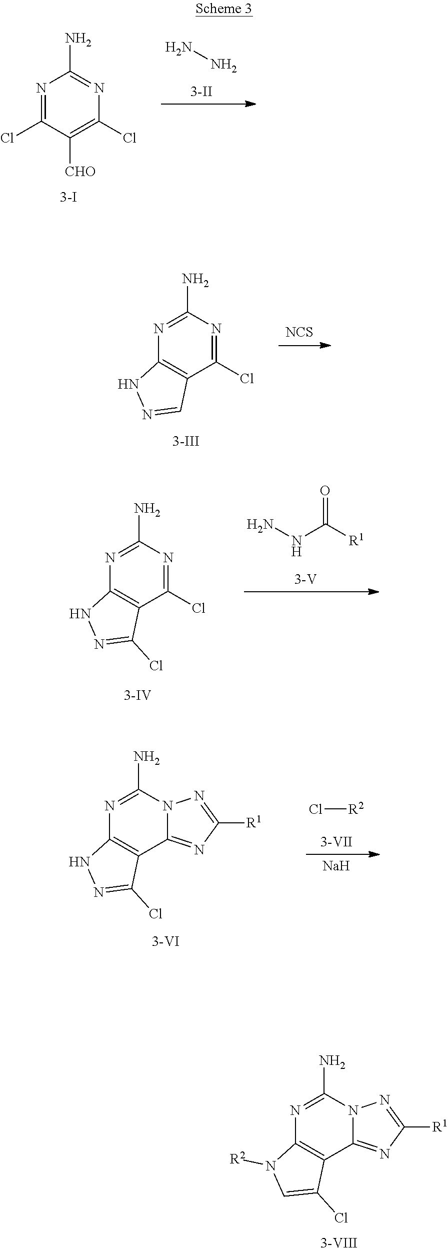

In some embodiments, the preparation of compounds described herein begins with appropriately substituted aldehyde 3-I. In some embodiments, the pyrazolo[3,4-d]pyrimidine analog 3-III is prepared by an intramolecular cyclization of 3-I with hydrazine 3-II in a solvent, such as DMF, and base, such as N, N-diisopropylethylamine (DIPEA). In some embodiments, the pyrazolo[3,4-d]pyrimidine analog 3-III is halogenated with a halogenating reagent, such as N-chlorosuccinimide in a solvent, such as DMF. In some embodiments, the halide, such as chlorine, on the pyrimidine ring of 3-IV is displaced with a hydrazide, such as 3-V (where R¹ is 6-membered heteroaryl ring or alkyne as described herein), using a solvent, such as acetonitrile, and a base, such as DIPEA. Subsequently, a dehydrative rearrangement takes place upon subjecting the intermediate to hexamethyldisilazine (HMDS) and N, O-bis(trimethylsilyl)acetamide (BSA) followed by heating overnight, yielding compounds like 3-VI. In some embodiments, the indole NH is alkylated by treatment of 3-VI with, for example, NaH and an alkylating agent (3-VII, where R² is heteroalkyl substituted with phenyl or a monocyclic or bicyclic heteroaryl ring as described herein) in a solvent, such as DMF. In some further embodiments, the resulting N-alkylated analog (3-VIII) is further modified using standard chemical transformations.

An alternative route to preparing compounds described herein is shown in Scheme 4.

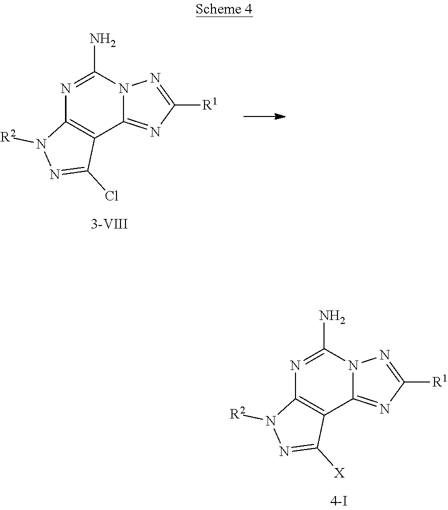

In some embodiments, the preparation of compounds described herein begins with appropriately substituted halogen-containing compounds like 3-VIII (where R¹ is 6-membered heteroaryl ring or alkyne as described herein and where R² is heteroalkyl substituted with phenyl or a monocyclic or bicyclic heteroaryl ring as described herein). In some embodiments, the X (where X may be OR, NRR′, or R″, where R and R′ may be H or C¹-C⁶ alkyl and R¹¹ may be C¹-C⁶ alkyl) of 4-I is formed through C—X (where X is halogen) functionalization using a phosphine ligand, such as t-BuBrettPhos, in the presence of heating, base, such as sodium t-butanolate, N,N,N,N-tetramethylethylenediamine, or sodium caprylate, a palladium-catalyst, such as [(2-di-tert-butylphosphino-3,6-dimethoxy-2′,4′,6′-triisopropyl-1,1′-biphenyl)-2-(2′-amino-1,1′-biphenyl)]palladium(II) methanesulfonate or C₄₇H₆₄NO₄PPdS, and solvent or solvent mixtures, such as water, ethanol, DMF, octanol, 1,4-dioxane, and, in some cases, zinc in water.

In some embodiments, compounds described herein are synthesized as outlined in the Examples.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers to a divalent alkyl radical. Any of the above mentioned monovalent alkyl groups may be an alkylene by abstraction of a second hydrogen atom from the alkyl. In some embodiments, an alkylene is a C₁-C₆alkylene. In other embodiments, an alkylene is a C₁-C₄alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. Non-limiting examples of an alkynyl group include —C≡CH, —C≡CCH₃—C≡CCH₂CH₃, —CH₂C≡CH.

An “alkoxy” group refers to a (alkyl)O— group, where alkyl is as defined herein.

The term “alkylamine” refers to —NH(alkyl), or —N(alkyl)₂.

The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e. skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicycle[1.1.1]pentyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, adamantyl, norbornyl, and decalinyl. In some embodiments, a cycloalkyl is a C₃-C₆cycloalkyl.

The term “halo” or, alternatively, “halogen” or “halide” means fluoro, chloro, bromo or iodo. In some embodiments, halo is fluoro, chloro, or bromo.

The term “fluoroalkyl” refers to an alkyl in which one or more hydrogen atoms are replaced by a fluorine atom. In one aspect, a fluoroalkyl is a C₁-C₆fluoroalkyl.

The term “heteroalkyl” refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl.

Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, and —CH₂SCH₃.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” or “heteroalicyclic” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. The term heteroalicyclic also includes all ring forms of the carbohydrates, including but not limited to the monosaccharides, the disaccharides and the oligosaccharides. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H,—CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy,—SC1-C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target. In some embodiments, “modulate” means to interact with a target either directly or indirectly so as to decrease or inhibit receptor activity,

The term “modulator” as used herein, refers to a molecule that interacts with a target either directly or indirectly. The interactions include, but are not limited to, the interactions of an agonist, partial agonist, an inverse agonist, antagonist, or combinations thereof. In some embodiments, a modulator is an antagonist. Receptor antagonists are inhibitors of receptor activity. Antagonists mimic ligands that bind to a receptor and prevent receptor activation by a natural ligand. Preventing activation may have many effects. If a natural agonist binding to a receptor leads to an increase in cellular function, an antagonist that binds and blocks this receptor decreases the function.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “kit” and “article of manufacture” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

It should be understood that in addition to the ingredients particularly mentioned above, the compounds and compositions described herein may include other agents conventional in the art having regard to the type of formulation in question, for example those suitable for oral administration may include flavoring agents.

### Methods of Treatment, Dosing and Treatment Regimens

The compounds disclosed herein, or pharmaceutically acceptable salts, solvates, or stereoisomers thereof, are useful for the modulation of A_{2A} adenosine receptors.

Provided herein are antagonists of the A_{2A} adenosine receptor that are useful in the treatment of one or more diseases or disorders associated with A_{2A} adenosine receptor activity or that would benefit from administration of one of the A_{2A} adenosine receptor antagonists described herein.

In some embodiments, described herein are methods for treating a disease or disorder, wherein the disease or disorder is cancer, a hyperproliferative disorder, an autoimmune disorder, or inflammatory disorder.

In one embodiment, the compounds described herein, or a pharmaceutically acceptable salt thereof, are used in the preparation of medicaments for the treatment of diseases or conditions in a mammal that would benefit from inhibition or reduction of A_{2A} adenosine receptor activity. Methods for treating any of the diseases or conditions described herein in a mammal in need of such treatment, involves administration of pharmaceutical compositions that include at least one compound described herein or a pharmaceutically acceptable salt, active metabolite, prodrug, or pharmaceutically acceptable solvate thereof, in therapeutically effective amounts to said mammal.

In certain embodiments, the compositions containing the compound(s) described herein are administered for prophylactic and/or therapeutic treatments. In certain therapeutic applications, the compositions are administered to a mammal already suffering from a disease or condition, in an amount sufficient to cure or at least partially arrest at least one of the symptoms of the disease or condition. Amounts effective for this use depend on the severity and course of the disease or condition, previous therapy, the mammal's health status, weight, and response to the drugs, and the judgment of a healthcare practitioner. Therapeutically effective amounts are optionally determined by methods including, but not limited to, a dose escalation and/or dose ranging clinical trial.

In prophylactic applications, compositions containing the compounds described herein are administered to a mammal susceptible to or otherwise at risk of a particular disease, disorder or condition. Such an amount is defined to be a “prophylactically effective amount or dose.” In this use, the precise amounts also depend on the mammal's state of health, weight, and the like. When used in mammals, effective amounts for this use will depend on the severity and course of the disease, disorder or condition, previous therapy, the mammal's health status and response to the drugs, and the judgment of a healthcare professional. In one aspect, prophylactic treatments include administering to a mammal, who previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments wherein the mammal's condition does not improve, upon the discretion of a healthcare professional the administration of the compounds are administered chronically, that is, for an extended period of time, including throughout the duration of the mammal's life in order to ameliorate or otherwise control or limit the symptoms of the mammal's disease or condition.

In certain embodiments wherein a mammal's status does improve, the dose of drug being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the mammal requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

In certain instances, it is appropriate to administer at least one compound described herein, or a pharmaceutically acceptable salt thereof, in combination with one or more other therapeutic agents. In certain embodiments, the pharmaceutical composition further comprises one or more anti-cancer agents.

In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In one specific embodiment, a compound described herein, or a pharmaceutically acceptable salt thereof, is co-administered with a second therapeutic agent, wherein the compound described herein, or a pharmaceutically acceptable salt thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply be additive of the two therapeutic agents or the patient experiences a synergistic benefit.

In certain embodiments, different therapeutically-effective dosages of the compounds disclosed herein will be utilized in formulating pharmaceutical composition and/or in treatment regimens when the compounds disclosed herein are administered in combination with one or more additional agent, such as an additional therapeutically effective drug, an adjuvant or the like. Therapeutically-effective dosages of drugs and other agents for use in combination treatment regimens is optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a compound described herein, or a pharmaceutically acceptable salt thereof, is initiated prior to, during, or after treatment with a second agent described herein, and continues until any time during treatment with the second agent or after termination of treatment with the second agent. It also includes treatments in which a compound described herein, or a pharmaceutically acceptable salt thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the disease(s) for which relief is sought, is modified in accordance with a variety of factors (e.g. the disease or disorder from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

In some embodiments, a compound described herein, or a pharmaceutically acceptable salt thereof, is administered in combination with chemotherapy, radiation therapy, monoclonal antibodies, or combinations thereof.

Chemotherapy includes the use of anti-cancer agents.

### EXAMPLES

The following examples are provided for illustrative purposes only and not to limit the scope of the claims provided herein.

### Example 1: 5-amino-7-[2-[4-(2-fluoro-5-oxazol-2-yl-phenyl)piperazin-1-yl]ethyl]-2-(2-pyridyl)-[1,2,4]triazolopyrrolopyrimidine-8-carbonitrile

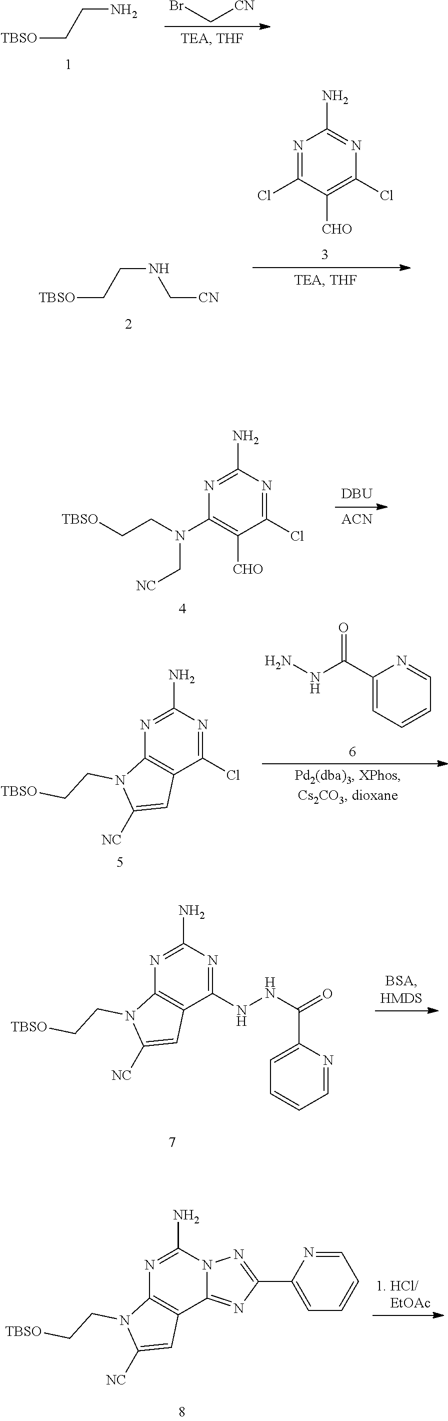

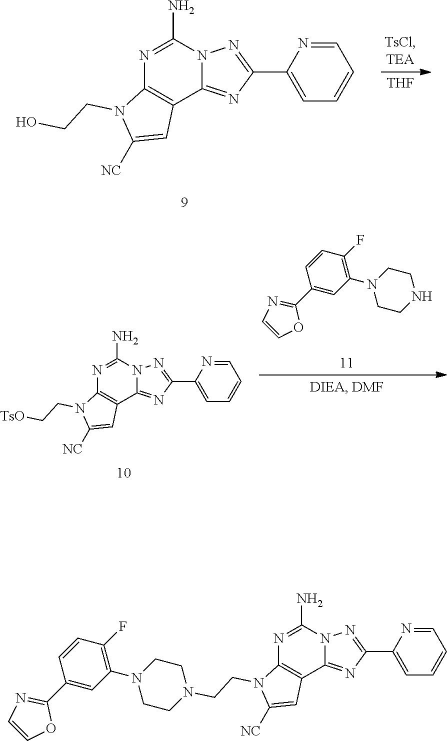 [SMILES: N#Cc1cc2c(nc(N)n3nc(-c4ccccn4)nc23)n1CCO ; Cc1ccc(S(=O)(=O)OCCn2c(C#N)cc3c2nc(N)n2nc(-c4ccccn4)nc32)cc1 ; Fc1ccc(-c2ncco2)cc1N1CCNCC1 ; "THF" ; N#Cc1cc2c(nc(N)n3nc(-c4ccccn4)nc23)n1CCN1CCN(c2cc(-c3ncco3)ccc2F)CC1]

### Step 1: Synthesis of Compound 2

To a solution of 2-[tert-butyl (dimethyl) silyl]oxyethanamine (2 g, 11.4 mmol, 1 eq) and 2-bromoacetonitrile (1.50 g, 12.6 mmol, 836 uL, 1.1 eq) in THF (20 mL) was added TEA (2.31 g, 22.8 mmol, 3.18 mL, 2 eq). The mixture was stirred at 40° C. for 4 h. TLC showed the reaction was completed. The mixture was concentrated and the residue was purified by column chromatography (SiO₂, Petroleum ether/Ethyl acetate=100/1 to 1/1) to obtain 2-[2-[tert-butyl (dimethyl) silyl]oxyethylamino]acetonitrile (2 g, 8.86 mmol, 78% yield, 95% purity) as yellow oil. ¹H NMR (400 MHz, CHLOROFORM-d) δ=3.80-3.72 (m, 2H), 3.65 (s, 2H), 2.87-2.80 (m, 2H), 1.69 (br s, 1H), 0.96-0.85 (m, 9H), 0.08 (s, 5H).

### Step 2: Synthesis of Compound 4

To a solution of 2-[2-[tert-butyl (dimethyl) silyl]oxyethylamino]acetonitrile (2.4 g, 11.2 mmol, 1 eq) and TEA (1.89 g, 16.8 mmol, 2.60 mL, 90% purity, 1.5 eq) in THE (10 mL) was added 2-amino-4,6-dichloro-pyrimidine-5-carbaldehyde (2.15 g, 11.2 mmol, 1 eq). The mixture was stirred at 40° C. for 12 h. TLC showed the reaction was completed. The mixture was concentrated and the residue was purified by column chromatography (SiO₂, Petroleum ether/Ethyl acetate=10/1 to 1/1) to obtain 2-[(2-amino-6-chloro-5-formyl-pyrimidin-4-yl)-[2-[tert-butyl (dimethyl) silyl]oxyethyl]amino]acetonitrile (1.5 g, 3.73 mmol, 33% yield, 92% purity) as white solid. ¹H NMR (400 MHz, DMSO-d6) δ=9.91 (s, 1H), 7.89 (br s, 1H), 7.75 (br s, 1H), 4.51 (s, 2H), 3.82 (t, J=5.07 Hz, 2H), 3.57 (t, J=5.07 Hz, 2H), 0.74-0.83 (m, 9H).

### Step 3: Synthesis of Compound 5

To a solution of 2-[(2-amino-6-chloro-5-formyl-pyrimidin-4-yl)-[2-[tert-butyl (dimethyl)silyl]oxyethyl]amino]acetonitrile (50 g, 135 mmol, 1 eq) in ACN (1 L) was added DBU (25.7 g, 169 mmol, 25.5 mL, 1.25 eq) at 20° C. The reaction was stirred at 80° C. for 12 h. TLC showed consumption of starting material and one major new spot with lower polarity was detected. The mixture was concentrated and the residue was purified by column chromatography (SiO₂, Petroleum ether/Ethyl acetate=100/1 to 2/1) to give 2-amino-7-[2-[tert-butyl (dimethyl) silyl]oxyethyl]-4-chloro-pyrrolo[2,3-d]pyrimidine-6-carbonitrile (24.2 g, 66.7 mmol, 49% yield, 97% purity) as yellow solid. LCMS for product (ESI+): m/z 352.2 (M+H⁺), Rt: 1.122 min.

LC/MS (The column used for chromatography was a HALO AQ-C18 2.1*30 mm 2.7 um. Detection methods are diode array (DAD). MS mode was positive electrospray ionization. MS range was 100-1000. Mobile phase A was 0.037% Trifluoroacetic acid in water, and mobile phase B was 0.018%.

Trifluoroacetic acid in HPLC grade acetonitrile. The gradient was 5-95% B in 2.00 min 0.5% B in 0.01 min, 5-95% B (0.01-1.00 min), 95-100% B (1.00-1.80 min), 5% B in 1.81 min with a hold at 5% B for 0.19 min. The flow rate was 1.0 mL/min (0.00-1.80 min) and 1.2 mL/min (1.81-2.00 min).

¹H NMR (400 MHz, DMSO-d6) δ=8.21 (s, 1H), 7.67-7.51 (m, 2H), 7.46-7.23 (m, 2H), 3.56-3.43 (m, 4H), 3.08-2.99 (m, 4H), 1.43 (s, 9H)

### Step 4: Synthesis of Compound 7

To a solution of 2-amino-7-[2-[tert-butyl (dimethyl) silyl]oxyethyl]-4-chloro-pyrrolo[2,3-d]pyrimidine-6-carbonitrile (3 g, 8.53 mmol, 1 eq) and pyridine-2-carbohydrazide (1.17 g, 8.53 mmol, 1 eq) in dioxane (60 mL) was added Pd₂(dba)₃ (781 mg, 853 umol, 0.1 eq), XPhos (813 mg, 1.71 mmol, 0.2 eq) and Cs₂CO₃ (8.33 g, 25.6 mmol, 3 eq) under N₂. The mixture was stirred at 80° C. for 12 h. LCMS showed the starting material was completely consumed and one main peak with desired mass was detected. The mixture was filtered and the solid was washed with 1,4-dioxane (10 mL×3) to give a crude product. The crude product was suspended in H₂O (100 mL) and filtered. The solid was suspended in DMF (50 mL) and filtered. The filtrate was concentrate under reduced pressure to give N′-[2-amino-7-[2-[tert-butyl (dimethyl) silyl]oxyethyl]-6-cyano-pyrrolo[2,3-d]pyrimidin-4-yl]pyridine-2-carbo hydrazide (1.1 g, 2.19 mmol, 26% yield, 90% purity) as brown solid. ¹H NMR (400 MHz, DMSO-d₆) δ=11.44-10.38 (m, 1H), 9.97-9.13 (m, 1H), 8.72 (br s, 1H), 8.04 (br d, J=5.6 Hz, 2H), 7.67 (br s, 1H), 7.32 (br s, 1H), 6.31 (br s, 2H), 4.11 (br s, 2H), 3.85 (br s, 2H), 0.78 (s, 9H), −0.14 (s, 6H)

### Step 5: Synthesis of Compound 8

A mixture of N′-[2-amino-7-[2-[tert-butyl (dimethyl) silyl]oxyethyl]-6-cyano-pyrrolo[2,3-d]pyrimidin-4-yl]pyridine-2-carbohydrazide (3 g, 6.63 mmol, 1 eq) in BSA (20 mL) and HMDS (60 mL) was stirred at 130° C. for 4 h. LC-MS showed disappearance of starting material and one main peak with desired mass was detected. The mixture was concentrated under reduced pressure to give a crude product. The crude product was suspended in hot water (80 mL) and filtered. The solid was dried under reduced pressure to afford 5-amino-7-[2-[tert-butyl (dimethyl) silyl]oxyethyl]-2-(2-pyridyl)-[1,2,4]triazolopyrrolopyrimidine-8-carbo nitrile (7 g, 14.5 mmol, 73% yield, 90% purity) as yellow solid. ¹H NMR (400 MHz, DMSO-d₆) δ=8.75 (d, J=4.2 Hz, 1H), 8.30 (d, J=7.5 Hz, 1H), 8.17 (br s, 2H), 8.01 (dt, J=1.5, 7.7 Hz, 1H), 7.67 (s, 1H), 7.55 (dd, J=5.2, 6.9 Hz, 1H), 4.32 (br t, J=4.9 Hz, 2H), 3.96 (t, J=5.0 Hz, 2H), 0.73 (s, 9H), −0.17 (s, 6H)

### Step 6: Synthesis of Compound 9

5-amino-7-[2-[tert-butyl (dimethyl) silyl]oxyethyl]-2-(2-pyridyl)-[1,2,4]triazolopyrrolopyrimidine-8-carbonitrile (7 g, 16.1 mmol, 1 eq) was suspended in HCl/EtOAc (4 M, 300 mL, 74.5 eq). The reaction was stirred at 20° C. for 2 h. LC-MS showed complete consumption of staring material and one main peak with desired mass was detected. The mixture was filtered and the solid was washed with EA (20 mL×3) to give

5-amino-7-(2-hydroxyethyl)-2-(2-pyridyl)-[1,2,4]triazolopyrrolopyrimidine-8-carbonitrile (5 g, crude) as yellow solid. ¹H NMR (400 MHz, DMSO-d₆) δ=8.79 (dd, J=0.8, 5.0 Hz, 1H), 8.41 (d, J=7.8 Hz, 1H), 8.21 (dt, J=1.7, 7.8 Hz, 1H), 7.72 (ddd, J=1.1, 5.1, 7.6 Hz, 1H), 7.64 (s, 1H), 4.27 (t, J=5.6 Hz, 2H), 3.76 (t, J=5.6 Hz, 2H)

### Step 7: Synthesis of Compound 10

To a mixture of 5-amino-7-(2-hydroxyethyl)-2-(2-pyridyl)-[1,2,4]triazolopyrrolopyrimidine-8-carbonitrile (4 g, 12.5 mmol, 1 eq) in pyridine (157 g, 1.98 mol, 160 mL, 159 eq) was added 4-methylbenzenesulfonyl chloride (7.14 g, 37.5 mmol, 3 eq) at 0-10° C. The reaction was stirred at 40° C. for 8 hr. LC-MS showed starting material was completely consumed and one main peak with desired mass was detected. The mixture was concentrated under reduced pressure to give a crude product. The crude product was triturated in H₂O at 20° C. for 10 min. Then the solid was purified by re-crystallization from EA (100 mL) at 20° C. to afford 2-[5-amino-8-cyano-2-(2-pyridyl)-[1,2,4]triazolopyrrolopyrimidin-7-yl]ethyl 4-methylbenzenesulfonate (3.5 g, 7.38 mmol, 47% yield) as brown solid.

### Step 8: Synthesis of 5-amino-7-[2-[4-(2-fluoro-5-oxazol-2-yl-phenyl)piperazin-1-yl]ethyl]-2-(2-pyridyl)-[1,2,4]triazolopyrrolopyrimidine-8-carbonitrile

To a solution of 2-[5-amino-8-cyano-2-(2-pyridyl)-[1,2,4]triazolopyrrolopyrimidin-7-yl]ethyl 4-methylbenzenesulfonate (500 mg, 1.05 mmol, 1 eq) in DMF (20 mL) was added DIEA (204 mg, 1.58 mmol, 275 uL, 1.5 eq) and 2-(4-fluoro-3-piperazin-1-yl-phenyl) oxazole (261 mg, 1.05 mmol, 1 eq). The reaction was stirred at 80° C. for 12 hr. LCMS showed showed both starting material remained and the desired product.

The reaction was concentrated under reduced pressure to obtain a crude product. The residue was purified by prep-HPLC (neutral condition) to give 5-amino-7-[2-[4-(2-fluoro-5-oxazol-2-yl-phenyl) piperazin-1-yl]ethyl]-2-(2-pyridyl)-[1,2,4]triazolopyrrolopyrimidine-8-carbonitrile (60 mg, 104 umol, 9.8% yield, 95% purity) as white solid.

LCMS for product (ESI+): m/z 550.2 (M+H)⁺, Rt: 2.809 min.

LC/MS (The gradient was 15-90% B in 3.40 min and 90-100% B at 3.40-3.85 min, 100-15% B in 0.01 min, and then held at 15% for 0.64 min, the flow rate was 0.80 ml/min. Mobile phase A was 10 mM Ammonium bicarbonate, mobile phase B was HPLC grade acetonitrile. The column used for chromatography was a 2.1*50 mm Xbridge Shield RPC18 column (5 um particles). Detection methods are diode array (DAD) and evaporative light scattering (ELSD) detection as well as positive electrospray ionization. MS range was 100-1000.

¹H NMR (400 MHz, DMSO-d₆) δ ppm 2.67 (br s, 4H) 2.78 (br s, 2H) 3.06 (br s, 4H) 4.31-4.42 (m, 2H) 7.29 (dd, J=12.53, 8.25 Hz, 1H) 7.35 (s, 1H) 7.55 (br t, J=8.07 Hz, 3H) 7.68 (s, 1H) 8.01 (t, J=7.70 Hz, 1H) 8.19 (s, 2H) 8.30 (d, J=7.95 Hz, 1H) 8.75 (br d, J=3.67 Hz, 1H)

### Example 2: Synthesis of 5-amino-7-[2-[4-(2-fluoro-5-oxazol-2-yl-phenyl)piperazin-1-yl]ethyl]-2-(2-pyridyl)-[1,2,4]triazolopyrrolopyrimidine-8-carboxamide

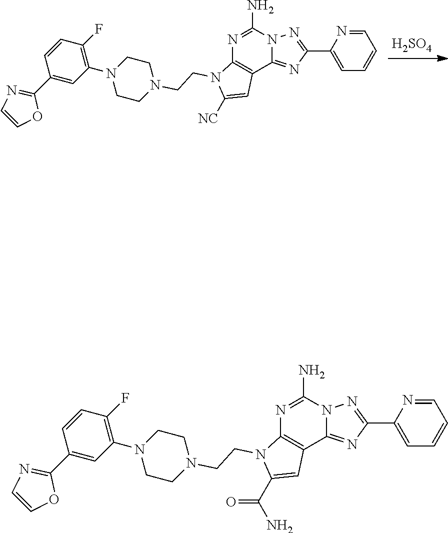

5-amino-7-[2-[4-(2-fluoro-5-oxazol-2-yl-phenyl) piperazin-1-yl]ethyl]-2-(2-pyridyl)-[1,2,4]triazolopyrrolo pyrimidine-8-carbonitrile (90 mg, 164 umol, 1 eq) was dissolved in H₂SO₄ (1.84 g, 18.8 mmol, 1 mL, 115 eq). The reaction was stirred at 20° C. for 0.5 hr. The mixture was dropwise added to an aqueous NH₃H₂O (20 mL) and filtered. The filtrate was concentrated under reduced pressure to give a crude product. The residue was purified by prep-HPLC (neutral condition) to give 5-amino-7-[2-[4-(2-fluoro-5-oxazol-2-yl-phenyl)piperazin-1-yl]ethyl]-2-(2-pyridyl)-[1,2,4]triazolopyrrolopyrimidine-8-carboxamide (10 mg, 17.6 umol, 11% yield, 100% purity) as white solid. LCMS for product (ESI+): m/z 568.2 (M+H)⁺, Rt: 2.365 min.

LC/MS (The gradient was 15-90% B in 3.40 min and 90-100% B at 3.40-3.85 min, 100-15% B in 0.01 min, and then held at 15% for 0.64 min, the flow rate was 0.80 ml/min. Mobile phase A was 10 mM Ammonium bicarbonate, mobile phase B was HPLC grade acetonitrile. The column used for chromatography was a 2.1*50 mm Xbridge Shield RPC18 column (5 um particles). Detection methods are diode array (DAD) and evaporative light scattering (ELSD) detection as well as positive electrospray ionization. MS range was 100-1000.

¹H NMR (400 MHz, DMSO-d₆) δ=8.75 (d, J=4.8 Hz, 1H), 8.30 (d, J=7.9 Hz, 1H), 8.19 (d, J=0.6 Hz, 1H), 8.00 (dt, J=1.8, 7.8 Hz, 1H), 7.88 (br s, 3H), 7.59-7.52 (m, 3H), 7.43 (s, 1H), 7.35 (d, J=0.7 Hz, 1H), 7.29 (dd, J=8.3, 12.7 Hz, 1H), 7.22 (br s, 1H), 4.74 (br t, J=6.6 Hz, 2H), 3.05 (br s, 4H), 2.69-2.62 (m, 6H)

### Examples 3: Synthesis of 5-amino-7-[2-[4-(2-fluoro-5-oxazol-2-yl-phenyl)piperazin-1-yl]ethyl]-9-methyl-2-(2-pyridyl)-[1,2,4]triazolopyrrolopyrimidine-8-carboxylic acid

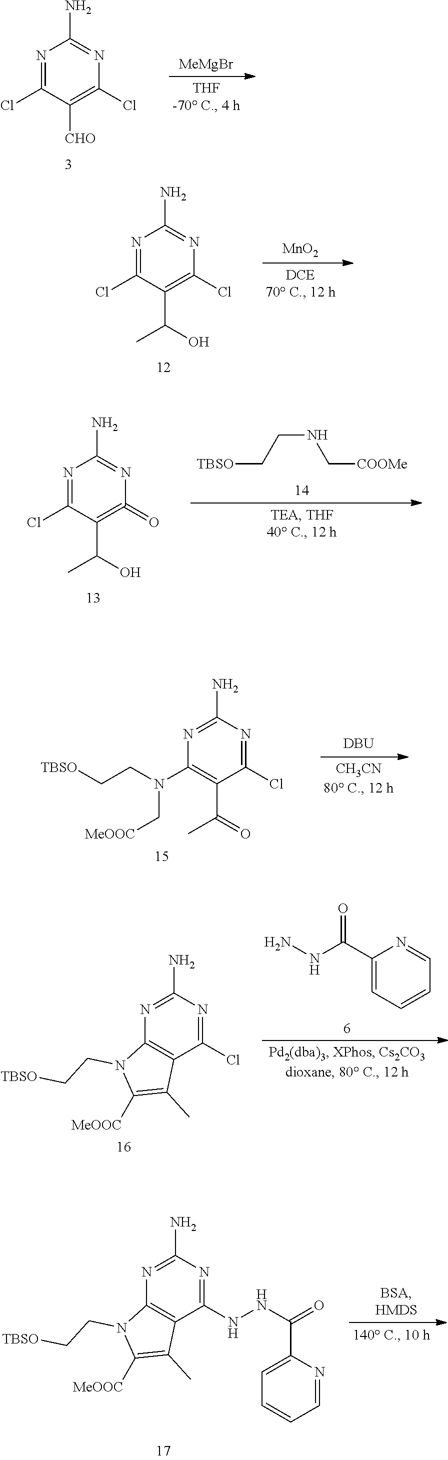

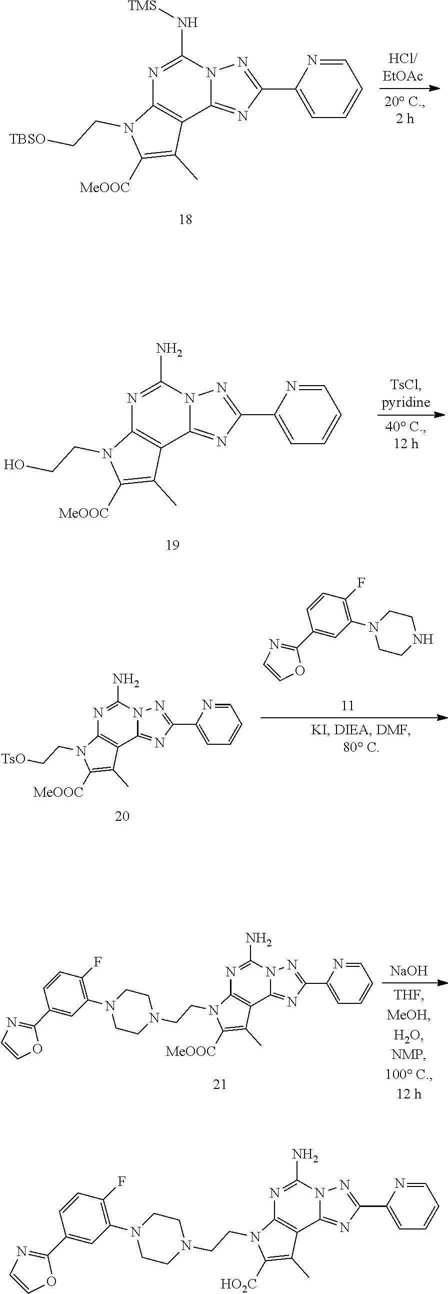

### Step 1: Synthesis of Compound 12

To a solution of 2-amino-4,6-dichloro-pyrimidine-5-carbaldehyde (20 g, 104 mmol, 1 eq) in tetrahydrofuran (1200 mL) was added MeMgBr (3 M, 173 mL, 5 eq) at −70° C. The mixture was stirred at −70° C. for 4 h. TLC (petroleum ether:Ethyl acetate=1:1, Rf=0.43) indicated 15% of starting material remained, and one major new spot with a higher polarity was detected. The reaction mixture was quenched by addition of water (1000 mL) at 0° C. and extracted with ethyl acetate (2*800 mL). The combined organic layers were dried over anhydrous sodium sulfate, filtered and concentrated under reduced pressure to give a yellow solid. The yellow solid 1-(2-amino-4,6-dichloro-pyrimidin-5-yl)ethanol (20 g, 96.1 mmol, 92% yield) was used in the next step without further purification.

### Step 2: Synthesis of Compound 13

To a solution of 1-(2-amino-4,6-dichloro-pyrimidin-5-yl)ethanol (20 g, 96.1 mmol, 1 eq) in 1,2-dichloroethane (2000 mL) was added MnO₂ (200 g, 2.31 mol, 24 eq). The mixture was stirred at 70° C. for 12 h. TLC (petroleum ether:ethyl acetate=1:1, Rf=0.43) indicated complete consumption of starting material. The reaction mixture was filtered and concentrated under reduced pressure to give a white solid. The white solid product 1-(2-amino-4,6-dichloro-pyrimidin-5-yl)ethanone (10 g, 48.5 mmol, 50% yield) was used in the next step without further purification. ¹H NMR (400 MHz, DMSO-d₆) δ=7.89 (s, 2H), 2.52 (s, 3H)

### Step 3: Synthesis of Compound 15

To a solution of methyl 2-[2-[tert-butyl(dimethyl)silyl]oxyethylamino]acetate (11.4 g, 46.1 mmol, 1 eq) and 1-(2-amino-4,6-dichloro-pyrimidin-5-yl)ethanone (9.5 g, 46.1 mmol, 1 eq) in tetrahydrofuran (95 mL) was added triethylamine (7.00 g, 69.1 mmol, 9.63 mL, 1.5 eq). The mixture was stirred at 40° C. for 12 h. TLC (petroleum ether:ethyl acetate=1:1, Rf=0.4) indicated completion of the reaction. The reaction mixture was filtered and concentrated under reduced pressure to give yellow oil. The yellow oil methyl 2-[(5-acetyl-2-amino-6-chloro-pyrimidin-4-yl)-[2-[tert butyl(dimethyl)silyl]oxyethyl]amino]acetate (19 g, 45.5 mmol, 98% yield) was used in the next step without further purification. ¹H NMR (400 MHz, CHLOROFORM-d) δ=4.95 (s, 2H), 4.29 (s, 2H), 3.74 (s, 3H), 3.46 (t, J=5.4 Hz, 2H), 2.60 (s, 3H), 0.87 (s, 9H), 0.04 (s, 6H)

### Step 4: Synthesis of Compound 16

To a solution of methyl 2-[(5-acetyl-2-amino-6-chloro-pyrimidin-4-yl)-[2-[tert-butyl(dimethyl)silyl]oxyethyl]amino]acetate (19 g, 45.5 mmol, 1 eq) in acetonitrile (450 mL) was added 1,8-diazabicyclo[5.4.0]undec-7-ene (6.94 g, 45.5 mmol, 6.87 mL, 1 eq). The mixture was stirred at 80° C. for 12 h. TLC (petroleum ether:ethyl acetate=2:1, Rf=0.75) indicated completion of the reaction. The reaction mixture was concentrated under reduced pressure. The residue was purified by column chromatography (SiO₂, Petroleum ether/Ethyl acetate=10/1 to 5/1). to afford methyl 2-amino-7-[2-[tert-butyl(dimethyl)silyl]oxyethyl]-4-chloro-5-methyl-pyrrolo[2,3-d]pyrimidine-6-carboxylate (12.1 g, 30.3 mmol, 66% yield) as a white solid.

¹H NMR (400 MHz, Chloroform-d) δ=5.07 (s, 2H), 4.61 (t, J=6.0 Hz, 2H), 3.92 (s, 3H), 3.82 (t, J=5.8 Hz, 2H), 2.69 (s, 3H), 0.78 (s, 9H), −0.10 (s, 6H)

### Step 5: Synthesis of Compound 17

A mixture of methyl 2-amino-7-[2-[tert-butyl(dimethyl)silyl]oxyethyl]-4-chloro-5-methyl-pyrrolo[2,3-d]pyrimidine-6-carboxylate (10 g, 25.0 mmol, 1 eq), pyridine-2-carbohydrazide (4.12 g, 30.0 mmol, 1.2 eq), Cs₂CO₃ (24.5 g, 75.20 mmol, 3 eq), tris(dibenzylideneacetone)dipalladium(0) (2.30 g, 2.51 mmol, 0.1 eq) and 2-di-tert-butylphosphino-2,4,6-triisopropylbiphenyl (2.39 g, 5.01 mmol, 0.2 eq) in dioxane (250 mL) was degassed and purged with N₂ 3 times. The mixture was then stirred at 80° C. for 12 h under N₂ atmosphere. TLC (ethyl acetate:methanol=10:1, Rf=0.7) s indicated completion of the reaction. The reaction mixture was filtered and concentrated under reduced pressure.

The residue was purified by column chromatography (SiO₂, Petroleum ether/Ethyl acetate=10/1 to 0/1) to afford methyl 2-amino-7-[2-[tert-butyl(dimethyl)silyl]oxyethyl]-5-methyl-4-[2-(pyridine-2-carbonyl)hydrazino]pyrrolo[2,3-d]pyrimidine-6-carboxylate (10 g, 20.0 mmol, 79% yield) as red solid. ¹H NMR (400 MHz, DMSO-d₆) δ=10.54 (br s, 1H), 8.73-8.65 (m, 2H), 8.11-8.00 (m, 2H), 7.69-7.62 (m, 1H), 6.17 (br s, 2H), 4.44 (br s, 2H), 3.80 (s, 3H), 3.75-3.67 (m, 2H), 2.62 (s, 3H), 0.79 (s, 9H), −0.09 (s, 6H)

### Step 6: Synthesis of Compound 18

To a solution of methyl 2-amino-7-[2-[tert-butyl(dimethyl)silyl]oxyethyl]-5-methyl-4-[2-(pyridine-2-carbonyl)hydrazino]pyrrolo[2,3-d]pyrimidine-6-carboxylate (8.5 g, 17.0 mmol, 1 eq) in hexamethyl disilylamine (40 mL) was added N,O-Bis(trimethylsilyl)acetamide (42.9 g, 210 mmol, 52.1 mL, 12.4 eq). The mixture was stirred at 140° C. for 10 h. TLC (petroleum ether:ethyl acetate=1:1, Rf=0.6) indicated completion of the reaction. The reaction mixture was concentrated under reduced pressure. The crude product was triturated in methanol (85 mL) at 20° C. for 30 min to yield methyl 7-[2-[tert-butyl(dimethyl)silyl]oxyethyl]-9-methyl-2-(2-pyridyl)-5-(trimethylsilylamino)-[1,2,4]triazolopyrrolopyrimidine-8-carboxylate (6 g, 10.8 mmol, 63% yield) as red solid.

¹H NMR (400 MHz, Chloroform-d) δ=8.83 (d, J=4.4 Hz, 1H), 8.47 (d, J=7.8 Hz, 1H), 7.93-7.86 (m, 1H), 7.41 (dd, J=5.4, 6.8 Hz, 1H), 6.09 (s, 1H), 4.77 (t, J=6.1 Hz, 2H), 3.93 (s, 3H), 3.91-3.88 (m, 2H), 2.88 (s, 3H), 0.75 (s, 9H), 0.47 (s, 9H), −0.15 (s, 6H)

### Step 7: Synthesis of Compound 19

Methyl 7-[2-[tert-butyl(dimethyl)silyl]oxyethyl]-9-methyl-2-(2-pyridyl)-5-(trimethylsilylamino)-[1,2,4]triazolopyrrolopyrimidine-8-carboxylate (7.30 g, 13.1 mmol, 1 eq) was added to a solution of HCl in ethyl acetate (4 M, 317 mL, 96.3 eq). The mixture was stirred at 20° C. for 1 h. LC-MS indicated completion of the reaction. The reaction mixture was filtered, and the filter cake was dried under reduced pressure and was used next step without purification. Methyl 5-amino-7-(2-hydroxyethyl)-9-methyl-2-(2-pyridyl)-[1,2,4]triazolopyrrolopyrimidine-8-carboxylate (4.5 g, 11.1 mmol, 84% yield, HCl) was obtained as yellow solid.

¹H NMR (400 MHz, DMSO-d₆) δ=8.80 (d, J=4.2 Hz, 1H), 8.40 (d, J=7.9 Hz, 1H), 8.20 (dt, J=1.5, 7.7 Hz, 1H), 8.00 (br s, 2H), 7.70 (br dd, J=5.1, 6.6 Hz, 1H), 4.56 (br t, J=6.5 Hz, 2H), 3.84 (s, 3H), 3.62 (t, J=6.4 Hz, 2H), 2.75 (s, 3H)

### Step 8: Synthesis of Compound 20

To a solution of methyl 5-amino-7-(2-hydroxyethyl)-9-methyl-2-(2-pyridyl)-[1,2,4]triazolopyrrolopyrimidine-8-carboxylate (4.5 g, 11.1 mmol, 1 eq, HCl) in pyridine (110 mL) was added p-toluenesulfornyl chloride (6.37 g, 33.4 mmol, 3 eq) at 0° C. Then the mixture was stirred at 40° C. for 12 h. LC-MS indicated completion of the reaction. The reaction mixture was concentrated under reduced pressure to give a gray solid. The gray solid was triturated in ethyl acetate (45 mL) and water (45 mL) at 20° C. for 60 min to yield methyl 5-amino-9-methyl-7-[2-(p-tolylsulfonyloxy)ethyl]-2-(2-pyridyl)-[1,2,4]triazolopyrrolopyrimidine-8-carboxylate (7 g, 10.0 mmol, 90% yield, 75% purity) as brown solid. ¹H NMR (400 MHz, DMSO-d₆) δ=8.77 (br d, J=4.2 Hz, 1H), 8.33 (d, J=7.7 Hz, 1H), 8.07-7.94 (m, 3H), 7.60-7.52 (m, 1H), 7.31 (d, J=8.2 Hz, 2H), 7.04 (d, J=8.2 Hz, 2H), 4.69 (br t, J=4.7 Hz, 2H), 4.45 (br t, J=4.7 Hz, 2H), 3.80 (s, 3H), 2.68 (s, 3H), 2.14 (s, 3H).

### Step 9: Synthesis of Compound 21

A mixture of methyl 5-amino-9-methyl-7-[2-(p-tolylsulfonyloxy)ethyl]-2-(2-pyridyl)-[1,2,4]triazolopyrrolopyrimidine-8-carboxylate (0.3 g, 575 umol, 1 eq), 2-(4-fluoro-3-piperazin-1-yl-phenyl)oxazole (284 mg, 1.15 mmol, 2 eq), potassium iodide (95 mg, 575 umol, 1 eq) and ethyldiisopropylamine (297 mg, 2.30 mmol, 400 uL, 4 eq) in dimethylamine (5.7 mL) was degassed and purged with N₂ 3 times. Then the mixture was stirred at 80° C. for 12 h under N₂ atmosphere. LC-MS indicated completion of the reaction. 1 M hydrochloric acid (3 mL) was added to the reaction mixture which became a clear solution upon addition of the acid. The product was purified by prep-HPLC (HCl condition). column: Phenomenex luna C18 250*50 mm*10 um; mobile phase: [water (0.05% HCl)-ACN]; B %: 15%-45%, 10 min to give methyl 5-amino-7-[2-[4-(2-fluoro-5-oxazol-2-yl-phenyl)piperazin-1-yl]ethyl]-9-methyl-2-(2-pyridyl)-[1,2,4]triazolopyrrolopyrimidine-8-carboxylate (45 mg, 68.6 umol, 11% yield, 91% purity) as yellow solid. LCMS for product (ESI+): m/z 597.2 (M+H)⁺, Rt: 3.722 min.

LC/MS (The gradient was 30-50% B in 6.00 min, 50%-100% B in 2.00 min, the flow rate was 1.00 mL/min. Mobile phase A was 0.037% Trifluoroacetic Acid in water, mobile phase B was 0.018% Trifluoroacetic Acid in acetonitrile. The column used for chromatography was Ascentis Express HPLC Column C18 10 cm*4.6 mm (2.7 um particles). Detection methods are diode array (DAD) and evaporative light scattering (ELSD) detection as well as positive electrospray ionization. MS range was 100-1000.

¹H NMR (400 MHz, DMSO-d₆) δ=10.14 (br s, 1H), 8.77 (d, J=4.9 Hz, 1H), 8.34 (d, J=7.8 Hz, 1H), 8.26-8.16 (m, 3H), 8.06 (dt, J=1.5, 7.8 Hz, 1H), 7.67-7.63 (m, 1H), 7.62-7.57 (m, 2H), 7.44-7.32 (m, 2H), 4.85 (br t, J=5.6 Hz, 2H), 4.03 (br d, J=10.8 Hz, 2H), 3.89 (s, 3H), 3.71-3.59 (m, 4H), 3.35 (br d, J=10.8 Hz, 2H), 3.26-3.15 (m, 2H), 2.80 (s, 3H)

### Step 10: Synthesis of 5-amino-7-[2-[4-(2-fluoro-5-oxazol-2-yl-phenyl)piperazin-1-yl]ethyl]-9-methyl-2-(2-pyridyl)-[1,2,4]triazolopyrrolopyrimidine-8-carboxylic acid

To a suspension of methyl 5-amino-7-[2-[4-(2-fluoro-5-oxazol-2-yl-phenyl)piperazin-1-yl]ethyl]-9-methyl-2-(2-pyridyl)-[1,2,4]triazolopyrrolopyrimidine-8-carboxylate (30 mg, 50.2 umol, 1 eq) in tetrahydrofuran (0.6 mL), N-Methyl-2-pyrrolidone (1.5 mL) and methanol (0.6 mL) was added NaOH (14.0 mg, 351 umol, 7 eq) in H₂O (0.3 mL). The mixture was stirred at 100° C. for 12 h. LC-MS indicated completion of the reaction. The reaction mixture was concentrated under reduced pressure to yield a red liquid. The red liquid was purified by prep-HPLC (neutral condition). column: Welch Ultimate C18 150*25 mm*5 um; mobile phase: [water (10 mM NH4HCO3)-ACN]; B %: 20%-50%, 10 min to give 5-amino-7-[2-[4-(2-fluoro-5-oxazol-2-yl-phenyl)piperazin-1-yl]ethyl]-9-methyl-2-(2-pyridyl)-[1,2,4]triazolopyrrolopyrimidine-8-carboxylic acid (4 mg, 6.87 umol, 13% yield) as white powder. LCMS for product (ESI+): m/z 583.2 (M+H)⁺, Rt: 2.100 min.

LC/MS (The gradient was 10-100% B in 3.40 min with a hold at 100% B for 0.45 min, 100-10% B in 0.01 min, and then held at 10% for 0.64 min, the flow rate was 0.80 mL/min. Mobile phase A was 0.037% Trifluoroacetic Acid in water, mobile phase B was 0.018% Trifluoroacetic Acid in acetonitrile. The column used for chromatography was a Luna-C18(2) 2.0*50 mm column (5 um particles). Detection methods are diode array (DAD) and evaporative light scattering (ELSD) detection as well as positive electrospray ionization. MS range was 100-1000.

¹H NMR (400 MHz, DMSO-d₆) δ=8.75 (d, J=4.4 Hz, 1H), 8.31 (d, J=7.8 Hz, 1H), 8.20 (s, 1H), 8.01 (t, J=8.1 Hz, 1H), 7.93 (br s, 1H), 7.55 (br d, J=3.9 Hz, 3H), 7.36 (s, 1H), 7.29 (dd, J=8.3, 12.7 Hz, 1H), 4.71-4.64 (m, 2H), 3.31 (br s, 2H), 3.05 (br s, 4H), 2.76 (s, 3H), 2.67 (br s, 6H).

### Examples 4: Synthesis of 5-amino-7-[2-[4-(2-fluoro-5-oxazol-2-yl-phenyl)piperazin-1-yl]ethyl]-N,9-dimethyl-2-(2-pyridyl)-[1,2,4]triazolopyrrolopyrimidine-8-carboxamide

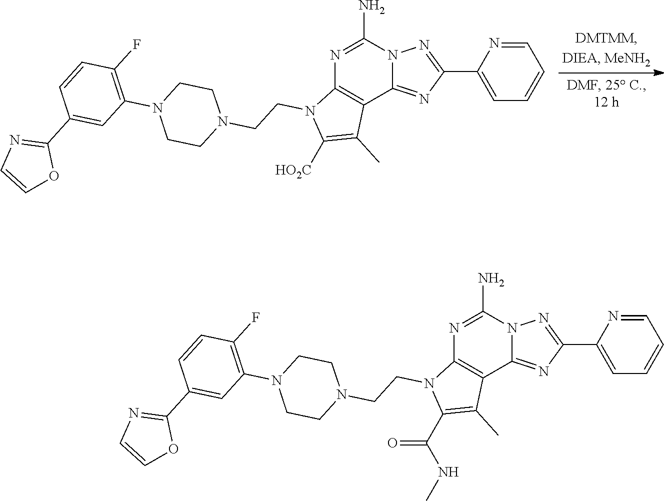 [SMILES: Cc1c(C(=O)O)n(CCN2CCN(c3cc(-c4ncco4)ccc3F)CC2)c2nc(N)n3nc(-c4ccccn4)nc3c12 ; CNC(=O)c1c(C)c2c(nc(N)n3nc(-c4ccccn4)nc23)n1CCN1CCN(c2cc(-c3ncco3)ccc2F)CC1]

To a solution of 5-amino-7-[2-[4-(2-fluoro-5-oxazol-2-yl-phenyl)piperazin-1-yl]ethyl]-9-methyl-2-(2-pyridyl)-[1,2,4]triazolopyrrolopyrimidine-8-carboxylic acid (15 mg, 25.7 umol, 1 eq) in N,N-dimethylformamide (1 mL) was added 4-(4,6-dimethoxy-1,3,5-triazin-2-yl)-4-methyl-morpholin-4-ium; tetrafluoroborate (16.8 mg, 51.4 umol, 2 eq) and N,N-diisopropylethylamine (16.6 mg, 128 umol, 22.4 uL, 5 eq). The mixture was stirred at 25° C. for 2 h. Then a solution of methylamine in tetrahydrofuran (2 M, 51.4 uL, 4 eq) was added to the mixture and the stirring was kept at 25° C. for 10 h.

N,N-dimethylformamide (1 mL) was added to the reaction mixture. The reaction mixture was purified by prep-HPLC (HCl condition). column: Phenomenex Luna C18 150*30 mm*5 um; mobile phase: [water (0.04% HCl)-ACN]; B %: 25%-50%, 10 min to afford 5-amino-7-[2-[4-(2-fluoro-5-oxazol-2-yl-phenyl)piperazin-1-yl]ethyl]-N, 9-dimethyl-2-(2-pyridyl)-[1,2,4]triazolopyrrolopyrimidine-8-carboxamide (4 mg, 6.33 umol, 24% yield, 100% purity, HCl) as orange solid. LCMS for product (ESI+): m/z 596.1 (M+H)⁺, Rt: 1.953 min.

LC/MS (The gradient was 5% B at 0-0.35 min, 5-95% B at 0.35-2.00 min and 95-100% B at 2.0-3.8 min, 100-5% B in 0.01 min, and then held at 5% B for 0.49 min, the flow rate was 0.80 mL/min. Mobile phase A was 0.037% Trifluoroacetic Acid in water, mobile phase B was 0.018% Trifluoroacetic Acid in acetonitrile. The column used for chromatography was a Luna-C18(2) 2.0*50 mm column (5 um particles). Detection methods are diode array (DAD) and evaporative light scattering (ELSD) detection as well as positive electrospray ionization. MS range was 100-1000.

¹H NMR (400 MHz, METHANOL-d₄) δ=9.00-8.91 (m, 2H), 8.80 (dt, J=1.5, 7.8 Hz, 1H), 8.24-8.18 (m, 1H), 8.00 (s, 1H), 7.76-7.70 (m, 2H), 7.33-7.23 (m, 2H), 4.75 (br t, J=5.1 Hz, 2H), 3.85-3.76 (m, 4H), 3.71 (br d, J=13.2 Hz, 2H), 3.50-3.41 (m, 2H), 3.30-3.24 (m, 2H), 3.04 (s, 3H), 2.79 (s, 3H)

### Example 5: Synthesis of 5-amino-7-[2-[4-(2-fluoro-5-oxazol-2-yl-phenyl)piperazin-1-yl]ethyl]-N-cyclopropyl-9-methyl-2-(2-pyridyl)-[1,2,4]triazolopyrrolopyrimidine-8-carboxamide

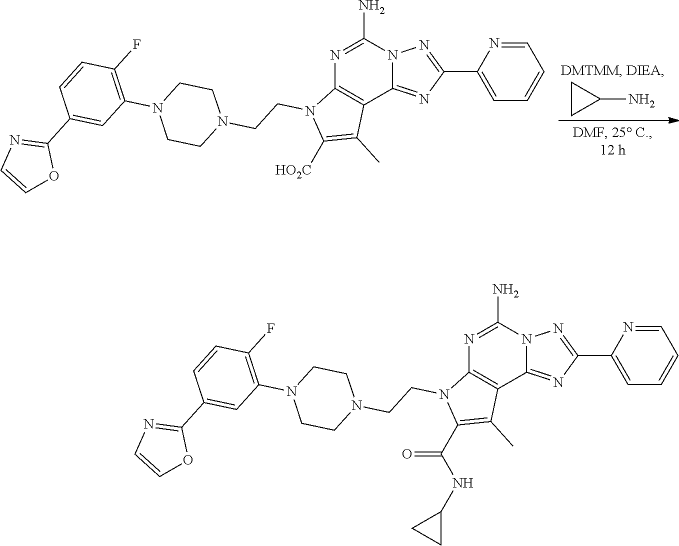

The title compound was prepared following the procedures outlined in Example 4.

### Example 6: Synthesis of 5-amino-7-[2-[4-(2-fluoro-5-oxazol-2-yl-phenyl)piperazin-1-yl]ethyl]-9-methyl-2-(pyridin-2-yl)-7H-pyrrolo[3,2-e][1,2,4]triazolo[1,5-c]pyrimidin-8-yl)(azetidin-1-yl)methanone

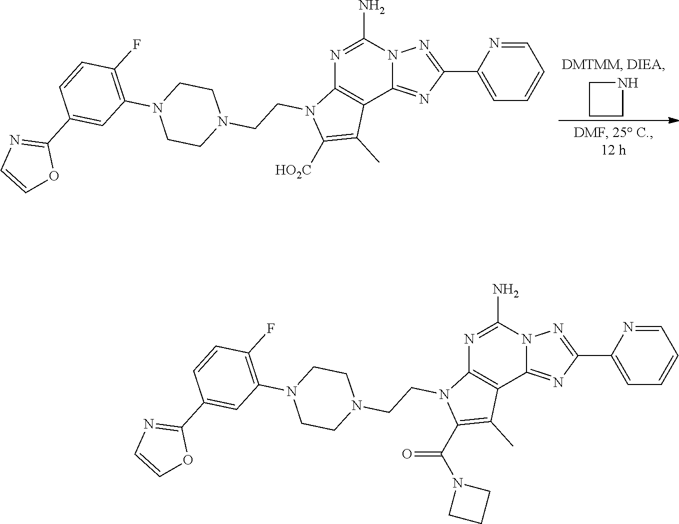

The title compound was prepared following the procedures outlined in Example 4.

### Example 7: Synthesis of 5-amino-9-chloro-7-[2-[4-(6-fluoro-3-methyl-1,2-benzoxazol-5-yl)piperazin-1-yl]ethyl]-2-(2-pyridyl)-[1,2,4]triazolopyrrolopyrimidine-8-carbonitrile

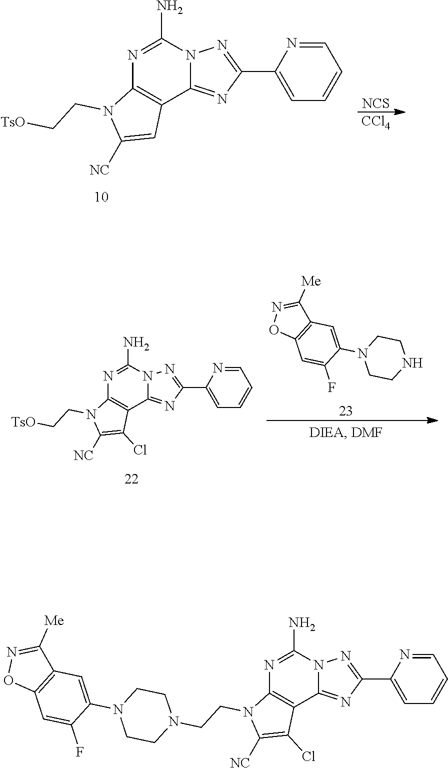

### Step 1: Synthesis of Compound 22

To a solution of 2-[5-amino-8-cyano-2-(2-pyridyl)-[1,2,4]triazolopyrrolopyrimidin-7-yl]ethyl 4-methylbenzenesulfonate (100 mg, 211 umol, 1 eq) in CCl₄ (5 mL) was added N-chlorosuccinimide (56.3 mg, 422 umol, 2 eq). The reaction was stirred at 80° C. for 3 h. LCMS indicated completion of the reaction. The mixture was concentrated under reduced pressure to afford a crude product. The crude product was purified by re-crystallization from methyl tertiary butyl ether (20 mL) at 60° C. to yield 2-[5-amino-9-chloro-8-cyano-2-(2-pyridyl)-[1,2,4]triazolopyrrolopyrimidin-7-yl]ethyl 4-methylbenzenesulfonate (120 mg, 189 umol, 60% yield, 80% purity) as a white solid.

### Step 2: Synthesis of 5-amino-9-chloro-7-[2-[4-(6-fluoro-3-methyl-1,2-benzoxazol-5-yl)piperazin-1-yl]ethyl]-2-(2-pyridyl)-[1,2,4]triazolopyrrolopyrimidine-8-carbonitrile

To a solution of 2-[5-amino-9-chloro-8-cyano-2-(2-pyridyl)-[1,2,4]triazolopyrrolopyrimidin-7-yl]ethyl 4-methylbenzenesulfonate (115 mg, 226 umol, 1 eq) and 6-fluoro-3-methyl-5-piperazin-1-yl-1,2-benzoxazole (53.2 mg, 226 umol, 1 eq) in dimethyl formamide (4 mL) was added diisopropylethylamine (43.8 mg, 339 umol, 59.0 uL, 1.5 eq). The mixture was stirred at 80° C. for 48 hr under N₂.

LC-MS showed all of 2-[5-amino-9-chloro-8-cyano-2-(2-pyridyl)-[1,2,4]triazolopyrrolopyrimidin-7-yl]ethyl 4-methylbenzenesulfonate was consumed. Several new peaks were observed on LC-MS, ˜45% of side-products and ˜30% of desired compound were detected. The mixture was filtered. The filtrate was purified using prep-HPLC (neutral condition) to yield 5-amino-9-chloro-7-[2-[4-(6-fluoro-3-methyl-1,2-benzoxazol-5-yl)piperazin-1-yl]ethyl]-2-(2-pyridyl)-[1,2,4]triazolopyrrolopyrimidine-8-carbonitrile (1.9 mg, 3.25 umol, 1.4% yield, 97.8% purity) as brown solid. LCMS for product (ESI+): m/z 572.1 (M+H)⁺, Rt: 3.042 min.

LC/MS (The gradient was 15-90% B in 3.40 min and 90-100% B at 3.40-3.85 min, 100-15% B in 0.01 min, and then held at 15% for 0.64 min, the flow rate was 0.80 mL/min. Mobile phase A was 10 mM ammonium bicarbonate, mobile phase B was HPLC grade acetonitrile. The column used for chromatography was a 2.1*50 mm Xbridge Shield RPC18 column (5 um particles). Detection methods are diode array (DAD) and evaporative light scattering (ELSD) detection as well as positive electrospray ionization. MS range was 100-1000.

¹H NMR (400 MHz, DMSO-d₆) δ=8.76 (br d, J=4.3 Hz, 1H), 8.36 (br s, 1H), 8.30 (d, J=7.9 Hz, 1H), 8.03 (t, J=7.8 Hz, 1H), 7.64 (d, J=11.7 Hz, 1H), 7.59-7.54 (m, 1H), 7.40 (d, J=8.1 Hz, 1H), 4.35 (br s, 2H), 2.98 (br s, 4H), 2.78 (br d, J=5.1 Hz, 2H), 2.67 (br s, 4H), 2.56-2.54 (m, 3H).

### Example 8: Synthesis of 2-[4-[4-[2-[5-amino-8-cyano-2-(2-pyridyl)-[1,2,4]triazolopyrrolopyrimidin-7-yl]ethyl]piperazin-1-yl]phenoxy]acetic acid

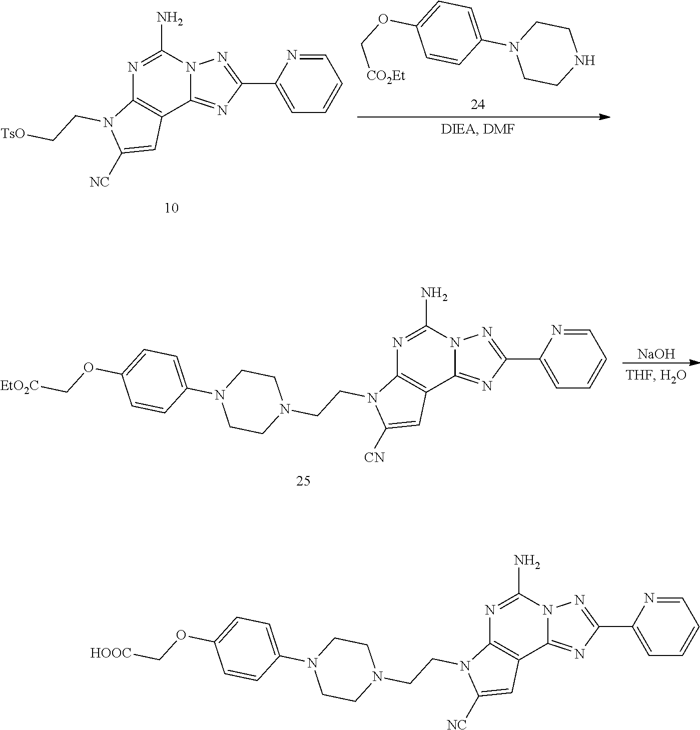

### Step 1: Synthesis of ethyl 2-[4-[4-[2-[5-amino-8-cyano-2-(2-pyridyl)-[1,2,4]triazolopyrrolopyrimidin-7-yl]ethyl]piperazin-1-yl]phenoxy]acetate (Compound 25)

To a solution of 2-[5-amino-8-cyano-2-(2-pyridyl)-[1,2,4]triazolopyrrolopyrimidin-7-yl]ethyl 4-methylbenzenesulfonate (500 mg, 1.05 mmol, 1 eq) and ethyl 2-(4-piperazin-1-ylphenoxy) acetate (279 mg, 1.05 mmol, 1 eq) in DMF (20 mL) was added DIEA (204 mg, 1.58 mmol, 275 uL, 1.5 eq). The reaction was stirred at 80° C. for 12 hr. LCMS showed the starting material remained and the desired product was formed.

The reaction was concentrated under reduced pressure and residue was purified by prep-HPLC (neutral condition) to give ethyl 2-[4-[4-[2-[5-amino-8-cyano-2-(2-pyridyl)-[1,2,4]triazolopyrrolopyrimidin-7-yl]ethyl]piperazin-1-yl]phenoxy]acetate (300 mg, 529 umol, 50% yield) as white solid that was used without further purification.

### Step 2: Synthesis of 2-[4-[4-[2-[5-amino-8-cyano-2-(2-pyridyl)-[1,2,4]triazolopyrrolopyrimidin-7-yl]ethyl]piperazin-1-yl]phenoxy]acetic acid

Ethyl 2-[4-[4-[2-[5-amino-8-cyano-2-(2-pyridyl)-[1,2,4]triazolopyrrolopyrimidin-7-yl]ethyl]piperazin-1-yl]phenoxy]acetate (330 mg, 582 umol, 1 eq) was dissolved in a solution of NaOH (160 mg, 4.00 mmol, 6.87 eq) in H₂O (4 mL) and THF (6 mL). The reaction was stirred at 20° C. for 1 hr. The mixture was acidified to pH 7 with acetic acid and then concentrated under reduced pressure to give crude product. The crude product was purified by prep-HPLC (neutral condition) to give 2-[4-[4-[2-[5-amino-8-cyano-2-(2-pyridyl)-[1,2,4]triazolopyrrolopyrimidin-7-yl]ethyl]piperazin-1-yl]phenoxy]acetic acid (150 mg, 267 umol, 46% yield, 95.7% purity) as white solid. LCMS for product (ESI+): m/z 539.2 (M+H)⁺, Rt: 1.808 min.

LC/MS(The gradient was 15-90% B in 3.40 min and 90-100% B at 3.40-3.85 min, 100-15% B in 0.01 min, and then held at 15% for 0.64 min, the flow rate was 0.80 ml/min. Mobile phase A was 10 mM Ammonium bicarbonate, mobile phase B was HPLC grade acetonitrile. The column used for chromatography was a 2.1*50 mm Xbridge Shield RPC18 column (5 um particles). Detection methods are diode array (DAD) and evaporative light scattering (ELSD) detection as well as positive electrospray ionization. MS range was 100-1000.

¹H NMR (400 MHz, DMSO-d₆) δ=8.75 (d, J=4.2 Hz, 1H), 8.30 (d, J=7.8 Hz, 1H), 8.16 (br s, 1H), 8.01 (dt, J=1.7, 7.8 Hz, 1H), 7.66 (s, 1H), 7.55 (dd, J=5.3, 7.0 Hz, 1H), 6.80 (d, J=9.2 Hz, 2H), 6.70 (d, J=9.0 Hz, 2H), 4.35 (br t, J=6.0 Hz, 2H), 4.20 (s, 2H), 2.96 (br s, 4H), 2.75 (br t, J=6.1 Hz, 2H), 2.59 (br s, 4H)

### Example 9: Synthesis of 5-amino-9-chloro-7-[2-[4-(6-fluoro-3-methyl-1,2-benzoxazol-5-yl)piperazin-1-yl]ethyl]-2-(2-pyridyl)-[1,2,4]triazolo[1,5-c]pyrrolo[3,2-e]pyrimidine-8-carboxamide

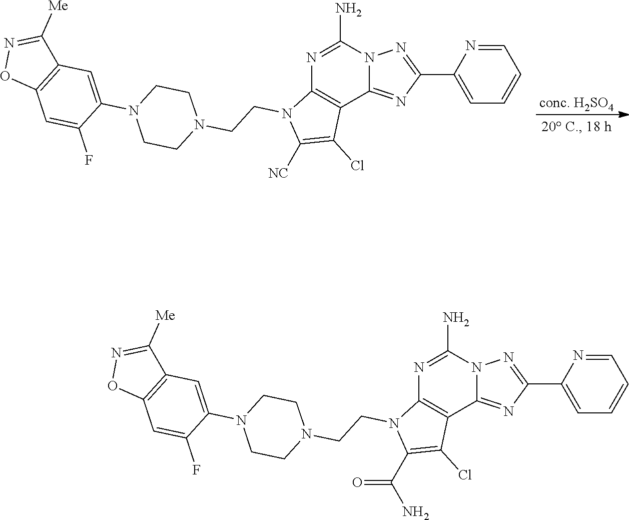

A solution of 5-amino-9-chloro-7-[2-[4-(6-fluoro-3-methyl-1,2-benzoxazol-5-yl)piperazin-1-yl]ethyl]-2-(2-pyridyl)-[1,2,4]triazolo[BLAH]pyrrolo[BLAH]pyrimidine-8-carbonitrile (10.0 mg, 16.4 umol, 1.00 eq, HCl) in H₂SO₄ (18.4 M, 0.7 mL) was stirred at 20° C. for 18 h. LC-MS showed 5-amino-9-chloro-7-[2-[4-(6-fluoro-3-methyl-1,2-benzoxazol-5-yl)piperazin-1-yl]ethyl]-2-(2-pyridyl)-[1,2,4]triazolopyrrolopyrimidine-8-carbonitrile was consumed and one main peak with desired mass was detected. The reaction mixture was added to saturated aqueous NH₄OH (7 M, 5 mL) at 0° C. The mixture was concentrated to give a white solid.

The solid was purified by prep-HPLC (HCl condition; column: Phenomenex Luna C18 150*30 mm*5 um; mobile phase: [water (0.04% HCl)-ACN]; B %: 15%-45%, 10 min.) to afford 5-amino-9-chloro-7-[2-[4-(6-fluoro-3-methyl-1,2-benzoxazol-5-yl)piperazin-1-yl]ethyl]-2-(2-pyridyl)-[1,2,4]triazolo[1,5-c]pyrrolo[3,2-e]pyrimidine-8-carboxamide (7.40 mg, 11.2 umol, 68% yield, 94.7% purity, HCl) was obtained as a yellow solid. LCMS for product (ESI+): m/z 590.1 (M+H)⁺, Rt: 2.112 min.

LC/MS(The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.037% trifluoroacetic acid in water, mobile phase B was 0.018% trifluoroacetic acid in acetonitrile. The column used for chromatography was a Kinetex C18 50*2.1 mm column (Sum particles). Detection methods are diode array (DAD) and evaporative light scattering (ELSD) detection as well as positive electrospray ionization. MS range was 100-1000.

¹H NMR (400 MHz, METHANOL-d₄) δ=8.92 (d, J=5.3 Hz, 1H), 8.83 (d, J=7.9 Hz, 1H), 8.62 (dt, J=1.3, 7.9 Hz, 1H), 8.07 (t, J=6.2 Hz, 1H), 7.47 (s, 1H), 7.45 (d, J=3.7 Hz, 1H), 4.92 (br s, 2H), 3.93 (br d, J=12.0 Hz, 2H), 3.85 (br t, J=5.3 Hz, 2H), 3.63 (br d, J=12.7 Hz, 2H), 3.52-3.43 (m, 2H), 3.29-3.20 (m, 2H), 2.55 (s, 3H)

### Example 10: Synthesis of 2-[4-[4-[2-[5-amino-8-carbamoyl-2-(2-pyridyl)-[1,2,4]triazolo[1,5-e]pyrrolo[3,2-e]pyrimidin-7-yl]ethyl]piperazin-1-yl]phenoxy]acetic acid

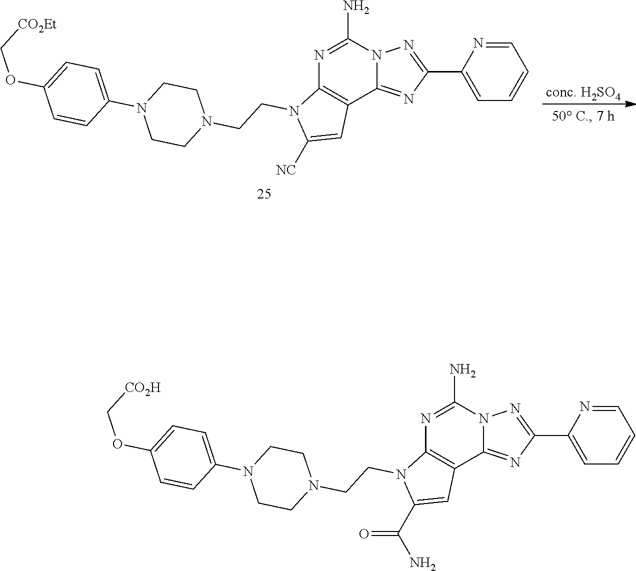

A solution of ethyl 2-[4-[4-[2-[5-amino-8-cyano-2-(2-pyridyl)-[1,2,4]triazolopyrrolopyrimidin-7-yl]ethyl]piperazin-1-yl]phenoxy]acetate (35.0 mg, 61.8 umol, 1.00 eq) in H₂SO₄ (2 mL) was stirred at 50° C. for 7 h. LC-MS showed ethyl 2-[4-[4-[2-[5-amino-8-cyano-2-(2-pyridyl)-[1,2,4]triazolo[1,5-e]pyrrolo[3,2-e]pyrimidin-7-yl]ethyl]piperazin-1-yl]phenoxy]acetate was consumed and one main peak with desired mass was detected. The reaction mixture was added to an aqueous NH₄OH (7M, 10 mL) at 0° C. dropwise. The mixture was concentrated to give a white solid. The solid was purified by prep-HPLC to afford 2-[4-[4-[2-[5-amino-8-carbamoyl-2-(2-pyridyl)-[1,2,4]triazolo[1,5-e]pyrrolo[3,2-e]pyrimidin-7-yl]ethyl]piperazin-1-yl]phenoxy]acetic acid (38.0 mg, 68.3 umol, 55% yield, 100% purity) as a yellow solid. (HCl condition; column: Phenomenex Luna C18 150*30 mm*5 um; mobile phase: [water (0.04% HCl)-ACN]; B %: 5%-30%, 10 min). LCMS for product (ESI+): m/z 557.2 (M+H)⁺, Rt: 1.637 min.

LC/MS: 5_95AB_6 min-220-254-ELSD: LC/MS(The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.037% trifluoroacetic acid in water, mobile phase B was 0.018% trifluoroacetic acid in acetonitrile. The column used for chromatography was a Kinetex C18 50*2.1 mm column (5 um particles). Detection methods are diode array (DAD) and evaporative light scattering (ELSD) detection as well as positive electrospray ionization. MS range was 100-1000.

¹H NMR (400 MHz, DMSO-d₆) δ=9.91 (br s, 1H), 8.77 (br d, J=4.3 Hz, 1H), 8.34 (d, J=7.8 Hz, 1H), 8.12-8.03 (m, 2H), 8.14-8.01 (m, 1H), 7.64-7.58 (m, 2H), 7.42 (br s, 1H), 6.95 (br d, J=8.9 Hz, 2H), 6.88-6.82 (m, 2H), 4.90 (br s, 2H), 4.59 (s, 2H), 3.88-3.86 (m, 3H), 3.74-3.61 (m, 3H), 3.28 (br s, 2H), 2.98 (br t, J=11.9 Hz, 2H)

### Example 11: Synthesis of 9-chloro-7-[2-[4-(6-fluoro-3-methyl-1,2-benzoxazol-5-yl)piperazin-1-yl]ethyl]-2-(2-pyridyl)-[1,2,4]triazolo-pyrazolo-pyrimidin-5-amine

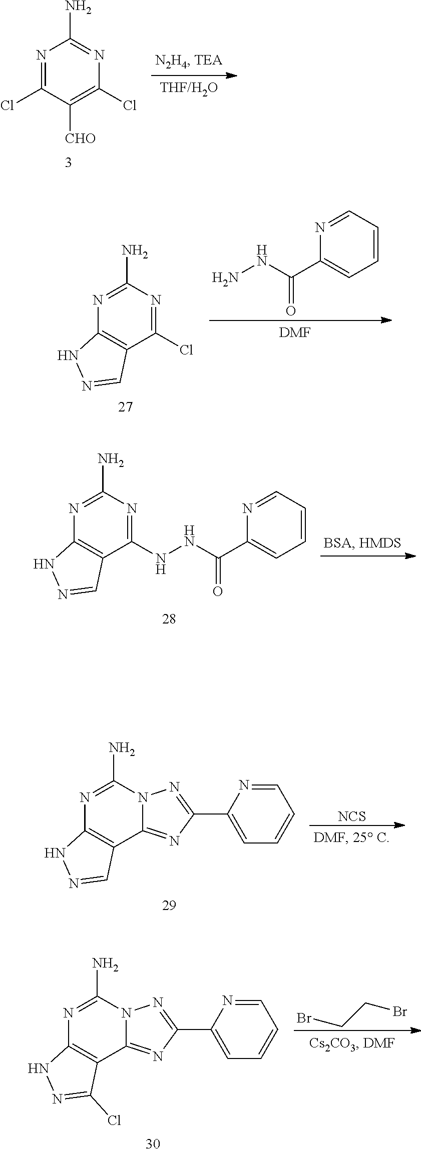

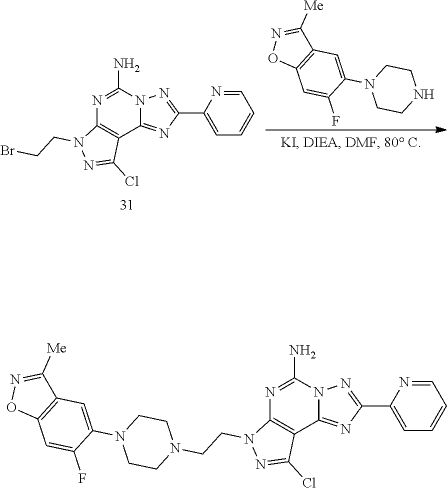 [SMILES: Nc1nc2c(c(Cl)nn2CCBr)c2nc(-c3ccccn3)nn12 ; Cc1noc2cc(F)c(N3CCNCC3)cc12 ; Cc1noc2cc(F)c(N3CCN(CCn4nc(Cl)c5c4nc(N)n4nc(-c6ccccn6)nc54)CC3)cc12]

### Step 1: Synthesis of Compound 27

To a mixture of 2-amino-4,6-dichloro-pyrimidine-5-carbaldehyde (50 g, 260 mmol, 1 eq) and TEA (30.3 g, 299 mmol, 41.7 mL, 1.15 eq) in THE (1.04 L) and H₂O (104 mL) was added NH₂NH₂.H₂O (13.0 g, 260 mmol, 12.7 mL, 1 eq). The mixture was stirred at 20° C. for 15 hr. LCMS showed the desired peak was detected. (a sample diluted with DMF)

The reaction mixture was separated by decantation after the reaction. The liquid was concentrated to give a solid. The solid was triturated with H₂O (1.5 L) at 25° C. for 30 min to give 4-chloro-1H-pyrazolo[3,4-d]pyrimidin-6-amine (45 g, crude) as yellow solid.

¹H NMR (400 MHz, DMSO-d₆) δ=13.24 (br s, 1H), 7.94 (d, J=1.2 Hz, 1H), 7.13 (br s, 2H)

### Step 2: Synthesis of Compound 28

To a mixture of pyridine-2-carbohydrazide (12.9 g, 94.3 mmol, 1 eq) in DMF (160 mL) was added 4-chloro-1H-pyrazolo[3,4-d]pyrimidin-6-amine (16 g, 94.3 mmol, 1 eq). The mixture was stirred for 16 h at 110° C. LCMS showed main desired MS was detected.

The reaction mixture was concentrated to dryness. The crude product was triturated with EA (20 mL). The mixture was filtered and the filter cake was dried under vacuum to give N′-(6-amino-1H-pyrazolo[3,4-d]pyrimidin-4-yl)pyridine-2-carbohydrazide (20 g, 74.0 mmol, 78% yield) as white solid. ¹H NMR (400 MHz, DMSO-d₆) δ=8.77-8.65 (m, 1H), 8.46 (s, 1H), 8.37 (br s, 2H), 8.12-7.95 (m, 2H), 7.90 (s, 1H), 7.70-7.55 (m, 1H)

### Step 3: Synthesis of Compound 29

To a mixture of N′-(6-amino-1H-pyrazolo[3,4-d]pyrimidin-4-yl)pyridine-2-carbohydrazide (900 mg, 3.33 mmol, 1 eq) in HMDS (18 mL) was added BSA (4.95 g, 24.3 mmol, 6.01 mL, 7.3 eq). The mixture was stirred at 130° C. for 16 h. LCMS showed the desired MS was detected. The reaction was concentrated to give a pasty residue. The residue was concentrated in high vacuo and then triturated in MeOH (3 mL) to give 2-(2-pyridyl)-7H-[1,2,4]triazolo-pyrazolo-pyrimidin-5-amine (400 mg, 1.59 mmol, 47% yield) as brown solid.

¹H NMR (400 MHz, DMSO-d₆) δ=8.77-8.65 (m, 1H), 8.46 (s, 1H), 8.37 (br s, 2H), 8.12-7.95 (m, 2H), 7.90 (s, 1H), 7.70-7.55 (m, 1H)

### Step 4: Synthesis of Compound 30

To a solution of 2-(2-pyridyl)-7H-[1,2,4]triazolo-pyrazolo-pyrimidin-5-amine (3.9 g, 15.4 mmol, 1 eq) in DMF (700 mL) was added NCS (3.92 g, 29.4 mmol, 1.9 eq). The mixture was stirred at 25° C. for 20 hr.

HPLC (product: RT=2.86 min; starting material: RT=2.41 min) showed the starting material was consumed completely. The reaction was quenched by addition of an aqueous solution of Na₂SO₃ (30 mL) at 0° C. After quenching the reaction, the reaction mixture was concentrated under reduced pressure to remove DMF (700 mL). Then H₂O (30 mL) was added and the suspension was stirred for 30 min. The solid was collected by filtering. The crude product was purified by re-crystallization from MeCN (25 mL). Compound 9-chloro-2-(2-pyridyl)-7H-[1,2,4]triazolo-pyrazolo-pyrimidin-5-amine (3.4 g, 11.8 mmol, 76% yield) was obtained as a a yellow solid.

¹H NMR (400 MHz, DMSO-d₆) δ=13.55 (s, 1H), 8.76 (d, J=4 Hz, 1H), 8.30 (d, J=8 Hz, 1H), 8.20 (brs, 2H), 8.05-8.02 (m, 1H), 7.58-7.54 (m, 1H)

### Step 5: Synthesis of Compound 31

To a solution of 9-chloro-2-(2-pyridyl)-7H-[1,2,4]triazolo-pyrazolo-pyrimidin-5-amine (1.5 g, 5.23 mmol, 1 eq) and 1,2-dibromoethane (1.28 g, 6.80 mmol, 1.3 eq) in DMF (15 mL) was added Cs₂CO₃ (3.41 g, 10.4 mmol, 2 eq). The mixture was stirred at 25° C. for 2 hr. LCMS showed main desired MS was detected. The reaction mixture was added into H₂O (100 mL) and stirred 30 min. The solid was collected by filtering to give crude 7-(2-bromoethyl)-9-chloro-2-(2-pyridyl)-[1,2,4]triazolo-pyrazolo-pyrimidin-5-amine (1.7 g, 4.32 mmol, 82% yield) was obtained as a brown solid. ¹H NMR (400 MHz, DMSO-d₆) δ=8.76 (d, J=4.4 Hz, 1H), 8.40 (brs, 1H), 8.31 (d, J=8 Hz, 1H), 8.25 (brs, 1H), 8.04-8.00 (m, 1H), 7.58-7.55 (m, 1H), 4.63 (t, J=6.0 Hz, 2H), 3.94 (t, J=6.0 Hz, 2H)

### Step 6: Synthesis of 9-chloro-7-[2-[4-(6-fluoro-3-methyl-1,2-benzoxazol-5-yl)piperazin-1-yl]ethyl]-2-(2-pyridyl)-[1,2,4]triazolo-pyrazolo-pyrimidin-5-amine

To a solution of 7-(2-bromoethyl)-9-chloro-2-(2-pyridyl)-[1,2,4]triazolo-pyrazolo-pyrimidin-5-amine (100 mg, 254 umol, 1 eq) and 6-fluoro-3-methyl-5-piperazin-1-yl-1,2-benzoxazole (59.9 mg, 254 umol, 1 eq) in DMF (3 mL) was added NaI (38.1 mg, 254 umol, 1 eq) and DIEA (65.8 mg, 509 umol, 88.7 uL, 2 eq). The mixture was stirred at 80° C. for 16 hr.

LCMS showed main desired MS was detected. The reaction mixture was filtered and the filtration was concentrated under reduced pressure to remove DMF (3 mL) to give a residue. The crude product was triturated with EtOAc (2 mL) and MeOH (1 mL) at 0° C. for 30 min.

To give compound 9-chloro-7-[2-[4-(6-fluoro-3-methyl-1,2-benzoxazol-5-yl)piperazin-1-yl]ethyl]-2-(2-pyridyl)-[1,2,4]triazolo-pyrazolo-pyrimidin-5-amine (6 mg, 10.6 umol, 4% yield, 96.7% purity) was obtained as a white solid. LCMS: 96.7% purity, m/z=548.1 (M+1), t_{R}=2.755 min.

LC/MS (The gradient was 10-100% B in 3.4 min with a hold at 100% B for 0.45 min, 100-10% B in 0.01 min, and then held at 10% B for 0.65 min (0.8 mL/min flow rate). Mobile phase A was 0.0375% CF₃COOH in water, mobile phase B was 0.018% CF₃COOH in CH₃CN. The column used for the chromatography was a 2.0×50 mm phenomenex Luna-C18 column (5 μm particles). Detection methods are diode array (DAD) and evaporative light scattering (ELSD) detection as well as positive/negative electrospray ionization.)

¹H NMR (400 MHz, DMSO-d₆) δ=8.76 (d, J=4.0 Hz, 1H), 8.35 (brs, 2H), 8.31 (d, J=8 Hz, 1H), 8.04-8.02 (m, 1H), 7.64 (d, J=11.6 Hz, 1H), 7.58-7.55 (m, 1H), 7.39 (d, J=8.4 Hz, 1H), 4.40 (t, J=6.8 Hz, 2H), 2.98-2.93 (m, 4H), 2.86 (t, J=6.0 Hz, 2H), 2.69-2.65 (m, 4H), 2.52 (s, 3H).

### Example 12: Synthesis of 3-[2-[4-(2-fluoro-5-oxazol-2-yl-phenyl) piperazin-1-yl]ethyl]-8-prop-1-ynyl-[1,2,4]triazolo[5,1-f]purin-5-amine

### Step 1: Synthesis of 6-chloro-9-(2,2-diethoxyethyl) purin-2-amine

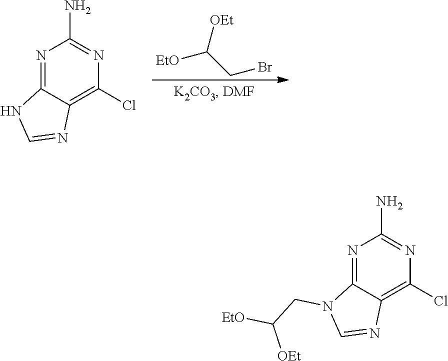 [SMILES: Nc1nc(Cl)c2nc[nH]c2n1 ; CCOC(CBr)OCC ; CCOC(Cn1cnc2c(Cl)nc(N)nc21)OCC]

To a solution of 6-chloro-9H-purin-2-amine (20 g, 117.94 mmol, 1 eq) in DMF (400 mL) was added 2-bromo-1,1-diethoxy-ethane (25.57 g, 129.74 mmol, 19.52 mL, 1.1 eq) and K₂CO₃ (17.93 g, 129.74 mmol, 1.1 eq). The mixture was stirred at 140° C. for 2 h. The reaction mixture was poured into water, extracted with ethyl acetate (3×300 mL). The organic phase was washed with brine, dried over Na₂SO₄ and concentrated to give crude product. The crude product was purified by column chromatography (SiO₂, petroleum ether/ethyl acetate=100:1 to 1:2). Compound 6-chloro-9-(2,2-diethoxyethyl) purin-2-amine (16.5 g, 57.75 mmol, 48.96% yield) was obtained as a white solid. ¹H NMR (400 MHz, DMSO-d₆) δ=8.04 (s, 1H), 6.93 (br s, 2H), 4.80 (t, J=5.3 Hz, 1H), 4.13 (d, J=5.3 Hz, 2H), 3.64 (qd, J=7.0, 9.6 Hz, 2H), 3.48-3.37 (m, 2H), 1.03 (t, J=7.0 Hz, 6H).

### Step 2: Synthesis of 9-(2,2-diethoxyethyl)-6-hydrazino-purin-2-amine

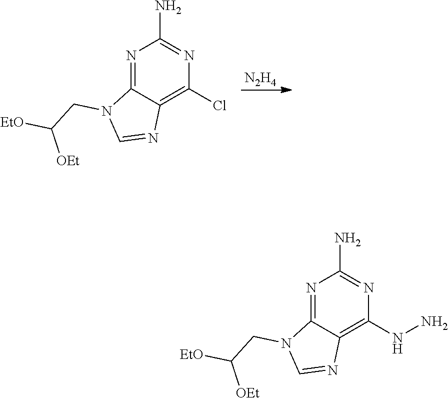

To a mixture of 6-chloro-9-(2,2-diethoxyethyl) purin-2-amine (15 g, 52.50 mmol, 1 eq) in EtOH (30 mL) was added N₂H₄.H₂O (26.82 g, 524.97 mmol, 26.04 mL, 98% purity, 10 eq). The mixture was stirred at 60° C. for 2 h. The mixture was cooled to 25° C. Solid was precipitated out. The resulting solid was collected by filtration, dried under high vacuum to give 9-(2,2-diethoxyethyl)-6-hydrazino-purin-2-amine (10 g, 35.55 mmol, 67.71% yield) as a white solid. Compound 9-(2,2-diethoxyethyl)-6-hydrazino-purin-2-amine (10 g, 35.55 mmol, 67.71% yield) was obtained as a white solid. ¹H NMR (400 MHz, DMSO-d₆) δ=8.49 (br s, 1H), 7.63 (s, 1H), 5.97 (s, 2H), 4.77 (t, J=5.4 Hz, 1H), 4.42 (br s, 2H), 4.04 (d, J=5.4 Hz, 2H), 3.68-3.56 (m, 2H), 3.44-3.34 (m, 2H), 1.03 (t, J=7.0 Hz, 6H). LCMS for product (ESI+): m/z 282.1 [M+H]⁺, Rt: 0.679 min.

### Step 3: Synthesis of N′-[2-amino-9-(2,2-diethoxyethyl) purin-6-yl]but-2-ynehydrazide

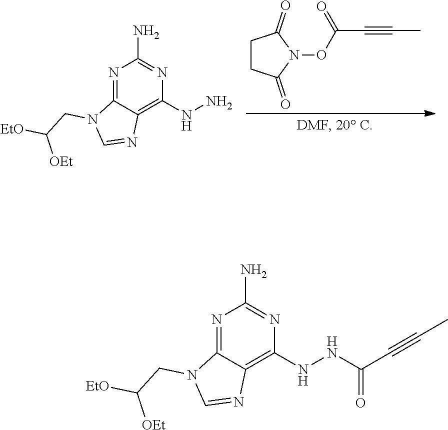 [SMILES: CCOC(Cn1cnc2c(NN)nc(N)nc21)OCC ; CC#CC(=O)ON1C(=O)CCC1=O ; CC#CC(=O)NNc1nc(N)nc2c1ncn2CC(OCC)OCC]

To a solution of 9-(2,2-diethoxyethyl)-6-hydrazino-purin-2-amine (1.3 g, 4.62 mmol, 1 eq) in DMF (13 mL) was added (2,5-dioxopyrrolidin-1-yl) but-2-ynoate (1.55 g, 6.01 mmol, 1.3 eq). The mixture was stirred at 20° C. for 12 h. The reaction mixture was poured into water (80 mL), extracted with ethyl acetate (3×100 mL). The organic phase was washed with brine, dried over Na₂SO₄ and concentrated to give residue. The residue was purified by prep-HPLC. Compound N′-[2-amino-9-(2,2-diethoxyethyl) purin-6-yl]but-2-ynehydrazide (0.5 g, 1.44 mmol, 31.15% yield) was obtained as a yellow solid.

### Step 4: Synthesis of 3-(2,2-diethoxyethyl)-8-prop-1-ynyl-[1,2,4]triazolo[5,1-f]purin-5-amine

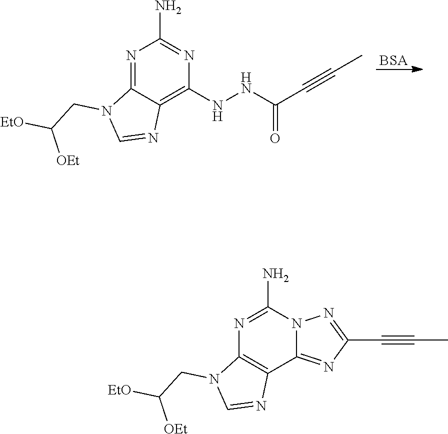

A solution of N′-[2-amino-9-(2,2-diethoxyethyl) purin-6-yl]but-2-ynehydrazide (450 mg, 1.30 mmol, 1 eq) in BSA (5.27 g, 25.91 mmol, 6.40 mL, 20 eq) was stirred at 120° C. for 12 h. The mixture was concentrated to give residue. The residue was purified by prep-HPLC. Compound 3-(2,2-diethoxyethyl)-8-prop-1-ynyl-[1,2,4]triazolo[5,1-f]purin-5-amine (150 mg, 455.43 umol, 35.16% yield) was obtained as a white solid. LCMS for product (ESI+): m/z 330.1 [M+H]⁺, Rt: 0.980 min.

### Step 5: Synthesis of 2-(5-amino-8-prop-1-ynyl-[1,2,4]triazolo[5,1-f]purin-3-yl) acetaldehyde

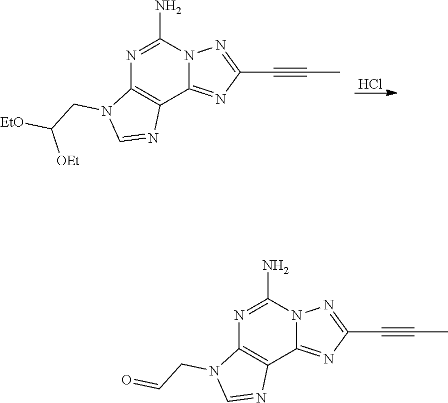 [SMILES: CC#Cc1nc2c3ncn(CC(OCC)OCC)c3nc(N)n2n1 ; Cl ; CC#Cc1nc2c3ncn(CC=O)c3nc(N)n2n1]

A solution of 3-(2,2-diethoxyethyl)-8-prop-1-ynyl-[1,2,4]triazolo[5,1-f]purin-5-amine (100 mg, 303.62 umol, 1 eq) in HCl (1 M, 10.00 mL, 32.94 eq) was stirred at 100° C. for 2 h. The mixture was concentrated. Compound 2-(5-amino-8-prop-1-ynyl-[1,2,4]triazolo[5,1-f]purin-3-yl) acetaldehyde (70 mg, 274.26 umol, 90.33% yield) was obtained as a white solid. LCMS for product (ESI+): m/z 274.1 [M+H]⁺, Rt: 0.707 min.

### Step 6: Synthesis of 3-[2-[4-(2-fluoro-5-oxazol-2-yl-phenyl) piperazin-1-yl]ethyl]-8-prop-1-ynyl-[1,2,4]triazolo[5,1-f]purin-5-amine

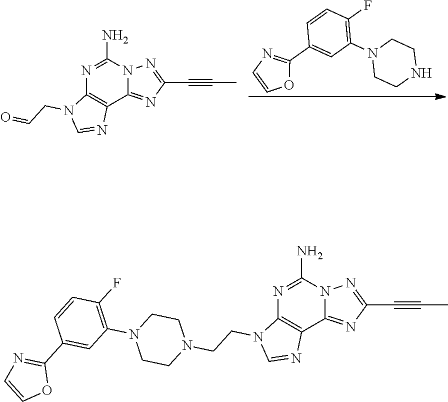

To a solution of 2-(5-amino-8-prop-1-ynyl-[1,2,4]triazolo[5,1-f]purin-3-yl) acetaldehyde (60 mg, 141.05 umol, 1 eq) in MeOH (1 mL) was added NaBH₃CN (26.59 mg, 423.14 umol, 3 eq) and 2-(4-fluoro-3-piperazin-1-yl-phenyl) oxazole (34.88 mg, 141.05 umol, 1 eq). The mixture was stirred at 25° C. for 12 h. The reaction mixture was filtered, and the filtrate was purified by prep-HPLC. Compound 3-[2-[4-(2-fluoro-5-oxazol-2-yl-phenyl) piperazin-1-yl]ethyl]-8-prop-1-ynyl-[1,2,4]triazolo[5,1-f]purin-5-amine (15 mg, 30.18 umol, 21.40% yield, 97.9% purity) was obtained as a white solid. ¹H NMR (400 MHz, DMSO-d₆) δ=8.20 (s, 1H), 8.09 (s, 1H), 7.84 (br s, 2H), 7.60-7.51 (m, 2H), 7.36 (s, 1H), 7.29 (br dd, J=8.7, 12.3 Hz, 1H), 4.29 (br t, J=5.7 Hz, 2H), 3.06 (br s, 4H), 2.78 (br t, J=5.3 Hz, 2H), 2.65 (br s, 4H), 2.14 (s, 3H). LCMS for product (ESI+): m/z 487.2 [M+H]⁺, Rt: 1.961 min.

### Example 13: Synthesis of methyl 2-(4-(4-(2-(5-amino-8-(prop-1-yn-1-yl)-3H-[1,2,4]triazolo[5,1-i]purin-3-yl)ethyl)piperazin-1-yl)phenoxy)-2-methylpropanoate

### Step 1: Synthesis of methyl 2-(4-(4-(2-(5-amino-8-(prop-1-yn-1-yl)-3H-[1,2,4]triazolo[5,1-i]purin-3-yl)ethyl)piperazin-1-yl)phenoxy)-2-methylpropanoate

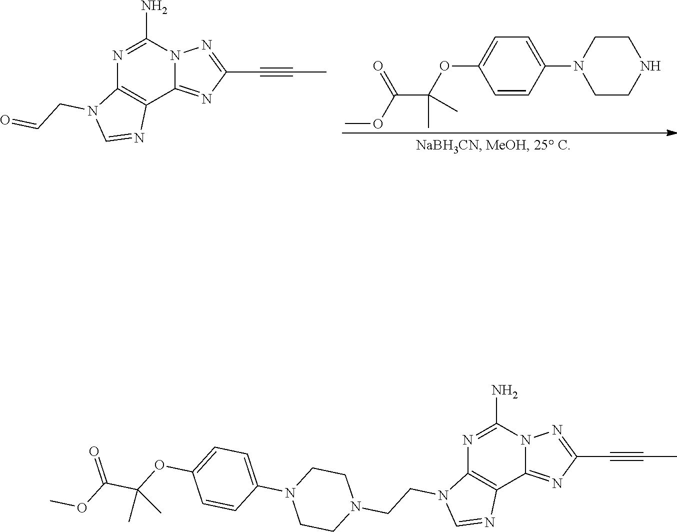 [SMILES: CC#Cc1nc2c3ncn(CC=O)c3nc(N)n2n1 ; COC(=O)C(C)(C)Oc1ccc(N2CCNCC2)cc1 ; CC#Cc1nc2c3ncn(CCN4CCN(c5ccc(OC(C)(C)C(=O)OC)cc5)CC4)c3nc(N)n2n1]

To a mixture of 2-(5-amino-8-prop-1-ynyl-[1,2,4]triazolo[5,1-f]purin-3-yl)acetaldehyde (50 mg, 195.90 umol, 1 eq) in MeOH (0.5 mL) was added methyl 2-methyl-2-(4-piperazin-1-ylphenoxy)propanoate (54.53 mg, 195.90 umol, 1 eq), NaOAc (16.07 mg, 195.90 umol, 1 eq) and NaBH₃CN (36.93 mg, 587.69 umol, 3 eq). The mixture was stirred at 25° C. for 2 h. LCMS showed the completion of the reaction. The mixture was poured into water (10 mL). The mixture was extracted with EtOAc (30 mL). The organic layer was washed with brine, dried over Na₂SO₄, filtered, and concentrated to give methyl 2-(4-(4-(2-(5-amino-8-(prop-1-yn-1-yl)-3H-[1,2,4]triazolo[5,1-i]purin-3-yl)ethyl)piperazin-1-yl)phenoxy)-2-methylpropanoate as a brown solid (used without further purification). LCMS for product (ESI+): m/z 518.3 [M+H]⁺. Rt: 1.131 min.

### Step 2: Synthesis of 2-(4-(4-(2-(5-amino-8-(prop-1-yn-1-yl)-3H-[1,2,4]triazolo[5,1-i]purin-3-yl)ethyl)piperazin-1-yl)phenoxy)-2-methylpropanoic acid

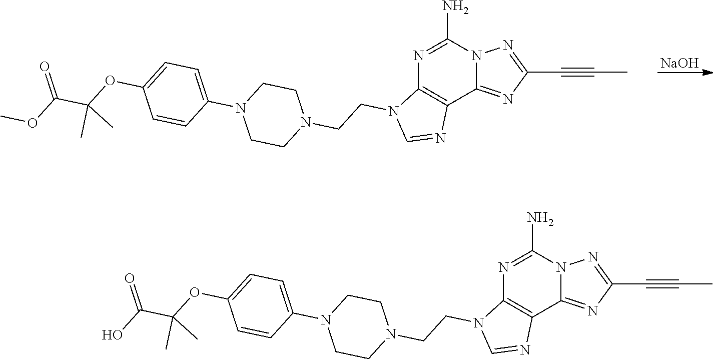

To a mixture of methyl 2-[4-[4-[2-(5-amino-8-prop-1-ynyl-[1,2,4]triazolo[5,1-f]purin-3-yl)ethyl]piperazin-1-yl]phenoxy]-2-methyl-propanoate (50 mg, 96.60 umol, 1 eq) in THF (0.6 mL), MeOH (0.4 mL) and H₂O (0.2 mL) was added NaOH (11.59 mg, 289.81 umol, 3 eq), the mixture was stirred at 25° C. for 3 h. LCMS showed completion of the reaction. The mixture was filtered and concentrated. The residue that was purified using prep-HPLC to yield 2-(4-(4-(2-(5-amino-8-(prop-1-yn-1-yl)-3H-[1,2,4]triazolo[5,1-i]purin-3-yl)ethyl)piperazin-1-yl)phenoxy)-2-methylpropanoic acid as a white solid (11 mg, 21.84 umol). LCMS for product (ESI+): m/z 504.3 [M+H]⁺, Rt: 1.911 min. ¹H NMR (400 MHz, DMSO-d₆) δ=8.06 (s, 1H), 7.82 (br s, 2H), 6.85-6.78 (m, 2H), 6.78-6.72 (m, 2H), 4.28 (br t, J=6.0 Hz, 2H), 2.99 (br s, 4H), 2.75 (br t, J=6.0 Hz, 2H), 2.58 (br s, 4H), 2.14 (s, 3H), 1.41 (s, 6H).

### Example 14: Synthesis of 3-(2-(3-(oxetan-3-yl)-7,8-dihydro-1,6-naphthyridin-6(5H)-yl)ethyl)-8-(pyridin-2-yl)-3H-[1,2,4]triazolo[5,1-i]purin-5-amine

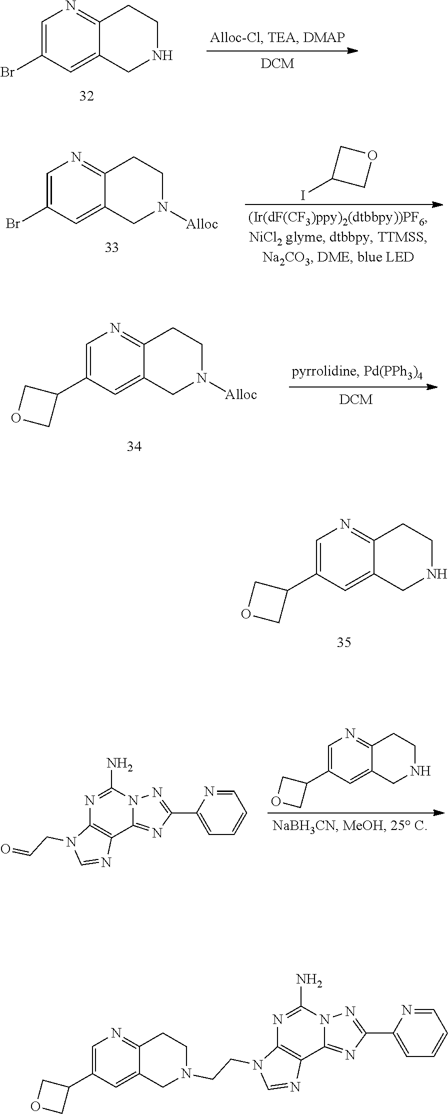

### Step 1: Synthesis of Compound 33

To a mixture of 3-bromo-5,6,7,8-tetrahydro-1,6-naphthyridine (100 mg, 349.66 umol, 1 eq, 2HCl), DMAP (4.27 mg, 34.97 umol, 0.1 eq) and Et₃N (42.46 mg, 419.59 umol, 58.40 uL, 1.2 eq) in DCM (0.5 mL) was added allyl carbonochloridate (63.22 mg, 524.48 umol, 55.45 uL, 1.5 eq) at 0° C. The mixture was stirred at 25° C. for 2 h. TLC (PE:EA=5:1) indicated completion of the reaction. The mixture was concentrated, and the residue was purified using prep-TLC (PE:EA=5:1) to afford allyl 3-bromo-7,8-dihydro-1,6-naphthyridine-6(5H)-carboxylate (63 mg, 212.02 umol) as a white solid. ¹H NMR (400 MHz, CDCl₃) δ=8.49 (s, 1H), 7.57 (s, 1H), 6.03-5.89 (m, 1H), 5.33 (br d, J=17.3 Hz, 1H), 5.24 (d, J=10.4 Hz, 1H), 4.65 (br s, 4H), 3.82 (t, J=6.0 Hz, 2H), 2.98 (t, J=5.9 Hz, 2H).

### Step 2: Synthesis of Compound 34

To an 8 mL tube equipped with a stirrer bar was added allyl 3-bromo-7,8-dihydro-5H-1,6-naphthyridine-6-carboxylate (30 mg, 100.96 umol, 1 eq) and 3-iodooxetane (18.57 mg, 100.96 umol, 1 eq). The tube was placed in a glove box. Photocatalyst Ir[dF(CF₃)ppy]₂(dtbpy)(PF₆) (1.13 mg, 1.01 umol, 0.01 eq), TTMSS (25.10 mg, 100.96 umol, 31.15 uL, 1 eq) and Na₂CO₃ (21.40 mg, 201.92 umol, 2 eq) were added. The tube was sealed in the glove box before DME (0.5 mL) was added. NiCl₂.glyme (110.91 ug, 5.05e-1 umol, 0.005 eq) and dtbbpy (135.49 ug, 5.05e-1 umol, 0.005 eq) were added as a stock solution in DME (0.5 mL) (sonicated for 5 minutes before addition). The reaction mixture was removed from glove box and irradiated with a 34 W blue LED lamp. The mixture was stirred at 25° C. for 12 h. LCMS indicated completion of the reaction. The mixture was filtered, concentrated; and the residue that was purified using prep-HPLC to obtain afford allyl 3-(oxetan-3-yl)-7,8-dihydro-1,6-naphthyridine-6(5H)-carboxylate (5 mg, 18.23 umol) as a white solid. LCMS for product (ESI+): m/z 275.1 [M+H]⁺, Rt: 1.517 min. ¹H NMR (400 MHz, DMSO-d₆) δ=8.34 (d, J=1.4 Hz, 1H), 7.77 (s, 1H), 6.01-5.89 (m, 1H), 5.31 (dd, J=1.4, 17.3 Hz, 1H), 5.20 (d, J=10.5 Hz, 1H), 4.93 (dd, J=5.9, 8.3 Hz, 2H), 4.69-4.54 (m, 6H), 4.26 (quin, J=7.6 Hz, 1H), 3.73 (br s, 2H), 2.88 (br t, J=5.9 Hz, 2H).

### Step 3: Synthesis of Compound 35

To a mixture of allyl 3-(oxetan-3-yl)-7,8-dihydro-5H-1,6-naphthyridine-6-carboxylate (4.9 mg, 17.86 umol, 1 eq) in DCM (0.5 mL) was added pyrrolidine (6.35 mg, 89.31 umol, 7.46 uL, 5 eq) and Pd(PPh₃)₄(20.64 mg, 17.86 umol, 1 eq) under N₂. The mixture was stirred at 25° C. for 2. LCMS showed the reaction completion of the reaction. The mixture was filtered, concentrated the residue was purified using prep-HPLC to obtain 3-(oxetan-3-yl)-5,6,7,8-tetrahydro-1,6-naphthyridine (1.2 mg, 6.3) as a white solid. LCMS for product (ESI+): m/z 191.1 [M+H]⁺, Rt: 1.336 min.

### Step 4: Synthesis of 3-(2-(3-(oxetan-3-yl)-7,8-dihydro-1,6-naphthyridin-6(5H)-yl)ethyl)-8-(pyridin-2-yl)-3H-[1,2,4]triazolo[5,1-i]purin-5-amine

To a mixture of 2-[5-amino-8-(2-pyridyl)-[1,2,4]triazolo[5,1-f]purin-3-yl]acetaldehyde (1.70 mg, 5.78 umol, 1 eq) in MeOH (0.5 mL) was added 3-(oxetan-3-yl)-5,6,7,8-tetrahydro-1,6-naphthyridine (1.1 mg, 5.78 umol, 1 eq), NaOAc (474.33 ug, 5.78 umol, 1 eq) and NaBH₃CN (1.09 mg, 17.35 umol, 3 eq). The mixture was stirred at 25° C. for 2 h. LCMS indicated completion of the reaction. The mixture was filtered, and the residue was purified using prep-HPLC to yield 3-(2-(3-(oxetan-3-yl)-7,8-dihydro-1,6-naphthyridin-6(5H)-yl)ethyl)-8-(pyridin-2-yl)-3H-[1,2,4]triazolo[5,1-i]purin-5-amine (2.2 mg, 4.70 umol) as a white solid. LCMS for product (ESI+): m/z 469.3 [M+H]⁺, Rt: 2.102 min. ¹H NMR (400 MHz, DMSO-d₆) δ=8.79-8.72 (m, 1H), 8.38-8.25 (m, 2H), 8.13-8.06 (m, 1H), 8.05-7.97 (m, 1H), 7.86 (br s, 2H), 7.59-7.51 (m, 2H), 4.91 (dd, J=5.9, 8.3 Hz, 2H), 4.58 (t, J=6.3 Hz, 2H), 4.39 (br t, J=5.9 Hz, 2H), 4.27-4.16 (m, 1H), 3.71 (s, 2H), 2.95 (br t, J=5.9 Hz, 2H), 2.86 (br dd, J=4.5, 9.4 Hz, 4H).

### Example 15: Synthesis of 3-(2-(3-(oxetan-3-yl)-7,8-dihydro-1,6-naphthyridin-6(5H)-yl)ethyl)-8-(pyridin-2-yl)-3H-[1,2,4]triazolo[5,1-i]purin-5-amine

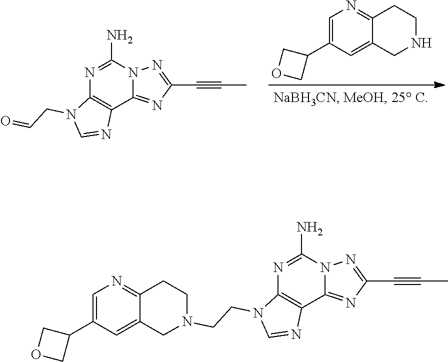

### Step 1: Synthesis of 3-(2-(3-(oxetan-3-yl)-7,8-dihydro-1,6-naphthyridin-6(5H)-yl)ethyl)-8-(pyridin-2-yl)-3H-[1,2,4]triazolo[5,1-i]purin-5-amine

To a mixture of 2-(5-amino-8-prop-1-ynyl-[1,2,4]triazolo[5,1-f]purin-3-yl)acetaldehyde (5 mg, 19.59 umol, 1 eq) in MeOH (0.5 mL) was added 3-(oxetan-3-yl)-5,6,7,8-tetrahydro-1,6-naphthyridine (3.73 mg, 19.59 umol, 1 eq), NaOAc (1.61 mg, 19.59 umol, 1 eq) and NaBH₃CN (3.69 mg, 58.77 umol, 3 eq). The mixture was stirred at 25° C. for 2 h. LCMS indicated completion of the reaction. The mixture was filtered, and the residue was purified by prep-HPLC to afford 3-(2-(3-(oxetan-3-yl)-7,8-dihydro-1,6-naphthyridin-6(5H)-yl)ethyl)-8-(pyridin-2-yl)-3H-[1,2,4]triazolo[5,1-i]purin-5-amine (4 mg, 9.31 umol) as a white solid. LCMS for product (ESI+): m/430.2 [M+H]⁺, Rt: 2.180 min. ¹H NMR (400 MHz, DMSO-d₆) δ=8.27 (d, J=2.1 Hz, 1H), 8.05 (s, 1H), 7.85 (br s, 2H), 7.56 (d, J=2.0 Hz, 1H), 4.90 (dd, J=5.9, 8.3 Hz, 2H), 4.57 (t, J=6.3 Hz, 2H), 4.35 (br t, J=6.0 Hz, 2H), 4.20 (quin, J=7.6 Hz, 1H), 3.68 (s, 2H), 2.91 (br t, J=6.0 Hz, 2H), 2.84 (br dd, J=4.0, 7.8 Hz, 4H), 2.13 (s, 3H).

### Example 16: Synthesis of 3-(2-(4-(2-fluoro-5-(1H-pyrazol-1-yl)phenyl)piperazin-1-yl)ethyl)-8-(Prop-1-yn-1-yl)-3H-[1,2,4]triazolo[5,1-i]purin-5-amine

### Step 1: Synthesis of Compound 37

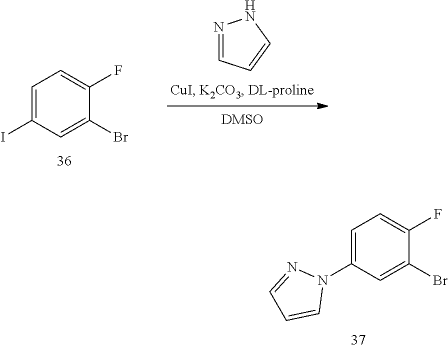 [SMILES: Fc1ccc(I)cc1Br ; c1cn[nH]c1 ; Fc1ccc(-n2cccn2)cc1Br]

A mixture of 2-bromo-1-fluoro-4-iodo-benzene (1 g, 3.32 mmol, 1 eq), 1H-pyrazole (226.25 mg, 3.32 mmol, 1 eq), K₂CO₃ (688.98 mg, 4.99 mmol, 1.5 eq) and proline, 1-[[(7, 7-dimethyl-2-oxobicyclo [2. 2. 1]hept-1-yl) methyl]sulfonyl]-(109.48 mg, 332.34 umol, 0.1 eq) in dimethyl sulfoxide (10 mL) was degassed and purged with N₂ for 3 times and the mixture was added CuI (31.65 mg, 166.17 umol, 0.05 eq). The mixture was stirred at 80° C. for 2 h under N₂ atmosphere. LC-MS showed that the reactant was completely consumed and one new main peak with desired mass was detected. The mixture was diluted with water 100 mL and extracted with ethyl acetate (3×30 mL). The combined organic layer was washed with brine 40 mL, dried over Na₂SO₄, filtered, and concentrated under reduced pressure. The residue that was purified by column chromatography (SiO₂, Petroleum ether/Ethyl acetate=10/1 to 5/1) to afford 1-(3-bromo-4-fluorophenyl)-1H-pyrazole (0.3 g, 1.24 mmol) as a white solid. LCMS for product (ESI+): m/z 241.0 [M+H]⁺, Rt: 1.014 min.

### Step 2: Synthesis of Compound 38

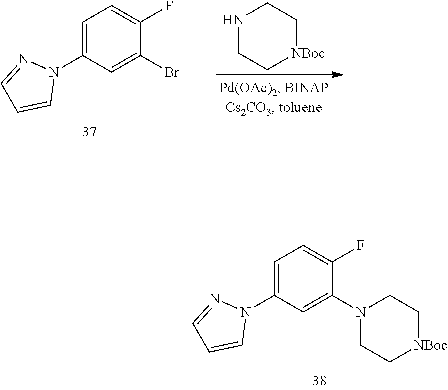 [SMILES: Fc1ccc(-n2cccn2)cc1Br ; CC(C)(C)OC(=O)N1CCNCC1 ; CC(C)(C)OC(=O)N1CCN(c2cc(-n3cccn3)ccc2F)CC1]

A mixture of 1-(3-bromo-4-fluoro-phenyl) pyrazole (280 mg, 1.16 mmol, 1 eq), tert-butyl piperazine-1-carboxylate (281.24 mg, 1.51 mmol, 1.3 eq), Cs₂CO₃ (1.89 g, 5.81 mmol, 5 eq), BINAP (216.98 mg, 348.46 umol, 0.3 eq) and Pd(OAc)₂ (52.15 mg, 232.31 umol, 0.2 eq) in toluene (3 mL) was degassed and purged with N₂ for 3 times. The mixture was stirred at 85° C. for 2 h under N₂ atmosphere. LC-MS showed completion of the reaction and one new main peak with desired mass was detected. The reaction mixture was diluted with water 5 mL and extracted with ethyl acetate (3×3 mL). The combined organic layer was washed with brine (3 mL), dried over Na₂SO₄, filtered, and concentrated under reduced pressure. The residue that was purified by column chromatography (SiO₂, Petroleum ether/Ethyl acetate=10/1 to 3/1) to afford tert-butyl 4-(2-fluoro-5-(1H-pyrazol-1-yl)phenyl)piperazine-1-carboxylate (280 mg, 808.32 umol) as a yellow oil. LCMS for product (ESI+): m/z 347.2 [M+H]⁺, Rt: 1.109 min. ¹H NMR (400 MHz, DMSO-d₆) δ=8.48 (d, J=2.1 Hz, 1H), 7.72 (s, 1H), 7.51-7.37 (m, 2H), 7.27 (dd, J=8.6, 12.2 Hz, 1H), 6.53 (s, 1H), 3.49 (br s, 4H), 3.08-3.01 (m, 4H), 1.43 (s, 9H).

### Step 3: Synthesis of Compound 39

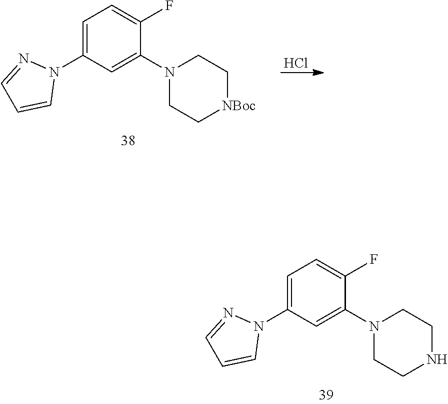 [SMILES: CC(C)(C)OC(=O)N1CCN(c2cc(-n3cccn3)ccc2F)CC1 ; Cl ; Fc1ccc(-n2cccn2)cc1N1CCNCC1]

To a solution of tert-butyl 4-(2-fluoro-5-pyrazol-1-yl-phenyl) piperazine-1-carboxylate (280 mg, 808.32 umol, 1 eq) in methanol (1.5 mL) was added HCl/MeOH (4 M, 1.5 mL, 7.42 eq). The mixture was stirred at 25° C. for 2 h. LC-MS showed completion of the reaction and appearance of one main peak with desired mass was detected. The mixture was concentrated under reduced pressure to afford 1-(2-fluoro-5-(1H-pyrazol-1-yl)phenyl)piperazine (180 mg, 730.87 umol) that was used without further purification. LCMS for product (ESI+): m/z 247.1 [M+H]⁺, Rt: 0.275 min.

### Step 4: Synthesis of 3-(2-(4-(2-fluoro-5-(1H-pyrazol-1-yl)phenyl)piperazin-1-yl)ethyl)-8-(prop-1-yn-1-yl)-3H-[1,2,4]triazolo[5,1-i]purin-5-amine

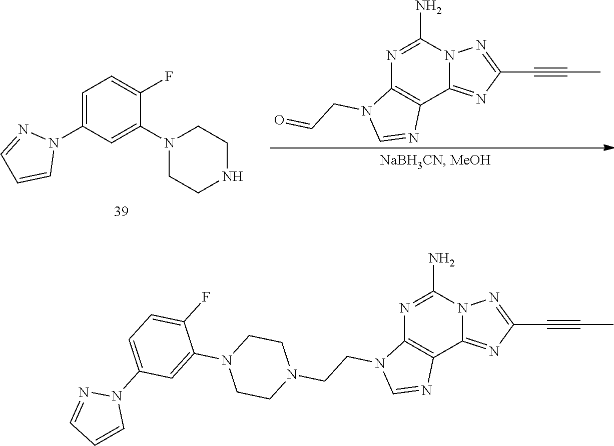 [SMILES: Fc1ccc(-n2cccn2)cc1N1CCNCC1 ; CC#Cc1nc2c3ncn(CC=O)c3nc(N)n2n1 ; CC#Cc1nc2c3ncn(CCN4CCN(c5cc(-n6cccn6)ccc5F)CC4)c3nc(N)n2n1]

To a solution of 1-(2-fluoro-5-pyrazol-1-yl-phenyl) piperazine (30 mg, 121.81 umol, 1 eq) and 2-(5-amino-8-prop-1-ynyl-[1, 2, 4]triazolo [5, 1-f]purin-3-yl) acetaldehyde (31.09 mg, 121.81 umol, 1 eq) in methyl alcohol (1 mL) was acidified by NaOAc (99.93 mg, 1.22 mmol, 10 eq) to pH 7. The mixture was added NaBH₃CN (22.96 mg, 365.43 umol, 3 eq). The mixture was stirred at 25° C. for 2 h. LC-MS showed completion of the reaction. The mixture was filtered, and the filtrate was purified by prep-HPLC (neutral condition) to obtain 3-(2-(4-(2-fluoro-5-(1H-pyrazol-1-yl)phenyl)piperazin-1-yl)ethyl)-8-(prop-1-yn-1-yl)-3H-[1,2,4]triazolo[5,1-i]purin-5-amine (9 mg, 18.54 umol) as a white solid. LCMS for product (ESI+): m/z 486.3 [M+H]⁺, Rt: 2.681 min. ¹H NMR (400 MHz, DMSO-d₆) δ=8.47 (d, J=2.4 Hz, 1H), 8.08 (s, 1H), 7.86 (br s, 2H), 7.71 (d, J=1.4 Hz, 1H), 7.43-7.34 (m, 2H), 7.24 (dd, J=8.7, 12.3 Hz, 1H), 6.51 (t, J=2.1 Hz, 1H), 4.29 (br t, J=6.0 Hz, 2H), 3.06 (br s, 4H), 2.78 (br t, J=6.1 Hz, 2H), 2.64 (br s, 4H), 2.14 (s, 3H).

### Example 17: Synthesis of 8-(prop-1-yn-1-yl)-3-(2-(3-(thiazol-2-yl)-7,8-dihydro-1,6-naphthyridin-6(5H)-yl)ethyl)-3H-[1,2,4]triazolo[5,1-i]purin-5-amine

### Step 1: Synthesis of Compound 41

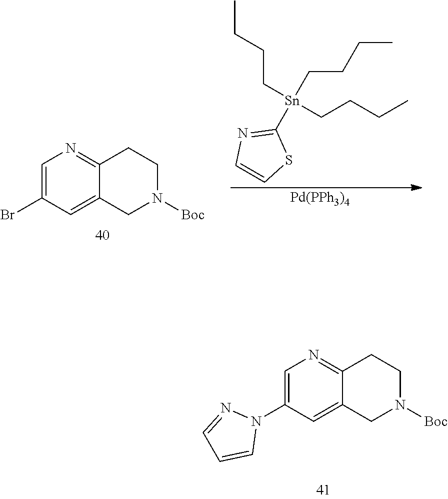

To a mixture of tert-butyl 3-bromo-7,8-dihydro-5H-1,6-naphthyridine-6-carboxylate (64 mg, 204.35 umol, 1 eq) and tributyl(thiazol-2-yl)stannane (91.75 mg, 245.22 umol, 1.2 eq) in toluene (1 mL) was added Pd(PPh₃)₄(23.61 mg, 20.43 umol, 0.1 eq) under N₂. The mixture was stirred at 120° C. for 2 h. LCMS showed completion of the reaction. The mixture was poured into water (5 mL). The aqueous layer was extracted with EtOAc (3×10 mL). The combined organic layer were washed with brine, dried over Na₂SO₄, concentrated and used without purification to afford tert-butyl 3-(thiazol-2-yl)-7,8-dihydro-1,6-naphthyridine-6(5H)-carboxylate (100 mg, crude) as a black solid. LCMS for product (ESI+): m/z 318.1 [M+H]⁺, Rt: 1.753 min.

### Step 2: Synthesis of Compound 42

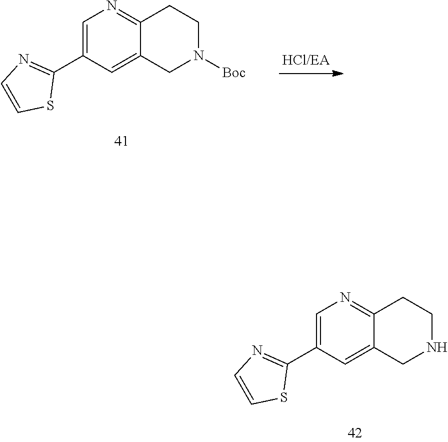 [SMILES: CC(C)(C)OC(=O)N1CCc2ncc(-c3nccs3)cc2C1 ; c1csc(-c2cnc3c(c2)CNCC3)n1]

A mixture of tert-butyl 3-thiazol-2-yl-7,8-dihydro-5H-1,6-naphthyridine-6-carboxylate (100 mg, 315.05 umol, 1 eq) in HCl/EtOAc (4 M, 4 mL, 50.78 eq) was stirred at 25° C. for 2 h. TLC (EtOAc) showed that starting material was consumed. The resulting solid 2-(5,6,7,8-tetrahydro-1,6-naphthyridin-3-yl)thiazole (43 mg, crude) was collected by filtration and used without further purification.

### Step 3: Synthesis of 8-(prop-1-yn-1-yl)-3-(2-(3-(thiazol-2-yl)-7,8-dihydro-1,6-naphthyridin-6(5H)-yl)ethyl)-3H-[1,2,4]triazolo[5,1-i]purin-5-amine

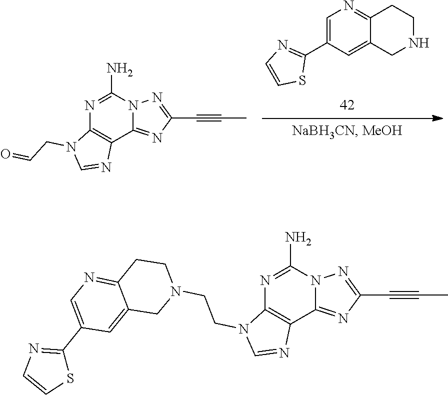 [SMILES: CC#Cc1nc2c3ncn(CC=O)c3nc(N)n2n1 ; c1csc(-c2cnc3c(c2)CNCC3)n1 ; CC#Cc1nc2c3ncn(CCN4CCc5ncc(-c6nccs6)cc5C4)c3nc(N)n2n1]

A solution of 2-(5-amino-8-prop-1-ynyl-[1,2,4]triazolo[5,1-f]purin-3-yl)acetaldehyde (50 mg, 195.90 umol, 1 eq) and 2-(5,6,7,8-tetrahydro-1,6-naphthyridin-3-yl)thiazole (42.57 mg, 195.90 umol, 1 eq) in MeOH (1 mL) was acidified with NaOAc (16.07 mg, 195.90 umol, 1 eq) to pH 7. To the mixture was added NaBH₃CN (36.93 mg, 587.69 umol, 3 eq). The mixture was stirred at 25° C. for 2 h. LCMS showed completion of the reaction. The mixture was purified by prep-HPLC (neutral condition, column: Waters Xbridge BEH C18 100*30 mm*10 um; mobile phase: [water (10 mM NH₄HCO₃)-ACN]; B %: 15%-45%, 8 min) to afford 8-(prop-1-yn-1-yl)-3-(2-(3-(thiazol-2-yl)-7,8-dihydro-1,6-naphthyridin-6(5H)-yl)ethyl)-3H-[1,2,4]triazolo[5,1-i]purin-5-amine (9.8 mg, 21.47 umol) as a white solid. LCMS for product (ESI+): m/z 457.2 [M+H]⁺, Rt: 2.383 min. ¹H NMR (400 MHz, CDCl₃) δ=8.95 (d, J=2.0 Hz, 1H), 7.95-7.86 (m, 3H), 7.39 (d, J=3.3 Hz, 1H), 5.76 (s, 2H), 4.39 (t, J=6.1 Hz, 2H), 3.81 (s, 2H), 3.12-2.93 (m, 6H), 2.15 (s, 3H).

### Example 18: Synthesis of 3-(2-(4-(2-fluoro-5-(1H-pyrazol-1-yl)phenyl)piperazin-1-yl)ethyl)-8-(pyridin-2-yl)-3H-[1,2,4]triazolo[5,1-i]purin-5-amine

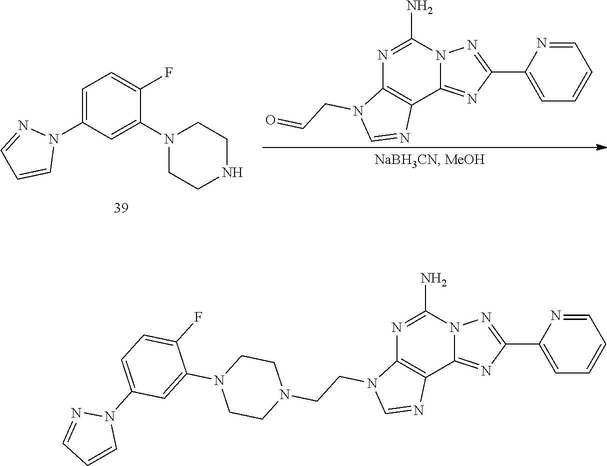 [SMILES: Fc1ccc(-n2cccn2)cc1N1CCNCC1 ; Nc1nc2c(ncn2CC=O)c2nc(-c3ccccn3)nn12 ; Nc1nc2c(ncn2CCN2CCN(c3cc(-n4cccn4)ccc3F)CC2)c2nc(-c3ccccn3)nn12]

A solution of 1-(2-fluoro-5-pyrazol-1-yl-phenyl) piperazine (30 mg, 121.81 umol, 1 eq) and 2-[5-amino-8-(2-pyridyl)-[1, 2, 4]triazolo [5,1-f]purin-3-yl]acetaldehyde (35.85 mg, 121.81 umol, 1 eq) in methyl alcohol (1 mL) was acidified by NaOAc (99.93 mg, 1.22 mmol, 10 eq) to pH 7. To the mixture was added NaBH₃CN (22.96 mg, 365.43 umol, 3 eq). The mixture was stirred at 25° C. for 2 h. LC-MS showed consumption of the starting material. The mixture was filtered, and the filtrate was purified by prep-HPLC (neutral condition) to afford 3-(2-(4-(2-fluoro-5-(1H-pyrazol-1-yl)phenyl)piperazin-1-yl)ethyl)-8-(pyridin-2-yl)-3H-[1,2,4]triazolo[5,1-i]purin-5-amine (15.7 mg, 29.93 umol) as a white solid. LCMS for product (ESI+): m/z 525.3 [M+H]⁺, Rt: 2.583 min. ¹H NMR (400 MHz, DMSO-d₆) δ=8.75 (br d, J=3.8 Hz, 1H), 8.47 (br d, J=1.1 Hz, 1H), 8.34 (br d, J=7.6 Hz, 1H), 8.11 (s, 1H), 8.02 (br t, J=7.4 Hz, 1H), 7.89 (br s, 2H), 7.70 (s, 1H), 7.60-7.51 (m, 1H), 7.45-7.33 (m, 2H), 7.24 (br dd, J=8.8, 12.1 Hz, 1H), 6.51 (br s, 1H), 4.33 (br s, 2H), 3.07 (br s, 4H), 2.81 (br s, 2H), 2.67 (br s, 4H).

### Example 19: Synthesis of 8-(pyridin-2-yl)-3-(2-(3-(thiazol-2-yl)-7,8-dihydro-1,6-naphthyridin-6(5H)-yl)ethyl)-3H-[1,2,4]triazolo[5,1-i]purin-5-amine

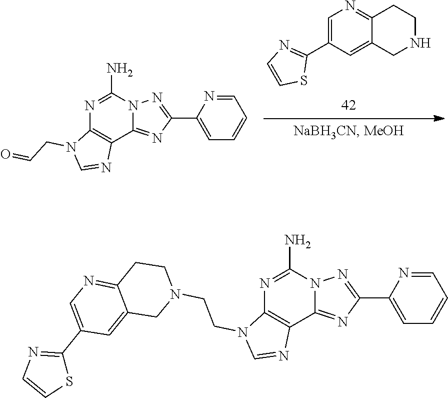 [SMILES: Nc1nc2c(ncn2CC=O)c2nc(-c3ccccn3)nn12 ; c1csc(-c2cnc3c(c2)CNCC3)n1 ; Nc1nc2c(ncn2CCN2CCc3ncc(-c4nccs4)cc3C2)c2nc(-c3ccccn3)nn12]

A solution of 2-[5-amino-8-(2-pyridyl)-[1,2,4]triazolo[5,1-f]purin-3-yl]acetaldehyde (50 mg, 169.91 umol, 1 eq) and 2-(5,6,7,8-tetrahydro-1,6-naphthyridin-3-yl)thiazole (36.92 mg, 169.91 umol, 1 eq) in MeOH (0.5 mL) was acidified by adding NaOAc (13.94 mg, 169.91 umol, 1 eq) to pH 7. To the mixture was added NaBH₃CN (32.03 mg, 509.73 umol, 3 eq). The mixture was stirred at 25° C. for 2 h. LCMS showed that starting material was consumed and a new peak with the desired product mass was detected. The mixture was purified by prep-HPLC (neutral condition) to afford 8-(pyridin-2-yl)-3-(2-(3-(thiazol-2-yl)-7,8-dihydro-1,6-naphthyridin-6(5H)-yl)ethyl)-3H-[1,2,4]triazolo[5,1-i]purin-5-amine (30 mg, 6.0 umol) as a white solid. LCMS for product (ESI+): m/z 496.2 [M+H]⁺, Rt: 2.303 min. ¹H NMR (400 MHz, CDCl₃) δ=8.96 (s, 1H), 8.83 (br d, J=4.6 Hz, 1H), 8.56 (d, J=7.9 Hz, 1H), 7.97-7.85 (m, 4H), 7.47-7.37 (m, 2H), 5.99 (br s, 2H), 4.42 (t, J=6.2 Hz, 2H), 3.84 (s, 2H), 3.13-2.98 (m, 6H).

### Example 20: Synthesis of 3-(2-(4-(6-fluoro-3-methylbenzo[d]isoxazol-5-yl)piperazin-1-yl)ethyl)-8-(prop-1-yn-1-yl)-3H-[1,2,4]triazolo[5,1-i]purin-5-amine

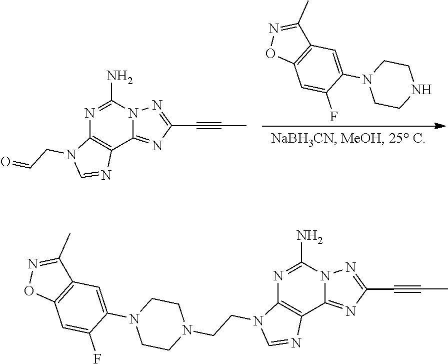 [SMILES: CC#Cc1nc2c3ncn(CC=O)c3nc(N)n2n1 ; Cc1noc2cc(F)c(N3CCNCC3)cc12 ; CC#Cc1nc2c3ncn(CCN4CCN(c5cc6c(C)noc6cc5F)CC4)c3nc(N)n2n1]

To a solution of 2-(5-amino-8-prop-1-ynyl-[1,2,4]triazolo[5,1-f]purin-3-yl)acetaldehyde (10 mg, 39.18 umol, 1 eq) in MeOH (1 mL) was added NaBH₃CN (7.39 mg, 117.54 umol, 3 eq), 6-fluoro-3-methyl-5-piperazin-1-yl-1,2-benzoxazole (10.65 mg, 39.18 umol, 1 eq, HCl) and NaOAc (3.21 mg, 39.18 umol, 1 eq) at 25° C. The mixture was stirred at 25° C. for 12 h. LCMS showed that the starting material was consumed and a new peak corresponding to the desired product was detected. The reaction mixture was filtered and concentrated under reduced pressure. The residue was purified by prep-HPLC to afford 3-(2-(4-(6-fluoro-3-methylbenzo[d]isoxazol-5-yl)piperazin-1-yl)ethyl)-8-(prop-1-yn-1-yl)-3H-[1,2,4]triazolo[5,1-i]purin-5-amine (3.0 mg, 6.30 umol) as a white solid. LCMS for product (ESI+): m/z 475.3 [M+H]⁺, Rt: 2.635 min. ¹H NMR (400 MHz, CDCl₃) δ=7.96 (s, 1H), 7.24 (s, 1H), 7.08 (br d, J=7.6 Hz, 1H), 5.77 (br s, 2H), 4.31 (br t, J=5.7 Hz, 2H), 3.07 (br s, 4H), 2.87 (br t, J=5.6 Hz, 2H), 2.74 (br s, 4H), 2.56 (s, 3H), 2.16 (s, 3H).

### Example 21: Synthesis of 3-(2-(4-(2-fluoro-5-(oxazol-2-yl)phenyl)piperazin-1-yl)ethyl)-8-(pyridin-2-yl)-3H-[1,2,4]triazolo[5,1-i]purin-5-amine

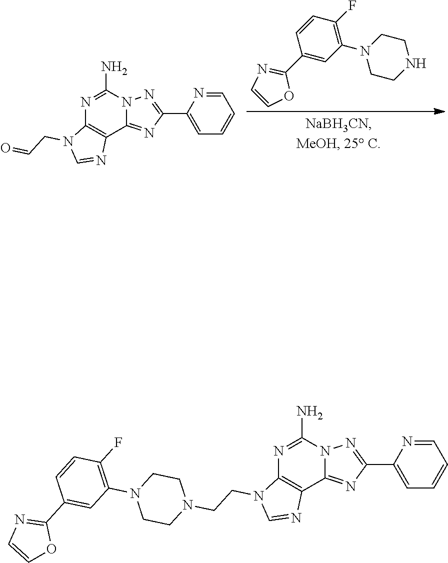

To a solution of 2-[5-amino-8-(2-pyridyl)-[1,2,4]triazolo[5,1-f]purin-3-yl]acetaldehyde (120 mg, 407.79 umol, 1 eq) and 2-(4-fluoro-3-piperazin-1-yl-phenyl)oxazole (115.70 mg, 407.79 umol, 1 eq, HCl) in MeOH (2 mL) was added NaOAc (33.45 mg, 407.79 umol, 1 eq) and NaBH₃CN (76.88 mg, 1.22 mmol, 3 eq). The mixture was stirred at 25° C. for 12 h. LCMS showed starting material was consumed and the desired product peak was detected. The mixture was concentrated, and the residue was purified by prep-HPLC (neutral condition) to afford the 3-(2-(4-(2-fluoro-5-(oxazol-2-yl)phenyl)piperazin-1-yl)ethyl)-8-(pyridin-2-yl)-3H-[1,2,4]triazolo[5,1-i]purin-5-amine (50 mg, 91.72 umol) as a white solid. LCMS for product (ESI+): m/z 526.3 [M+H]⁺, Rt: 2.572 min. ¹H NMR (400 MHz, CDCl₃) δ=8.83 (d, J=4.1 Hz, 1H), 8.57 (d, J=8.0 Hz, 1H), 7.96 (s, 1H), 7.91 (dt, J=1.7, 7.7 Hz, 1H), 7.70 (s, 1H), 7.67-7.60 (m, 2H), 7.43 (dd, J=5.3, 7.0 Hz, 1H), 7.22 (s, 1H), 7.11 (dd, J=8.6, 12.2 Hz, 1H), 6.03 (br s, 2H), 4.34 (t, J=6.1 Hz, 2H), 3.19 (br d, J=4.4 Hz, 4H), 2.89 (t, J=6.2 Hz, 2H), 2.78-2.68 (m, 4H).

### Example 22: Synthesis of 3-(2-(4-(6-fluoro-3-methylbenzo[d]isoxazol-5-yl)piperazin-1-yl)ethyl)-8-(pyridin-2-yl)-3H-[1,2,4]triazolo[5,1-i]purin-5-amine

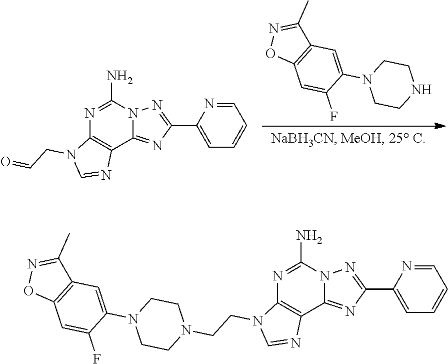 [SMILES: Nc1nc2c(ncn2CC=O)c2nc(-c3ccccn3)nn12 ; Cc1noc2cc(F)c(N3CCNCC3)cc12 ; Cc1noc2cc(F)c(N3CCN(CCn4cnc5c4nc(N)n4nc(-c6ccccn6)nc54)CC3)cc12]

To a solution of 2-[5-amino-8-(2-pyridyl)-[1,2,4]triazolo[5,1-f]purin-3-yl]acetaldehyde (110 mg, 373.80 umol, 1 eq) and 6-fluoro-3-methyl-5-piperazin-1-yl-1,2-benzoxazole (101.57 mg, 373.80 umol, 1 eq, HCl) in MeOH (2 mL) was added NaOAc (30.66 mg, 373.80 umol, 1 eq) and NaBH₃CN (70.47 mg, 1.12 mmol, 3 eq). The mixture was stirred at 25° C. for 12 h. LCMS showed starting material was consumed and the desired product was detected. The mixture was concentrated, and the residue was purified by prep-HPLC (neutral condition) to afford 3-(2-(4-(6-fluoro-3-methylbenzo[d]isoxazol-5-yl)piperazin-1-yl)ethyl)-8-(pyridin-2-yl)-3H-[1,2,4]triazolo[5,1-i]purin-5-amine (41 mg, 78.80 umol) as a white solid. LCMS for product (ESI+): m/z 514.3 [M+H]⁺, Rt: 2.538 min. ¹H NMR (400 MHz, CDCl₃) δ=8.86-8.78 (m, 1H), 8.56 (d, J=7.9 Hz, 1H), 7.97 (s, 1H), 7.91 (dt, J=1.8, 7.8 Hz, 1H), 7.47-7.39 (m, 1H), 7.25 (d, J=11.0 Hz, 1H), 7.08 (d, J=7.9 Hz, 1H), 6.06 (s, 2H), 4.34 (t, J=6.0 Hz, 2H), 3.08 (br s, 4H), 2.90 (t, J=6.0 Hz, 2H), 2.76 (br s, 4H), 2.55 (s, 3H).

### Example 23: Synthesis of 2-(4-(4-(2-(5-amino-8-(prop-1-yn-1-yl)-3H-[1,2,4]triazolo[5,1-i]purin-3-yl)ethyl)piperazin-1-yl)-3-fluorophenoxy)-2-methylpropanoic acid

### Step 1: Synthesis of methyl 2-(4-(4-(2-(5-amino-8-(prop-1-yn-1-yl)-3H-[1,2,4]triazolo[5,1-i]purin-3-yl)ethyl)piperazin-1-yl)-3-fluorophenoxy)-2-methylpropanoate

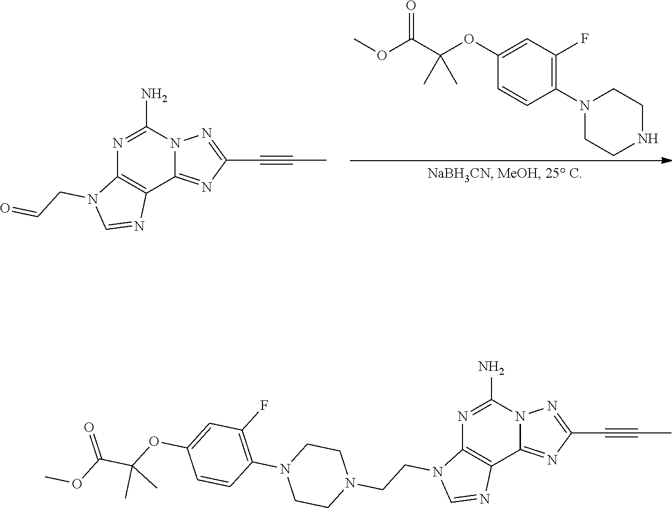 [SMILES: CC#Cc1nc2c3ncn(CC=O)c3nc(N)n2n1 ; COC(=O)C(C)(C)Oc1ccc(N2CCNCC2)c(F)c1 ; CC#Cc1nc2c3ncn(CCN4CCN(c5ccc(OC(C)(C)C(=O)OC)cc5F)CC4)c3nc(N)n2n1]

To a mixture of 2-(5-amino-8-prop-1-ynyl-[1,2,4]triazolo[5,1-f]purin-3-yl)acetaldehyde (50 mg, 195.90 umol, 1 eq) in MeOH (0.5 mL) was added methyl 2-(3-fluoro-4-piperazin-1-yl-phenoxy)-2-methyl-propanoate (58.05 mg, 195.90 umol, 1 eq), NaOAc (16.07 mg, 195.90 umol, 1 eq) and NaBH₃CN (36.93 mg, 587.69 umol, 3 eq). The mixture was stirred at 25° C. for 2 h. LCMS showed completion of the reaction. The mixture was poured into water (10 mL) and extracted with EtOAc (20 mL). The combined organic layer was washed with brine, dried over Na2SO4, filtered, and concentrated to afford methyl 2-(4-(4-(2-(5-amino-8-(prop-1-yn-1-yl)-3H-[1,2,4]triazolo[5,1-i]purin-3-yl)ethyl)piperazin-1-yl)-3-fluorophenoxy)-2-methylpropanoate (40 mg) as a yellow solid. LCMS for product (ESI+): m/z 536.3 [M+H]⁺, Rt: 1.149 min.

### Step 2: Synthesis of 2-(4-(4-(2-(5-amino-8-(prop-1-yn-1-yl)-3H-[1,2,4]triazolo[5,1-i]purin-3-yl)ethyl)piperazin-1-yl)-3-fluorophenoxy)-2-methylpropanoic acid

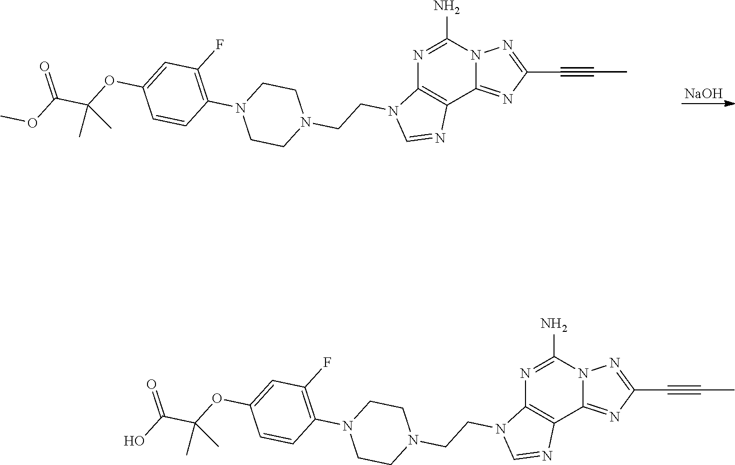 [SMILES: CC#Cc1nc2c3ncn(CCN4CCN(c5ccc(OC(C)(C)C(=O)OC)cc5F)CC4)c3nc(N)n2n1 ; "NaOH" ; CC#Cc1nc2c3ncn(CCN4CCN(c5ccc(OC(C)(C)C(=O)O)cc5F)CC4)c3nc(N)n2n1]

To a mixture of methyl 2-[4-[4-[2-(5-amino-8-prop-1-ynyl-[1,2,4]triazolo[5,1-f]purin-3-yl)ethyl]piperazin-1-yl]-3-fluoro-phenoxy]-2-methyl-propanoate (40 mg, 74.69 umol, 1 eq) in THF (0.6 mL), MeOH (0.4 mL) and H₂O (0.2 mL) was added NaOH (8.96 mg, 224.06 umol, 3 eq). The mixture was stirred at 25° C. for 3 h. LCMS showed completion of the reaction. The mixture was purified by prep-HPLC (neutral condition) to afford 2-(4-(4-(2-(5-amino-8-(prop-1-yn-1-yl)-3H-[1,2,4]triazolo[5,1-i]purin-3-yl)ethyl)piperazin-1-yl)-3-fluorophenoxy)-2-methylpropanoic acid (23 mg, 44.10 umol) as a white solid. LCMS for product (ESI+): m/z 522.3 [M+H]⁺, Rt: 1.981 min. ¹H NMR (400 MHz, DMSO-d6) δ=8.06 (s, 1H), 7.81 (br s, 2H), 6.90 (t, J=9.6 Hz, 1H), 6.66 (dd, J=2.7, 14.2 Hz, 1H), 6.58 (dd, J=2.3, 8.7 Hz, 1H), 4.27 (br t, J=6.2 Hz, 2H), 2.88 (br s, 4H), 2.76 (br t, J=6.0 Hz, 2H), 2.60 (br s, 4H), 2.14 (s, 3H), 1.43 (s, 6H).

### Example 24: Synthesis of 2-(4-(4-(2-(5-amino-8-(pyridin-2-yl)-3H-[1,2,4]triazolo[5,1-i]purin-3-yl)ethyl)piperazin-1-yl)-3-fluorophenoxy)-2-methylpropanoic acid

### Step 1: Synthesis of methyl 2-(4-(4-(2-(5-amino-8-(pyridin-2-yl)-3H-[1,2,4]triazolo[5,1-i]purin-3-yl)ethyl)piperazin-1-yl)-3-fluorophenoxy)-2-methylpropanoate

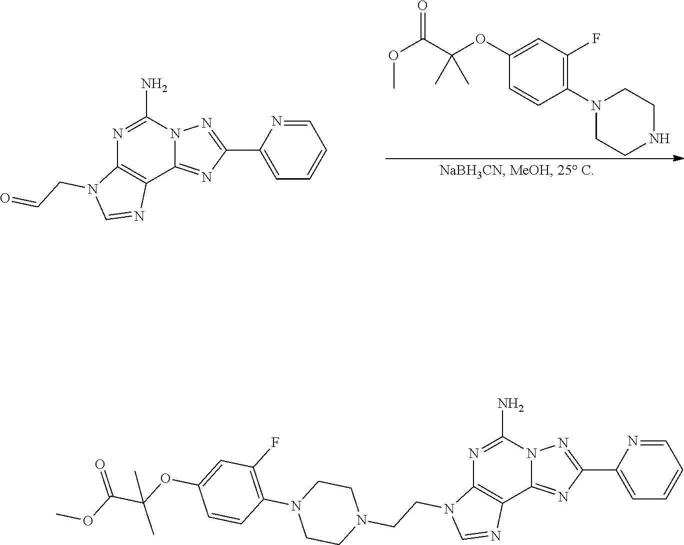 [SMILES: Nc1nc2c(ncn2CC=O)c2nc(-c3ccccn3)nn12 ; COC(=O)C(C)(C)Oc1ccc(N2CCNCC2)c(F)c1 ; COC(=O)C(C)(C)Oc1ccc(N2CCN(CCn3cnc4c3nc(N)n3nc(-c5ccccn5)nc43)CC2)c(F)c1]

To a mixture of 2-[5-amino-8-(2-pyridyl)-[1,2,4]triazolo[5,1-f]purin-3-yl]acetaldehyde (105 mg, 356.81 umol, 1 eq) and methyl 2-(3-fluoro-4-piperazin-1-yl-phenoxy)-2-methyl-propanoate (105.74 mg, 356.81 umol, 1 eq) in MeOH (1 mL) was added NaOAc (29.27 mg, 356.81 umol, 1 eq) and NaBH₃CN (67.27 mg, 1.07 mmol, 3 eq). The mixture was stirred at 25° C. for 12 h. LCMS showed the reaction was completed. The mixture was concentrated. The residue was purified by prep-HPLC (neutral condition) to afford methyl 2-(4-(4-(2-(5-amino-8-(pyridin-2-yl)-3H-[1,2,4]triazolo[5,1-i]purin-3-yl)ethyl)piperazin-1-yl)-3-fluorophenoxy)-2-methylpropanoate (100 mg, 174.03 umol) as a white solid. LCMS for product (ESI+): m/z 575.2 [M+H]⁺, Rt: 0.898 min.

Step 2: Synthesis of 2-(4-(4-(2-(5-amino-8-(pyridin-2-yl)-3H-[1,2,4]triazolo[5,1-i]purin-3-yl)ethyl)piperazin-1-yl)-3-fluorophenoxy)-2-methylpropanoic acid

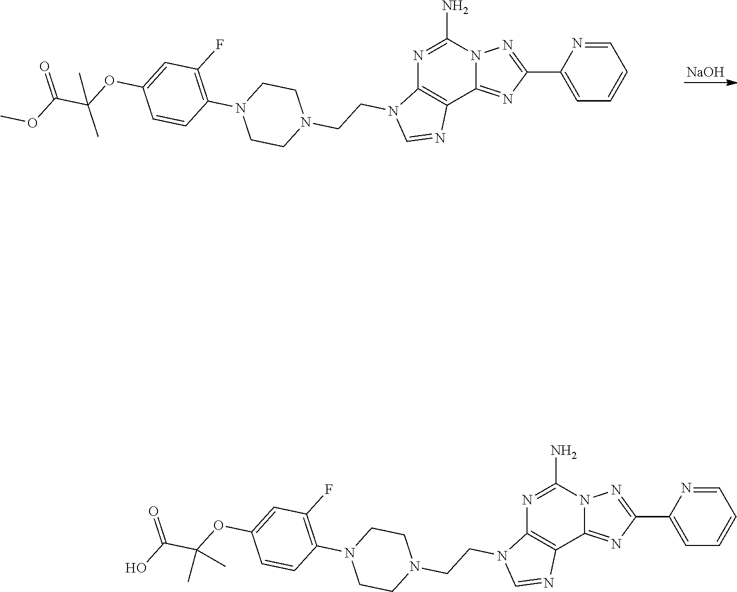 [SMILES: COC(=O)C(C)(C)Oc1ccc(N2CCN(CCn3cnc4c3nc(N)n3nc(-c5ccccn5)nc43)CC2)c(F)c1 ; "NaOH" ; CC(C)(Oc1ccc(N2CCN(CCn3cnc4c3nc(N)n3nc(-c5ccccn5)nc43)CC2)c(F)c1)C(=O)O]

To a mixture of methyl 2-[4-[4-[2-[5-amino-8-(2-pyridyl)-[1,2,4]triazolo[5,1-f]purin-3-yl]ethyl]piperazin-1-yl]-3-fluoro-phenoxy]-2-methyl-propanoate (95 mg, 165.33 umol, 1 eq) in THF (1.2 mL), MeOH (0.8 mL) and H₂O (0.4 mL) was added NaOH (19.84 mg, 495.99 umol, 3 eq). The mixture was stirred at 25° C. for 3 h. LCMS showed the reaction was completed. The mixture was concentrated, and the residue was purified by prep-HPLC (neutral condition) to afford 2-(4-(4-(2-(5-amino-8-(pyridin-2-yl)-3H-[1,2,4]triazolo[5,1-i]purin-3-yl)ethyl)piperazin-1-yl)-3-fluorophenoxy)-2-methylpropanoic acid (23 mg, 41.03 umol) as a white solid. LCMS for product (ESI+): m/z 561.3 [M+H]⁺, Rt: 1.955 min. ¹H NMR (400 MHz, DMSO-d₆) δ=8.79-8.69 (m, 1H), 8.34 (d, J=7.9 Hz, 1H), 8.10 (s, 1H), 8.02 (dt, J=1.8, 7.8 Hz, 1H), 7.93-7.77 (m, 2H), 7.55 (ddd, J=1.2, 4.8, 7.5 Hz, 1H), 6.97-6.86 (m, 1H), 6.66 (dd, J=2.8, 14.0 Hz, 1H), 6.59 (dd, J=2.2, 8.8 Hz, 1H), 4.31 (br t, J=6.0 Hz, 2H), 2.90 (br s, 4H), 2.79 (br t, J=6.1 Hz, 2H), 2.62 (br s, 4H), 1.45 (s, 6H).

### Example 25: Synthesis of 8-(cyclopropylethynyl)-3-(2-(4-(2-fluoro-5-(oxazol-2-yl)phenyl)piperazin-1-yl)ethyl)-3H-[1,2,4]triazolo[5,1-i]purin-5-amine

### Step 1: Synthesis of N′-(2-amino-9-(2,2-diethoxyethyl)-9H-purin-6-yl)-3-cyclopropylpropiolohydrazide

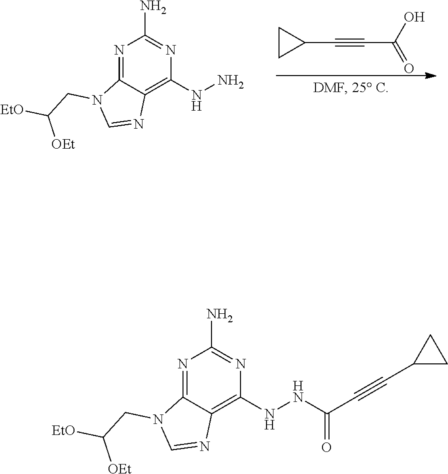 [SMILES: CCOC(Cn1cnc2c(NN)nc(N)nc21)OCC ; O=C(O)C#CC1CC1 ; CCOC(Cn1cnc2c(NNC(=O)C#CC3CC3)nc(N)nc21)OCC]

To a solution of 9-(2,2-diethoxyethyl)-6-hydrazino-purin-2-amine (1 g, 3.55 mmol, 1 eq) in DMF (10 mL) was added 3-cyclopropylprop-2-ynoic acid (587.12 mg, 5.33 mmol, 1.5 eq) and DIEA (1.38 g, 10.66 mmol, 1.9 mL, 3 eq) at 0° C. A solution of T3P (4.52 g, 7.11 mmol, 4.23 mL, 50% purity, 2 eq) in DMF (5 mL) was added into the mixture. The mixture was stirred at 25° C. for 2 h. LCMS showed consumption of starting material and formation of N′-(2-amino-9-(2,2-diethoxyethyl)-9H-purin-6-yl)-3-cyclopropylpropiolohydrazide. The mixture was poured into water (100 mL), extracted with ethyl acetate (3×50 mL), separated, the organic layer was washed with brine (50 mL), dried over Na₂SO₄ and concentrated. The residue was triturated in petroleum ether/ethyl acetate (5:1, 20 mL) and the resulting solid was filtered and used without further purification (1 g, 2.68 mmol) as a white solid. LCMS for product (ESI+): m/z 374.2 [M+H]⁺, Rt: 1.074 min. ¹H NMR (400 MHz, CDCl₃) δ=7.59 (br s, 1H), 5.41-4.80 (m, 2H), 4.67 (t, J=5.3 Hz, 1H), 4.10 (d, J=5.3 Hz, 2H), 3.73 (dd, J=7.1, 9.3 Hz, 2H), 3.58-3.45 (m, 2H), 1.42-1.31 (m, 1H), 1.18 (t, J=7.1 Hz, 6H), 0.96-0.84 (m, 4H).

### Step 2: Synthesis of 8-(cyclopropylethynyl)-3-(2,2-diethoxyethyl)-3H-[1,2,4]triazolo[5,1-i]purin-5-amine

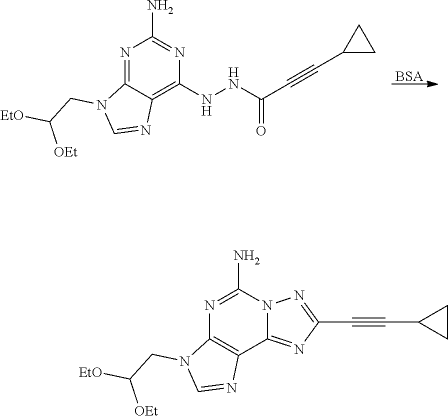

To a solution of N′-[2-amino-9-(2,2-diethoxyethyl)purin-6-yl]-3-cyclopropyl-prop-2-ynehydrazide (500 mg, 1.34 mmol, 1 eq) in BSA (4.12 g, 20.23 mmol, 5.00 mL, 15.11 eq). The mixture was stirred at 120° C. for 2 h. LCMS showed 60% starting material was remaining and 40% product formation. The mixture was concentrated, and the residue was poured into water (50 mL), extracted with ethyl acetate (3×10 mL), separated and the organic layer was washed with brine (10 mL), dried over Na₂SO₄ and concentrated. The resulting residue was purified by prep-TLC (Ethyl acetate/Methanol=5:1) to afford 8-(cyclopropylethynyl)-3-(2,2-diethoxyethyl)-3H-[1,2,4]triazolo[5,1-i]purin-5-amine as a white solid. LCMS for product (ESI+): m/z 356.2 [M+H]⁺, Rt: 0.913 min.

### Step 3: Synthesis of 2-(5-amino-8-(cyclopropylethynyl)-3H-[1,2,4]triazolo[5,1-i]purin-3-yl)acetaldehyde

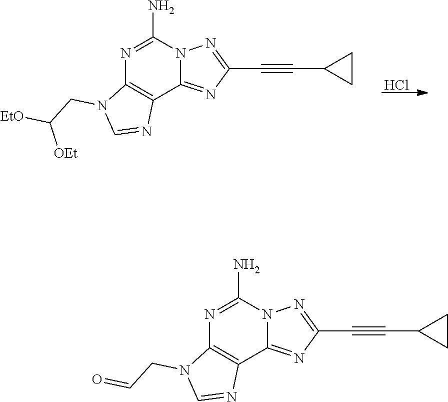 [SMILES: CCOC(Cn1cnc2c1nc(N)n1nc(C#CC3CC3)nc21)OCC ; Cl ; Nc1nc2c(ncn2CC=O)c2nc(C#CC3CC3)nn12]

A solution of 8-(2-cyclopropylethynyl)-3-(2,2-diethoxyethyl)-[1,2,4]triazolo[5,1-f]purin-5-amine (100 mg, 281.38 umol, 1 eq) in HCl (1 M, 2.3 mL, 8.20 eq) was stirred at 40° C. for 24 h. LCMS showed starting material was consumed and a new peak corresponding to the desired MS.

The mixture was concentrated to afford 2-(5-amino-8-(cyclopropylethynyl)-3H-[1,2,4]triazolo[5,1-i]purin-3-yl)acetaldehyde (70 mg, 248.87 umol) as a yellow solid (used without further purification). LCMS for product (ESI+): m/z 300.3 [M+19]⁺, Rt: 0.807 min.

### Step 4: Synthesis of 8-(cyclopropylethynyl)-3-(2-(4-(2-fluoro-5-(oxazol-2-yl)phenyl)piperazin-1-yl)ethyl)-3H-[1,2,4]triazolo[5,1-i]purin-5-amine

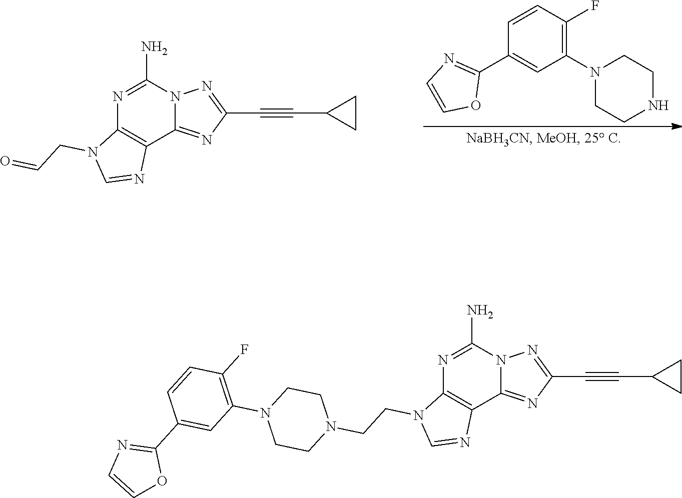 [SMILES: Nc1nc2c(ncn2CC=O)c2nc(C#CC3CC3)nn12 ; Fc1ccc(-c2ncco2)cc1N1CCNCC1 ; Nc1nc2c(ncn2CCN2CCN(c3cc(-c4ncco4)ccc3F)CC2)c2nc(C#CC3CC3)nn12]

To a solution of 2-[5-amino-8-(2-cyclopropylethynyl)-[1,2,4]triazolo[5,1-f]purin-3-yl]acetaldehyde (70 mg, 248.87 umol, 1 eq) in MeOH (1 mL) was added 2-(4-fluoro-3-piperazin-1-yl-phenyl)oxazole (56.49 mg, 199.10 umol, 0.8 eq, HCl) and NaOAc (40.83 mg, 497.74 umol, 2 eq) and NaBH₃CN (46.92 mg, 746.61 umol, 3 eq) at 25° C. The mixture was stirred at 25° C. for 12 h. LCMS showed starting material was consumed and formation of 8-(cyclopropylethynyl)-3-(2-(4-(2-fluoro-5-(oxazol-2-yl)phenyl)piperazin-1-yl)ethyl)-3H-[1,2,4]triazolo[5,1-i]purin-5-amine. The mixture was filtered, and the filtrate was purified by prep-HPLC to afford 8-(cyclopropylethynyl)-3-(2-(4-(2-fluoro-5-(oxazol-2-yl)phenyl)piperazin-1-yl)ethyl)-3H-[1,2,4]triazolo[5,1-i]purin-5-amine (6.2 mg, 11.49 umol) as a white solid. LCMS for product (ESI+): m/z 513.2 [M+H]⁺, Rt: 1.978 min. ¹H NMR (400 MHz, CDCl₃) δ=7.94 (s, 1H), 7.70 (s, 1H), 7.63 (br d, J=9.9 Hz, 2H), 7.22 (s, 1H), 7.10 (br dd, J=8.8, 12.2 Hz, 1H), 5.78 (br s, 2H), 4.31 (br t, J=5.9 Hz, 2H), 3.18 (br s, 4H), 2.86 (br t, J=6.1 Hz, 2H), 2.73 (br s, 4H), 1.57-1.52 (m, 1H), 1.03-0.89 (m, 4H).

### Example 26: Synthesis of methyl 5-amino-7-(2-(4-(2-fluoro-5-(oxazol-2-yl)phenyl)piperazin-1-yl)ethyl)-9-methyl-2-(pyridin-2-yl)-7H-pyrrolo[3,2-e][1,2,4]triazolo[1,5-c]pyrimidine-8-carboxylate

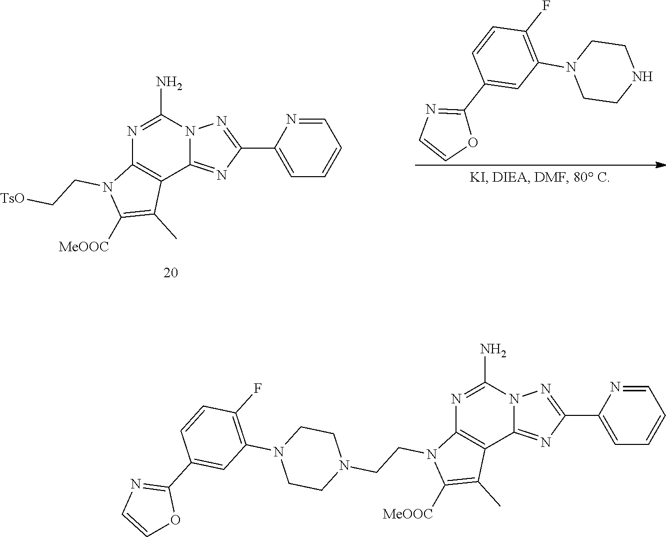 [SMILES: COC(=O)c1c(C)c2c(nc(N)n3nc(-c4ccccn4)nc23)n1CCOS(=O)(=O)c1ccc(C)cc1 ; Fc1ccc(-c2ncco2)cc1N1CCNCC1 ; COC(=O)c1c(C)c2c(nc(N)n3nc(-c4ccccn4)nc23)n1CCN1CCN(c2cc(-c3ncco3)ccc2F)CC1]

A mixture of methyl 5-amino-9-methyl-7-[2-(p-tolylsulfonyloxy)ethyl]-2-(2-pyridyl)-[1,2,4]triazolopyrrolopyrimidine-8-carboxylate (0.3 g, 575 umol, 1 eq), 2-(4-fluoro-3-piperazin-1-yl-phenyl)oxazole (284 mg, 1.15 mmol, 2 eq), potassium iodide (95 mg, 575 umol, 1 eq) and N,N-ethyldiisopropylamine (297 mg, 2.30 mmol, 400 uL, 4 eq) in N,N-dimethylformamide (5.7 mL) was degassed and purged with N₂ 3 times. The mixture was stirred at 80° C. for 12 h under N₂ atmosphere. LC-MS indicated disappearance of starting material and formation of a new peak with the desired mass. 1M hydrochloric acid (3 mL) was added to the reaction mixture to make a clear solution. The reaction solution was purified by prep-HPLC (HCl condition) to afford 5-amino-7-(2-(4-(2-fluoro-5-(oxazol-2-yl)phenyl)piperazin-1-yl)ethyl)-9-methyl-2-(pyridin-2-yl)-7H-pyrrolo[3,2-e][1,2,4]triazolo[1,5-c]pyrimidine-8-carboxylate (45 mg, 68.6 umol) as a yellow solid. LCMS for product (ESI+): m/z 597.2 (M+H)⁺, Rt: 3.722 min. ¹H NMR (400 MHz, DMSO-d₆) δ=10.14 (br s, 1H), 8.77 (d, J=4.9 Hz, 1H), 8.34 (d, J=7.8 Hz, 1H), 8.26-8.16 (m, 3H), 8.06 (dt, J=1.5, 7.8 Hz, 1H), 7.67-7.63 (m, 1H), 7.62-7.57 (m, 2H), 7.44-7.32 (m, 2H), 4.85 (br t, J=5.6 Hz, 2H), 4.03 (br d, J=10.8 Hz, 2H), 3.89 (s, 3H), 3.71-3.59 (m, 4H), 3.35 (br d, J=10.8 Hz, 2H), 3.26-3.15 (m, 2H), 2.80 (s, 3H).

### Example 27: Synthesis of 5-amino-7-(2-(4-(2-fluoro-5-(oxazol-2-yl)phenyl)piperazin-1-yl)ethyl)-9-methyl-2-(pyridin-2-yl)-7H-pyrrolo[3,2-e][1,2,4]triazolo[1,5-c]pyrimidine-8-carboxylic acid

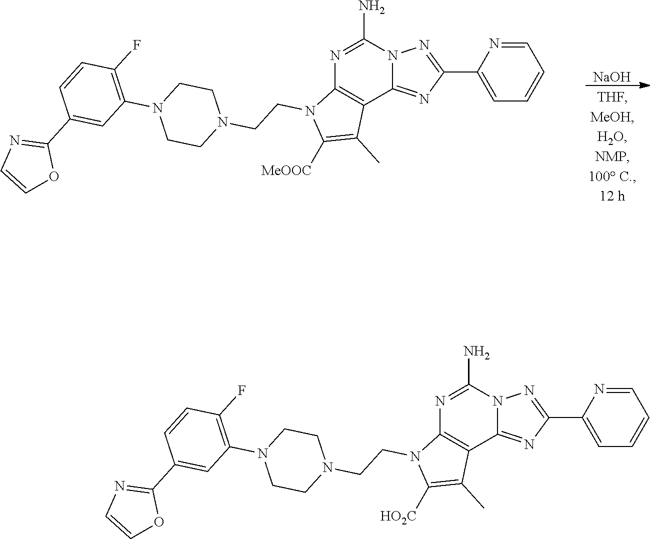

To a suspension of methyl 5-amino-7-[2-[4-(2-fluoro-5-oxazol-2-yl-phenyl)piperazin-1-yl]ethyl]-9-methyl-2-(2-pyridyl)-[1,2,4]triazolopyrrolopyrimidine-8-carboxylate (30 mg, 50.2 umol, 1 eq) in tetrahydrofuran (0.6 mL), N-methyl-2-pyrrolidone (1.5 mL) and methanol (0.6 mL) was added a solution of NaOH (14.0 mg, 351 umol, 7 eq) in H₂O (0.3 mL). The mixture was stirred at 100° C. for 12 h. LC-MS indicated disappearance of starting material and formation of a new peak with the desired mass. The reaction mixture was concentrated under reduced pressure to yield a red liquid. The red liquid was purified by prep-HPLC (neutral condition) to afford 5-amino-7-(2-(4-(2-fluoro-5-(oxazol-2-yl)phenyl)piperazin-1-yl)ethyl)-9-methyl-2-(pyridin-2-yl)-7H-pyrrolo[3,2-e][1,2,4]triazolo[1,5-c]pyrimidine-8-carboxylic acid (4 mg, 6.87 umol) as a white powder. LCMS for product (ESI+): m/z 583.2 (M+H)⁺, Rt: 2.10 min. ¹H NMR (400 MHz, DMSO-d₆) δ=8.75 (d, J=4.4 Hz, 1H), 8.31 (d, J=7.8 Hz, 1H), 8.20 (s, 1H), 8.01 (t, J=8.1 Hz, 1H), 7.93 (br s, 1H), 7.55 (br d, J=3.9 Hz, 3H), 7.36 (s, 1H), 7.29 (dd, J=8.3, 12.7 Hz, 1H), 4.71-4.64 (m, 2H), 3.31 (br s, 2H), 3.05 (br s, 4H), 2.76 (s, 3H), 2.67 (br s, 6H).

### Example 28: Synthesis of 5-amino-7-(2-(4-(2-fluoro-5-(oxazol-2-yl)phenyl)piperazin-1-yl)ethyl)-N,9-dimethyl-2-(pyridin-2-yl)-7H-pyrrolo[3,2-e][1,2,4]triazolo[1,5-c]pyrimidine-8-carboxamide

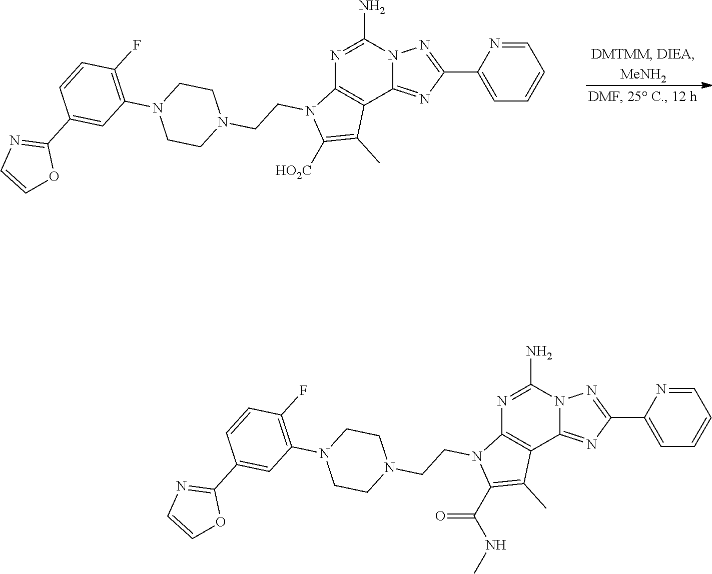 [SMILES: Cc1c(C(=O)O)n(CCN2CCN(c3cc(-c4ncco4)ccc3F)CC2)c2nc(N)n3nc(-c4ccccn4)nc3c12 ; "DMTMM, DIEA,MeNH2" ; CNC(=O)c1c(C)c2c(nc(N)n3nc(-c4ccccn4)nc23)n1CCN1CCN(c2cc(-c3ncco3)ccc2F)CC1]

To a solution of 5-amino-7-[2-[4-(2-fluoro-5-oxazol-2-yl-phenyl)piperazin-1-yl]ethyl]-9-methyl-2-(2-pyridyl)-[1,2,4]triazolopyrrolopyrimidine-8-carboxylic acid (15 mg, 25.7 umol, 1 eq) in N,N-dimethylformamide (1 mL) was added 4-(4,6-dimethoxy-1,3,5-triazin-2-yl)-4-methyl-morpholin-4-ium; tetrafluoroborate (16.8 mg, 51.4 umol, 2 eq) and N,N-diisopropylethylamine (16.6 mg, 128 umol, 22.4 uL, 5 eq). The mixture was stirred at 25° C. for 2 h. A solution of methylamine in tetrahydrofuran (2 M, 51.4 uL, 4 eq) was added to the mixture and the stirring was continued at 25° C. for 10 h. LC-MS indicated disappearance of starting material and formation of a new peak with the desired mass. N, N-dimethylformamide (1 mL) was added to the reaction mixture and the mixture was purified by prep-HPLC (HCl condition) to afford 5-amino-7-(2-(4-(2-fluoro-5-(oxazol-2-yl)phenyl)piperazin-1-yl)ethyl)-N,9-dimethyl-2-(pyridin-2-yl)-7H-pyrrolo[3,2-e][1,2,4]triazolo[1,5-c]pyrimidine-8-carboxamide (4 mg, 6.33 umol, HCl) as an orange solid. LCMS for product (ESI+): m/z 596.1 (M+H)⁺, Rt: 1.953 min. ¹H NMR (400 MHz, METHANOL-d₄) δ=9.00-8.91 (m, 2H), 8.80 (dt, J=1.5, 7.8 Hz, 1H), 8.24-8.18 (m, 1H), 8.00 (s, 1H), 7.76-7.70 (m, 2H), 7.33-7.23 (m, 2H), 4.75 (br t, J=5.1 Hz, 2H), 3.85-3.76 (m, 4H), 3.71 (br d, J=13.2 Hz, 2H), 3.50-3.41 (m, 2H), 3.30-3.24 (m, 2H), 3.04 (s, 3H), 2.79 (s, 3H).

### Example 29: Synthesis of 5-amino-7-(2-(4-(2-fluoro-5-(oxazol-2-yl)phenyl)piperazin-1-yl)ethyl)-9-methyl-2-(pyridin-2-yl)-7H-pyrrolo[3,2-e][1,2,4]triazolo[1,5-c]pyrimidine-8-carboxamide

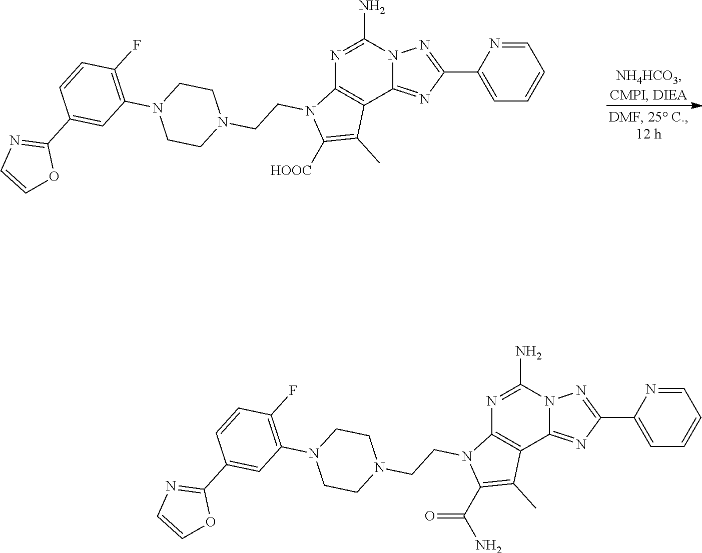 [SMILES: Cc1c(C(=O)O)n(CCN2CCN(c3cc(-c4ncco4)ccc3F)CC2)c2nc(N)n3nc(-c4ccccn4)nc3c12 ; "NH4HCO3,CMPI, DIEA" ; Cc1c(C(N)=O)n(CCN2CCN(c3cc(-c4ncco4)ccc3F)CC2)c2nc(N)n3nc(-c4ccccn4)nc3c12]

To a solution of 5-amino-7-[2-[4-(2-fluoro-5-oxazol-2-yl-phenyl)piperazin-1-yl]ethyl]-9-methyl-2-(2-pyridyl)-[1,2,4]triazolo pyrrolo pyrimidine-8-carboxylic acid (15 mg, 25.7 umol, 1 eq) in N,N-dimethylformamide (1 mL) was added N,N-diisopropylethylamine (16.6 mg, 128 umol, 22.4 uL, 5 eq) and NH₄HCO₃ (3.05 mg, 38.6 umol, 3.18 uL, 1.5 eq). The mixture was stirred at 25° C. for 1 h. Then 2-chloro-1-methyl-pyridin-1-ium; iodide (13.1 mg, 51.4 umol, 2 eq) was added to the mixture and stirred at 25° C. for 11 h. LC-MS indicated disappearance of starting material and formation of a new peak with the desired mass. N,N-dimethylformamide (1 mL) was added to the reaction mixture and the mixture was purified by prep-HPLC (HCl condition) to afford the 5-amino-7-(2-(4-(2-fluoro-5-(oxazol-2-yl)phenyl)piperazin-1-yl)ethyl)-9-methyl-2-(pyridin-2-yl)-7H-pyrrolo[3,2-e][1,2,4]triazolo[1,5-c]pyrimidine-8-carboxamide (4 mg, 6.39 umol, HCl) as an orange solid. LCMS for product (ESI+): m/z 582.1 (M+H)⁺, Rt: 1.892 min. ¹H NMR (400 MHz, METHANOL-d₄) δ=9.01-8.92 (m, 2H), 8.80 (dt, J=1.5, 7.8 Hz, 1H), 8.20 (t, J=7.3 Hz, 1H), 8.02-7.98 (m, 1H), 7.75-7.70 (m, 2H), 7.35-7.23 (m, 2H), 4.81-4.76 (m, 2H), 3.85-3.75 (m, 4H), 3.70 (br d, J=13.7 Hz, 2H), 3.49-3.41 (m, 2H), 3.28-3.22 (m, 2H), 2.85 (s, 3H).

### Example 30: Synthesis of 5-amino-N-cyclopropyl-7-(2-(4-(2-fluoro-5-(oxazol-2-yl)phenyl)piperazin-1-yl)ethyl)-9-methyl-2-(pyridin-2-yl)-7H-pyrrolo[3,2-e][1,2,4]triazolo[1,5-c]pyrimidine-8-carboxamide

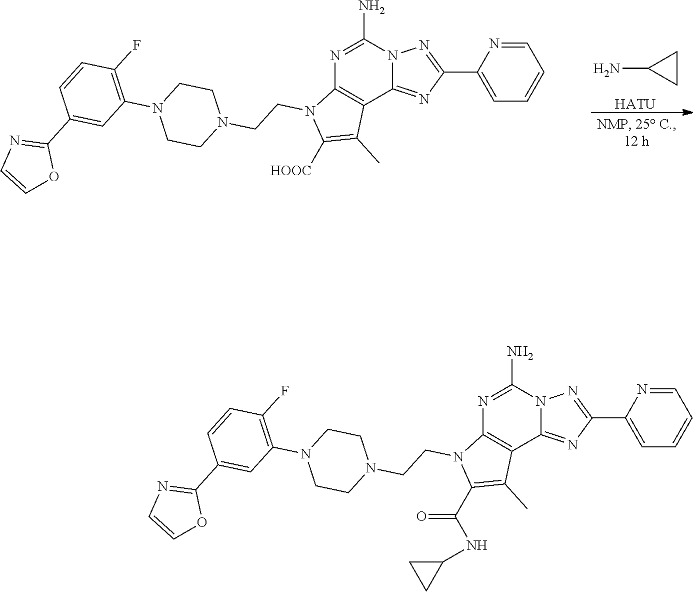

To a solution of 5-amino-7-[2-[4-(2-fluoro-5-oxazol-2-yl-phenyl)piperazin-1-yl]ethyl]-9-methyl-2-(2-pyridyl)-[1,2,4]triazolo pyrrolo pyrimidine-8-carboxylic acid (25 mg, 42.9 umol, 1 eq) and cyclopropylamine (7.35 mg, 128 umol, 8.92 uL, 3 eq) in 1-methyl-2-pyrrolidinone (2 mL) was added O-(7-Azabenzotriazol-1-yl)-N,N,N′,N′-tetramethyluronium hexafluorophosphate (24.4 mg, 64.3 umol, 1.5 eq). The mixture was stirred at 25° C. for 12 h. LC-MS indicated disappearance of starting material and formation of a new peak with the desired mass. The reaction mixture was purified by prep-HPLC (HCl condition) to afford 5-amino-N-cyclopropyl-7-(2-(4-(2-fluoro-5-(oxazol-2-yl)phenyl)piperazin-1-yl)ethyl)-9-methyl-2-(pyridin-2-yl)-7H-pyrrolo[3,2-e][1,2,4]triazolo[1,5-c]pyrimidine-8-carboxamide (20 mg, 30.4 umol, HCl) as a yellow solid. LCMS for product (ESI+): m/z 622.2 (M+H)⁺, Rt: 1.890 min. ¹H NMR (400 MHz, DMSO-d₆) δ=9.83 (br s, 1H), 8.76 (br d, J=4.0 Hz, 1H), 8.36-8.28 (m, 2H), 8.23 (s, 1H), 8.09-7.96 (m, 3H), 7.70-7.54 (m, 3H), 7.44-7.32 (m, 2H), 4.61 (br s, 2H), 3.94 (br d, J=11.0 Hz, 2H), 3.68 (br d, J=12.8 Hz, 4H), 3.38 (br s, 2H), 3.21-3.09 (m, 2H), 2.91 (br s, 1H), 2.62 (s, 3H), 0.77 (br d, J=4.9 Hz, 2H), 0.65 (br s, 2H).

### Example 31: Synthesis of (5-amino-7-(2-(4-(2-fluoro-5-(oxazol-2-yl)phenyl)piperazin-1-yl)ethyl)-9-methyl-2-(pyridin-2-yl)-7H-pyrrolo[3,2-e][1,2,4]triazolo[1,5-c]pyrimidin-8-yl)(azetidin-1-yl)methanone

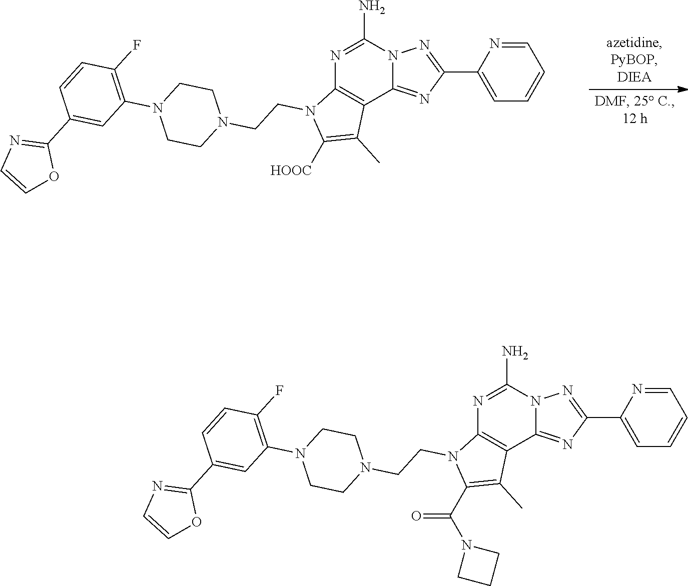 [SMILES: Cc1c(C(=O)O)n(CCN2CCN(c3cc(-c4ncco4)ccc3F)CC2)c2nc(N)n3nc(-c4ccccn4)nc3c12 ; Cc1c(C(=O)N2CCC2)n(CCN2CCN(c3cc(-c4ncco4)ccc3F)CC2)c2nc(N)n3nc(-c4ccccn4)nc3c12]

To a solution of 5-amino-7-[2-[4-(2-fluoro-5-oxazol-2-yl-phenyl)piperazin-1-yl]ethyl]-9-methyl-2-(2-pyridyl)-[1,2,4]triazolo pyrrolo pyrimidine-8-carboxylic acid (25 mg, 42.9 umol, 1 eq) and azetidine (9.80 mg, 171 umol, 11.5 uL, 4 eq) in N,N-dimethylformamide (1 mL) was added N,N-diisopropylethylamine (13.8 mg, 107 umol, 18.6 uL, 2.5 eq) and benzotriazol-1-yloxy(tripyrrolidin-1-yl)phosphonium; hexafluorophosphate (26.8 mg, 51.4 umol, 1.2 eq). The mixture was stirred at 25° C. for 12 h. LC-MS indicated disappearance of starting material and formation of a new peak with the desired mass. N, N-dimethylformamide (1 mL) was added to the reaction mixture and the mixture was purified by prep-HPLC (HCl condition) to afford (5-amino-7-(2-(4-(2-fluoro-5-(oxazol-2-yl)phenyl)piperazin-1-yl)ethyl)-9-methyl-2-(pyridin-2-yl)-7H-pyrrolo[3,2-e][1,2,4]triazolo[1,5-c]pyrimidin-8-yl)(azetidin-1-yl)methanone (12 mg, 18.1 umol, HCl) as a yellow solid. LCMS for product (ESI+): m/z 622.2 (M+H)⁺, Rt: 2.008 min. ¹H NMR (400 MHz, METHANOL-d₄) δ=9.00-8.93 (m, 2H), 8.86-8.79 (m, 1H), 8.26-8.19 (m, 1H), 8.01 (s, 1H), 7.78-7.70 (m, 2H), 7.33 (s, 1H), 7.27 (dd, J=8.7, 12.9 Hz, 1H), 4.73 (td, J=5.2, 10.6 Hz, 2H), 4.34 (br t, J=7.3 Hz, 2H), 3.86-3.63 (m, 8H), 3.51-3.40 (m, 2H), 3.29-3.23 (m, 2H), 2.82-2.68 (m, 3H), 2.47 (br t, J=7.7 Hz, 1H), 2.17 (t, J=6.5 Hz, 1H).

### Example 32: Synthesis of 5-amino-7-(2-(4-(6-fluoro-3-methylbenzo[d]isoxazol-5-yl)piperazin-1-yl)ethyl)-9-methyl-2-(pyridin-2-yl)-7H-pyrrolo[3,2-e][1,2,4]triazolo[1,5-c]pyrimidine-8-carboxylic acid

### Step 1: Synthesis of methyl 5-amino-7-(2-(4-(6-fluoro-3-methylbenzo[d]isoxazol-5-yl)piperazin-1-yl)ethyl)-9-methyl-2-(pyridin-2-yl)-7H-pyrrolo[3,2-e][1,2,4]triazolo[1,5-c]pyrimidine-8-carboxylate

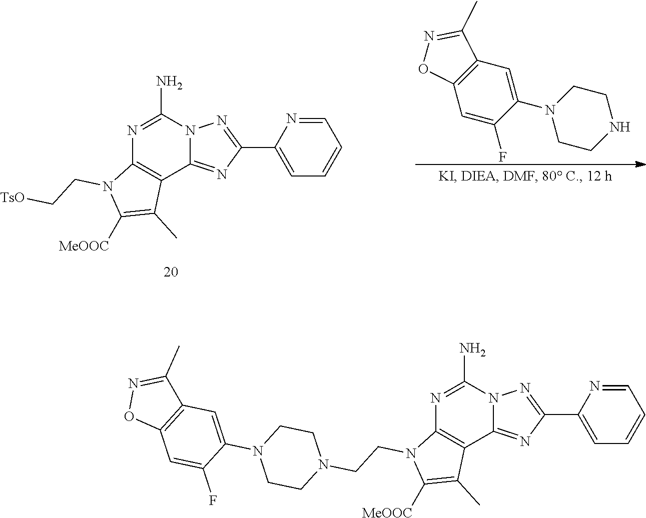 [SMILES: COC(=O)c1c(C)c2c(nc(N)n3nc(-c4ccccn4)nc23)n1CCOS(=O)(=O)c1ccc(C)cc1 ; Cc1noc2cc(F)c(N3CCNCC3)cc12 ; COC(=O)c1c(C)c2c(nc(N)n3nc(-c4ccccn4)nc23)n1CCN1CCN(c2cc3c(C)noc3cc2F)CC1]

To a solution of methyl 5-amino-9-methyl-7-[2-(p-tolylsulfonyloxy)ethyl]-2-(2-pyridyl)-[1,2,4]triazolo pyrrolo pyrimidine-8-carboxylate (0.5 g, 958 umol, 1 eq) and 6-fluoro-3-methyl-5-piperazin-1-yl-1,2-benzoxazole (270 mg, 1.15 mmol, 1.2 eq) in N,N-dimethylformamide (3.8 mL) was added KI (159 mg, 958 umol, 1 eq) and N,N-diisopropylethylamine (371 mg, 2.88 mmol, 500 uL, 3 eq). The mixture was stirred at 80° C. for 12 h. LC-MS indicated disappearance of starting material and formation of a new peak with the desired mass. The reaction mixture was concentrated under reduced pressure and residue was purified by prep-HPLC (TFA condition) to afford methyl 5-amino-7-(2-(4-(6-fluoro-3-methylbenzo[d]isoxazol-5-yl)piperazin-1-yl)ethyl)-9-methyl-2-(pyridin-2-yl)-7H-pyrrolo[3,2-e][1,2,4]triazolo[1,5-c]pyrimidine-8-carboxylate (200 mg, 286 umol, TFA) as a yellow solid. ¹H NMR (400 MHz, DMSO-d₆) δ=9.21 (br s, 1H), 8.76 (d, J=4.9 Hz, 1H), 8.33 (d, J=8.3 Hz, 1H), 8.17 (br s, 2H), 8.03 (t, J=7.8 Hz, 1H), 7.75 (d, J=11.7 Hz, 1H), 7.59-7.54 (m, 1H), 7.50 (d, J=7.8 Hz, 1H), 4.84 (br s, 2H), 4.08 (br s, 2H), 3.90 (s, 3H), 3.68 (br s, 2H), 3.54 (br s, 2H), 3.03 (br s, 2H), 2.81 (s, 3H), 2.52 (br s, 5H).

### Step 2: Synthesis of 5-amino-7-(2-(4-(6-fluoro-3-methylbenzo[d]isoxazol-5-yl)piperazin-1-yl)ethyl)-9-methyl-2-(pyridin-2-yl)-7H-pyrrolo[3,2-e][1,2,4]triazolo[1,5-c]pyrimidine-8-carboxylic acid

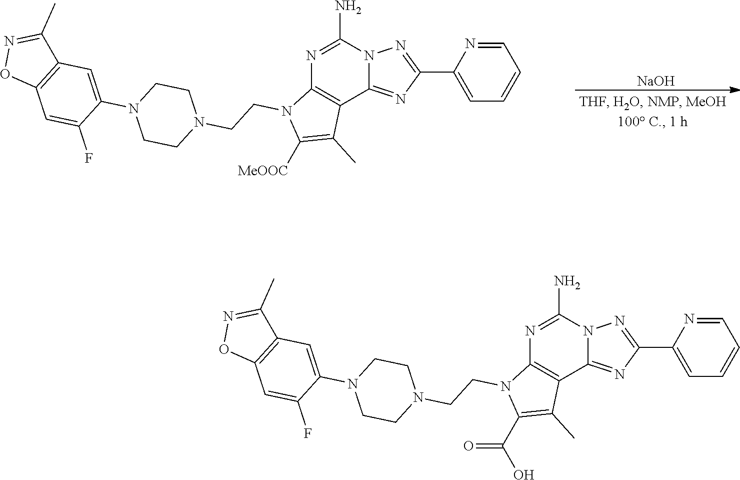 [SMILES: COC(=O)c1c(C)c2c(nc(N)n3nc(-c4ccccn4)nc23)n1CCN1CCN(c2cc3c(C)noc3cc2F)CC1 ; "NaOH" ; Cc1noc2cc(F)c(N3CCN(CCn4c(C(=O)O)c(C)c5c4nc(N)n4nc(-c6ccccn6)nc54)CC3)cc12]

To a solution of methyl 5-amino-7-[2-[4-(6-fluoro-3-methyl-1,2-benzoxazol-5-yl)piperazin-1-yl]ethyl]-9-methyl-2-(2-pyridyl)-[1,2,4]triazolo pyrrolo pyrimidine-8-carboxylate (40 mg, 68.4 umol, 1 eq) in a mixture of methanol (0.4 mL), tetrahydrofuran (0.4 mL) and 1-methyl-2-pyrrolidinone (1 mL) was added NaOH (19.1 mg, 478 umol, 7 eq) in water (0.2 mL). The mixture was stirred at 100° C. for 0.5 h. LC-MS indicated disappearance of starting material and formation of a new peak with the desired mass. Three additional vials were set up as described above and all four reaction mixtures were combined. To the combined reaction mixture was added trifluoroacetic acid (0.5 mL) to form a clear solution and the solution was purified by prep-HPLC (neutral condition) to afford 5-amino-7-(2-(4-(6-fluoro-3-methylbenzo[d]isoxazol-5-yl)piperazin-1-yl)ethyl)-9-methyl-2-(pyridin-2-yl)-7H-pyrrolo[3,2-e][1,2,4]triazolo[1,5-c]pyrimidine-8-carboxylic acid (60 mg, 105 umol) as a white solid.

LCMS for product (ESI+): m/z 571.3 (M+H)⁺, Rt: 2.178 min. ¹H NMR (400 MHz, DMSO-d₆) δ=8.75 (d, J=4.2 Hz, 1H), 8.31 (d, J=7.5 Hz, 1H), 8.01 (t, J=6.8 Hz, 1H), 7.92 (br s, 2H), 7.65 (d, J=11.5 Hz, 1H), 7.57-7.51 (m, 1H), 7.38 (d, J=8.4 Hz, 1H), 4.71-4.64 (m, 2H), 2.97 (br s, 4H), 2.77 (s, 3H), 2.68 (br d, J=5.1 Hz, 7H).

### Example 33: Synthesis of 5-amino-7-(2-(4-(6-fluoro-3-methylbenzo[d]isoxazol-5-yl)piperazin-1-yl)ethyl)-9-methyl-2-(pyridin-2-yl)-7H-pyrrolo[3,2-e][1,2,4]triazolo[1,5-c]pyrimidine-8-carboxamide

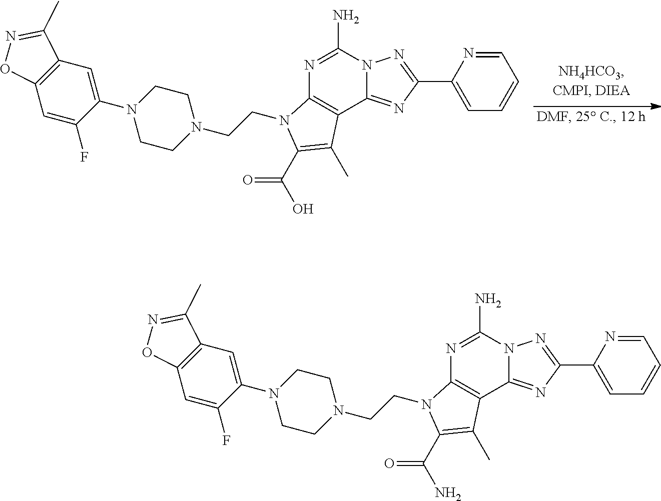

To a solution of 5-amino-9-chloro-7-[2-[4-(2-fluoro-5-oxazol-2-yl-phenyl)piperazin-1-yl]ethyl]-2-(2-pyridyl)-[1,2,4]triazolo pyrrolo pyrimidine-8-carboxylic acid (40 mg, 66.3 umol, 1 eq) and NH₄HCO₃ (10.4 mg, 132 umol, 10.9 uL, 2 eq) in N,N-dimethylformamide (3 mL) was added 2-bromo-1-ethyl-pyridin-1-ium; tetrafluoroborate (21.8 mg, 79.6 umol, 1.2 eq) and N,N-diisopropylethylamine (21.4 mg, 165 umol, 28.9 uL, 2.5 eq). The mixture was stirred at 25° C. for 12 h. LC-MS indicated disappearance of starting material and formation of a new peak with the desired mass. To the reaction mixture (suspension) was added 1N aqueous HCl to afford a clear solution that was purified by prep-HPLC (HCl condition) to yield 5-amino-7-(2-(4-(6-fluoro-3-methylbenzo[d]isoxazol-5-yl)piperazin-1-yl)ethyl)-9-methyl-2-(pyridin-2-yl)-7H-pyrrolo[3,2-e][1,2,4]triazolo[1,5-c]pyrimidine-8-carboxamide (6 mg, 9.40 umol, HCl) as a yellow solid. LCMS for product (ESI+): m/z 570.3 (M+H)⁺, Rt: 1.895 min. ¹H NMR (400 MHz, DMSO-d₆) δ=8.75 (d, J=2.9 Hz, 1H), 8.31 (d, J=7.3 Hz, 1H), 8.04-7.97 (m, 1H), 7.79 (br s, 2H), 7.68-7.51 (m, 4H), 7.40 (d, J=8.2 Hz, 1H), 4.55 (s, 2H), 2.98 (br s, 4H), 2.67 (s, 5H), 2.63 (br s, 4H).

### Example 34: Synthesis of 5-amino-7-(2-(4-(6-fluoro-3-methylbenzo[d]isoxazol-5-yl)-1,4-diazepan-1-yl)ethyl)-9-methyl-2-(pyridin-2-yl)-7H-pyrrolo[3,2-e][1,2,4]triazolo[1,5-c]pyrimidine-8-carboxamide

### Step 1: Synthesis of methyl 5-amino-7-(2-(4-(6-fluoro-3-methylbenzo[d]isoxazol-5-yl)-1,4-diazepan-1-yl)ethyl)-9-methyl-2-(pyridin-2-yl)-7H-pyrrolo[3,2-e][1,2,4]triazolo[1,5-c]pyrimidine-8-carboxylate

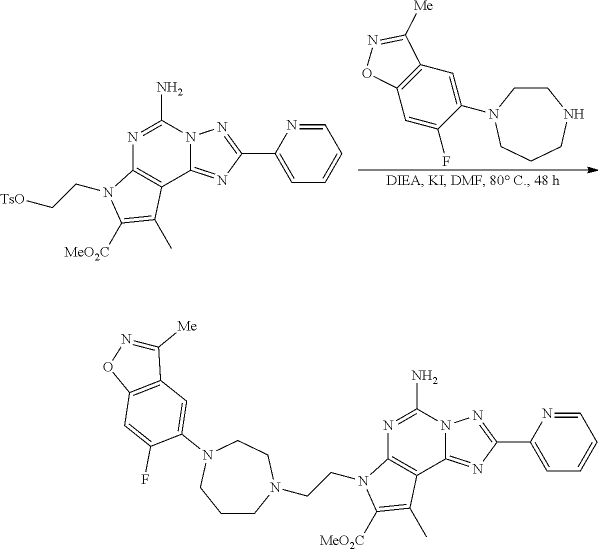 [SMILES: COC(=O)c1c(C)c2c(nc(N)n3nc(-c4ccccn4)nc23)n1CCOS(=O)(=O)c1ccc(C)cc1 ; Cc1noc2cc(F)c(N3CCCNCC3)cc12 ; "DIEA, KI, DMF, 80° C., 48 h" ; COC(=O)c1c(C)c2c(nc(N)n3nc(-c4ccccn4)nc23)n1CCN1CCCN(c2cc3c(C)noc3cc2F)CC1]

To a solution of methyl 5-amino-9-methyl-7-[2-(p-tolylsulfonyloxy)ethyl]-2-(2-pyridyl) [1,2,4]triazolo[BLAH]pyrrolo[BLAH]pyrimidine-8-carboxylate (150 mg, 287 umol, 1.00 eq) and 5-(1,4-diazepan-1-yl)-6-fluoro-3-methyl-1,2-benzoxazole (143 mg, 575 umol, 2.00 eq) in dimethyl formamide (4.50 mL) was added diisopropylethylamine (148 mg, 1.15 mmol, 200 uL, 4.00 eq) and KI (38.1 mg, 230 umol, 0.80 eq). The mixture was stirred at 80° C. for 48 h. LC-MS showed consumption of starting material and one main peak corresponding to the desired product was detected. The reaction mixture was filtered and the filtrate was purified by prep-HPLC (TFA condition) to afford methyl 5-amino-7-(2-(4-(6-fluoro-3-methylbenzo[d]isoxazol-5-yl)-1,4-diazepan-1-yl)ethyl)-9-methyl-2-(pyridin-2-yl)-7H-pyrrolo[3,2-e][1,2,4]triazolo[1,5-c]pyrimidine-8-carboxylate (56.0 mg, 78.5 umol, TFA) as a yellow solid. LCMS for product (ESI+): m/z 599.4 (M+H)⁺

### Step 2: Synthesis of 5-amino-7-(2-(4-(6-fluoro-3-methylbenzo[d]isoxazol-5-yl)-1,4-diazepan-1-yl)ethyl)-9-methyl-2-(pyridin-2-yl)-7H-pyrrolo[3,2-e][1,2,4]triazolo[1,5-c]pyrimidine-8-carboxylic acid

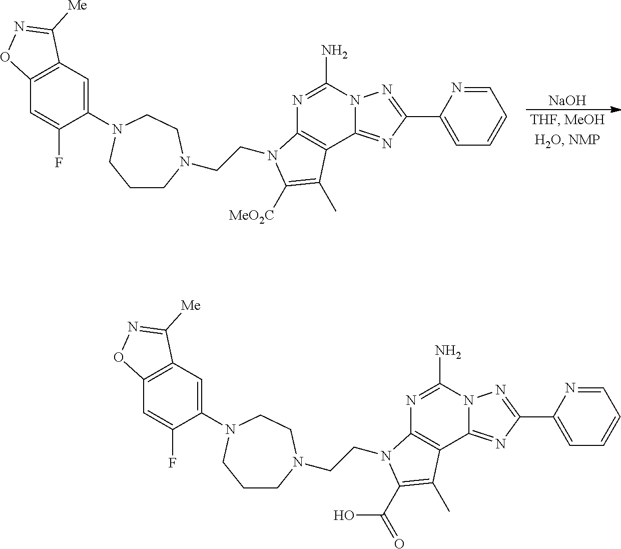

To a solution of methyl 5-amino-7-[2-[4-(6-fluoro-3-methyl-1,2-benzoxazol-5-yl)-1,4-diazepan-1-yl]ethyl]-9-methyl-2-(2-pyridyl)-[1,2,4]triazolo[BLAH]pyrrolo[BLAH]pyrimidine-8-carboxylate (14.0 mg, 19.6 umol, 1.00eq, TFA) in tetrahydrofuran (7.00 mL), N-methyl pyrrolidone (3.50 mL) and water (0.49 mL) was added NaOH (11.7 mg, 294 umol, 15.0 eq). The mixture was stirred at 80° C. for 12 h. LC-MS showed consumption of the starting material and appearance of one main peak with desired m/z. The reaction mixture was filtered and The filtrate was purified by prep-HPLC (neutral condition) to afford 5-amino-7-(2-(4-(6-fluoro-3-methylbenzo[d]isoxazol-5-yl)-1,4-diazepan-1-yl)ethyl)-9-methyl-2-(pyridin-2-yl)-7H-pyrrolo[3,2-e][1,2,4]triazolo[1,5-c]pyrimidine-8-carboxylic acid (4.00 mg, 6.84 umol) as a white solid. LCMS (ESI) m/z=585.2, Rt=0.978 min.

### Step 3: Synthesis of 5-amino-7-(2-(4-(6-fluoro-3-methylbenzo[d]isoxazol-5-yl)-1,4-diazepan-1-yl)ethyl)-9-methyl-2-(pyridin-2-yl)-7H-pyrrolo[3,2-e][1,2,4]triazolo[1,5-c]pyrimidine-8-carboxamide

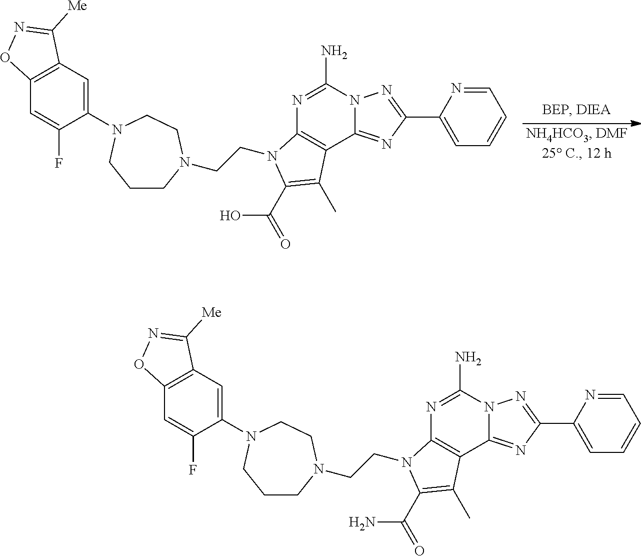 [SMILES: Cc1noc2cc(F)c(N3CCCN(CCn4c(C(=O)O)c(C)c5c4nc(N)n4nc(-c6ccccn6)nc54)CC3)cc12 ; "NH4HCO3, DMF25° C., 12 h" ; Cc1noc2cc(F)c(N3CCCN(CCn4c(C(N)=O)c(C)c5c4nc(N)n4nc(-c6ccccn6)nc54)CC3)cc12]

To a solution of 5-amino-7-[2-[4-(6-fluoro-3-methyl-1,2-benzoxazol-5-yl)-1,4-diazepan-1-yl]ethyl]-9-methyl-2-(2-pyridyl)-[1,2,4]triazolo[BLAH]pyrrolo[BLAH]pyrimidine-8-carboxylic acid (3.00 mg, 5.13 umol, 1.00 eq) in dimethyl formamide (0.30 mL) was added NH₄HCO₃ (811 ug, 10.2 umol, 2.00 eq), 2-bromo-1-ethyl-pyridin-1-ium; tetrafluoroborate (1.69 mg, 6.16 umol, 1.20 eq) and diisopropylethylamine (1.66 mg, 12.8 umol, 2.23 uL, 2.50 eq). The mixture was stirred at 25° C. for 12 h. LC-MS showed that starting material was completely consumed and one main peak with desired m/z was detected. The reaction mixture was filtered and the filtrate was purified by prep-HPLC (HCl condition) to afford 5-amino-7-(2-(4-(6-fluoro-3-methylbenzo[d]isoxazol-5-yl)-1,4-diazepan-1-yl)ethyl)-9-methyl-2-(pyridin-2-yl)-7H-pyrrolo[3,2-e][1,2,4]triazolo[1,5-c]pyrimidine-8-carboxamide (1.00 mg, 1.61 umol, HCl) as a yellow solid. LCMS (ESI) m/z=584.3, Rt=1.841 min). 1H NMR (400 MHz, METHANOL-d4) δ=8.84 (d, J=5.3 Hz, 1H), 8.63 (dd, J=1.1, 8.2 Hz, 1H), 8.41-8.35 (m, 1H), 7.86 (dd, J=6.2, 6.8 Hz, 1H), 7.34-7.27 (m, 2H), 4.81-4.75 (m, 2H), 3.87 (br d, J=13.0 Hz, 2H), 3.71-3.65 (m, 2H), 3.63-3.47 (m, 4H), 3.44-3.37 (m, 2H), 2.83 (s, 3H), 2.50 (s, 3H), 2.37 (dt, J=1.3, 2.5 Hz, 1H), 2.33-2.24 (m, 1H).

### Example 35: Synthesis of 5-amino-7-(2-(4-(6-fluoro-3-methylbenzo[d]isoxazol-5-yl)-1,4-diazepan-1-yl)ethyl)-9-methyl-2-(pyridin-2-yl)-7H-pyrrolo[3,2-e][1,2,4]triazolo[1,5-c]pyrimidine-8-carboxamide

### Step 1: Synthesis of methyl 5-amino-7-(2-(4-(2,4-difluorophenyl)piperazin-1-yl)ethyl)-9-methyl-2-(pyridin-2-yl)-7H-pyrrolo[3,2-e][1,2,4]triazolo[1,5-c]pyrimidine-8-carboxylate

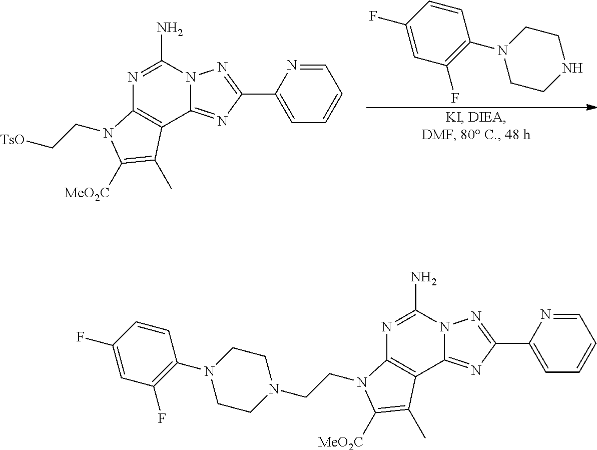

To a mixture of methyl 5-amino-9-methyl-7-[2-(p-tolylsulfonyloxy)ethyl]-2-(2-pyridyl)-[1,2,4]triazolo pyrrolo pyrimidine-8-carboxylate (250 mg, 479 umol, 1 eq) and 1-(2,4-difluorophenyl)piperazine (190 mg, 958 umol, 2 eq) in N,N-dimethylformamide (4.7 mL) was added KI (79.5 mg, 479 umol, 1 eq) and N,N-diisopropylethylamine (371 mg, 2.88 mmol, 500 uL, 6 eq). The mixture was stirred at 80° C. for 48 h. LC-MS showed completion of the reaction. The reaction mixture was filtered and resulting cake was dried in vacuum to give a gray solid methyl 5-amino-7-(2-(4-(2,4-difluorophenyl)piperazin-1-yl)ethyl)-9-methyl-2-(pyridin-2-yl)-7H-pyrrolo[3,2-e][1,2,4]triazolo[1,5-c]pyrimidine-8-carboxylate that was used without further purification (80 mg, 146 umol.) LCMS for product (ESI+): m/z 548.1 (M+H)⁺, Rt: 1.101 min.

### Step 2: Synthesis of 5-amino-7-(2-(4-(2,4-difluorophenyl)piperazin-1-yl)ethyl)-9-methyl-2-(pyridin-2-yl)-7H-pyrrolo[3,2-e][1,2,4]triazolo[1,5-c]pyrimidine-8-carboxylic acid

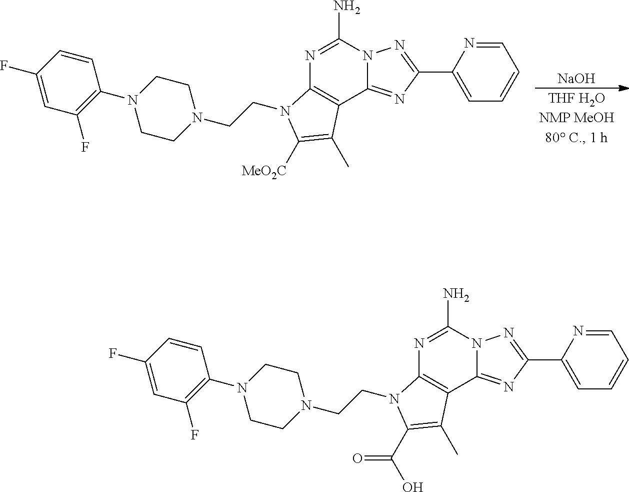

To a solution of methyl 5-amino-7-[2-[4-(2,4-difluorophenyl)piperazin-1-yl]ethyl]-9-methyl-2-(2-pyridyl)-[1,2,4]triazolo pyrrolo pyrimidine-8-carboxylate (10 mg, 18.2 umol, 1 eq) in methanol (0.1 mL), tetrahydrofuran (0.1 mL) and 1-methyl-2-pyrrolidinone (0.25 mL) was added NaOH (5.11 mg, 127 umol, 7 eq) in water (0.05 mL). The mixture was stirred at 80° C. for 1 h. LC-MS showed completion of the reaction. Five additional vials were set up as described above and all six reaction mixtures were combined. To the combined reaction mixture (suspension) was added trifluoroacetic acid (0.5 mL) to yield a clear solution that was purified by prep-HPLC (neutral condition) to afford 5-amino-7-(2-(4-(2,4-difluorophenyl)piperazin-1-yl)ethyl)-9-methyl-2-(pyridin-2-yl)-7H-pyrrolo[3,2-e][1,2,4]triazolo[1,5-c]pyrimidine-8-carboxylic acid (20 mg, 35.9 umol) as a white solid. LCMS for product (ESI+): m/z 534.3 (M+H)⁺, Rt: 2.208 min. ¹H NMR (400 MHz, DMSO-d₆) δ=8.75 (d, J=4.2 Hz, 1H), 8.31 (d, J=7.7 Hz, 1H), 8.01 (t, J=7.6 Hz, 1H), 7.88 (br s, 2H), 7.57-7.51 (m, 1H), 7.17 (br dd, J=9.4, 12.5 Hz, 1H), 7.06-6.94 (m, 2H), 4.66 (s, 2H), 2.91 (br s, 4H), 2.75 (s, 3H), 2.68-2.62 (m, 6H).

### Example 36: Synthesis of 5-amino-7-(2-(4-(2,4-difluorophenyl)piperazin-1-yl)ethyl)-9-methyl-2-(pyridin-2-yl)-7H-pyrrolo[3,2-e][1,2,4]triazolo[1,5-c]pyrimidine-8-carboxamide

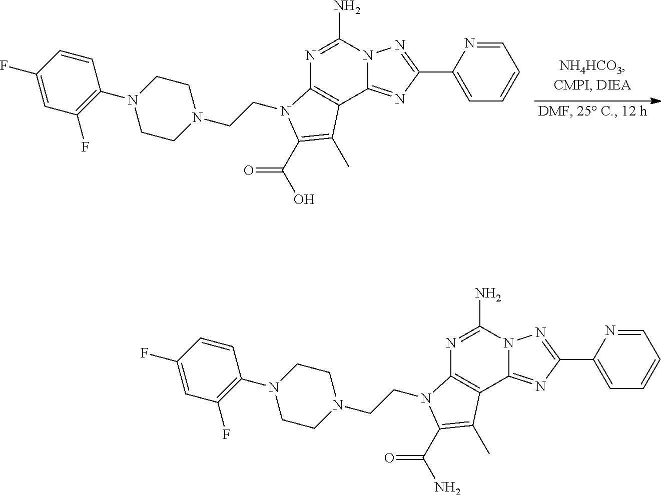 [SMILES: Cc1c(C(=O)O)n(CCN2CCN(c3ccc(F)cc3F)CC2)c2nc(N)n3nc(-c4ccccn4)nc3c12 ; "DMF, 25° C., 12 h" ; Cc1c(C(N)=O)n(CCN2CCN(c3ccc(F)cc3F)CC2)c2nc(N)n3nc(-c4ccccn4)nc3c12]

To a solution of 5-amino-7-[2-[4-(2,4-difluorophenyl)piperazin-1-yl]ethyl]-9-methyl-2-(2-pyridyl)-[1,2,4]triazolo pyrrolo pyrimidine-8-carboxylic acid (15 mg, 28.1 umol, 1 eq) and NH₄HCO₃ (8.89 mg, 112 umol, 9.26 uL, 4 eq) in N,N-dimethylformamide (1.5 mL) was added 2-chloro-1-methylpyridinium iodide (14.3 mg, 56.2 umol, 2 eq) and N,N-diisopropylethylamine (18.1 mg, 140 umol, 24.4 uL, 5 eq). The mixture was stirred at 25° C. for 12 h. LC-MS showed completion of the reaction. The reaction mixture (suspension) was treated with trifluoroacetic acid (0.1 mL) and the resulting clear solution was purified by prep-HPLC (HCl condition) to afford 5-amino-7-(2-(4-(2,4-difluorophenyl)piperazin-1-yl)ethyl)-9-methyl-2-(pyridin-2-yl)-7H-pyrrolo[3,2-e][1,2,4]triazolo[1,5-c]pyrimidine-8-carboxamide (7 mg, 12.3 umol, HCl) as a yellow solid. LCMS for product (ESI+): m/z 533.3 (M+H)⁺, Rt: 1.198 min. ¹H NMR (400 MHz, METHANOL-d₄) δ=9.00-8.91 (m, 2H), 8.80 (t, J=7.9 Hz, 1H), 8.21 (t, J=7.3 Hz, 1H), 7.16-7.07 (m, 1H), 7.02-6.95 (m, 1H), 6.92 (br t, J=8.5 Hz, 1H), 4.80-4.74 (m, 2H), 3.81-3.72 (m, 4H), 3.51 (br d, J=13.5 Hz, 2H), 3.41 (br t, J=11.6 Hz, 2H), 3.21-3.13 (m, 2H), 2.84 (s, 3H).

### Example 37: Synthesis of 5-amino-9-methyl-2-(pyridin-2-yl)-7-(2-(4-(pyridin-4-yl)piperazin-1-yl)ethyl)-7H-pyrrolo[3,2-e][1,2,4]triazolo[1,5-c]pyrimidine-8-carboxylic acid

### Step 1: Synthesis of methyl 5-amino-9-methyl-2-(pyridin-2-yl)-7-(2-(4-(pyridin-4-yl)piperazin-1-yl)ethyl)-7H-pyrrolo[3,2-e][1,2,4]triazolo[1,5-c]pyrimidine-8-carboxylate

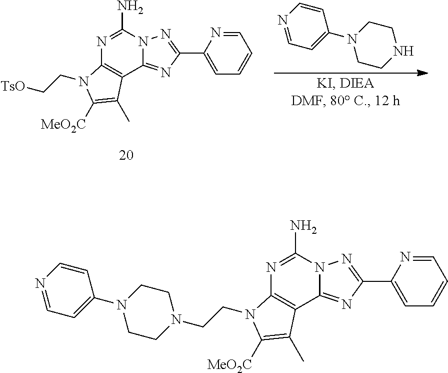

To a solution of methyl 5-amino-9-methyl-7-[2-(p-tolylsulfonyloxy)ethyl]-2-(2-pyridyl)-[1,2,4]triazolo pyrrolo pyrimidine-8-carboxylate (250 mg, 479 umol, 1 eq) and 1-(4-pyridyl)piperazine (156 mg, 958 umol, 2 eq) in N,N-dimethylformamide (4.7 mL) was added KI (79.5 mg, 479 umol, 1 eq) and N,N-diisopropylethylamine (371 mg, 2.88 mmol, 500 uL, 6 eq). The mixture was stirred at 80° C. for 12 h. LC-MS showed the reaction was completed. The reaction mixture was filtered and resulting cake was dried in vacuum to yield a gray solid that was used without purification (80 mg, 156 umol). LCMS for product (ESI+): m/z 513.1 (M+H)⁺, Rt: 0.933 min.

### Step 2: Synthesis of 5-amino-9-methyl-2-(pyridin-2-yl)-7-(2-(4-(pyridin-4-yl)piperazin-1-yl)ethyl)-7H-pyrrolo[3,2-e][1,2,4]triazolo[1,5-c]pyrimidine-8-carboxylic acid

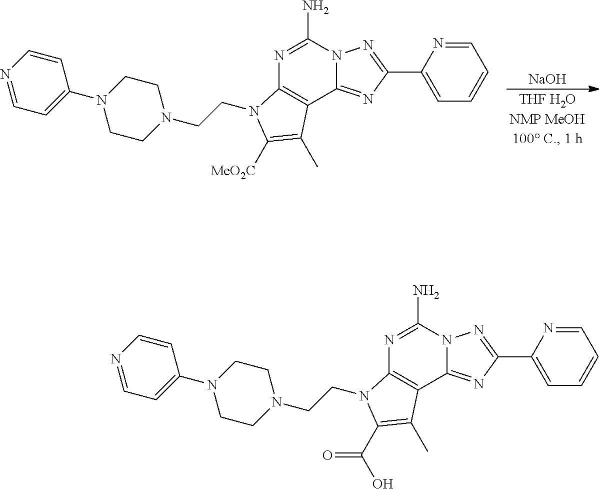

To a solution of methyl 5-amino-9-methyl-2-(2-pyridyl)-7-[2-[4-(4-pyridyl)piperazin-1-yl]ethyl]-[1,2,4]triazolo pyrrolo pyrimidine-8-carboxylate (10 mg, 19.5 umol, 1 eq) in 1-methyl-2-pyrrolidinone (0.25 mL), methanol (0.1 mL) and tetrahydrofuran (0.1 mL) was added NaOH (5.46 mg, 136 umol, 7 eq) in water (0.05 mL). The mixture was stirred at 80° C. for 1 h. LC-MS showed the reaction was completed. Six additional vials were set up as described above and the reaction mixtures were combined. To the combined reaction mixture (suspension) was added trifluoroacetic acid (0.5 mL) to afford a clear solution that was purified by prep-HPLC (neutral condition) to yield the 5-amino-9-methyl-2-(pyridin-2-yl)-7-(2-(4-(pyridin-4-yl)piperazin-1-yl)ethyl)-7H-pyrrolo[3,2-e][1,2,4]triazolo[1,5-c]pyrimidine-8-carboxylic acid (22 mg, 43.2 umol) as a white solid. LCMS for product (ESI+): m/z 499.3 (M+H)⁺, Rt: 1.881 min. ¹H NMR (400 MHz, DMSO-d₆) δ=8.75 (d, J=5.3 Hz, 1H), 8.31 (d, J=7.9 Hz, 1H), 8.14 (br d, J=5.7 Hz, 2H), 8.04-7.91 (m, 3H), 7.57-7.51 (m, 1H), 6.79 (br d, J=6.2 Hz, 2H), 4.69-4.62 (m, 2H), 3.26 (br s, 4H), 2.76 (s, 3H), 2.65 (br d, J=8.4 Hz, 2H), 2.58 (br s, 4H).

### Example 38: Synthesis of 5-amino-9-methyl-2-(pyridin-2-yl)-7-(2-(4-(pyridin-4-yl)piperazin-1-yl)ethyl)-7H-pyrrolo[3,2-e][1,2,4]triazolo[1,5-c]pyrimidine-8-carboxamide

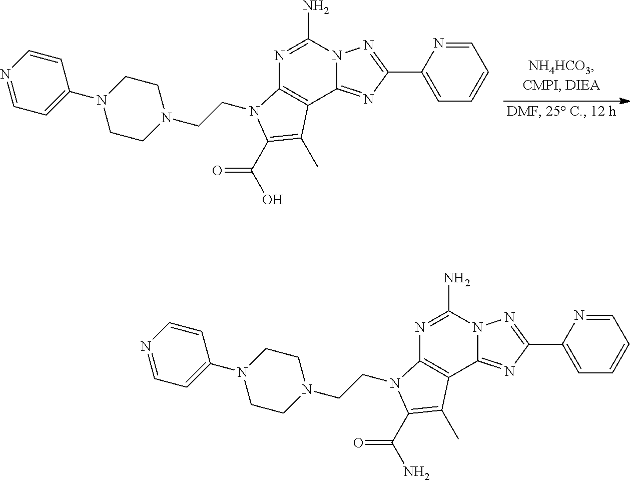 [SMILES: Cc1c(C(=O)O)n(CCN2CCN(c3ccncc3)CC2)c2nc(N)n3nc(-c4ccccn4)nc3c12 ; "DMF, 25° C., 12 h" ; Cc1c(C(N)=O)n(CCN2CCN(c3ccncc3)CC2)c2nc(N)n3nc(-c4ccccn4)nc3c12]

To a solution of 5-amino-9-methyl-2-(2-pyridyl)-7-[2-[4-(4-pyridyl)piperazin-1-yl]ethyl]-[1,2,4]triazolo pyrrolo pyrimidine-8-carboxylic acid (17 mg, 34.1 umol, 1 eq) and NH₄HCO₃ (10.7 mg, 136 umol, 11.2 uL, 4 eq) in N,N-dimethylformamide (0.2 mL) was added 2-chloro-1-methylpyridinium iodide (17.4 mg, 68.2 umol, 2 eq) and N,N-diisopropylethylamine (22.0 mg, 170 umol, 29.7 uL, 5 eq). The mixture was stirred at 25° C. for 12 h. LC-MS showed the reaction was completed. To the reaction mixture (suspension) was added trifluoroacetic acid (0.1 mL) and the clear solution was purified by prep-HPLC (HCl condition) to afford 5-amino-9-methyl-2-(pyridin-2-yl)-7-(2-(4-(pyridin-4-yl)piperazin-1-yl)ethyl)-7H-pyrrolo[3,2-e][1,2,4]triazolo[1,5-c]pyrimidine-8-carboxamide (4 mg, 7.49 umol, HCl) as a yellow solid. LCMS for product (ESI+): m/z 498.3 (M+H)⁺, Rt: 1.504 min. ¹H NMR (400 MHz, METHANOL-d₄) δ=8.96 (d, J=5.4 Hz, 1H), 8.91 (d, J=7.8 Hz, 1H), 8.80-8.73 (m, 1H), 8.27 (d, J=7.8 Hz, 2H), 8.18 (t, J=6.8 Hz, 1H), 7.31 (d, J=7.3 Hz, 2H), 4.80-4.77 (m, 2H), 4.51 (br s, 2H), 3.94-3.57 (m, 6H), 3.55-3.34 (m, 2H), 2.84 (s, 3H).

### Example 39: Synthesis of 5-amino-7-(2-(4-(4-cyano-2-fluorophenyl)piperazin-1-yl)ethyl)-9-methyl-2-(pyridin-2-yl)-7H-pyrrolo[3,2-e][1,2,4]triazolo[1,5-c]pyrimidine-8-carboxylic acid

### Step 1: Synthesis of methyl 5-amino-7-(2-(4-(4-cyano-2-fluorophenyl)piperazin-1-yl)ethyl)-9-methyl-2-(pyridin-2-yl)-7H-pyrrolo[3,2-e][1,2,4]triazolo[1,5-c]pyrimidine-8-carboxylate

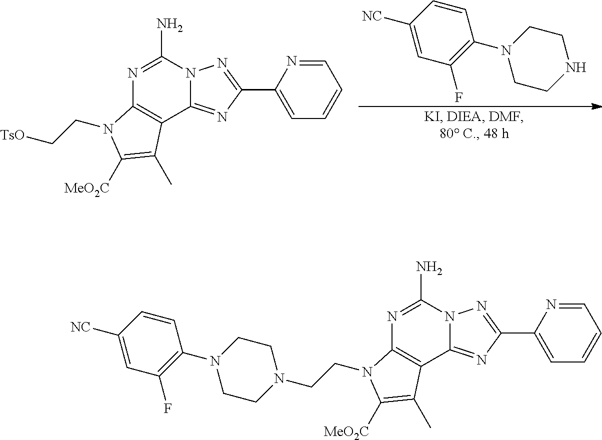

Methyl 5-amino-9-methyl-7-[2-(p-tolylsulfonyloxy)ethyl]-2-(2-pyridyl)-[1,2,4]triazolo pyrrolopyrimidine-8-carboxylate (250 mg, 479 umol, 1 eq) and 3-fluoro-4-piperazin-1-yl-benzonitrile (197 mg, 959 umol, 2.0 eq) were dissolved in DMF (8 mL). DIEA (372 mg, 2.88 mmol, 501 uL, 6.0 eq) and KI (79.6 mg, 479 umol, 1 eq) was added to the reaction mixture. The solution was stirred at 80° C. for 48 h. LCMS showed the starting material was consumed and the appearance of a major peak with the desired MS. The suspension was cooled to 0° C. and stirred for 5 min. The resulting white solid was filtrated and washed with methyl tertiary-butyl ether (10 mL) to afford methyl 5-amino-7-(2-(4-(4-cyano-2-fluorophenyl)piperazin-1-yl)ethyl)-9-methyl-2-(pyridin-2-yl)-7H-pyrrolo[3,2-e][1,2,4]triazolo[1,5-c]pyrimidine-8-carboxylate (70 mg, 126 umol) as yellow solid. The solid was used in the next step without further purification. LCMS (ESI+): m/z 555.1 (M+H)⁺, Rt: 1.106 min.

### Step 2: Synthesis of 5-amino-7-(2-(4-(4-cyano-2-fluorophenyl)piperazin-1-yl)ethyl)-9-methyl-2-(pyridin-2-yl)-7H-pyrrolo[3,2-e][1,2,4]triazolo[1,5-c]pyrimidine-8-carboxylic acid

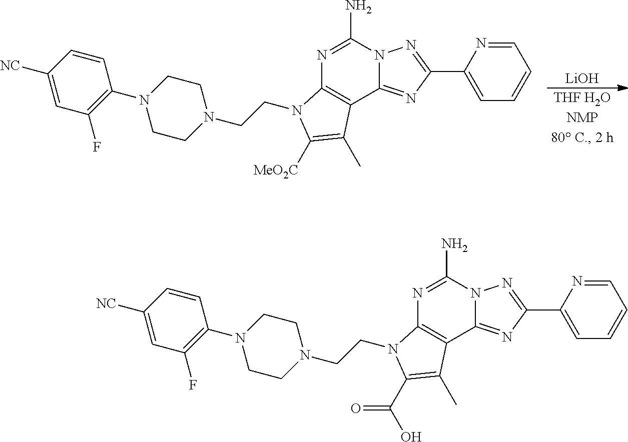

Methyl 5-amino-7-[2-[4-(4-cyano-2-fluoro-phenyl)piperazin-1-yl]ethyl]-9-methyl-2-(2-pyridyl)-[1,2,4]triazolo pyrrolo pyrimidine-8-carboxylate (0.01 g, 18.0 umol, 1 eq) was dissolved in NMP (1 mL), THE (2 mL) and H₂O (1 mL). LiOH.H₂O (5.30 mg, 126 umol, 7.0 eq) was added to the reaction mixture. The solution was stirred at 80° C. for 2 h. LCMS showed consumption of the starting material and the appearance a major peak with desired MS. Four additional vials were set up as described above and the reaction mixtures were combined. The solvent was removed under reduced pressure and the residue was purified by prep-HPLC to afford 5-amino-7-(2-(4-(4-cyano-2-fluorophenyl)piperazin-1-yl)ethyl)-9-methyl-2-(pyridin-2-yl)-7H-pyrrolo[3,2-e][1,2,4]triazolo[1,5-c]pyrimidine-8-carboxylic acid (30 mg, 47.1 umol, HCl) as yellow solid. LCMS (ESI+): m/z 541.3 (M+H)⁺, Rt: 1.913 min. ¹H NMR (400 MHz, METHANOL-d₄) δ=8.89 (d, J=4.4 Hz, 1H), 8.75 (d, J=7.8 Hz, 1H), 8.55-8.47 (m, 1H), 8.00-7.93 (m, 1H), 7.57-7.47 (m, 2H), 7.18 (t, J=8.6 Hz, 1H), 5.00 (t, J=5.6 Hz, 2H), 4.14-4.00 (m, 2H), 3.92-3.76 (m, 4H), 3.55-3.36 (m, 2H), 3.27-3.14 (m, 2H), 3.29-3.13 (m, 4H), 2.92 (s, 3H).

### Example 40: Synthesis of 5-amino-7-(2-(4-(4-cyano-2-fluorophenyl)piperazin-1-yl)ethyl)-9-methyl-2-(pyridin-2-yl)-7H-pyrrolo[3,2-e][1,2,4]triazolo[1,5-c]pyrimidine-8-carboxamide

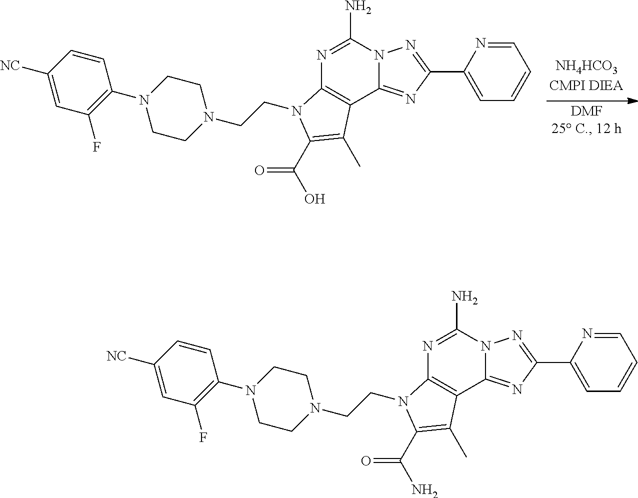 [SMILES: Cc1c(C(=O)O)n(CCN2CCN(c3ccc(C#N)cc3F)CC2)c2nc(N)n3nc(-c4ccccn4)nc3c12 ; "DMF25° C., 12 h" ; Cc1c(C(N)=O)n(CCN2CCN(c3ccc(C#N)cc3F)CC2)c2nc(N)n3nc(-c4ccccn4)nc3c12]

5-amino-7-[2-[4-(4-cyano-2-fluoro-phenyl)piperazin-1-yl]ethyl]-9-methyl-2-(2-pyridyl)-[1,2,4]triazolo pyrrolo pyrimidine-8-carboxylic acid (15 mg, 27.8umol, 1.0 eq), NH₄HCO₃ (8.77 mg, 111 umol, 9.14 uL, 4 eq) were dissolved in DMF (1 mL). CMPI (14.2 mg, 55.5 umol, 2 eq) and DIEA (17.9 mg, 138 umol, 24.2 uL, 5 eq) were added to the reaction mixture. The solution was stirred at 20° C. for 12 h. LCMS showed the completion of the reaction and formation a new peak with desired MS. The reaction mixture was purified by prep-HPLC to yield 5-amino-7-(2-(4-(4-cyano-2-fluorophenyl)piperazin-1-yl)ethyl)-9-methyl-2-(pyridin-2-yl)-7H-pyrrolo[3,2-e][1,2,4]triazolo[1,5-c]pyrimidine-8-carboxamide (6 mg, 11.1 umol) as a yellow solid. LCMS (ESI+): m/z 540.3 (M+H)⁺, Rt: 1.855 min. ¹H NMR (400 MHz, METHANOL-d₄) δ=8.89 (d, J=4.4 Hz, 1H), 8.73 (d, J=7.8 Hz, 1H), 8.47 (dt, J=1.5, 7.8 Hz, 1H), 7.94 (dd, J=5.4, 6.4 Hz, 1H), 7.57-7.51 (m, 2H), 7.22-7.17 (m, 1H), 4.79-4.75 (m, 2H), 3.85-3.73 (m, 6H), 3.48-3.38 (m, 2H), 3.31-3.24 (m, 2H), 2.85 (s, 3H).

### Example 41: Synthesis of 5-amino-9-methyl-2-(pyridin-2-yl)-7-(2-(4-(pyrimidin-2-yl)piperazin-1-yl)ethyl)-7H-pyrrolo[3,2-e][1,2,4]triazolo[1,5-c]pyrimidine-8-carboxylic acid

### Step 1: Synthesis of methyl 5-amino-9-methyl-2-(pyridin-2-yl)-7-(2-(4-(pyrimidin-2-yl)piperazin-1-yl)ethyl)-7H-pyrrolo[3,2-e][1,2,4]triazolo[1,5-c]pyrimidine-8-carboxylate

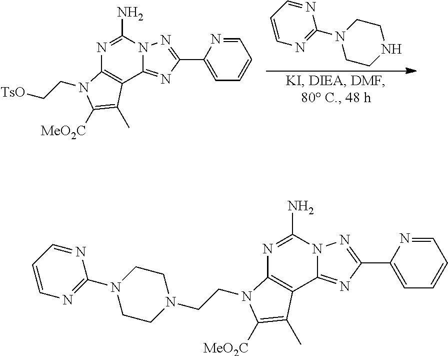

Methyl 5-amino-9-methyl-7-[2-(p-tolylsulfonyloxy)ethyl]-2-(2-pyridyl)-[1,2,4]triazolo pyrrolopyrimidine-8-carboxylate (250 mg, 479 umol, 1 eq) and 2-piperazin-1ylpyrimidine-dihydrochloride (227 mg, 959 umol, 2.0 eq) were dissolved in DMF (5 mL). DIEA (372 mg, 2.88 mmol, 501 uL, 6.0 eq) and KI (79.6 mg, 479 umol, 1 eq) were added to the reaction mixture. The solution was stirred at 80° C. for 48 h. LCMS showed completion of the reaction and formation of a new peak with desired MS. The suspension was cooled to 0° C. and stirred for 5 min and the resulting white solid was collected by filtration, washed with methyl tertiary-butyl ether (10 mL) and air-dried to yield methyl 5-amino-9-methyl-2-(pyridin-2-yl)-7-(2-(4-(pyrimidin-2-yl)piperazin-1-yl)ethyl)-7H-pyrrolo[3,2-e][1,2,4]triazolo[1,5-c]pyrimidine-8-carboxylate (80 mg, 156 umol) as a white solid (used without further purification). LCMS (ESI+): m/z 514.2 (M+H)⁺, Rt: 1.036 min.

### Step 2: Synthesis of 5-amino-9-methyl-2-(pyridin-2-yl)-7-(2-(4-(pyrimidin-2-yl)piperazin-1-yl)ethyl)-7H-pyrrolo[3,2-e][1,2,4]triazolo[1,5-c]pyrimidine-8-carboxylic acid

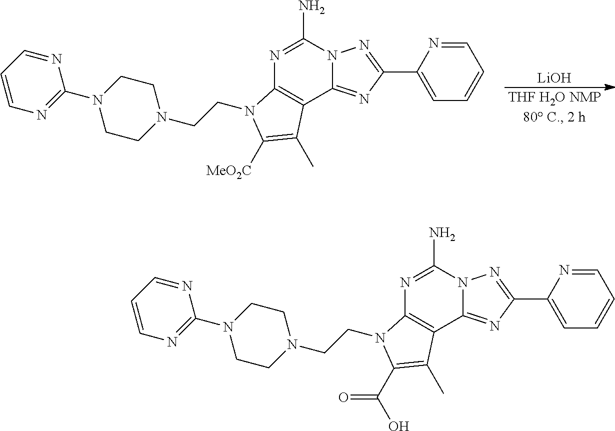

Methyl 5-amino-9-methyl-2-(2-pyridyl)-7-[2-(4-pyrimidin-2-ylpiperazin-1-yl)ethyl]-[1,2,4]triazolo pyrrolo pyrimidine-8-carboxylate (20 mg, 38.9 umol, 1 eq) was dissolved in H₂O (1 mL), THE (2 mL) and NMP (2 mL). LiOH.H₂O (11.4 mg, 272 umol, 7.0 eq) was added to the reaction mixture. The solution was stirred at 80° C. for 2 h. LCMS showed the completion of the reaction and the formation of a new major peak with desired MS. Two additional vials were set up as described above and the reaction mixtures were combined. The solvent was removed under reduced pressure and the residue was purified by prep-HPLC to yield 5-amino-9-methyl-2-(pyridin-2-yl)-7-(2-(4-(pyrimidin-2-yl)piperazin-1-yl)ethyl)-7H-pyrrolo[3,2-e][1,2,4]triazolo[1,5-c]pyrimidine-8-carboxylic acid (25 mg, 46.6 umol, HCl) as a yellow solid. LCMS (ESI+): m/z 500.2 (M+H)⁺, Rt: 1.764 min. ¹H NMR (400 MHz, METHANOL-d₄) δ=8.93 (d, J=4.9 Hz, 1H), 8.81 (d, J=8.3 Hz, 1H), 8.60 (t, J=7.8 Hz, 1H), 8.44 (d, J=4.9 Hz, 2H), 8.07-8.00 (m, 1H), 6.76 (t, J=4.9 Hz, 1H), 5.05-4.95 (m, 4H), 4.12-4.01 (m, 2H), 3.78 (t, J=5.6 Hz, 2H), 3.30-3.19 (m, 4H), 2.94 (s, 3H).

### Example 42: Synthesis of 5-amino-9-methyl-2-(pyridin-2-yl)-7-(2-(4-(pyrimidin-2-yl)piperazin-1-yl)ethyl)-7H-pyrrolo[3,2-e][1,2,4]triazolo[1,5-c]pyrimidine-8-carboxamide

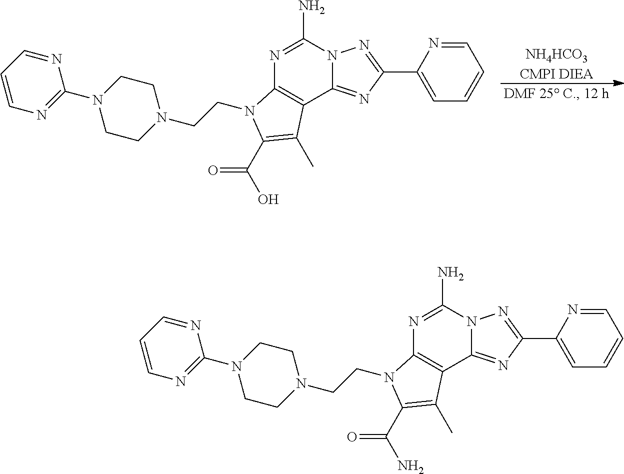 [SMILES: Cc1c(C(=O)O)n(CCN2CCN(c3ncccn3)CC2)c2nc(N)n3nc(-c4ccccn4)nc3c12 ; "DMF 25° C., 12 h" ; "NH4HCO3 CMPI DIEA" ; Cc1c(C(N)=O)n(CCN2CCN(c3ncccn3)CC2)c2nc(N)n3nc(-c4ccccn4)nc3c12]

5-amino-9-methyl-2-(2-pyridyl)-7-[2-(4-pyrimidin-2-ylpiperazin-1-yl)ethyl]-[1,2,4]triazolo pyrrolopyrimidine-8-carboxylic acid (10 mg, 20.0 umol, 1 eq) was dissolved in DMF (1 mL). CMPI (10.2 mg, 40.0 umol, 2 eq), DIEA (13.0 mg, 100 umol, 17.4 uL, 5 eq) and NH₄HCO₃ (6.33 mg, 80.1 umol, 6.59 uL, 4 eq) were added to the reaction mixture. The solution was stirred at 25° C. for 12 h. LCMS showed the completion of the reaction and the formation of a new major peak with desired MS. The reaction mixture was purified by prep-HPLC to afford 5-amino-9-methyl-2-(pyridin-2-yl)-7-(2-(4-(pyrimidin-2-yl)piperazin-1-yl)ethyl)-7H-pyrrolo[3,2-e][1,2,4]triazolo[1,5-c]pyrimidine-8-carboxamide (2 mg, 3.74 umol, HCl) as a yellow solid. LCMS (ESI+): m/z 499.3 (M+H)⁺, Rt: 1.691 min ¹H NMR (400 MHz, METHANOL-d₄) δ=9.00-8.91 (m, 2H), 8.83-8.74 (m, 1H), 8.44 (d, J=4.9 Hz, 2H), 8.20 (ddd, J=1.2, 6.0, 7.5 Hz, 1H), 6.79 (t, J=4.9 Hz, 1H), 4.97-4.88 (m, 2H), 4.79-4.75 (m, 2H), 3.84-3.65 (m, 4H), 3.52-3.33 (m, 2H), 3.26-3.16 (m, 2H), 2.85 (s, 3H).

### Example 43: Synthesis of 5-amino-7-(2-(4-(5-fluoro-2-methylpyridin-4-yl)piperazin-1-yl)ethyl)-9-methyl-2-(pyridin-2-yl)-7H-pyrrolo[3,2-e][1,2,4]triazolo[1,5-c]pyrimidine-8-carboxylic acid

### Step 1: Synthesis of methyl 5-amino-7-(2-(4-(5-fluoro-2-methylpyridin-4-yl)piperazin-1-yl)ethyl)-9-methyl-2-(pyridin-2-yl)-7H-pyrrolo[3,2-e][1,2,4]triazolo[1,5-c]pyrimidine-8-carboxylate

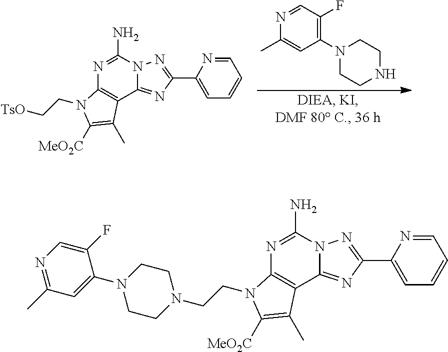

To a solution of methyl 5-amino-9-methyl-7-[2-(p-tolylsulfonyloxy)ethyl]-2-(2-pyridyl)-[1,2,4]triazolo[BLAH]pyrrolo[BLAH]pyrimidine-8-carboxylate (150 mg, 288 umol, 1.00 eq) in DMF (3 mL) was added DIEA (223 mg, 1.73 mmol, 301 uL, 6.00 eq), KI (38.2 mg, 230 umol, 0.80 eq) and 1-(5-fluoro-2-methyl-4-pyridyl)piperazine (112 mg, 575. umol, 2.00 eq). The mixture was stirred at 80° C. for 36 h. LC-MS showed consumption of starting material and formation of one new main peak with desired mass. The reaction mixture was directly purified by prep-HPLC (TFA condition) to afford 5-amino-7-(2-(4-(5-fluoro-2-methylpyridin-4-yl)piperazin-1-yl)ethyl)-9-methyl-2-(pyridin-2-yl)-7H-pyrrolo[3,2-e][1,2,4]triazolo[1,5-c]pyrimidine-8-carboxylic acid (40 mg, 66.1 umol).

### Step 2: Synthesis of 5-amino-7-(2-(4-(5-fluoro-2-methylpyridin-4-yl)piperazin-1-yl)ethyl)-9-methyl-2-(pyridin-2-yl)-7H-pyrrolo[3,2-e][1,2,4]triazolo[1,5-c]pyrimidine-8-carboxylic acid

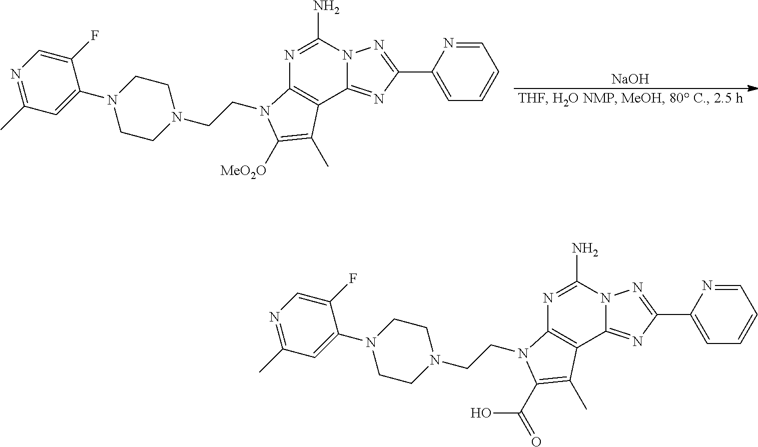

To a solution of methyl 5-amino-7-[2-[4-(5-fluoro-2-methyl-4-pyridyl)piperazin-1-yl]ethyl]-9-methyl-2-(2-pyridyl)-[1,2,4]triazolo[BLAH]pyrrolo[BLAH]pyrimidine-8-carboxylate (27 mg, 49.6 umol, 1.00 eq) in H₂O (0.4 mL), THF (0.4 mL), MeOH (0.4 mL) and NMP (0.4 mL) was added NaOH (13.9 mg, 347 umol, 7.00 eq). The mixture was stirred at 80° C. for 2.5 h. LC-MS indicated completion of the reaction and formation of a new major peak with desired mass was detected. One additional vial was set up as described above and the reaction mixtures were combined. The reaction mixture was filtered, and the filtrate was purified by prep-HPLC (neutral condition) to afford 5-amino-7-(2-(4-(5-fluoro-2-methylpyridin-4-yl)piperazin-1-yl)ethyl)-9-methyl-2-(pyridin-2-yl)-7H-pyrrolo[3,2-e][1,2,4]triazolo[1,5-c]pyrimidine-8-carboxylic acid (22 mg, 41.1 umol) as a white solid. LCMS for product (ESI+): m/z 531.3 (M+H)⁺, Rt: 1.615 min. ¹H NMR (400 MHz, DMSO-d₆) δ=8.75 (br d, J=4.8 Hz, 1H), 8.31 (d, J=8 Hz, 1H), 8.07 (d, J=6 Hz, 1H), 8.00 (dt, J=1.6, 7.6 Hz, 1H), 7.92 (s, 2H), 7.55 (dd, J=4.9, 6.7 Hz, 1H), 6.76 (d, J=8 Hz, 1H), 4.65 (br t, J=6.4 Hz, 2H), 3.28 (br s, 2H), 3.16 (br s, 2H), 2.76 (s, 3H), 2.67-2.66 (m, 3H), 2.63-2.60 (m, 3H), 2.33 (s, 3H).

### Example 44: Synthesis of 5-amino-7-(2-(4-(5-fluoro-2-methylpyridin-4-yl)piperazin-1-yl)ethyl)-9-methyl-2-(pyridin-2-yl)-7H-pyrrolo[3,2-e][1,2,4]triazolo[1,5-c]pyrimidine-8-carboxamide

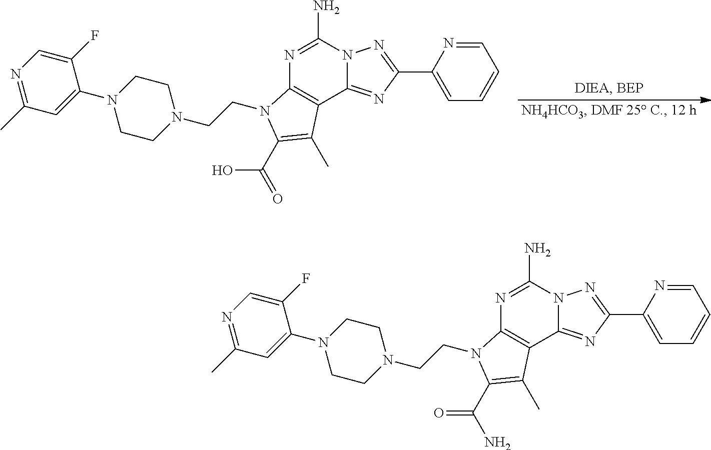

To a solution of 5-amino-7-[2-[4-(5-fluoro-2-methyl-4-pyridyl)piperazin-1-yl]ethyl]-9-methyl-2-(2-pyridyl)-[1,2,4]triazolo[BLAH]pyrrolo[BLAH]pyrimidine-8-carboxylic acid (10 mg, 18.9 umol, 1.00 eq) in DMF (0.6 mL) was added DIEA (6.09 mg, 47.1 umol, 8.21 uL, 2.50 eq), NH₄HCO₃ (2.98 mg, 37.7 umol, 3.10 uL, 2.00 eq) and 2-bromo-1-ethyl-pyridin-1-ium; tetrafluoroborate (6.19 mg, 22.6 umol, 1.20 eq). The mixture was stirred at 25° C. for 12 h. LC-MS showed completion of the reaction and formation of a new major peak with desired mass. The reaction was filtered, and the filtrate was purified by prep-HPLC (HCl condition) to afford 5-amino-7-(2-(4-(5-fluoro-2-methylpyridin-4-yl)piperazin-1-yl)ethyl)-9-methyl-2-(pyridin-2-yl)-7H-pyrrolo[3,2-e][1,2,4]triazolo[1,5-c]pyrimidine-8-carboxamide (2 mg, 3.51 umol, HCl) as a white solid. LCMS for product (ESI+): m/z 530.3 (M+H)⁺, Rt: 1.526 min. ¹H NMR (400 MHz, METHANOL-d₄) δ=8.90 (br d, J=4.3 Hz, 1H), 8.79 (br d, J=8 Hz, 1H), 8.59 (br t, J=8 Hz, 1H), 8.44 (br d, J=8 Hz, 1H), 8.03 (br t, J=6.2 Hz, 1H), 7.31 (br s, 1H), 4.75 (br s, 4H), 4.00-3.72 (m, 5H), 3.72-3.52 (m, 3H), 2.83 (s, 3H), 2.59 (s, 3H).

### Example 45: Synthesis of 2-(4-(4-(2-(5-amino-8-(methoxycarbonyl)-9-methyl-2-(pyridin-2-yl)-7H-pyrrolo[3,2-e][1,2,4]triazolo[1,5-c]pyrimidin-7-yl)ethyl)piperazin-1-yl)phenoxyacetic acid

### Step 1: Synthesis of tert-butyl 4-(4-(2-(tert-butoxy)-2-oxoethoxy)phenyl)piperazine-1-carboxylate

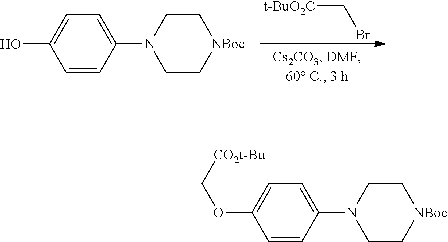

To a solution of tert-butyl 4-(4-hydroxyphenyl)piperazine-1-carboxylate (2 g, 7.19 mmol, 1 eq) in N,N-dimethylformamide (20 mL) was added Cs₂CO₃ (3.51 g, 10.7 mmol, 1.5 eq) and tert-butyl 2-bromoacetate (2.80 g, 14.3 mmol, 2.12 mL, 2 eq). The mixture was stirred at 60° C. for 3 h. TLC (petroleum ether:ethyl acetate=3:1, Rf=0.4) indicated completion of the reaction. The reaction mixture was poured into water (75 mL) and extracted with ethyl acetate (3×50 mL). The combined organic phase was concentrated in vacuo to yield tert-butyl 4-(4-(2-(tert-butoxy)-2-oxoethoxy)phenyl)piperazine-1-carboxylate as a red oil (3.5 g, 8.92 mmol). The product was used was used without further purification. ¹H NMR (400 MHz, CHLOROFORM-d) δ=6.87 (q, J=9.1 Hz, 4H), 4.47 (s, 2H), 3.61-3.54 (m, 4H), 3.06-2.98 (m, 4H), 1.49 (s, 18H).

### Step 2: Synthesis of tert-butyl 2-(4-(piperazin-1-yl)phenoxy)acetate

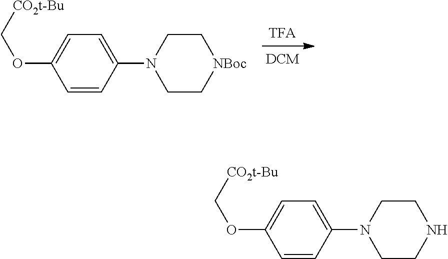 [SMILES: CC(C)(C)OC(=O)COc1ccc(N2CCN(C(=O)OC(C)(C)C)CC2)cc1 ; CC(C)(C)OC(=O)COc1ccc(N2CCNCC2)cc1]

To a solution of tert-butyl 4-[4-(2-tert-butoxy-2-oxo-ethoxy)phenyl]piperazine-1-carboxylate (3 g, 7.64 mmol, 1 eq) in dichloromethane (36 mL) was added trifluoroacetic acid (27.7 g, 243 mmol, 18.0 mL, 31.8 eq) at 0° C. The mixture was stirred at 0° C. for 1 h. TLC (petroleum ether:ethyl acetate=3:1, Rf=0.03) indicated completion of the reaction. The reaction mixture was quenched by addition of saturated sodium bicarbonate solution (280 mL) at 0° C. and then extracted with chloroform (3×100 mL). The combined organic layers were dried over anhydrous sodium sulfate, filtered, and concentrated under reduced pressure to afford tert-butyl 2-(4-(piperazin-1-yl)phenoxy)acetate as a red oil (1.4 g, 4.79 mmol). ¹H NMR (400 MHz, CHLOROFORM-d) δ=6.93-6.81 (m, 4H), 4.46 (s, 2H), 3.06 (s, 8H), 2.54 (br s, 1H), 1.49 (s, 9H).

### Step 3: Synthesis of methyl 5-amino-7-(2-(4-(4-(2-(tert-butoxy)-2-oxoethoxy)phenyl)piperazin-1-yl)ethyl)-9-methyl-2-(pyridin-2-yl)-7H-pyrrolo[3,2-e][1,2,4]triazolo[1,5-c]pyrimidine-8-carboxylate

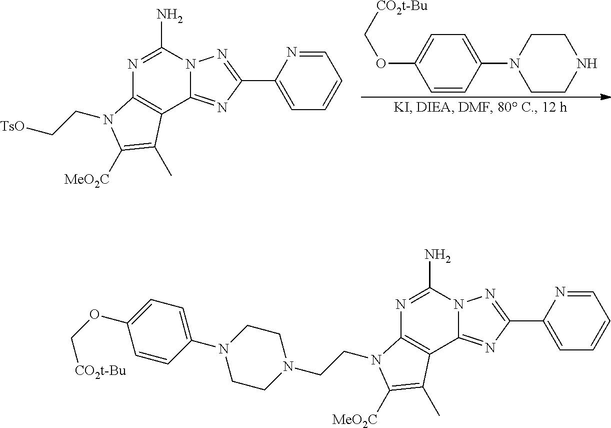

To a solution of methyl 5-amino-9-methyl-7-[2-(p-tolylsulfonyloxy)ethyl]-2-(2-pyridyl)-[1,2,4]triazolo pyrrolo pyrimidine-8-carboxylate (0.15 g, 287 umol, 1 eq) and tert-butyl 2-(4-piperazin-1-ylphenoxy) acetate (109 mg, 373 umol, 1.3 eq) in N,N-dimethylformamide (2.8 mL) was added N,N-diisopropylethylamine (111 mg, 862 umol, 150 uL, 3 eq) and KI (47.7 mg, 287 umol, 1 eq). The mixture was stirred at 80° C. for 12 h. LC-MS indicated completion of the reaction and formation of a new major peak with desired mass. The reaction mixture was concentrated under reduced pressure and the residue was purified by prep-HPLC (HCl condition) to afford methyl 5-amino-7-(2-(4-(4-(2-(tert-butoxy)-2-oxoethoxy)phenyl)piperazin-1-yl)ethyl)-9-methyl-2-(pyridin-2-yl)-7H-pyrrolo[3,2-e][1,2,4]triazolo[1,5-c]pyrimidine-8-carboxylate (90 mg, 25.0 umol, HCl) as a red solid. ¹H NMR (400 MHz, METHANOL-d₄) δ=8.75 (br s, 1H), 8.31 (d, J=7.9 Hz, 1H), 8.02 (br d, J=6.4 Hz, 3H), 7.58-7.52 (m, 1H), 6.88-6.75 (m, 4H), 4.64 (br s, 2H), 4.52 (s, 2H), 3.86 (s, 3H), 3.31-3.29 (m, 2H), 2.97 (br s, 4H), 2.76 (s, 3H), 2.61 (br s, 4H), 1.42 (s, 9H).

### Step 4: Synthesis of 2-(4-(4-(2-(5-amino-8-(methoxycarbonyl)-9-methyl-2-(pyridin-2-yl)-7H-pyrrolo[3,2-e][1,2,4]triazolo[1,5-c]pyrimidin-7-yl)ethyl)piperazin-1-yl)phenoxy)acetic acid

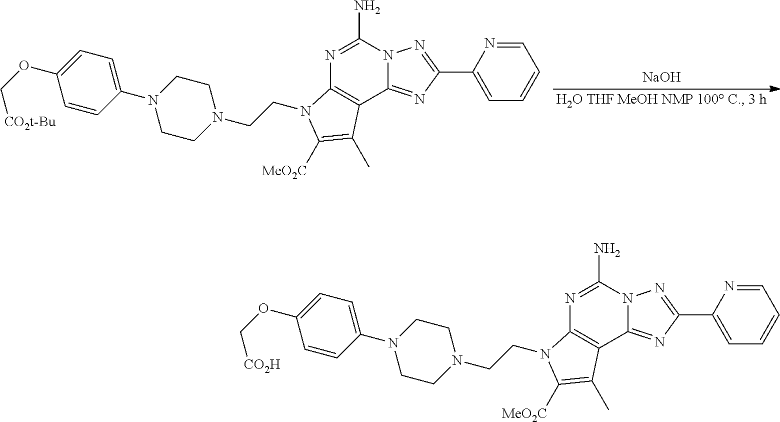

To a solution of methyl 5-amino-7-[2-[4-[4-(2-tert-butoxy-2-oxo-ethoxy)phenyl]piperazin-1-yl]ethyl]-9-methyl-2-(2-pyridyl)-[1,2,4]triazolo pyrrolo pyrimidine-8-carboxylate (90 mg, 132 umol, 1 eq, HCl) in tetrahydrofuran (1 mL), methanol (1 mL) and 1-methyl-2-pyrrolidinone (2 mL) was added NaOH (37.1 mg, 928 umol, 7 eq) in water (0.5 mL). The mixture was stirred at 100° C. for 3 h. LC-MS indicated completion of the reaction and formation of a new major peak with desired mass. The reaction mixture was concentrated under reduced pressure and the residue was purified by prep-HPLC (neutral condition) to afford 2-(4-(4-(2-(5-amino-8-(methoxycarbonyl)-9-methyl-2-(pyridin-2-yl)-7H-pyrrolo[3,2-e][1,2,4]triazolo[1,5-c]pyrimidin-7-yl)ethyl)piperazin-1-yl)phenoxy)acetic acid (20 mg, 34.9 umol) as a white solid. LCMS for product (ESI+): m/z 572.1 (M+H)⁺, Rt: 1.767 min. ¹H NMR (400 MHz, DMSO-d₆) δ=8.74 (d, J=4.4 Hz, 1H), 8.31 (d, J=7.8 Hz, 1H), 8.00 (t, J=7.6 Hz, 1H), 7.70 (br s, 2H), 7.57-7.49 (m, 1H), 6.80 (br d, J=9.3 Hz, 2H), 6.74-6.68 (m, 2H), 4.72 (br t, J=6.8 Hz, 2H), 4.16 (s, 2H), 2.94 (br s, 4H), 2.73 (s, 3H), 2.71-2.65 (m, 2H), 2.61 (br d, J=3.9 Hz, 4H).

### Example 46: Synthesis of 5-amino-7-(2-(4-(2-fluoro-5-(oxazol-2-yl)phenyl)-1,4-diazepan-1-yl)ethyl)-9-methyl-2-(pyridin-2-yl)-7H-pyrrolo[3,2-e][1,2,4]triazolo[1,5-c]pyrimidine-8-carboxylic acid

### Step 1: Synthesis of methyl 5-amino-7-(2-(4-(2-fluoro-5-(oxazol-2-yl)phenyl)-1,4-diazepan-1-yl)ethyl)-9-methyl-2-(pyridin-2-yl)-7H-pyrrolo[3,2-e][1,2,4]triazolo[1,5-c]pyrimidine-8-carboxylate

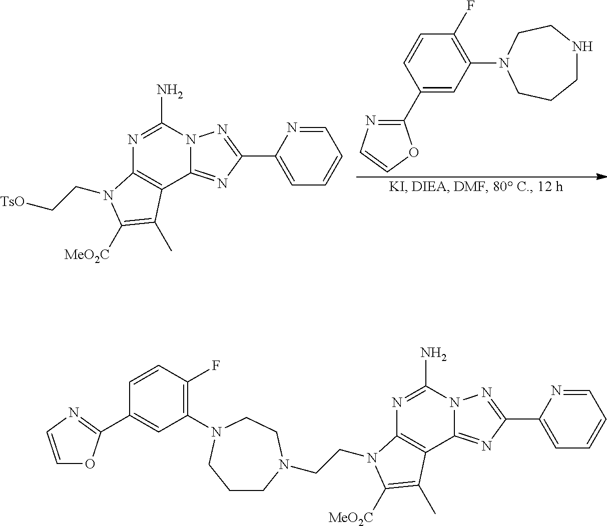

To a solution of methyl 5-amino-9-methyl-7-[2-(p-tolylsulfonyloxy)ethyl]-2-(2-pyridyl)-[1,2,4]triazolo pyrrolo pyrimidine-8-carboxylate (0.2 g, 383 umol, 1 eq) and 2-[3-(1,4-diazepan-1-yl)-4-fluoro-phenyl]oxazole (120 mg, 460 umol, 1.2 eq) in N,N-dimethylformamide (3.8 mL) was added KI (63.6 mg, 383 umol, 1 eq) and N,N-diisopropylethylamine (198 mg, 1.53 mmol, 267 uL, 4 eq). The mixture was stirred at 80° C. for 12 h. LC-MS showed consumption of starting material and detection of several new peaks including the desired compound. To the reaction mixture (suspension) was added 1N HCl (1 mL) and the clear solution was purified by prep-HPLC (HCl condition) to afford methyl 5-amino-7-(2-(4-(2-fluoro-5-(oxazol-2-yl)phenyl)-1,4-diazepan-1-yl)ethyl)-9-methyl-2-(pyridin-2-yl)-7H-pyrrolo[3,2-e][1,2,4]triazolo[1,5-c]pyrimidine-8-carboxylate (27 mg, 42.6 umol, HCl) as a yellow solid. ¹H NMR (400 MHz, METHANOL-d₄) δ=8.93 (d, J=4.9 Hz, 1H), 8.79 (d, J=7.9 Hz, 1H), 8.65 (dt, J=1.5, 7.9 Hz, 1H), 8.09 (ddd, J=1.2, 5.8, 7.5 Hz, 1H), 7.95 (d, J=0.7 Hz, 1H), 7.53 (dd, J=2.0, 8.6 Hz, 1H), 7.42 (ddd, J=2.1, 4.2, 8.4 Hz, 1H), 7.27 (d, J=0.7 Hz, 1H), 7.08 (dd, J=8.5, 13.1 Hz, 1H), 5.02 (br d, J=8.8 Hz, 2H), 4.00-3.91 (m, 5H), 3.85 (t, J=5.7 Hz, 2H), 3.78 (br d, J=13.0 Hz, 1H), 3.57 (br s, 3H), 3.48-3.36 (m, 2H), 2.88 (s, 3H), 2.47 (br s, 1H), 2.29 (br d, J=9.3 Hz, 1H).

### Step 2: Synthesis of 5-amino-7-(2-(4-(2-fluoro-5-(oxazol-2-yl)phenyl)-1,4-diazepan-1-yl)ethyl)-9-methyl-2-(pyridin-2-yl)-7H-pyrrolo[3,2-e][1,2,4]triazolo[1,5-c]pyrimidine-8-carboxylic acid

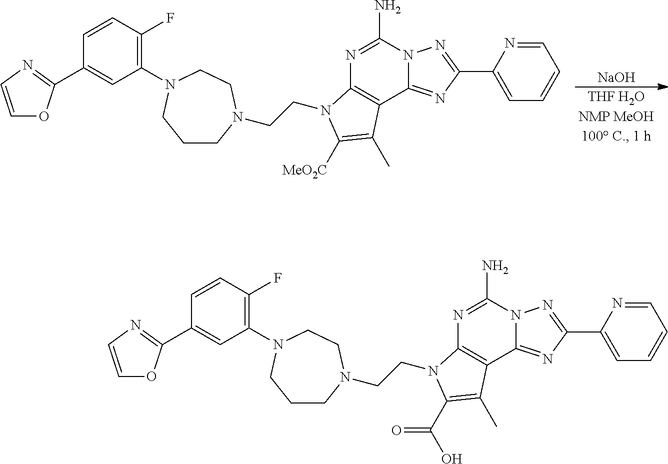

To a solution of methyl 5-amino-7-[2-[4-(2-fluoro-5-oxazol-2-yl-phenyl)-1,4-diazepan-1-yl]ethyl]-9-methyl-2-(2-pyridyl)-[1,2,4]triazolo pyrrolo pyrimidine-8-carboxylate (10 mg, 13.8 umol, 1 eq) in methanol (0.1 mL), tetrahydrofuran (0.1 mL) and 1-methyl-2-pyrrolidinone (0.25 mL) was added NaOH (3.86 mg, 96.6 umol, 7 eq) in water (0.05 mL). The mixture was stirred at 100° C. for 1 h. LC-MS indicated completion of the reaction and formation of a new major peak with desired mass. To the reaction mixture (suspension) was added trifluoroacetic acid (0.5 mL) and the clear solution was purified by prep-HPLC (neutral condition) to afford 5-amino-7-(2-(4-(2-fluoro-5-(oxazol-2-yl)phenyl)-1,4-diazepan-1-yl)ethyl)-9-methyl-2-(pyridin-2-yl)-7H-pyrrolo[3,2-e][1,2,4]triazolo[1,5-c]pyrimidine-8-carboxylic acid (30 mg, 49.5 umol) as a white solid. LCMS for product (ESI+): m/z 597.3 (M+H)⁺, Rt: 2.297 min. ¹H NMR (400 MHz, DMSO-d₆) δ=8.77-8.73 (m, 1H), 8.31 (d, J=7.8 Hz, 1H), 8.17 (s, 1H), 8.01 (dt, J=2.0, 7.8 Hz, 1H), 7.90 (br s, 2H), 7.57-7.51 (m, 1H), 7.46-7.42 (m, 1H), 7.36-7.30 (m, 2H), 7.19 (dd, J=8.3, 13.7 Hz, 1H), 4.61 (br t, J=6.8 Hz, 2H), 3.38 (br d, J=6.4 Hz, 4H), 2.91 (br s, 2H), 2.83 (br t, J=6.4 Hz, 2H), 2.74 (s, 5H), 1.88 (br s, 2H).

### Example 47: Synthesis of 7-(2-(4-(2-fluoro-5-(oxazol-2-yl)phenyl)-1,4-diazepan-1-yl)ethyl)-9-methyl-2-(pyridin-2-yl)-7H-pyrrolo[3,2-e][1,2,4]triazolo[1,5-c]pyrimidin-5-amine

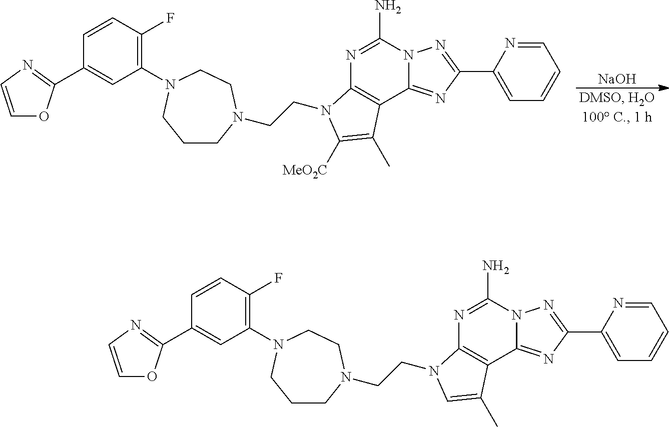

To a solution of methyl 5-amino-7-[2-[4-(2-fluoro-5-oxazol-2-yl-phenyl)-1,4-diazepan-1-yl]ethyl]-9-methyl-2-(2-pyridyl)-[1,2,4]triazolo pyrrolo pyrimidine-8-carboxylate (198 mg, 305 umol, 1 eq, HCl) in dimethyl sulfoxide (100 mL) was added NaOH (400 mg, 10.0 mmol, 32.6 eq) in water (10 mL). The mixture was stirred at 100° C. for 1 h. LC-MS indicated completion of the reaction and formation of a new major peak with desired mass. The reaction mixture was concentrated under reduced pressure and the residue was purified by prep-HPLC (neutral condition) to afford 7-(2-(4-(2-fluoro-5-(oxazol-2-yl)phenyl)-1,4-diazepan-1-yl)ethyl)-9-methyl-2-(pyridin-2-yl)-7H-pyrrolo[3,2-e][1,2,4]triazolo[1,5-c]pyrimidin-5-amine (20 mg, 35.4 umol) as a white solid. LCMS for product (ESI+): m/z 553.3 (M+H)⁺, Rt: 3.083 min. ¹H NMR (400 MHz, DMSO-d₆) δ=8.74 (d, J=4.4 Hz, 1H), 8.30 (d, J=7.8 Hz, 1H), 8.17 (s, 1H), 7.99 (dt, J=2.0, 7.8 Hz, 1H), 7.56-7.44 (m, 4H), 7.37-7.31 (m, 2H), 7.20 (dd, J=8.3, 13.7 Hz, 1H), 6.88 (s, 1H), 4.17 (br t, J=6.6 Hz, 2H), 3.44-3.38 (m, 4H), 2.88 (br d, J=6.4 Hz, 4H), 2.73 (br s, 2H), 2.40 (s, 3H), 1.88 (br s, 2H).

### Example 48: Synthesis of 5-amino-7-(2-(4-(2-fluoro-5-(oxazol-2-yl)phenyl)-1,4-diazepan-1-yl)ethyl)-N,9-dimethyl-2-(pyridin-2-yl)-7H-pyrrolo[3,2-e][1,2,4]triazolo[1,5-c]pyrimidine-8-carboxamide

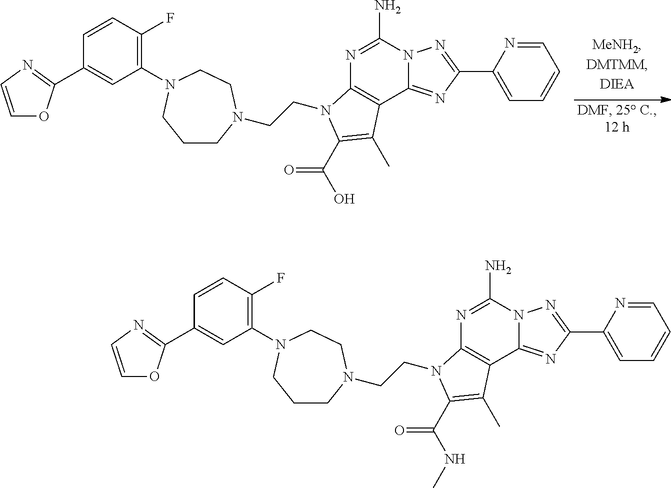 [SMILES: Cc1c(C(=O)O)n(CCN2CCCN(c3cc(-c4ncco4)ccc3F)CC2)c2nc(N)n3nc(-c4ccccn4)nc3c12 ; "DMF, 25° C.,12 h" ; CNC(=O)c1c(C)c2c(nc(N)n3nc(-c4ccccn4)nc23)n1CCN1CCCN(c2cc(-c3ncco3)ccc2F)CC1]

To a solution of 5-amino-7-[2-[4-(2-fluoro-5-oxazol-2-yl-phenyl)-1,4-diazepan-1-yl]ethyl]-9-methyl-2-(2-pyridyl)-[1,2,4]triazolo pyrrolo pyrimidine-8-carboxylic acid (25 mg, 41.9 umol, 1 eq) and methylamine (2 M, 83.8 uL, 4 eq) in N,N-dimethylformamide (1 mL) was added N,N-diisopropylethylamine (27.0 mg, 209 umol, 36.4 uL, 5 eq) and 4-(4,6-dimethoxy-1,3,5-triazin-2-yl)-4-methyl-morpholin-4-ium; tetrafluoroborate (27.4 mg, 83.8 umol, 2 eq). The mixture was stirred at 25° C. for 12 h. LC-MS indicated completion of the reaction and formation of a new major peak with desired mass. To the reaction mixture (suspension) was added trifluoroacetic acid (0.1 mL) and the clear solution was purified by prep-HPLC (HCl condition) to afford 5-amino-7-(2-(4-(2-fluoro-5-(oxazol-2-yl)phenyl)-1,4-diazepan-1-yl)ethyl)-N,9-dimethyl-2-(pyridin-2-yl)-7H-pyrrolo[3,2-e][1,2,4]triazolo[1,5-c]pyrimidine-8-carboxamide (13 mg, 20.1 umol, HCl) as a yellow solid. LCMS for product (ESI+): m/z 610.1 (M+H)⁺, Rt: 1.945 min. ¹H NMR (400 MHz, METHANOL-d₄) δ=8.98 (d, J=5.4 Hz, 1H), 8.93 (d, J=7.8 Hz, 1H), 8.85-8.80 (m, 1H), 8.23 (t, J=6.8 Hz, 1H), 7.98 (s, 1H), 7.58 (br d, J=8.8 Hz, 1H), 7.49 (td, J=2.1, 4.0 Hz, 1H), 7.30 (s, 1H), 7.16 (dd, J=8.6, 13.0 Hz, 1H), 4.76 (br t, J=5.1 Hz, 2H), 3.86 (br d, J=4.4 Hz, 2H), 3.72-3.38 (m, 8H), 2.98 (s, 3H), 2.77 (s, 3H), 2.39 (br s, 2H).

### Example 49: Synthesis of 2-(4-(4-(2-(5-amino-8-cyano-2-(pyridin-2-yl)-7H-pyrrolo[3,2-e][1,2,4]triazolo[1,5-c]pyrimidin-7-yl)ethyl)piperazin-1-yl)phenoxy)-N-(methylsulfonyl)acetamide

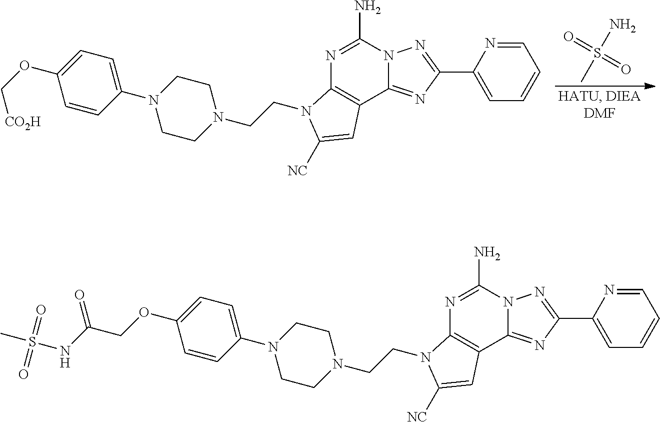

2-[4-[4-[2-[5-amino-8-cyano-2-(2-pyridyl)-[1,2,4]triazolo pyrrolo pyrimidin-7-yl]ethyl]piperazin-1-yl]phenoxy]acetic acid (10 mg, 18.57 umol, 1 eq) was dissolved in DMF (1 mL). Methane sulfonamide (3.53 mg, 37.1 umol, 2.0 eq), HATU (10.6 mg, 27.9 umol, 1.5 eq) and DIEA (7.20 mg, 55.7 umol, 9.70 uL, 3.0 eq) were added to the reaction mixture. The solution was stirred at 20° C. for 12 h. LC-MS indicated completion of the reaction and formation of a new major peak with desired mass. The reaction mixture was purified by prep-HPLC to afford 2-(4-(4-(2-(5-amino-8-cyano-2-(pyridin-2-yl)-7H-pyrrolo[3,2-e][1,2,4]triazolo[1,5-c]pyrimidin-7-yl)ethyl)piperazin-1-yl)phenoxy)-N-(methylsulfonyl)acetamide (2 mg, HCl) as a yellow solid. LCMS for product (ESI+): m/z 616.2 (M+H)⁺, Rt: 1.734 min. ¹H NMR (400 MHz, DMSO-d₆) δ=12.10 (br s, 1H), 10.04 (br s, 1H), 8.75 (d, J=4.4 Hz, 1H), 8.32-8.06 (m, 2H), 8.04-8.02 (m, 1H), 7.82 (s, 1H), 7.60-7.57 (m, 1H), 6.98-6.92 (m, 2H), 6.88-6.85 (m, 2H), 4.68-4.64 (m, 4H), 3.95 (br s, 2H), 3.72 (br s, 4H), 3.27 (s, 5H), 2.98 (s, 2H).

### Example 50: Synthesis of 5-amino-9-chloro-7-(2-(4-(6-fluoro-3-methylbenzo[d]isoxazol-5-yl)piperazin-1-yl)ethyl)-2-(pyridin-2-yl)-7H-pyrrolo[3,2-e][1,2,4]triazolo[1,5-c]pyrimidin-8-yl formate

### Step 1: Synthesis of ethyl 2-amino-7-[2-[tert-butyl(dimethyl)silyl]oxyethyl]-4-hydrazino-pyrrolo[2,3-d]pyrimidine-6-carboxylate

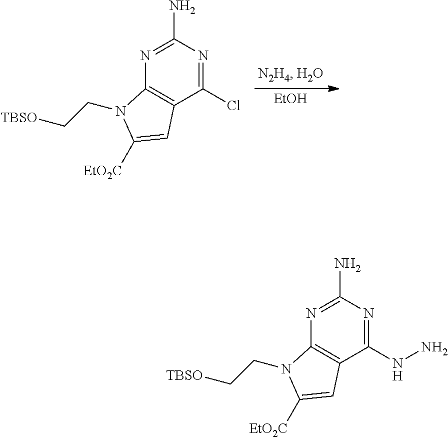 [SMILES: "EtOH" ; CCOC(=O)c1cc2c(Cl)nc(N)nc2n1CCO[Si](C)(C)C(C)(C)C ; CCOC(=O)c1cc2c(NN)nc(N)nc2n1CCO[Si](C)(C)C(C)(C)C]

To a solution of ethyl 2-amino-7-[2-[tert-butyl(dimethyl)silyl]oxyethyl]-4-chloro-pyrrolo[2,3-d]pyrimidine-6-carboxylate (30 g, 75.20 mmol, 1 eq) in EtOH (240 mL) was added N₂H₄.H₂O (38.41 g, 751.96 mmol, 37.29 mL, 10 eq). The mixture was stirred at 80° C. for 2 h. TLC (PE/EA=5:1) showed starting material was consumed and a new spot corresponding to the desired product. The resulting solid was collected by filtration and used without purification to afford ethyl 2-amino-7-[2-[tert-butyl(dimethyl)silyl]oxyethyl]-4-hydrazino-pyrrolo[2,3-d]pyrimidine-6-carboxylate (27 g, 68.43 mmol) as a white solid. ¹H NMR (400 MHz, DMSO-d₆) δ=8.91-8.48 (m, 1H), 7.30 (br d, J=3.9 Hz, 1H), 6.06 (br s, 2H), 4.45 (br t, J=6.1 Hz, 4H), 4.21 (q, J=7.1 Hz, 2H), 3.76 (t, J=6.1 Hz, 2H), 1.27 (t, J=7.1 Hz, 3H), 0.76 (s, 9H), −0.13 (s, 6H).

### Step 2: Synthesis of 2-amino-7-[2-[tert-butyl(dimethyl)silyl]oxyethyl]-4-[2-(pyridine-2-carbonyl)hydrazino]pyrrolo[2,3-d]pyrimidine-6-carboxylate

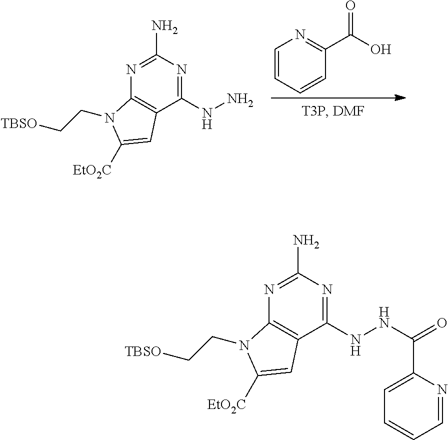 [SMILES: CCOC(=O)c1cc2c(NN)nc(N)nc2n1CCO[Si](C)(C)C(C)(C)C ; O=C(O)c1ccccn1 ; CCOC(=O)c1cc2c(NNC(=O)c3ccccn3)nc(N)nc2n1CCO[Si](C)(C)C(C)(C)C]

To a solution of ethyl 2-amino-7-[2-[tert-butyl(dimethyl)silyl]oxyethyl]-4-hydrazino-pyrrolo[2,3-d]pyrimidine-6-carboxylate (27 g, 68.43 mmol, 1 eq) and pyridine-2-carboxylic acid (12.64 g, 102.65 mmol, 1.5 eq) in DMF (300 mL) was added DIEA (26.53 g, 205.30 mmol, 35.76 mL, 3 eq) and T3P (87.10 g, 136.87 mmol, 81.40 mL, 50% purity, 2 eq). The mixture was stirred at 25° C. for 2 h. LCMS indicated disappearance of starting material and formation of a new peak with the desired mass. The mixture was poured into water (1.5 L) and the resulting solid was collected by filtration. The residue was triturated in EtOAc (300 mL) to afford 2-amino-7-[2-[tert-butyl(dimethyl)silyl]oxyethyl]-4-[2-(pyridine-2-carbonyl)hydrazino]pyrrolo[2,3-d]pyrimidine-6-carboxylate (30 g) as a yellow solid. LCMS for product (ESI+): m/z 500.2 [M+H]⁺, Rt: 1.112 min.

### Step 3: Synthesis of ethyl 5-amino-7-[2-[tert-butyl(dimethyl)silyl]oxyethyl]-2-(2-pyridyl)-[1,2,4]triazolo[BLAH]pyrrolo[BLAH]pyrimidine-8-carboxylate

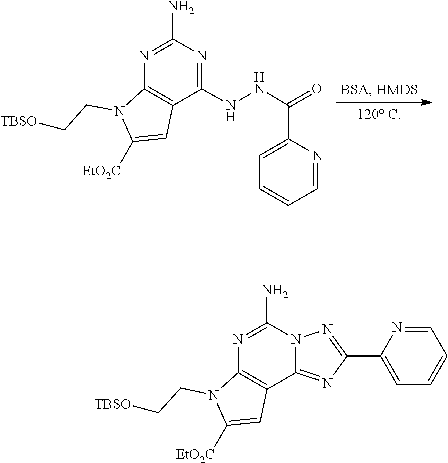 [SMILES: CCOC(=O)c1cc2c(NNC(=O)c3ccccn3)nc(N)nc2n1CCO[Si](C)(C)C(C)(C)C ; CCOC(=O)c1cc2c(nc(N)n3nc(-c4ccccn4)nc23)n1CCO[Si](C)(C)C(C)(C)C]

To a solution of ethyl 2-amino-7-[2-[tert-butyl(dimethyl)silyl]oxyethyl]-4-[2-(pyridine-2-carbonyl)hydrazino]pyrrolo[2,3-d]pyrimidine-6-carboxylate (15 g, 30.02 mmol, 1 eq) in HMDS (150 mL) was added BSA (73.29 g, 360.26 mmol, 89.05 mL, 12 eq). The mixture was stirred at 120° C. for 12 h. LCMS indicated disappearance of starting material and formation of a new peak with the desired mass. One additional vial was set up as described above and the the mixtures were combined and poured into water (1 L). The aqueous layer was extracted with EtOAc (3×500 mL). The combined organic layers were washed with brine (100 mL) and dried over Na₂SO₄. The mixture was concentrated, and the residue was triturated in EtOAc (200 mL) to afford ethyl 5-amino-7-[2-[tert-butyl(dimethyl)silyl]oxyethyl]-2-(2-pyridyl)-[1,2,4]triazolo[BLAH]pyrrolo[BLAH]pyrimidine-8-carboxylate (27 g, 56.06 mmol) as a white solid. LCMS for product (ESI+): m/z 368.1 [M+H]⁺, Rt: 0.951 min.

### Step 4: Synthesis of 5-amino-7-(2-hydroxyethyl)-2-(2-pyridyl)-[1,2,4]triazolo[BLAH]pyrrolo[BLAH]pyrimidine-8-carboxylate

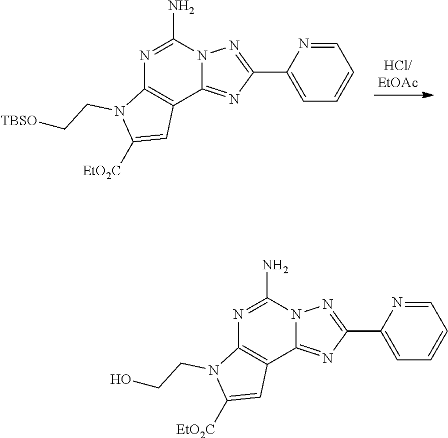 [SMILES: CCOC(=O)c1cc2c(nc(N)n3nc(-c4ccccn4)nc23)n1CCO[Si](C)(C)C(C)(C)C ; CCOC(=O)c1cc2c(nc(N)n3nc(-c4ccccn4)nc23)n1CCO]

A solution of ethyl 5-amino-7-[2-[tert-butyl(dimethyl)silyl]oxyethyl]-2-(2-pyridyl)-[1,2,4]triazolo[BLAH]pyrrolo[BLAH]pyrimidine-8-carboxylate (18 g, 37.37 mmol, 1 eq) in HCl/EtOAc (200 mL, 4 N) was stirred at 25° C. for 1 h. LCMS indicated disappearance of starting material and formation of a new peak with the desired mass. One additional vial was set up as described above and the mixtures were combined and concentrated to afford 5-amino-7-(2-hydroxyethyl)-2-(2-pyridyl)-[1,2,4]triazolo[BLAH]pyrrolo[BLAH]pyrimidine-8-carboxylate (30 g) as a white solid (used without further purification). LCMS for product (ESI+): m/z 368.1 [M+H]⁺, Rt: 0.954 min.

### Step 5: Synthesis of ethyl 5-amino-7-[2-(p-tolylsulfonyloxy)ethyl]-2-(2-pyridyl)-[1,2,4]triazolo[BLAH]pyrrolo[BLAH]pyrimidine-8-carboxylate

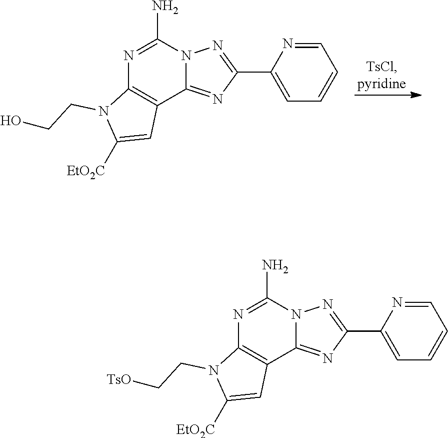 [SMILES: CCOC(=O)c1cc2c(nc(N)n3nc(-c4ccccn4)nc23)n1CCO ; CCOC(=O)c1cc2c(nc(N)n3nc(-c4ccccn4)nc23)n1CCOS(=O)(=O)c1ccc(C)cc1]

To a solution of ethyl 5-amino-7-(2-hydroxyethyl)-2-(2-pyridyl)-[1,2,4]triazolo[BLAH]pyrrolo[BLAH]pyrimidine-8-carboxylate (10 g, 27.22 mmol, 1 eq) in PYRIDINE (100 mL) was added 4-methylbenzenesulfonyl chloride (15.57 g, 81.66 mmol, 3 eq) at 0° C. The mixture was stirred at 40° C. for 12 h. LCMS indicated disappearance of starting material and formation of a new peak with the desired mass. Two additional vials were set up as described above and the mixtures were combined and poured into water (1 L). The resulting solid was triturated in EtOAc (100 mL) and collected by filtration to afford ethyl 5-amino-7-[2-(p-tolylsulfonyloxy)ethyl]-2-(2-pyridyl)-[1,2,4]triazolo[BLAH]pyrrolo[BLAH]pyrimidine-8-carboxylate (35 g, 67.11 mmol) as a white solid. LCMS for product (ESI+): m/z 522.1 [M+H]⁺, Rt: 1.139 min. 1H NMR (400 MHz, DMSO-d₆) δ=8.77 (br d, J=4.1 Hz, 1H), 8.32 (d, J=7.9 Hz, 1H), 8.10-7.99 (m, 3H), 7.56 (dd, J=5.1, 7.2 Hz, 1H), 7.36-7.26 (m, 3H), 7.06 (d, J=8.1 Hz, 2H), 4.75 (br t, J=4.7 Hz, 2H), 4.49 (br t, J=4.8 Hz, 2H), 4.27 (q, J=7.1 Hz, 2H), 2.12 (s, 3H), 1.32 (t, J=7.1 Hz, 3H).

### Step 6: Synthesis of ethyl 5-amino-9-chloro-7-[2-(p-tolylsulfonyloxy)ethyl]-2-(2-pyridyl)-[1,2,4]triazolo[BLAH]pyrrolo[BLAH]pyrimidine-8-carboxylate

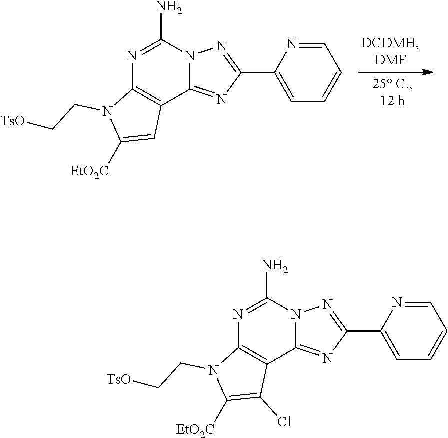 [SMILES: CCOC(=O)c1cc2c(nc(N)n3nc(-c4ccccn4)nc23)n1CCOS(=O)(=O)c1ccc(C)cc1 ; CCOC(=O)c1c(Cl)c2c(nc(N)n3nc(-c4ccccn4)nc23)n1CCOS(=O)(=O)c1ccc(C)cc1]

To a solution of ethyl 5-amino-7-[2-(p-tolylsulfonyloxy)ethyl]-2-(2-pyridyl)-[1,2,4]triazolo[BLAH]pyrrolo[BLAH]pyrimidine-8-carboxylate (6 g, 11.50 mmol, 1 eq) in DMF (500 mL) was added 1,3-dichloro-5,5-dimethyl-imidazolidine-2,4-dione (1.13 g, 5.75 mmol, 0.5 eq). The mixture was stirred at 25° C. for 12 h. LCMS indicated disappearance of starting material and formation of a new peak with the desired mass. One additional vial was set up as described above and the two reaction mixtures were combined and poured into water (3 L). The resulting solid was collected by filtration to afford ethyl 5-amino-9-chloro-7-[2-(p-tolylsulfonyloxy)ethyl]-2-(2-pyridyl)-[1,2,4]triazolo[BLAH]pyrrolo[BLAH]pyrimidine-8-carboxylate (10 g, 17.01 mmol) as a pink solid (used without further purification). LCMS for product (ESI+): m/z 556.1 [M+H]⁺, Rt: 1.185 min. ¹H NMR (400 MHz, DMSO-d₆) δ=8.78 (d, J=4.1 Hz, 1H), 8.33 (d, J=7.9 Hz, 1H), 8.23 (br s, 2H), 8.05 (dt, J=1.8, 7.8 Hz, 1H), 7.58 (ddd, J=0.9, 4.8, 7.5 Hz, 1H), 7.32 (d, J=8.3 Hz, 2H), 7.05 (d, J=8.1 Hz, 2H), 4.72 (t, J=4.8 Hz, 2H), 4.47 (t, J=4.8 Hz, 2H), 4.31 (q, J=7.1 Hz, 2H), 2.15 (s, 3H), 1.35 (t, J=7.1 Hz, 3H).

### Step 7: Synthesis of ethyl 5-amino-9-chloro-7-[2-[4-(6-fluoro-3-methyl-1,2-benzoxazol-5-yl)piperazin-1-yl]ethyl]-2-(2-pyridyl)-[1,2,4]triazolo[BLAH]pyrrolo[BLAH]pyrimidine-8-carboxylate

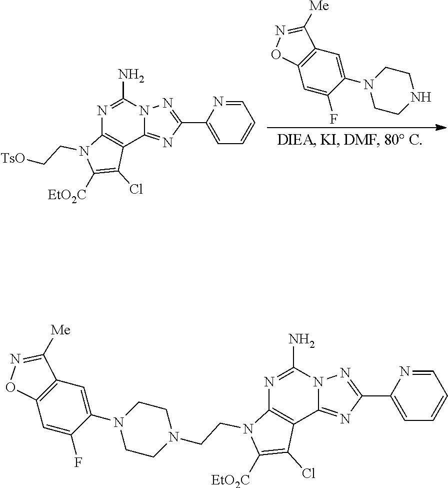 [SMILES: CCOC(=O)c1c(Cl)c2c(nc(N)n3nc(-c4ccccn4)nc23)n1CCOS(=O)(=O)c1ccc(C)cc1 ; Cc1noc2cc(F)c(N3CCNCC3)cc12 ; CCOC(=O)c1c(Cl)c2c(nc(N)n3nc(-c4ccccn4)nc23)n1CCN1CCN(c2cc3c(C)noc3cc2F)CC1]

To a solution of ethyl 5-amino-9-chloro-7-[2-(p-tolylsulfonyloxy)ethyl]-2-(2-pyridyl)-[1,2,4]triazolo[BLAH]pyrrolo[BLAH]pyrimidine-8-carboxylate (200 mg, 359.72 umol, 1 eq) and 6-fluoro-3-methyl-5-piperazin-1-yl-1,2-benzoxazole (101.55 mg, 431.66 umol, 1.2 eq) in DMF (5 mL) was added DIEA (139.47 mg, 1.08 mmol, 187.97 uL, 3 eq) and KI (119.43 mg, 719.43 umol, 2 eq). The mixture was stirred at 80° C. for 3 h. LCMS showed consumption of starting material and a new peak corresponding to the desired product mass.

One additional vial was set up as described above and the two reaction mixtures were combined. The mixture was poured into water (20 mL). The resulting solid was collected by filtration and purified by column chromatography (SiO2, Ethyl acetate/MeOH=100:1 to 5:1) to afford ethyl 5-amino-9-chloro-7-[2-[4-(6-fluoro-3-methyl-1,2-benzoxazol-5-yl)piperazin-1-yl]ethyl]-2-(2-pyridyl)-[1,2,4]triazolo[BLAH]pyrrolo[BLAH]pyrimidine-8-carboxylate (230 mg, 371.54 umol) as a brown solid. LCMS for product (ESI+): m/z 619.1 [M+H]⁺, Rt: 1.001 min.

### Step 8: Synthesis of 5-amino-9-chloro-7-(2-(4-(6-fluoro-3-methylbenzo[d]isoxazol-5-yl)piperazin-1-yl)ethyl)-2-(pyridin-2-yl)-7H-pyrrolo[3,2-e][1,2,4]triazolo[1,5-c]pyrimidin-8-yl formate

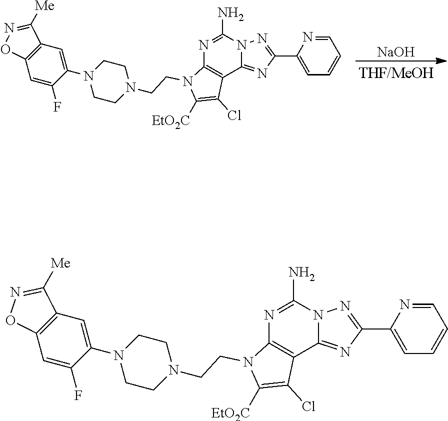

To a solution of ethyl 5-amino-9-chloro-7-[2-[4-(6-fluoro-3-methyl-1,2-benzoxazol-5-yl)piperazin-1-yl]ethyl]-2-(2-pyridyl)-[1,2,4]triazolo[BLAH]pyrrolo[BLAH]pyrimidine-8-carboxylate (110 mg, 177.69 umol, 1 eq) in THF (1 mL), MeOH (1 mL) and H₂O (0.5 mL) was added NaOH (49.75 mg, 1.24 mmol, 16.15 uL, 7 eq). The mixture was stirred at 90° C. for 3 h. LCMS showed starting consumption of the starting material and formation of the desired product. One additional vial was set up as described above and the two reaction mixtures were combined. The mixture was concentrated, acidified to pH 2 by dropwise addition of 2 N HCl and the resulting solid was collected by filtration to afford 5-amino-9-chloro-7-(2-(4-(6-fluoro-3-methylbenzo[d]isoxazol-5-yl)piperazin-1-yl)ethyl)-2-(pyridin-2-yl)-7H-pyrrolo[3,2-e][1,2,4]triazolo[1,5-c]pyrimidin-8-yl formate (140 mg, 236.89 umol) as a brown solid (used without further purification). LCMS for product (ESI+): m/z 591.1 [M+H]⁺, Rt: 1.456 min.

### Example 51: Synthesis of 5-amino-9-chloro-7-(2-(4-(6-fluoro-3-methylbenzo[d]isoxazol-5-yl)piperazin-1-yl)ethyl)-2-(pyridin-2-yl)-7H-pyrrolo[3,2-e][1,2,4]triazolo[1,5-c]pyrimidine-8-carboxamide

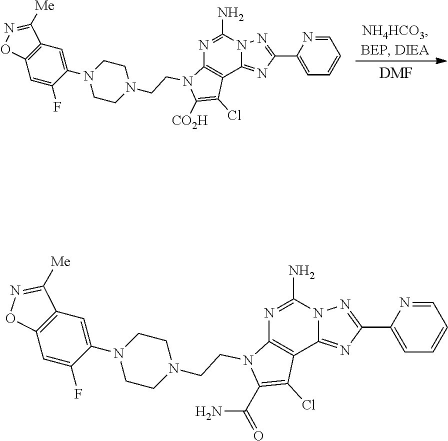

To a solution of 5-amino-9-chloro-7-[2-[4-(6-fluoro-3-methyl-1,2-benzoxazol-5-yl)piperazin-1-yl]ethyl]-2-(2-pyridyl)-[1,2,4]triazolo[BLAH]pyrrolo[BLAH]pyrimidine-8-carboxylic acid (120 mg, 203.05 umol, 1 eq) and NH₄HCO₃ (32.10 mg, 406.09 umol, 33.44 uL, 2 eq) in DMF (1 mL) was added DIEA (65.61 mg, 507.62 umol, 88.42 uL, 2.5 eq) and 2-bromo-1-ethyl-pyridin-1-ium; tetrafluoroborate (66.73 mg, 243.66 umol, 1.2 eq). The mixture was stirred at 25° C. for 3 h. LCMS showed consumption of starting material and formation of the desired product. The mixture was purified by prep-HPLC (HCl condition) to afford the desired product 5-amino-9-chloro-7-(2-(4-(6-fluoro-3-methylbenzo[d]isoxazol-5-yl)piperazin-1-yl)ethyl)-2-(pyridin-2-yl)-7H-pyrrolo[3,2-e][1,2,4]triazolo[1,5-c]pyrimidine-8-carboxamide (25 mg, 41.31 umol) as a yellow solid. LCMS for product (ESI+): m/z 590.2 [M+H]⁺, Rt: 1.913 min. ¹H NMR (400 MHz, DMSO-d₆) δ=9.94-9.70 (m, 1H), 8.80-8.74 (m, 1H), 8.32 (d, J=7.9 Hz, 1H), 8.21 (br s, 2H), 8.08-7.98 (m, 2H), 7.75 (d, J=11.5 Hz, 1H), 7.71-7.63 (m, 1H), 7.61-7.56 (m, 1H), 7.53 (d, J=8.3 Hz, 1H), 4.76 (br t, J=5.3 Hz, 2H), 3.97 (br d, J=11.4 Hz, 2H), 3.71 (br d, J=2.9 Hz, 2H), 3.46-3.30 (m, 4H), 3.17-3.06 (m, 2H), 2.53 (s, 3H).

### Example 52: Synthesis of 2-(4-(4-(2-(5-amino-8-carbamoyl-9-chloro-2-(pyridin-2-yl)-7H-pyrrolo[3,2-e][1,2,4]triazolo[1,5-c]pyrimidin-7-yl)ethyl)piperazin-1-yl)phenoxy)acetic acid

### Step 1: Synthesis of tert-butyl 4-(4-hydroxyphenyl)piperazine-1-carboxylate

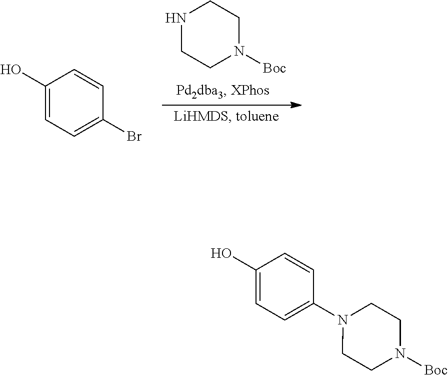 [SMILES: CC(C)(C)OC(=O)N1CCNCC1 ; Oc1ccc(Br)cc1 ; CC(C)(C)OC(=O)N1CCN(c2ccc(O)cc2)CC1]

To a mixture of 4-bromophenol (5 g, 28.90 mmol, 1 eq) in toluene (50 mL) as added tert-butyl piperazine-1-carboxylate (6.46 g, 34.68 mmol, 1.2 eq), XPhos (1.38 g, 2.89 mmol, 0.1 eq), Pd₂(dba)₃ (1.32 g, 1.45 mmol, 0.05 eq) under N₂ at 25° C. LiHMDS (1 M, 86.70 mL, 3 eq) was added to the mixture dropwise under N₂. The mixture was stirred at 80° C. for 2 h. TLC (PE/EA=2:1) showed the starting material was consumed and formation of a new spot. The mixture was poured into water (500 mL). The mixture was filtered, extracted with EtOAc (3×100 mL). The combined organic layer was washed with brine, dried over Na₂SO₄, filtered and concentrated. The residue was triturated in PE/EA (5:1, 30 mL). The mixture was filtered, and the solid was dried to give tert-butyl 4-(4-hydroxyphenyl) piperazine-1-carboxylate (6 g, 19.62 mmol) as a light-yellow solid. LCMS for product (ESI+): m/z 279.2 [M+H]⁺, Rt: 1.088 min.

### Step 2: Synthesis of tert-butyl 4-(4-(2-(tert-butoxy)-2-oxoethoxy)phenyl)piperazine-1-carboxylate

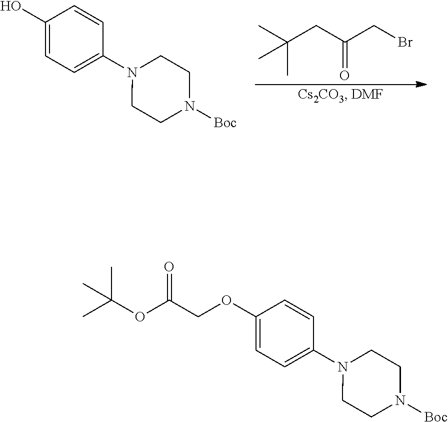

To a mixture of tert-butyl 4-(4-hydroxyphenyl) piperazine-1-carboxylate (1 g, 3.59 mmol, 1 eq) in DMF (10 mL) was added tert-butyl 2-bromoacetate (840.91 mg, 4.31 mmol, 637.05 uL, 1.2 eq) and Cs₂CO₃ (2.34 g, 7.19 mmol, 2 eq). The mixture was stirred at 60° C. for 2 h. TLC (PE/EA=1:1) indicated complete consumption of starting material and formation of a new spot. The mixture was poured into water (100 mL), extracted with ethyl acetate (3×20 mL), the combined organic layer was washed with brine, dried over Na₂SO₄, filtered and concentrated to afford tert-butyl 4-(4-(2-(tert-butoxy)-2-oxoethoxy)phenyl)piperazine-1-carboxylate (1.5 g) as a yellow solid (used without further purification).

### Step 3: Synthesis of tert-butyl 2-(4-(piperazin-1-yl)phenoxy)acetate

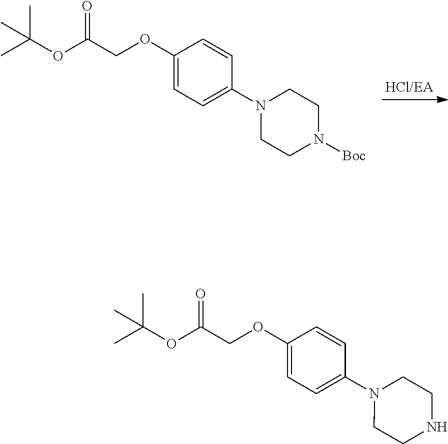 [SMILES: CC(C)(C)OC(=O)COc1ccc(N2CCN(C(=O)OC(C)(C)C)CC2)cc1 ; CC(C)(C)OC(=O)COc1ccc(N2CCNCC2)cc1]

To a mixture of tert-butyl 4-[4-(2-tert-butoxy-2-oxo-ethoxy) phenyl]piperazine-1-carboxylate (1.5 g, 3.82 mmol, 1 eq) in EtOAc (20 mL) was added HCl/EtOAc (4 M, 20 mL, 20.93 eq). The mixture was stirred at 25° C. for 2 h. TLC (PE/EA=1:1) indicated consumption of starting material and formation of a new spot. The mixture was filtered, and the solid was dried and dissolved in methanol. The pH o the solution was adjusted 7 with Amberlyst® A21 free base. The mixture was filtered and concentrated to yield tert-butyl 2-(4-(piperazin-1-yl)phenoxy)acetate (0.95 g, 3.25 mmol) as a yellow solid. ¹H NMR (400 MHz, DMSO-d₆) δ=6.94-6.89 (m, 2H), 6.84-6.79 (m, 2H), 4.55 (s, 2H), 3.23-3.18 (m, 4H), 3.15-3.11 (m, 4H), 1.42 (s, 9H).

### Step 4: Synthesis of ethyl 5-amino-7-(2-(4-(4-(2-(tert-butoxy)-2-oxoethoxy)phenyl)piperazin-1-yl)ethyl)-9-chloro-2-(pyridin-2-yl)-7H-pyrrolo[3,2-e][1,2,4]triazolo[1,5-c]pyrimidine-8-carboxylate

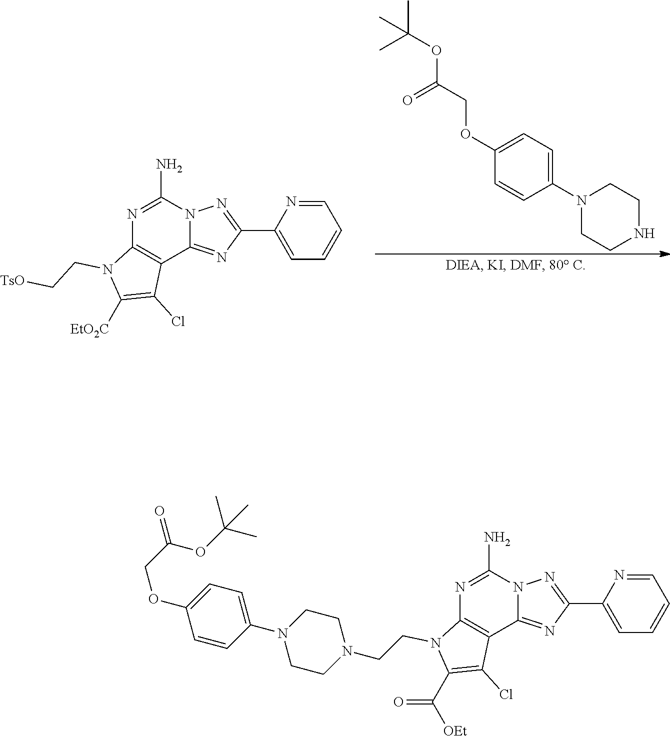 [SMILES: CCOC(=O)c1c(Cl)c2c(nc(N)n3nc(-c4ccccn4)nc23)n1CCOS(=O)(=O)c1ccc(C)cc1 ; CC(C)(C)OC(=O)COc1ccc(N2CCNCC2)cc1 ; CCOC(=O)c1c(Cl)c2c(nc(N)n3nc(-c4ccccn4)nc23)n1CCN1CCN(c2ccc(OCC(=O)OC(C)(C)C)cc2)CC1]

To a solution of ethyl 5-amino-9-chloro-7-[2-(p-tolylsulfonyloxy)ethyl]-2-(2-pyridyl)-[1,2,4]triazolo[BLAH]pyrrolo[BLAH]pyrimidine-8-carboxylate (600 mg, 1.08 mmol, 1 eq) and tert-butyl 2-(4-piperazin-1-ylphenoxy)acetate (473.27 mg, 1.62 mmol, 1.5 eq) in DMF (14 mL) was added DIEA (418.41 mg, 3.24 mmol, 563.89 uL, 3 eq) and KI (358.28 mg, 2.16 mmol, 2 eq). The mixture was stirred at 80° C. for 12 h. LCMS showed starting material was consumed and formation of a new peak with the desired mass. The mixture was poured into water (100 mL) and the resulting solid was collected by filtration to afford ethyl 5-amino-7-(2-(4-(4-(2-(tert-butoxy)-2-oxoethoxy)phenyl)piperazin-1-yl)ethyl)-9-chloro-2-(pyridin-2-yl)-7H-pyrrolo[3,2-e][1,2,4]triazolo[1,5-c]pyrimidine-8-carboxylate (700 mg, 1.04 mmol) as a brown solid (used without further purification).

### Step 5: Synthesis of 5-amino-7-(2-(4-(4-(carboxymethoxy)phenyl)piperazin-1-yl)ethyl)-9-chloro-2-(pyridin-2-yl)-7H-pyrrolo[3,2-e][1,2,4]triazolo[1,5-c]pyrimidine-8-carboxylic acid

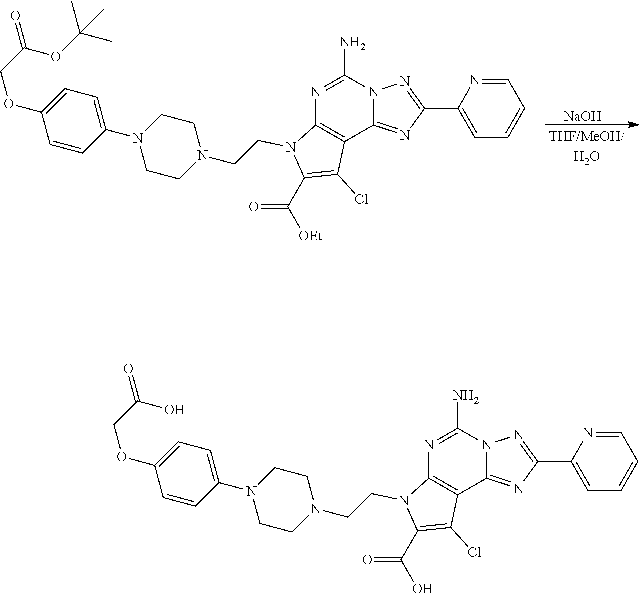

To a solution of ethyl 5-amino-7-[2-[4-[4-(2-tert-butoxy-2-oxo-ethoxy)phenyl]piperazin-1-yl]ethyl]-9-chloro-2-(2-pyridyl)-[1,2,4]triazolo[BLAH]pyrrolo[BLAH]pyrimidine-8-carboxylate (600 mg, 887.36 umol, 1 eq) in THE (18 mL), MeOH (18 mL) and H₂O (6 mL) was added NaOH (248.46 mg, 6.21 mmol, 129.41 uL, 7 eq). The mixture was stirred at 40° C. for 12 h. LCMS showed that starting material was consumed and formation of a new peak with the desired mass. The mixture was concentrated, and the pH was adjusted to 3 by dropwise addition of 2 N HCl. The resulting solid was filtered to afford 5-amino-7-(2-(4-(4-(carboxymethoxy)phenyl)piperazin-1-yl)ethyl)-9-chloro-2-(pyridin-2-yl)-7H-pyrrolo[3,2-e][1,2,4]triazolo[1,5-c]pyrimidine-8-carboxylic acid (430 mg, 726.35 umol) as a brown solid. LCMS for product (ESI+): m/z 592.2 [M+H]⁺, Rt: 0.861 min.

### Step 6: Synthesis of 5-amino-9-chloro-7-(2-(4-(4-(2-methoxy-2-oxoethoxy)phenyl)piperazin-1-yl)ethyl)-2-(pyridin-2-yl)-7H-pyrrolo[3,2-e][1,2,4]triazolo[1,5-c]pyrimidine-8-carboxylic acid

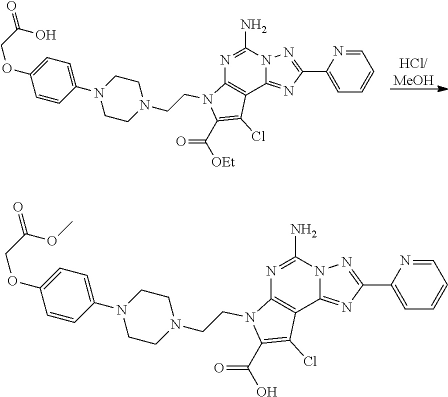 [SMILES: CCOC(=O)c1c(Cl)c2c(nc(N)n3nc(-c4ccccn4)nc23)n1CCN1CCN(c2ccc(OCC(=O)O)cc2)CC1 ; "HCl/MeOH" ; COC(=O)COc1ccc(N2CCN(CCn3c(C(=O)O)c(Cl)c4c3nc(N)n3nc(-c5ccccn5)nc43)CC2)cc1]

A solution of 5-amino-7-[2-[4-[4-(carboxymethoxy)phenyl]piperazin-1-yl]ethyl]-9-chloro-2-(2-pyridyl)-[1,2,4]triazolo[BLAH]pyrrolo[BLAH]pyrimidine-8-carboxylic acid (250 mg, 422.29 umol, 1 eq) in HCl/MeOH (4 M, 7.50 mL, 71.04 eq) was stirred at 25° C. for 1 h. LCMS showed that starting material was consumed and formation of a new peak with the desired mass. One additional vial was set up as described above and the two reaction mixtures were combined and concentrated. The residue was triturated in EtOAc (5 mL) and filtered to afford 5-amino-9-chloro-7-(2-(4-(4-(2-methoxy-2-oxoethoxy)phenyl)piperazin-1-yl)ethyl)-2-(pyridin-2-yl)-7H-pyrrolo[3,2-e][1,2,4]triazolo[1,5-c]pyrimidine-8-carboxylic acid (500 mg, 825.04 umol) as a yellow solid. LCMS for product (ESI+): m/z 606.2 [M+H]⁺, Rt: 1.351 min.

### Step 7: Synthesis of methyl 2-(4-(4-(2-(5-amino-8-carbamoyl-9-chloro-2-(pyridin-2-yl)-7H-pyrrolo[3,2-e][1,2,4]triazolo[1,5-c]pyrimidin-7-yl)ethyl)piperazin-1-yl)phenoxy)acetate

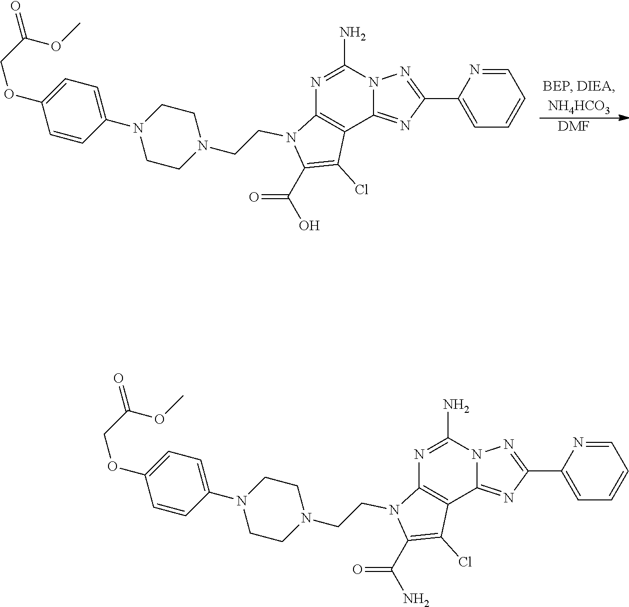

To a solution of 5-amino-9-chloro-7-[2-[4-[4-(2-methoxy-2-oxo-ethoxy)phenyl]piperazin-1-yl]ethyl]-2-(2-pyridyl)-[1,2,4]triazolo[BLAH]pyrrolo[BLAH]pyrimidine-8-carboxylic acid (250 mg, 412.52 umol, 1 eq) and NH₄HCO₃ (65.22 mg, 825.04 umol, 67.94 uL, 2 eq) in DMF (3 mL) was added 2-bromo-1-ethyl-pyridin-1-ium; tetrafluoroborate (135.57 mg, 495.02 umol, 1.2 eq) and DIEA (133.29 mg, 1.03 mmol, 179.63 uL, 2.5 eq). The mixture was stirred at 25° C. for 1 h. LCMS showed consumption of starting material and a new peak corresponding to the desired product. One additional vial was set up as described above and the two reaction mixtures were combined and poured into water (10 mL). The resulting solid was collected by filtration and used without purification to afford methyl 2-(4-(4-(2-(5-amino-8-carbamoyl-9-chloro-2-(pyridin-2-yl)-7H-pyrrolo[3,2-e][1,2,4]triazolo[1,5-c]pyrimidin-7-yl)ethyl)piperazin-1-yl)phenoxy)acetate (500 mg) as a brown solid. LCMS for product (ESI+): m/z 605.2 [M+H]⁺, Rt: 1.068 min.

### Step 8: Synthesis of 2-(4-(4-(2-(5-amino-8-carbamoyl-9-chloro-2-(pyridin-2-yl)-7H-pyrrolo[3,2-e][1,2,4]triazolo[1,5-c]pyrimidin-7-yl)ethyl)piperazin-1-yl)phenoxy)acetic acid

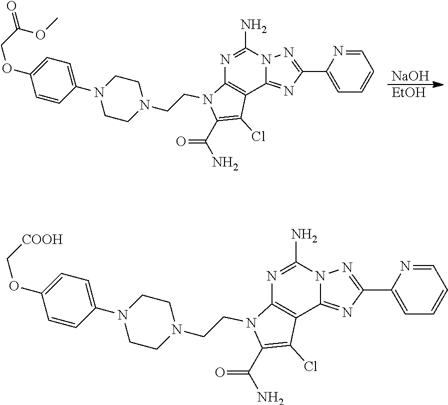

To a solution of methyl 2-[4-[4-[2-[5-amino-8-carbamoyl-9-chloro-2-(2-pyridyl)-[1,2,4]triazolo[BLAH]pyrrolo[BLAH]pyrimidin-7-yl]ethyl]piperazin-1-yl]phenoxy]acetate (250 mg, 413.19 umol, 1 eq) in EtOH (3 mL) was added NaOH (2 M, 413.19 uL, 2 eq). The mixture was stirred at 25° C. for 1 h. LCMS showed consumption of starting material and a new peak corresponding to the desired product mass. One additional vial was set up as described above and the reaction mixtures were combined and concentrated. The residue was purified by prep-HPLC (neutral condition) to afford 2-(4-(4-(2-(5-amino-8-carbamoyl-9-chloro-2-(pyridin-2-yl)-7H-pyrrolo[3,2-e][1,2,4]triazolo[1,5-c]pyrimidin-7-yl)ethyl)piperazin-1-yl)phenoxy)acetic acid (50 mg, 84.60 umol) as a white solid. LCMS for product (ESI+): m/z 591.3 [M+H]⁺, Rt: 2.005 min. ¹H NMR (400 MHz, DMSO-d₆) δ=8.75 (br d, J=4.1 Hz, 1H), 8.31 (d, J=7.9 Hz, 1H), 8.09-7.94 (m, 3H), 7.82 (br s, 1H), 7.70 (br s, 1H), 7.55 (dd, J=5.2, 6.9 Hz, 1H), 6.87-6.78 (m, 2H), 6.77-6.68 (m, 2H), 4.58 (br t, J=6.0 Hz, 2H), 4.41 (s, 2H), 2.96 (br s, 4H), 2.63 (br t, J=6.1 Hz, 2H), 2.56 (br s, 4H).

### Example 53: Synthesis of 5-amino-9-chloro-7-(2-(4-(2,4-difluorophenyl)piperazin-1-yl)ethyl)-2-(pyridin-2-yl)-7H-pyrrolo[3,2-e][1,2,4]triazolo[1,5-c]pyrimidine-8-carboxylic acid

### Step 1: Synthesis of methyl 5-amino-9-chloro-7-(2-(4-(2,4-difluorophenyl)piperazin-1-yl)ethyl)-2-(pyridin-2-yl)-7H-pyrrolo[3,2-e][1,2,4]triazolo[1,5-c]pyrimidine-8-carboxylate

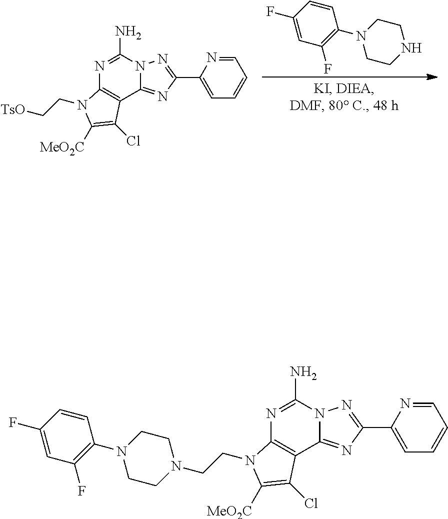 [SMILES: COC(=O)c1c(Cl)c2c(nc(N)n3nc(-c4ccccn4)nc23)n1CCOS(=O)(=O)c1ccc(C)cc1 ; Fc1ccc(N2CCNCC2)c(F)c1 ; COC(=O)c1c(Cl)c2c(nc(N)n3nc(-c4ccccn4)nc23)n1CCN1CCN(c2ccc(F)cc2F)CC1]

To a solution of methyl 5-amino-9-chloro-7-[2-(p-tolylsulfonyloxy) ethyl]-2-(2-pyridyl)-[1, 2, 4]triazolopyrrolo pyrimidine-8-carboxylate (150 mg, 277 umol, 1.00 eq) in DMF (4.5 mL) was added DIEA (215 mg, 1.66 mmol, 289 uL, 6.00 eq), KI (36.8 mg, 221 umol, 0.80 eq) and 1-(2,4-difluorophenyl) piperazine (110 mg, 554 umol, 2.00 eq). The mixture was stirred at 80° C. for 48 h. LC-MS showed disappearance of starting material and formation of a new peak corresponding to the desired product mass. The reaction was cooled to room temperature and filtered. The resulting solid was dried under reduce pressure to afford methyl 5-amino-9-chloro-7-(2-(4-(2,4-difluorophenyl)piperazin-1-yl)ethyl)-2-(pyridin-2-yl)-7H-pyrrolo[3,2-e][1,2,4]triazolo[1,5-c]pyrimidine-8-carboxylate (83 mg, 133 umol) as a white solid (used without further purification).

### Step 2: Synthesis of 5-amino-9-chloro-7-(2-(4-(2,4-difluorophenyl)piperazin-1-yl)ethyl)-2-(pyridin-2-yl)-7H-pyrrolo[3,2-e][1,2,4]triazolo[1,5-c]pyrimidine-8-carboxylic acid

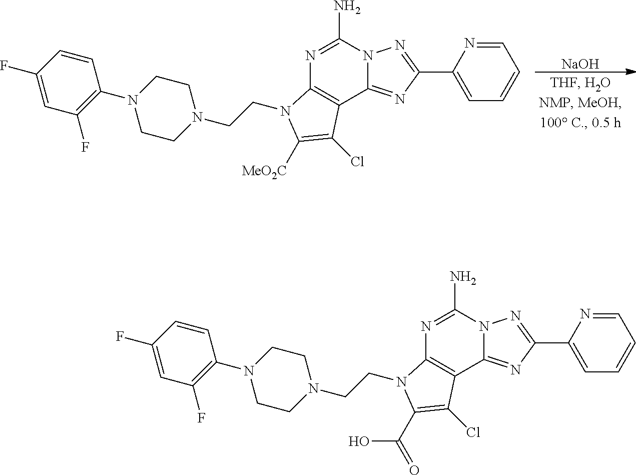

To a solution of methyl 5-amino-9-chloro-7-[2-[4-(2,4-difluorophenyl)piperazin-1-yl]ethyl]-2-(2-pyridyl)-[1,2,4]triazolo pyrrolo pyrimidine-8-carboxylate (83 mg, 146 umol, 1.00 eq) in H₂O (1 mL), THF (1 mL), MeOH (1 mL) and NMP (1 mL) was added NaOH (40.9 mg, 1.02 mmol, 7.00 eq). The mixture was stirred at 100° C. for 0.5 h. LC-MS indicated complete consumption of the starting material and formation of a new peak corresponding to the desired product mass. The reaction was filtered, and the solid was triturated in MTBE/MeOH (V/V=15/1) at 25° C. for 15 min. The solid was collected by filtration to afford 5-amino-9-chloro-7-(2-(4-(2,4-difluorophenyl)piperazin-1-yl)ethyl)-2-(pyridin-2-yl)-7H-pyrrolo[3,2-e][1,2,4]triazolo[1,5-c]pyrimidine-8-carboxylic acid. (68 mg, 117 umol) as a white solid (used without further purification). LCMS for product (ESI+): m/z 554.0 (M+H)⁺, Rt: 1.939 min. ¹H NMR (400 MHz, DMSO-d₆) δ=13.38 (br s, 1H), 9.54 (br d, J=10.1 Hz, 1H), 8.76 (br s, 1H), 8.29 (br s, 3H), 8.03 (br s, 1H), 7.57 (br s, 1H), 7.26 (br t, J=9.4 Hz, 1H), 7.14-7.03 (m, 2H), 4.85 (br s, 2H), 3.92 (br s, 2H), 3.70-3.49 (m, 4H), 3.07 (br s, 3H), 2.93 (br s, 1H).

### Example 54: Synthesis of 5-amino-9-chloro-7-(2-(4-(2,4-difluorophenyl)piperazin-1-yl)ethyl)-2-(pyridin-2-yl)-7H-pyrrolo[3,2-e][1,2,4]triazolo[1,5-c]pyrimidine-8-carboxamide

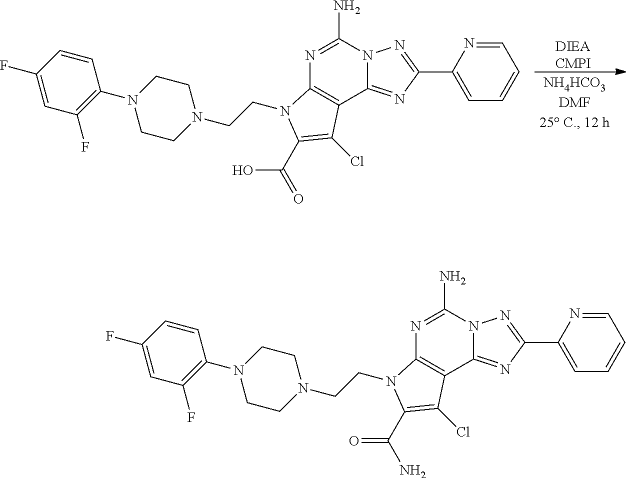 [SMILES: Nc1nc2c(c(Cl)c(C(=O)O)n2CCN2CCN(c3ccc(F)cc3F)CC2)c2nc(-c3ccccn3)nn12 ; "DIEACMPI" ; NC(=O)c1c(Cl)c2c(nc(N)n3nc(-c4ccccn4)nc23)n1CCN1CCN(c2ccc(F)cc2F)CC1]

To a solution of 5-amino-9-chloro-7-[2-[4-(2, 4-difluorophenyl) piperazin-1-yl]ethyl]-2-(2-pyridyl)-[1, 2, 4]triazolo pyrrolo pyrimidine-8-carboxylic acid (10 mg, 18.1 umol, 1.00 eq) in DMF (0.6 mL) was added NH₄HCO₃ (2.14 mg, 27.1 umol, 2.23 uL, 1.50 eq), DIEA (11.7 mg, 90.3 umol, 15.7 uL, 5.00 eq) and CMPI (9.22 mg, 36.1 umol, 2.00 eq). The mixture was stirred at 25° C. for 12 h. LC-MS showed complete consumption of the starting material and formation of a new peak corresponding to the desired product. The reaction was filtered, and the filtrate was purified by prep-HPLC (HCl condition) to afford 5-amino-9-chloro-7-(2-(4-(2,4-difluorophenyl)piperazin-1-yl)ethyl)-2-(pyridin-2-yl)-7H-pyrrolo[3,2-e][1,2,4]triazolo[1,5-c]pyrimidine-8-carboxamide (5 mg, 9.04 umol) as a light yellow solid. LCMS for product (ESI+): m/z 553.2 (M+H)⁺, Rt: 1.905 min. ¹H NMR (400 MHz, DEUTERIUM OXIDE) 6=8.83 (br s, 1H), 8.68-8.51 (m, 1H), 8.01 (br s, 1H), 7.16-7.06 (m, 1H), 7.04-6.97 (m, 1H), 6.95-6.87 (m, 1H), 4.85 (br s, 1H), 3.82 (br s, 1H), 3.77-3.70 (m, 2H), 3.60-3.33 (m, 3H), 3.27 (s, 2H), 3.14 (br s, 2H).

### Example 55: Synthesis of 5-amino-9-chloro-2-(pyridin-2-yl)-7-(2-(4-(pyridin-4-yl)piperazin-1-yl)ethyl)-7H-pyrrolo[3,2-e][1,2,4]triazolo[1,5-c]pyrimidine-8-carboxylic acid

### Step 1: Synthesis of methyl 5-amino-9-chloro-2-(pyridin-2-yl)-7-(2-(4-(pyridin-4-yl)piperazin-1-yl)ethyl)-7H-pyrrolo[3,2-e][1,2,4]triazolo[1,5-c]pyrimidine-8-carboxylate

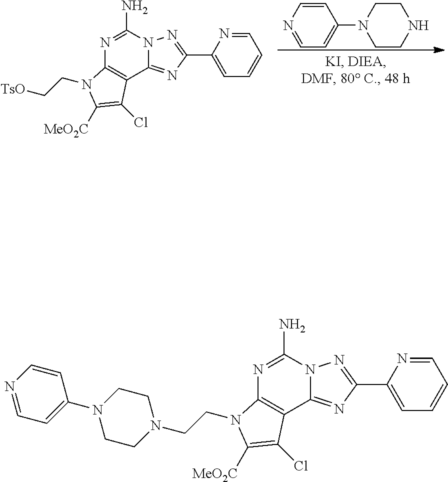 [SMILES: COC(=O)c1c(Cl)c2c(nc(N)n3nc(-c4ccccn4)nc23)n1CCOS(=O)(=O)c1ccc(C)cc1 ; c1cc(N2CCNCC2)ccn1 ; COC(=O)c1c(Cl)c2c(nc(N)n3nc(-c4ccccn4)nc23)n1CCN1CCN(c2ccncc2)CC1]

To a solution of methyl 5-amino-9-chloro-7-[2-(p-tolylsulfonyloxy)ethyl]-2-(2-pyridyl)-[1,2,4]triazolopyrrolo pyrimidine-8-carboxylate (150 mg, 277 umol, 1.00 eq) in DMF (4.5 mL) was added DIEA (215 mg, 1.66 mmol, 289 uL, 6.00 eq), KI (36.8 mg, 221 umol, 0.80 eq) and 1-(4-pyridyl)piperazine (90.4 mg, 554 umol, 2.00 eq). The mixture was stirred at 80° C. for 48 h. LC-MS showed complete consumption of starting material and formation of a new peak corresponding to the desired product. The reaction was filtered, and the solid was dried under pressure and purified by prep-HPLC (HCl condition) to afford methyl 5-amino-9-chloro-2-(pyridin-2-yl)-7-(2-(4-(pyridin-4-yl)piperazin-1-yl)ethyl)-7H-pyrrolo[3,2-e][1,2,4]triazolo[1,5-c]pyrimidine-8-carboxylate (50 mg, 80.7 umol) as a yellow solid. LCMS for product (ESI+): m/z 533.2 (M+H)⁺.

### Step 2: Synthesis of 5-amino-9-chloro-2-(pyridin-2-yl)-7-(2-(4-(pyridin-4-yl)piperazin-1-yl)ethyl)-7H-pyrrolo[3,2-e][1,2,4]triazolo[1,5-c]pyrimidine-8-carboxylic acid

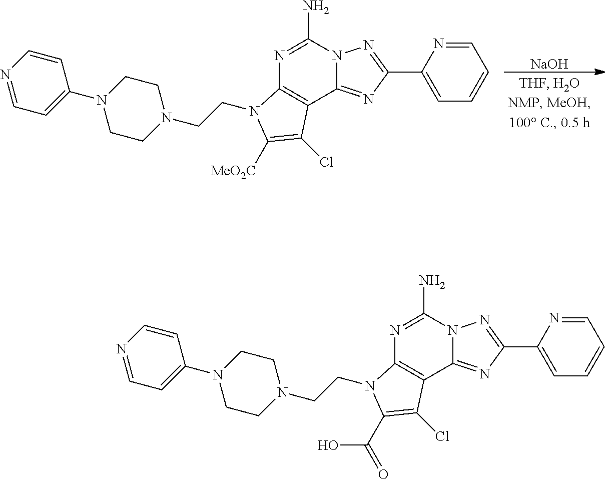

To a solution of methyl 5-amino-9-chloro-2-(2-pyridyl)-7-[2-[4-(4-pyridyl)piperazin-1-yl]ethyl]-[1,2,4]triazolo pyrrolo pyrimidine-8-carboxylate (50 mg, 93.8 umol, 1.00 eq) in H₂O (0.7 mL), THF (0.7 mL), MeOH (0.7 mL) and NMP (0.7 mL) was added NaOH (26.3 mg, 657 umol, 7.00 eq). The mixture was stirred at 100° C. for 0.5 h. LC-MS showed 5-amino-9-chloro-2-(pyridin-2-yl)-7-(2-(4-(pyridin-4-yl)piperazin-1-yl)ethyl)-7H-pyrrolo[3,2-e][1,2,4]triazolo[1,5-c]pyrimidine-8-carboxylic acid was completely consumed and one main peak with desired mass was detected. The reaction was filtered, and the filtrate was purified by prep-HPLC (HCl condition) to afford 5-amino-9-chloro-2-(pyridin-2-yl)-7-(2-(4-(pyridin-4-yl)piperazin-1-yl)ethyl)-7H-pyrrolo[3,2-e][1,2,4]triazolo[1,5-c]pyrimidine-8-carboxylic acid (24 mg, 40.5 umol, HCl) as a yellow solid. LCMS for product (ESI+): m/z 519.2 (M+H)⁺, Rt: 1.507 min. ¹H NMR (400 MHz, DEUTERIUM OXIDE) 6=8.41 (br s, 1H), 8.16 (br d, J=5.9 Hz, 3H), 7.93 (br s, 1H), 7.14 (br d, J=6.1 Hz, 3H), 4.69-4.47 (m, 2H), 4.69-4.46 (m, 2H), 3.98 (br s, 3H), 3.66 (br s, 7H).

### Example 56: Synthesis of 5-amino-9-chloro-2-(pyridin-2-yl)-7-(2-(4-(pyridin-4-yl)piperazin-1-yl)ethyl)-7H-pyrrolo[3,2-e][1,2,4]triazolo[1,5-c]pyrimidine-8-carboxamide

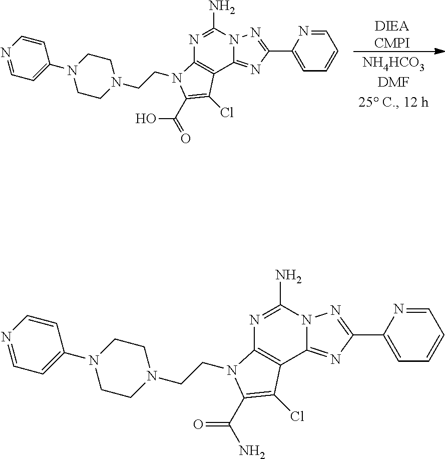 [SMILES: Nc1nc2c(c(Cl)c(C(=O)O)n2CCN2CCN(c3ccncc3)CC2)c2nc(-c3ccccn3)nn12 ; "DIEACMPI" ; NC(=O)c1c(Cl)c2c(nc(N)n3nc(-c4ccccn4)nc23)n1CCN1CCN(c2ccncc2)CC1]

To a solution of 5-amino-9-chloro-2-(2-pyridyl)-7-[2-[4-(4-pyridyl) piperazin-1-yl]ethyl]-[1, 2, 4]triazolopyrrolo pyrimidine-8-carboxylic acid (10 mg, 19.3 umol, 1.00 eq) in DMF (0.6 mL) was added NH₄HCO₃ (2.29 mg, 28.9 umol, 2.38 uL, 1.50 eq), DIEA (12.5 mg, 96.4 umol, 16.8 uL, 5.00 eq) and CMPI (9.85 mg, 38.5 umol, 2.00 eq). The mixture was stirred at 25° C. for 12 h. LC-MS showed starting material was completely consumed and one main peak with desired mass was detected. The reaction mixture was filtered, and he filtrate was purified by prep-HPLC (HCl condition) to afford 5-amino-9-chloro-2-(pyridin-2-yl)-7-(2-(4-(pyridin-4-yl)piperazin-1-yl)ethyl)-7H-pyrrolo[3,2-e][1,2,4]triazolo[1,5-c]pyrimidine-8-carboxamide (4 mg, 7.72 umol) as a yellow solid. LCMS for product (ESI+): m/z 518.2 (M+H)⁺, Rt: 1.499 min. ¹H NMR (400 MHz, DEUTERIUM OXIDE) δ=8.77 (br s, 1H), 8.45 (br d, J=6.1 Hz, 2H), 8.15 (d, J=7.6 Hz, 2H), 7.93 (br s, 1H), 7.13 (d, J=7.6 Hz, 2H), 4.83-4.79 (m, 2H), 3.97 (br s, 4H), 3.68 (br t, J=5.5 Hz, 3H), 3.61 (br s, 3H).

### Example 57: Synthesis of methyl 5-amino-9-chloro-7-(2-(4-(2-fluoro-5-(oxazol-2-yl)phenyl)piperazin-1-yl)ethyl)-2-(pyridin-2-yl)-7H-pyrrolo[3,2-e][1,2,4]triazolo[1,5-c]pyrimidine-8-carboxylate

Methyl-5-amino-9-chloro-7-[2-(p-tolylsulfonyloxy)ethyl]-2-(2-pyridyl)-[1,2,4]triazolo pyrrolo pyrimidine-8-carboxylate (160 mg, 295 umol, 1 eq) and DIEA (153 mg, 1.18 mmol, 205.68 uL, 4.0 eq) were dissolved in DMF (10 mL). 2-(4-fluoro-3-piperazin-1-yl-phenyl)oxazole (161 mg, 649 umol, 2.2 eq) was added to the reaction suspension. The suspension was stirred at 80° C. for 12 h. LCMS showed disappearance of starting material and formation of a major peak with desired MS. The solution was cooled to 0° C. The mixture was filtrated, and the white solid was washed with methyl tertiary-butyl ether (10 mL) to afford methyl 5-amino-9-chloro-7-(2-(4-(2-fluoro-5-(oxazol-2-yl)phenyl)piperazin-1-yl)ethyl)-2-(pyridin-2-yl)-7H-pyrrolo[3,2-e][1,2,4]triazolo[1,5-c]pyrimidine-8-carboxylate (100 mg, 162 umol) as white solid which was used in next step without purification. An analytical sample was purified by prep-HPLC to afford analytically pure methyl 5-amino-9-chloro-7-(2-(4-(2-fluoro-5-(oxazol-2-yl)phenyl)piperazin-1-yl)ethyl)-2-(pyridin-2-yl)-7H-pyrrolo[3,2-e][1,2,4]triazolo[1,5-c]pyrimidine-8-carboxylate (2 mg). LCMS for product (ESI+): m/z 617.2 (M+H)⁺, Rt: 1.912 min. ¹H NMR (400 MHz, METHANOL-d₄) δ=8.92 (d, J=5.4 Hz, 1H), 8.82 (d, J=7.8 Hz, 1H), 8.62 (dt, J=1.5, 7.8 Hz, 1H), 8.11-8.03 (m, 1H), 7.99 (s, 1H), 7.78-7.68 (m, 2H), 7.34-7.24 (m, 2H), 5.05 (t, J=5.6 Hz, 2H), 4.14 (br d, J=10.8 Hz, 2H), 4.01 (s, 3H), 3.85 (t, J=5.9 Hz, 2H), 3.80-3.73 (m, 2H), 3.56-3.47 (m, 2H), 3.25 (br d, J=12.2 Hz, 2H).

### Example 58: Synthesis of methyl 5-amino-7-(2-(4-(2-fluoro-5-(oxazol-2-yl)phenyl)piperazin-1-yl)ethyl)-2-(pyridin-2-yl)-7H-pyrrolo[3,2-e][1,2,4]triazolo[1,5-c]pyrimidine-8-carboxylate

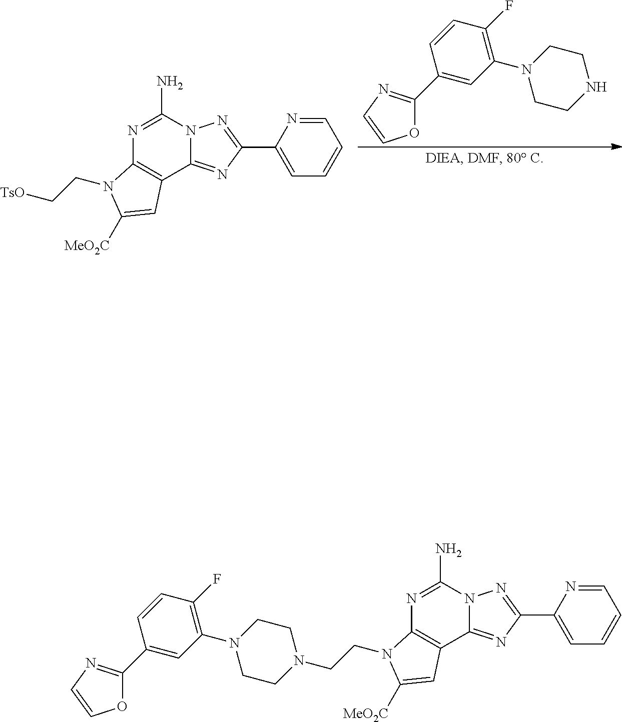

Methyl5-amino-7-[2-(p-tolylsulfonyloxy)ethyl]-2-(2-pyridyl)[1,2,4]triazolo pyrrolo pyrimidine-8-carboxylate (20 mg, 39.4 umol, 1 eq) and DIEA (20.4 mg, 0.158 mmol, 27.5 uL, 4.0 eq) were dissolved in DMF (0.5 mL). 2-(4-fluoro-3-piperazin-1-yl-phenyl)oxazole (11.7 mg, 47.3 umol, 1.2 eq) and KI (6.54 mg, 39.4 umol, 1.0 eq) were added to the reaction suspension. The suspension was stirred at 80° C. for 12 h. LCMS showed disappearance of starting material and formation of a major peak with desired MS. The solution was purified by prep-HPLC to afford methyl 5-amino-7-(2-(4-(2-fluoro-5-(oxazol-2-yl)phenyl)piperazin-1-yl)ethyl)-2-(pyridin-2-yl)-7H-pyrrolo[3,2-e][1,2,4]triazolo[1,5-c]pyrimidine-8-carboxylate (2 mg, 3.43 umol) as yellow solid. LCMS for product (ESI+): m/z 583.2 (M+H)⁺, Rt: 2.072 min. ¹H NMR (400 MHz, METHANOL-d₄) δ=8.84 (d, J=5.4 Hz, 1H), 8.58 (d, J=7.8 Hz, 1H), 8.32-8.30 (m, 1H), 7.98 (s, 1H), 7.81-7.80 (m, 1H), 7.73-7.67 (m, 2H), 7.63 (s, 1H), 7.30-7.25 (m, 2H), 5.05 (t, J=6.4 Hz, 2H), 4.13 (br s, 2H), 3.95 (s, 3H), 3.84 (t, J=6.4 Hz, 2H), 3.75 (br s, 2H), 3.51 (br s, 2H), 3.21 (br s, 2H).

### Example 59: Synthesis of 5-amino-7-(2-(4-(2-fluoro-5-(oxazol-2-yl)phenyl)piperazin-1-yl)ethyl)-2-(pyridin-2-yl)-7H-pyrrolo[3,2-e][1,2,4]triazolo[1,5-c]pyrimidine-8-carboxylic acid

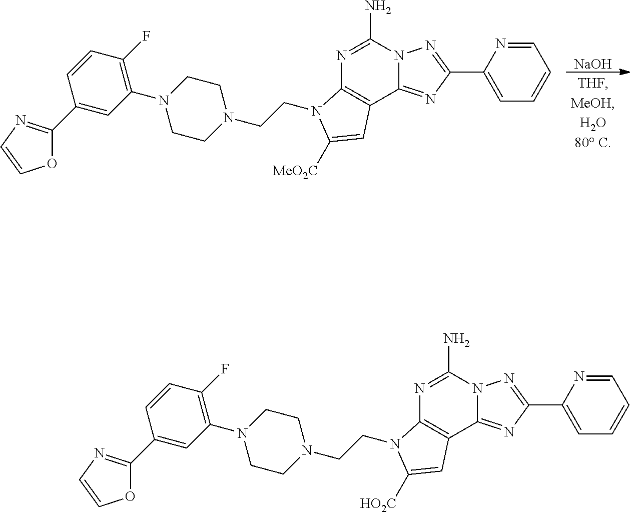

Methyl 5-amino-7-[2-[4-(2-fluoro-5-oxazol-2-yl-phenyl)piperazin-1-yl]ethyl]-2-(2-pyridyl)-[1,2,4]triazolopyrrolo pyrimidine-8-carboxylate (40 mg, 68.66 umol, 1 eq) was dissolved in THF (1 mL), MeOH (1 mL) and H₂O (0.5 mL). NaOH (19.2 mg, 0.481 mmol, 7.0 eq) was added to the suspension. The suspension was stirred at 100° C. for 3 h. LCMS showed disappearance of starting material and formation of a major peak with desired MS. The reaction solution was purified by prep-HPLC to afford 5-amino-7-(2-(4-(2-fluoro-5-(oxazol-2-yl)phenyl)piperazin-1-yl)ethyl)-2-(pyridin-2-yl)-7H-pyrrolo[3,2-e][1,2,4]triazolo[1,5-c]pyrimidine-8-carboxylic acid (10 mg, 16.5 umol, HCl) as yellow solid. LCMS for product (ESI+): m/z 569.3 (M+H)⁺, Rt: 2.122 min. ¹H NMR (400 MHz, METHANOL-d₄) δ=8.84 (d, J=4.4 Hz, 1H), 8.64 (d, J=8.0 Hz, 1H), 7.98 (s, 1H), 7.89-7.74 (m, 1H), 7.73-7.72 (m, 1H), 7.72-7.71 (m, 2H), 7.64 (s, 1H), 7.30-7.27 (s, 2H), 5.05 (t, J=5.6 Hz, 2H), 4.15-4.12 (m, 2H), 3.86-3.84 (m, 2H), 3.83-3.74 (m, 2H), 3.50-3.47 (m, 2H), 3.21-3.18 (m, 2H).

### Example 60: Synthesis of 5-amino-7-(2-(4-(2-fluoro-5-(oxazol-2-yl)phenyl)piperazin-1-yl)ethyl)-N-methyl-2-(pyridin-2-yl)-7H-pyrrolo[3,2-e][1,2,4]triazolo[1,5-c]pyrimidine-8-carboxamide

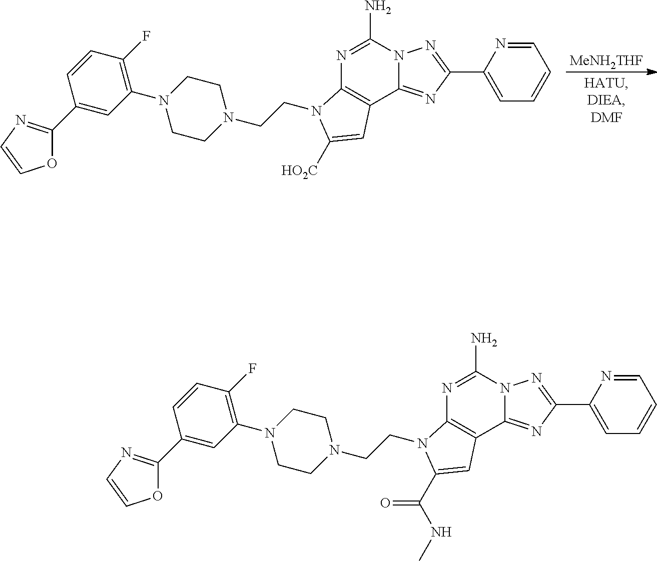 [SMILES: Nc1nc2c(cc(C(=O)O)n2CCN2CCN(c3cc(-c4ncco4)ccc3F)CC2)c2nc(-c3ccccn3)nn12 ; CNC(=O)c1cc2c(nc(N)n3nc(-c4ccccn4)nc23)n1CCN1CCN(c2cc(-c3ncco3)ccc2F)CC1 ; "MeNH2THF"]

5-amino-7-[2-[4-(2-fluoro-5-oxazol-2-yl-phenyl)piperazin-1-yl]ethyl]-2-(2-pyridyl)-[1,2,4]triazolo pyrrolo pyrimidine-8-carboxylic acid (80 mg, 0.123 mmol, 1 eq, HCl) and methylamine/THF (2 M, 0.123 mL, 2.0 eq) were dissolved in DMF (2 mL). DIEA (68.4 mg, 0.529 mmol, 92.1 uL, 4.0 eq) and HATU (101 mg, 264 umol, 2.0 eq) were added to the reaction solution. The solution was stirred at 20° C. for 12 h. LCMS showed disappearance of starting material and formation of a major peak with desired MS. The reaction mixture was purified by prep-HPLC to afford 5-amino-7-(2-(4-(2-fluoro-5-(oxazol-2-yl)phenyl)piperazin-1-yl)ethyl)-N-methyl-2-(pyridin-2-yl)-7H-pyrrolo[3,2-e][1,2,4]triazolo[1,5-c]pyrimidine-8-carboxamide (10 mg, 16.2 umol, HCl) as yellow solid. LCMS for product (ESI+): m/z 582.2 (M+H)⁺, Rt: 1.808 min. ¹H NMR (400 MHz, METHANOL-d₄) δ=8.88 (d, J=4.8 Hz, 1H), 8.75 (d, J=7.2 Hz, 1H), 8.58-8.55 (m, 1H), 8.02-7.99 (m, 2H), 7.75-7.72 (m, 2H), 7.46 (s, 1H), 7.31-7.26 (m, 2H), 4.97 (t, J=5.2 Hz, 2H), 3.97-3.87 (m,

### Example 61: Synthesis of 5-amino-9-chloro-7-(2-(4-(2-fluoro-5-(oxazol-2-yl)phenyl)piperazin-1-yl)ethyl)-2-(pyridin-2-yl)-7H-pyrrolo[3,2-e][1,2,4]triazolo[1,5-c]pyrimidine-8-carboxylic acid

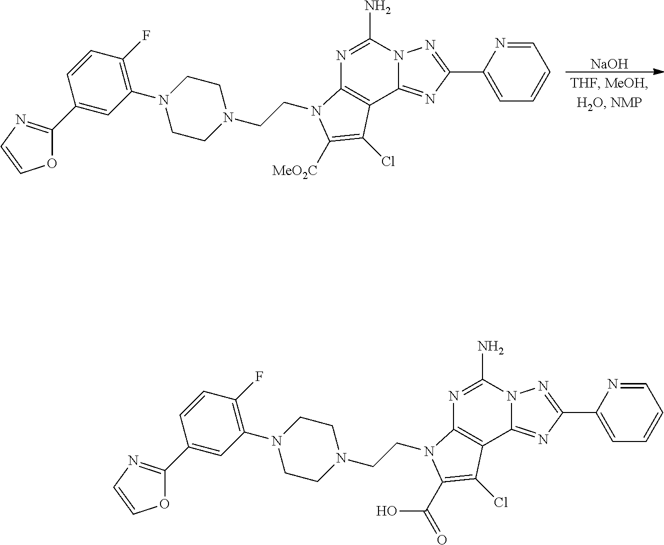

Methyl 5-amino-9-chloro-7-[2-[4-(2-fluoro-5-oxazol-2-yl-phenyl)piperazin-1-yl]ethyl]-2-(2-pyridyl)-[1,2,4]triazolo pyrrolo pyrimidine-8-carboxylate (120 mg, 194 umol, 1 eq) was dissolved in THF (5 mL), MeOH (5 mL) and H₂O (1 mL), NMP (5 mL). NaOH (77.8 mg, 1.94 mmol, 10 eq) was added to the suspension. The suspension was stirred at 100° C. for 3 h. LCMS showed consumption of starting material and a new major peak with the desired MS. The solution was cooled to 0° C. The pH of the reaction mixture was adjusted to 4-5 by addition of 6 N HCl. The reaction mixture was concentrated under reduced pressure and the residue was purified by prep-HPLC to afford 5-amino-9-chloro-7-(2-(4-(2-fluoro-5-(oxazol-2-yl)phenyl)piperazin-1-yl)ethyl)-2-(pyridin-2-yl)-7H-pyrrolo[3,2-e][1,2,4]triazolo[1,5-c]pyrimidine-8-carboxylic acid (80 mg, 132 umol, HCl) as yellow solid. LCMS for product (ESI+): m/z 603.0 (M+H)⁺, Rt: 1.913 min. ¹H NMR (400 MHz, DMSO-d₆) δ=9.77 (br s, 1H), 8.77 (br d, J=3.7 Hz, 1H), 8.42-8.29 (m, 3H), 8.22 (s, 1H), 8.05 (br t, J=6.9 Hz, 1H), 7.64 (br s, 1H), 7.61-7.56 (m, 2H), 7.44-7.33 (m, 2H), 4.88 (br s, 2H), 4.07 (br s, 2H), 3.68 (br s, 2H), 3.66-3.63 (m, 2H), 3.35 (br s, 2H), 3.16 (br s, 2H).

### Example 62: Synthesis of 5-amino-9-chloro-N-cyclopropyl-7-(2-(4-(2-fluoro-5-(oxazol-2-yl)phenyl)piperazin-1-yl)ethyl)-2-(pyridin-2-yl)-7H-pyrrolo[3,2-e][1,2,4]triazolo[1,5-c]pyrimidine-8-carboxamide

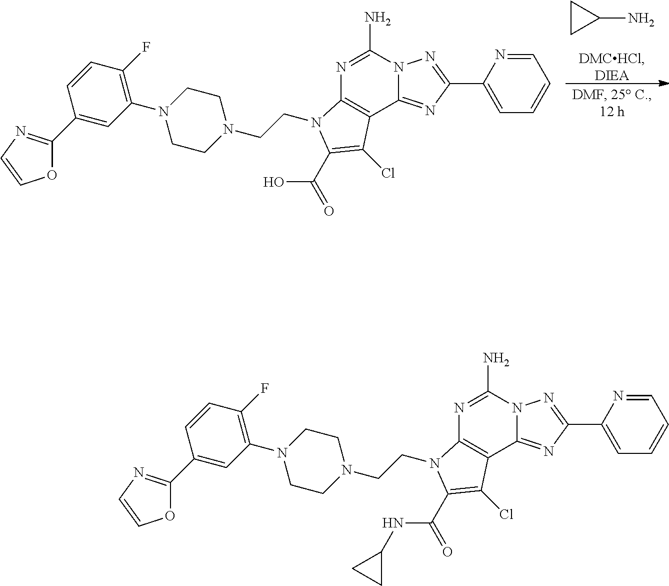

To a solution of 5-amino-9-chloro-7-[2-[4-(2-fluoro-5-oxazol-2-yl-phenyl)piperazin-1-yl]ethyl]-2-(2-pyridyl)-[1,2,4]triazolo pyrrolo pyrimidine-8-carboxylic acid (28 mg, 46.4 umol, 1 eq) and cyclopropylamine (7.95 mg, 139 umol, 9.65 uL, 3 eq) in N,N-dimethylformamide (1.5 mL) was added 2-chloro-1,3-dimethyl-4,5-dihydroimidazol-1-ium; chloride (11.7 mg, 69.6 umol, 1.5 eq) and N,N-diisopropylethylamine (15.0 mg, 116 umol, 20.2 uL, 2.5 eq). The mixture was stirred at 25° C. for 12 h. LCMS showed consumption of starting material and a new major peak with the desired MS. To the reaction mixture was added 1N HCl (0.2 mL) and the mixture was purified by prep-HPLC (HCl condition) to afford 5-amino-9-chloro-N-cyclopropyl-7-(2-(4-(2-fluoro-5-(oxazol-2-yl)phenyl)piperazin-1-yl)ethyl)-2-(pyridin-2-yl)-7H-pyrrolo[3,2-e][1,2,4]triazolo[1,5-c]pyrimidine-8-carboxamide (18 mg, 26.0 umol, HCl) as a yellow solid. LCMS for product (ESI+): m/z 642.1 (M+H)⁺, Rt: 2.052 min. ¹H NMR (400 MHz, DMSO-d₆) δ=9.82 (br s, 1H), 8.76 (br d, J=4.4 Hz, 1H), 8.46 (d, J=3.7 Hz, 1H), 8.32 (d, J=7.9 Hz, 1H), 8.23 (s, 3H), 8.04 (br t, J=7.8 Hz, 1H), 7.70-7.54 (m, 3H), 7.43-7.33 (m, 2H), 4.66 (br s, 2H), 3.98 (br d, J=11.2 Hz, 2H), 3.73 (br s, 2H), 3.69 (br d, J=14.3 Hz, 2H), 3.38 (br d, J=7.1 Hz, 2H), 3.17 (br t, J=12.3 Hz, 2H), 2.92 (br d, J=4.2 Hz, 1H), 0.78 (br d, J=5.3 Hz, 2H), 0.67 (br s, 2H).

### Example 63: Synthesis of 5-amino-9-chloro-7-(2-(4-(2-fluoro-5-(oxazol-2-yl)phenyl)piperazin-1-yl)ethyl)-2-(pyridin-2-yl)-7H-pyrrolo[3,2-e][1,2,4]triazolo[1,5-c]pyrimidine-8-carboxamide

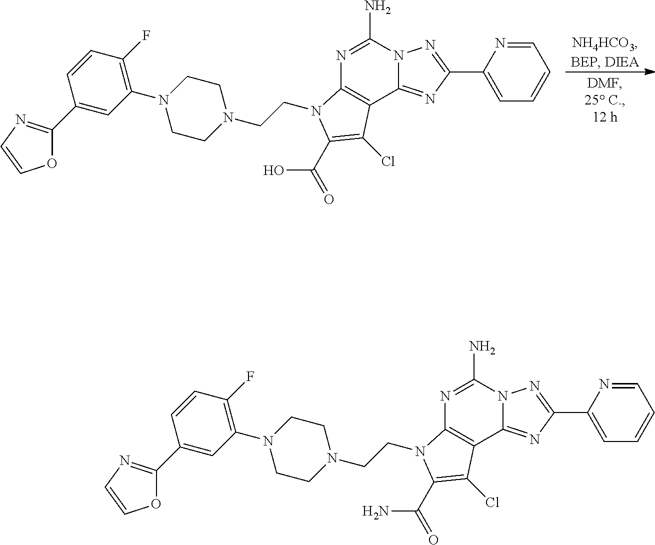 [SMILES: Nc1nc2c(c(Cl)c(C(=O)O)n2CCN2CCN(c3cc(-c4ncco4)ccc3F)CC2)c2nc(-c3ccccn3)nn12 ; "NH4HCO3,BEP, DIEA" ; NC(=O)c1c(Cl)c2c(nc(N)n3nc(-c4ccccn4)nc23)n1CCN1CCN(c2cc(-c3ncco3)ccc2F)CC1]

To a solution of 5-amino-9-chloro-7-[2-[4-(2-fluoro-5-oxazol-2-yl-phenyl)piperazin-1-yl]ethyl]-2-(2-pyridyl)-[1,2,4]triazolo pyrrolo pyrimidine-8-carboxylic acid (40 mg, 66.3 umol, 1 eq) and NH₄HCO₃ (10.4 mg, 132 umol, 10.9 uL, 2 eq) in N,N-dimethylformamide (3 mL) were added 2-bromo-1-ethyl-pyridin-1-ium; tetrafluoroborate (21.8 mg, 79.6 umol, 1.2 eq) and N,N-diisopropylethylamine (21.4 mg, 165 umol, 28.9 uL, 2.5 eq). The mixture was stirred at 25° C. for 12 h. LCMS showed consumption of starting material and a new major peak with desired MS. To the reaction mixture was added 1N HCl (0.2 mL) and the mixture was purified by prep-HPLC (HCl condition) to afford 5-amino-9-chloro-7-(2-(4-(2-fluoro-5-(oxazol-2-yl)phenyl)piperazin-1-yl)ethyl)-2-(pyridin-2-yl)-7H-pyrrolo[3,2-e][1,2,4]triazolo[1,5-c]pyrimidine-8-carboxamide (6 mg, 9.40 umol, HCl) as a yellow solid. LCMS for product (ESI+): m/z 602.1 (M+H)⁺, Rt: 1.895 min. ¹H NMR (400 MHz, METHANOL-d₄) δ=8.96 (t, J=7.8 Hz, 2H), 8.82 (dt, J=1.5, 7.9 Hz, 1H), 8.23 (dt, J=1.2, 6.8 Hz, 1H), 8.00 (d, J=0.9 Hz, 1H), 7.77-7.70 (m, 2H), 7.33-7.24 (m, 2H), 4.93 (br s, 2H), 3.96 (br d, J=11.9 Hz, 2H), 3.92-3.82 (m, 2H), 3.74 (br d, J=13.5 Hz, 2H), 3.48 (br t, J=11.5 Hz, 2H), 3.30-3.22 (m, 2H).

### Example 64: Synthesis of 5-amino-9-chloro-7-(2-(4-(2-fluoro-5-(oxazol-2-yl)phenyl)piperazin-1-yl)ethyl)-N-methyl-2-(pyridin-2-yl)-7H-pyrrolo[3,2-e][1,2,4]triazolo[1,5-c]pyrimidine-8-carboxamide

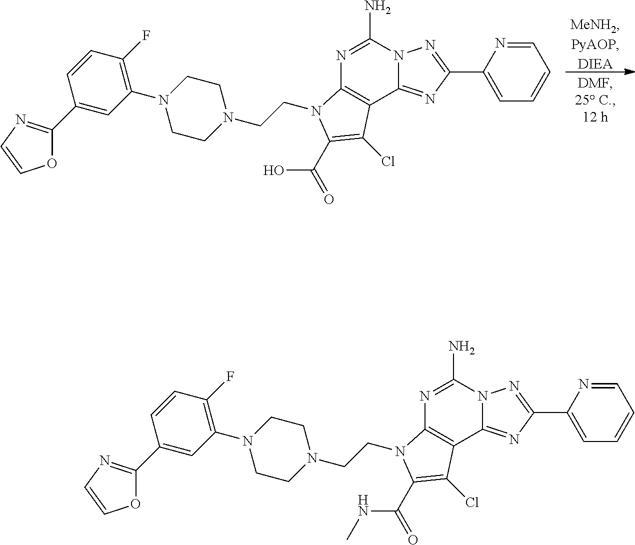 [SMILES: Nc1nc2c(c(Cl)c(C(=O)O)n2CCN2CCN(c3cc(-c4ncco4)ccc3F)CC2)c2nc(-c3ccccn3)nn12 ; "MeNH2,PyAOP,DIEA" ; CNC(=O)c1c(Cl)c2c(nc(N)n3nc(-c4ccccn4)nc23)n1CCN1CCN(c2cc(-c3ncco3)ccc2F)CC1]

To a solution of 5-amino-9-chloro-7-[2-[4-(2-fluoro-5-oxazol-2-yl-phenyl)piperazin-1-yl]ethyl]-2-(2-pyridyl)-[1,2,4]triazolo pyrrolo pyrimidine-8-carboxylic acid (28 mg, 46.4 umol, 1 eq) and methylamine/tetrahydrofuran (2 M, 69.6 uL, 3 eq) in N,N-dimethylformamide (1.5 mL) was added N,N-diisopropylethylamine (15.0 mg, 116 umol, 20.2 uL, 2.5 eq) and tripyrrolidin-1-yl(triazolo[4,5-b]pyridin-3-yloxy)phosphonium; hexafluorophosphate (29.0 mg, 55.7 umol, 1.2 eq). The mixture was stirred at 25° C. for 12 h. LCMS showed consumption of starting material and a new major peak with the desired MS. To the reaction mixture was added 1N HCl (0.2 mL) and the mixture was purified by prep-HPLC (HCl condition) to afford 5-amino-9-chloro-7-(2-(4-(2-fluoro-5-(oxazol-2-yl)phenyl)piperazin-1-yl)ethyl)-N-methyl-2-(pyridin-2-yl)-7H-pyrrolo[3,2-e][1,2,4]triazolo[1,5-c]pyrimidine-8-carboxamide (12 mg, 17.8 umol, HCl) as a yellow solid. LCMS for product (ESI+): m/z 616.1 (M+H)⁺, Rt: 1.972 min. ¹H NMR (400 MHz, DMSO-d₆) δ=9.98 (br s, 1H), 8.77 (d, J=4.0 Hz, 1H), 8.33 (d, J=7.7 Hz, 1H), 8.30-8.20 (m, 4H), 8.05 (dt, J=1.8, 7.7 Hz, 1H), 7.66 (ddd, J=2.0, 4.4, 8.4 Hz, 1H), 7.63-7.56 (m, 2H), 7.42-7.34 (m, 2H), 4.71 (br t, J=5.4 Hz, 2H), 3.98 (br d, J=11.2 Hz, 2H), 3.80-3.72 (m, 4H), 3.37 (br d, J=8.8 Hz, 2H), 3.19 (br t, J=11.2 Hz, 2H), 2.88 (d, J=4.6 Hz, 3H).

### Example 65: Synthesis of 5-amino-9-chloro-7-(2-(4-(4-cyano-2-fluorophenyl)piperazin-1-yl)ethyl)-2-(pyridin-2-yl)-7H-pyrrolo[3,2-e][1,2,4]triazolo[1,5-c]pyrimidine-8-carboxylic acid

### Step 1: Synthesis of methyl 5-amino-9-chloro-7-(2-(4-(4-cyano-2-fluorophenyl)piperazin-1-yl)ethyl)-2-(pyridin-2-yl)-7H-pyrrolo[3,2-e][1,2,4]triazolo[1,5-c]pyrimidine-8-carboxylate

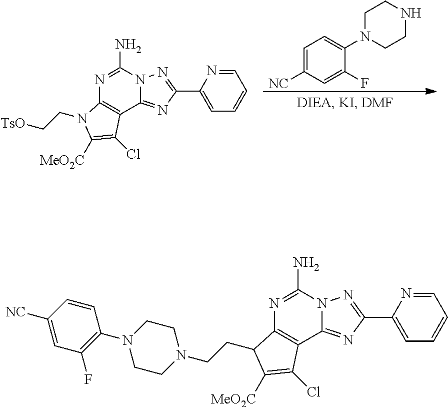 [SMILES: COC(=O)c1c(Cl)c2c(nc(N)n3nc(-c4ccccn4)nc23)n1CCOS(=O)(=O)c1ccc(C)cc1 ; N#Cc1ccc(N2CCNCC2)c(F)c1 ; COC(=O)C1=C(Cl)c2c(nc(N)n3nc(-c4ccccn4)nc23)C1CCN1CCN(c2ccc(C#N)cc2F)CC1]

To a solution of methyl 5-amino-9-chloro-7-[2-(p-tolylsulfonyloxy)ethyl]-2-(2-pyridyl)-[1,2,4]triazolo pyrrolo pyrimidine-8-carboxylate (150 mg, 276 umol, 1.00 eq) and 3-fluoro-4-piperazin-1-yl-benzonitrile (113 mg, 553 umol, 2.00 eq) in dimethyl formamide (4.50 mL) was added di-isopropylethylamine (143 mg, 1.10 mmol, 192 uL, 4.00 eq) and KI (36.8 mg, 221 umol, 0.80 eq). The mixture was stirred at 80° C. for 12 h. 3-fluoro-4-piperazin-1-yl-benzonitrile (56.8 mg, 276 umol, 1.00 eq) was added to the reaction mixture and stirring was continued at 80° C. for 12 h. LC-MS showed starting material was completely consumed and one main peak with desired m/z was detected. The reaction mixture was filtered and dried under vacuum to afford methyl 5-amino-9-chloro-7-(2-(4-(4-cyano-2-fluorophenyl)piperazin-1-yl)ethyl)-2-(pyridin-2-yl)-7H-pyrrolo[3,2-e][1,2,4]triazolo[1,5-c]pyrimidine-8-carboxylate (70.0 mg, 121 umol) as a yellow solid. LCMS (ESI): m/z 575.5, Rt: 0.893 min.

Step 2: Synthesis of 5-amino-9-chloro-7-(2-(4-(4-cyano-2-fluorophenyl)piperazin-1-yl)ethyl)-2-(pyridin-2-yl)-7H-pyrrolo[3,2-e][1,2,4]triazolo[1,5-c]pyrimidine-8-carboxylic acid

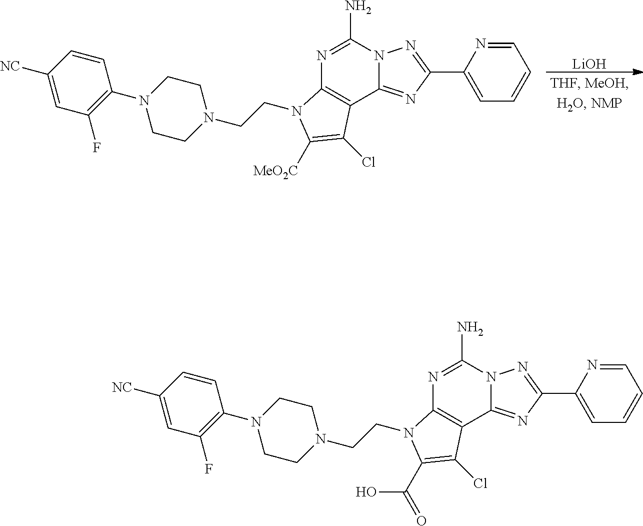

To a solution of methyl methyl 5-amino-9-chloro-7-[2-[4-(4-cyano-2-fluoro-phenyl)piperazin-1-yl]ethyl]-2-(2-pyridyl)-[1,2,4]triazolo pyrrolo pyrimidine-8-carboxylate (50.0 mg, 86.9 umol, 1.00 eq) in N-methyl pyrrolidone (1.50 mL), water (0.50 mL), tetrahydrofuran (1.00 mL) and methanol (1.00 mL) was added LiOH—H₂O (25.5 mg, 608 umol, 7.00 eq). The mixture was stirred at 70° C. for 2 h. LC-MS showed starting material was completely consumed and one main peak with desired m/z was detected. The reaction mixture was filtered, and the filtrate was purified by prep-HPLC (HCl condition) to afford 5-amino-9-chloro-7-(2-(4-(4-cyano-2-fluorophenyl)piperazin-1-yl)ethyl)-2-(pyridin-2-yl)-7H-pyrrolo[3,2-e][1,2,4]triazolo[1,5-c]pyrimidine-8-carboxylic acid (27.0 mg, 48.1 umol) as a yellow solid. LCMS (ESI): m/z 561.2, Rt: 1.861 min). ¹H NMR (400 MHz, DMSO-d₆) δ=9.56-9.50 (m, 1H), 8.77 (br d, J=4.6 Hz, 1H), 8.32 (br d, J=7.1 Hz, 2H), 8.06-8.01 (m, 1H), 7.83-7.80 (m, 1H), 7.79-7.77 (m, 1H), 7.65-7.61 (m, 1H), 7.60-7.55 (m, 1H), 7.21-7.16 (m, 1H), 4.89-4.83 (m, 2H), 4.10-4.01 (m, 2H), 3.82-3.73 (m, 2H), 3.70-3.64 (m, 2H), 3.36-3.26 (m, 2H), 3.21-3.11 (m, 2H).

### Example 66: Synthesis of 5-amino-9-chloro-7-(2-(4-(4-cyano-2-fluorophenyl)piperazin-1-yl)ethyl)-2-(pyridin-2-yl)-7H-pyrrolo[3,2-e][1,2,4]triazolo[1,5-c]pyrimidine-8-carboxamide

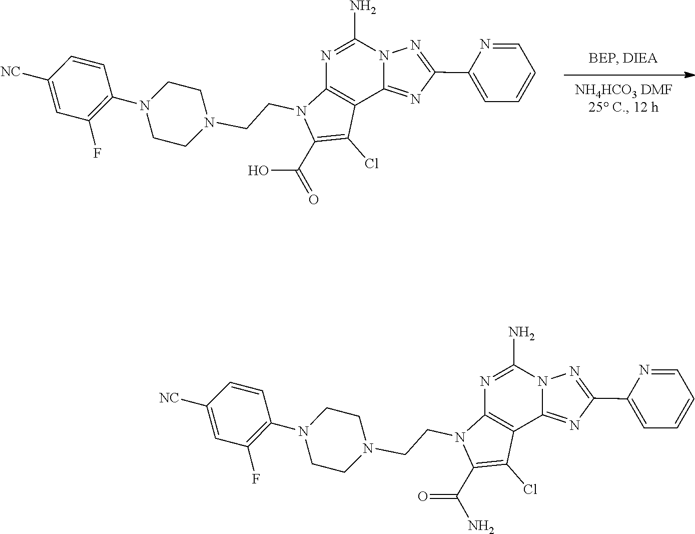 [SMILES: N#Cc1ccc(N2CCN(CCn3c(C(=O)O)c(Cl)c4c3nc(N)n3nc(-c5ccccn5)nc43)CC2)c(F)c1 ; N#Cc1ccc(N2CCN(CCn3c(C(N)=O)c(Cl)c4c3nc(N)n3nc(-c5ccccn5)nc43)CC2)c(F)c1]

To a solution of 5-amino-9-chloro-7-[2-[4-(4-cyano-2-fluoro-phenyl)piperazin-1-yl]ethyl]-2-(2-pyridyl)-[1,2,4]triazolo pyrrolo pyrimidine-8-carboxylic acid (10.0 mg, 16.7 umol, 1.00 eq, HCl) in dimethyl formamide (1.50 mL) was added NH₄HCO₃ (2.70 mg, 33.5 umol, 2.80 uL, 2.00 eq), 2-bromo-1-ethyl-pyridin-1-ium; tetrafluoroborate (5.50 mg, 20.1 umol, 1.20 eq) and diisopropylethylamine (5.40 mg, 41.8 umol, 7.30 uL, 2.50 eq). The mixture was stirred at 25° C. for 12 h. LC-MS showed starting material was completely consumed and one main peak with desired m/z was detected. The reaction mixture was filtered, and the filtrate was purified by prep-HPLC (HCl condition) to afford 5-amino-9-chloro-7-(2-(4-(4-cyano-2-fluorophenyl)piperazin-1-yl)ethyl)-2-(pyridin-2-yl)-7H-pyrrolo[3,2-e][1,2,4]triazolo[1,5-c]pyrimidine-8-carboxamide (3.90 mg, 6.50 umol, HCl) was as a yellow solid. LCMS (ESI): m/z 560.0, Rt: 1.854 min). ¹H NMR (400 MHz, METHANOL-d₄) δ=8.85 (br d, J=4.9 Hz, 1H), 8.67 (br d, J=8.3 Hz, 1H), 8.38 (s, 1H), 7.86 (br s, 1H), 7.60-7.50 (m, 2H), 7.19 (s, 1H), 4.90-4.90 (m, 2H), 3.83 (br s, 6H), 3.43 (br d, J=1.5 Hz, 2H), 3.28-3.22 (m, 2H).

### Example 67: Synthesis of 5-amino-9-chloro-7-(2-(4-(5-fluoro-2-methylpyridin-4-yl)piperazin-1-Yl)ethyl)-2-(pyridin-2-Yl)-7H-pyrrolo[3,2-e][1,2,4]triazolo[1,5-c]pyrimidine-8-carboxylic acid

### Step 1: Synthesis of methyl 5-amino-9-chloro-7-(2-(4-(5-fluoro-2-methylpyridin-4-yl)piperazin-1-yl)ethyl)-2-(pyridin-2-yl)-7H-pyrrolo[3,2-e][1,2,4]triazolo[1,5-c]pyrimidine-8-carboxylate

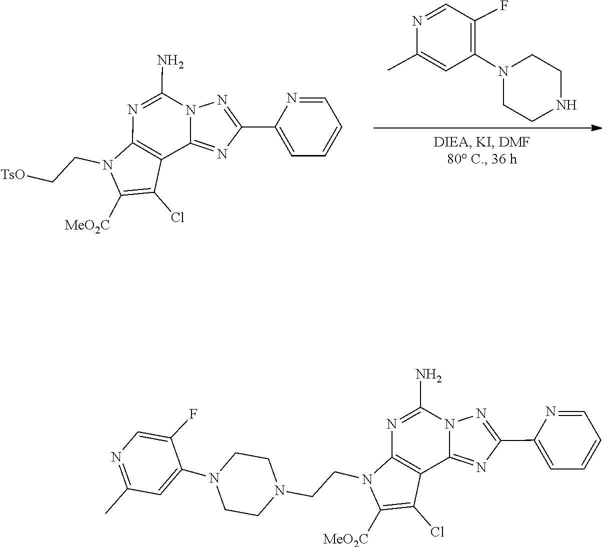 [SMILES: COC(=O)c1c(Cl)c2c(nc(N)n3nc(-c4ccccn4)nc23)n1CCOS(=O)(=O)c1ccc(C)cc1 ; Cc1cc(N2CCNCC2)c(F)cn1 ; COC(=O)c1c(Cl)c2c(nc(N)n3nc(-c4ccccn4)nc23)n1CCN1CCN(c2cc(C)ncc2F)CC1]

To a solution of methyl 5-amino-9-chloro-7-[2-(p-tolylsulfonyloxy) ethyl]-2-(2-pyridyl)-[1,2,4]triazolo[BLAH]pyrrolo[BLAH]pyrimidine-8-carboxylate (150 mg, 277 umol, 1.00 eq) in DMF (3 mL) was added DIEA (215 mg, 1.66 mmol, 289 uL, 6.00 eq), KI (36.8 mg, 221 umol, 0.80 eq) and 1-(5-fluoro-2-methyl-4-pyridyl)piperazine (108 mg, 554 umol, 2.00 eq). The mixture was stirred at 80° C. for 36 h. LC-MS showed starting material was completely consumed and one main peak with desired mass was detected. The reaction mixture was purified by prep-HPLC (TFA condition) to afford methyl 5-amino-9-chloro-7-[2-[4-(5-fluoro-2-methyl-4-pyridyl)piperazin-1-yl]ethyl]-2-(2-pyridyl)-[1,2,4]triazolo[BLAH]pyrrolo[BLAH]pyrimidine-8-carboxylate (60 mg, 106 umol) and methyl 5-amino-9-chloro-7-[2-[4-(5-fluoro-2-methyl-4-pyridyl)piperazin-1-yl]ethyl]-2-(2-pyridyl)-[1,2,4]triazolo[BLAH]pyrrolo[BLAH]pyrimidine-8-carboxylate (10 mg, 14.7 umol, TFA).

### Step 2: Synthesis of 5-amino-9-chloro-7-(2-(4-(5-fluoro-2-methylpyridin-4-yl)piperazin-1-yl)ethyl)-2-(pyridin-2-yl)-7H-pyrrolo[3,2-e][1,2,4]triazolo[1,5-c]pyrimidine-8-carboxylic acid

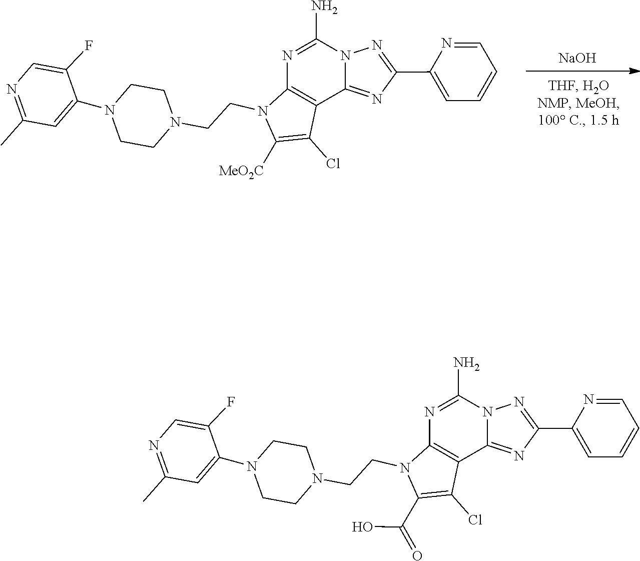 [SMILES: COC(=O)c1c(Cl)c2c(nc(N)n3nc(-c4ccccn4)nc23)n1CCN1CCN(c2cc(C)ncc2F)CC1 ; "NaOH" ; Cc1cc(N2CCN(CCn3c(C(=O)O)c(Cl)c4c3nc(N)n3nc(-c5ccccn5)nc43)CC2)c(F)cn1]

To a solution of methyl 5-amino-9-chloro-7-[2-[4-(5-fluoro-2-methyl-4-pyridyl)piperazin-1-yl]ethyl]-2-(2-pyridyl)-[1,2,4]triazolo[BLAH]pyrrolo[BLAH]pyrimidine-8-carboxylate (10 mg, 17.7 umol, 1.00 eq) in H₂O (0.1 mL), THF (0.1 mL), MeOH (0.1 mL) and NMP (0.1 mL) was added NaOH (4.96 mg, 124 umol, 7.00 eq). The mixture was stirred at 100° C. for 1.5 h. LC-MS showed starting material was consumed and one main peak with desired mass was detected. The mixture was purified by prep-HPLC (neutral condition) to afford 5-amino-9-chloro-7-(2-(4-(5-fluoro-2-methylpyridin-4-yl)piperazin-1-yl)ethyl)-2-(pyridin-2-yl)-7H-pyrrolo[3,2-e][1,2,4]triazolo[1,5-c]pyrimidine-8-carboxylic acid (4 mg, 7.22 umol) as a white solid. LCMS for product (ESI+): m/z 551.2 (M+H)⁺, Rt: 1.568 min. ¹H NMR (400 MHz, DMSO-d₆) δ=8.75 (br d, J=4.4 Hz, 1H), 8.30 (d, J=8 Hz, 1H), 8.09-8.06 (m, 3H), 8.02 (dt, J=1.6, 7.8 Hz, 1H), 7.55 (dd, J=4.9, 6.7 Hz, 1H), 6.75 (d, J=7.6 Hz, 1H), 4.67 (br t, J=6 Hz, 2H), 3.15 (br s, 2H), 2.69-2.66 (m, 4H), 2.61 (br s, 4H), 2.32 (s, 3H).

### Example 68: Synthesis of 5-amino-9-chloro-7-(2-(4-(5-fluoro-2-methylpyridin-4-yl)piperazin-1-yl)ethyl)-2-(pyridin-2-yl)-7H-pyrrolo[3,2-e][1,2,4]triazolo[1,5-c]pyrimidine-8-carboxamide

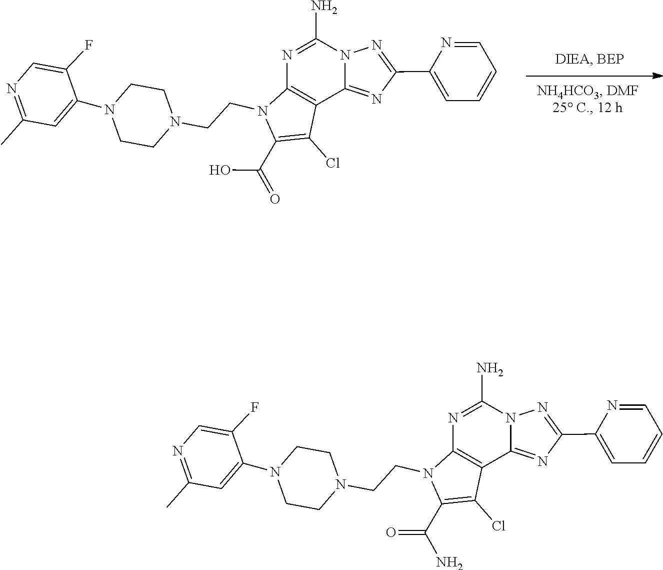 [SMILES: Cc1cc(N2CCN(CCn3c(C(=O)O)c(Cl)c4c3nc(N)n3nc(-c5ccccn5)nc43)CC2)c(F)cn1 ; Cc1cc(N2CCN(CCn3c(C(N)=O)c(Cl)c4c3nc(N)n3nc(-c5ccccn5)nc43)CC2)c(F)cn1]

To a solution of 5-amino-9-chloro-7-[2-[4-(5-fluoro-2-methyl-4-pyridyl)piperazin-1-yl]ethyl]-2-(2-pyridyl)-[1,2,4]triazolo[BLAH]pyrrolo[BLAH]pyrimidine-8-carboxylic acid (12 mg, 21.8 umol, 1.00 eq) in DMF (1 mL) was added NH₄HCO₃ (6.89 mg, 87.1 umol, 7.17 uL, 4.00 eq), 2-bromo-1-ethyl-pyridin-1-ium; tetrafluoroborate (11.9 mg, 43.6 umol, 2.00 eq) and DIEA (14.1 mg, 109 umol, 19.0 uL, 5.00 eq). The mixture was stirred at 25° C. for 12 h. LC-MS showed starting material was consumed and one main peak with desired mass was detected. The mixture was purified by prep-HPLC (HCl condition) to afford 5-amino-9-chloro-7-(2-(4-(5-fluoro-2-methylpyridin-4-yl)piperazin-1-yl)ethyl)-2-(pyridin-2-yl)-7H-pyrrolo[3,2-e][1,2,4]triazolo[1,5-c]pyrimidine-8-carboxamide (1.3 mg, 2.34 umol) as a white solid. LCMS for product (ESI+): m/z 550.2 (M+H)⁺, Rt: 1.565 min. ¹H NMR (400 MHz, DMSO-d₆) δ=8.75 (d, J=4.4 Hz, 1H), 8.30 (d, J=7.6 Hz, 1H), 8.06 (d, J=5.6 Hz, 1H), 8.01 (dt, J=2, 8 Hz, 3H), 7.79 (br s, 1H), 7.67 (br s, 1H), 7.55 (dd, J=5.6, 7.6 Hz, 1H), 6.76 (d, J=7.6 Hz, 1H), 4.57 (br t, J=6.0 Hz, 2H), 3.16 (br s, 2H), 2.66-2.62 (m, 3H), 2.56 (br s, 5H), 2.32 (s, 3H).

### Example 69: Synthesis of 5-amino-9-chloro-2-(pyridin-2-yl)-7-(2-(4-(pyrimidin-2-yl)piperazin-1-yl)ethyl)-7H-pyrrolo[3,2-e][1,2,4]triazolo[1,5-c]pyrimidine-8-carboxylic acid

### Step 1: Synthesis of methyl 5-amino-9-chloro-2-(pyridin-2-yl)-7-(2-(4-(pyrimidin-2-yl)piperazin-1-yl)ethyl)-7H-pyrrolo[3,2-e][1,2,4]triazolo[1,5-c]pyrimidine-8-carboxylate

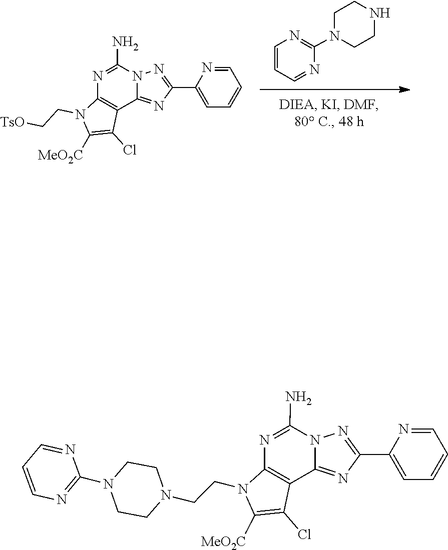 [SMILES: COC(=O)c1c(Cl)c2c(nc(N)n3nc(-c4ccccn4)nc23)n1CCOS(=O)(=O)c1ccc(C)cc1 ; c1cnc(N2CCNCC2)nc1 ; COC(=O)c1c(Cl)c2c(nc(N)n3nc(-c4ccccn4)nc23)n1CCN1CCN(c2ncccn2)CC1]

To a solution of methyl 5-amino-9-chloro-7-[2-(p-tolylsulfonyloxy)ethyl]-2-(2-pyridyl)-[1,2,4]triazolopyrrolo pyrimidine-8-carboxylate (150 mg, 276 umol, 1.00 eq) and 2-piperazin-1-ylpyrimidine (90.9 mg, 553 umol, 78.4 uL, 2.00 eq) in dimethyl formamide (4.50 mL) was added diisopropylethylamine (143 mg, 1.10 mmol, 192 uL, 4.00 eq) and KI (36.8 mg, 221 umol, 0.80 eq). The mixture was stirred at 80° C. for 48 h. LC-MS showed starting material was completely consumed and one main peak with the desired m/z was detected. The reaction mixture was filtered and the solid was dried to afford methyl 5-amino-9-chloro-2-(pyridin-2-yl)-7-(2-(4-(pyrimidin-2-yl)piperazin-1-yl)ethyl)-7H-pyrrolo[3,2-e][1,2,4]triazolo[1,5-c]pyrimidine-8-carboxylate (56 mg, 104 umol) as a white solid. LCMS (ESI) m/z 534.4, Rt: 0.813 min.

### Step 2: Synthesis of 5-amino-9-chloro-2-(pyridin-2-yl)-7-(2-(4-(pyrimidin-2-yl)piperazin-1-yl)ethyl)-7H-pyrrolo[3,2-e][1,2,4]triazolo[1,5-c]pyrimidine-8-carboxylic acid

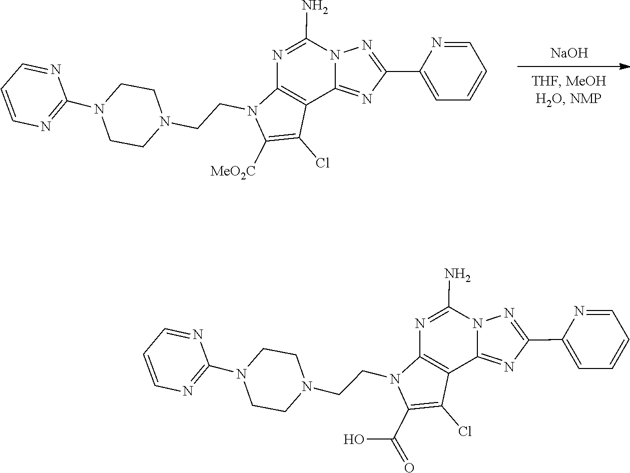 [SMILES: COC(=O)c1c(Cl)c2c(nc(N)n3nc(-c4ccccn4)nc23)n1CCN1CCN(c2ncccn2)CC1 ; "NaOH" ; Nc1nc2c(c(Cl)c(C(=O)O)n2CCN2CCN(c3ncccn3)CC2)c2nc(-c3ccccn3)nn12]

To a solution of methyl 5-amino-9-chloro-2-(2-pyridyl)-7-[2-(4-pyrimidin-2-ylpiperazin-1-yl)ethyl]-[1,2,4]triazolo pyrrolo pyrimidine-8-carboxylate (50.0 mg, 93.6 umol, 1.00 eq) in tetrahydrofuran (1.00 mL), N-methyl pyrrolidone (1.00 mL), methanol (1.00 mL) and water (1.00 mL) was added NaOH (26.2 mg, 655 umol, 7.00 eq). The mixture was stirred at 100° C. for 0.5 h. LC-MS showed starting material was completely consumed and one main peak with the desired m/z was detected. The reaction mixture was filtered, and the filtrate was purified by prep-HPLC (HCl condition) to afford 5-amino-9-chloro-2-(pyridin-2-yl)-7-(2-(4-(pyrimidin-2-yl)piperazin-1-yl)ethyl)-7H-pyrrolo[3,2-e][1,2,4]triazolo[1,5-c]pyrimidine-8-carboxylic acid (44.0 mg, 84.6 umol) as a yellow solid. LCMS (ESI) m/z 520.0, Rt: 0.709 min). ¹H NMR (400 MHz, DMSO-d₆) δ=9.59-9.54 (m, 1H), 8.78 (d, J=3.9 Hz, 1H), 8.46 (d, J=4.9 Hz, 2H), 8.33 (br d, J=7.8 Hz, 3H), 8.05 (dt, J=2.0, 7.8 Hz, 1H), 7.61-7.57 (m, 1H), 6.78 (t, J=4.9 Hz, 1H), 4.87 (br s, 2H), 4.75 (br d, J=12.2 Hz, 2H), 4.05 (br d, J=9.3 Hz, 2H), 3.63 (br s, 2H), 3.26 (br s, 2H), 3.16 (br dd, J=3.2, 5.6 Hz, 2H).

### Example 70: Synthesis of 5-amino-9-chloro-2-(pyridin-2-Yl)-7-(2-(4-(pyrimidin-2-yl)piperazin-1-Yl)ethyl)-7H-pyrrolo[3,2-e][1,2,4]triazolo[1,5-c]pyrimidine-8-carboxamide

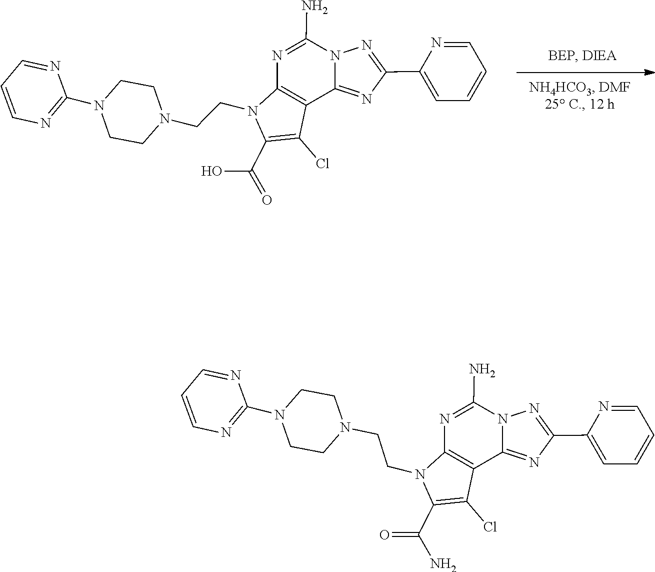 [SMILES: Nc1nc2c(c(Cl)c(C(=O)O)n2CCN2CCN(c3ncccn3)CC2)c2nc(-c3ccccn3)nn12 ; NC(=O)c1c(Cl)c2c(nc(N)n3nc(-c4ccccn4)nc23)n1CCN1CCN(c2ncccn2)CC1]

To a solution of 5-amino-9-chloro-2-(2-pyridyl)-7-[2-(4-pyrimidin-2-ylpiperazin-1-yl)ethyl]-[1,2,4]triazolo pyrrolo pyrimidine-8-carboxylic acid (20.0 mg, 38.5 umol, 1.00 eq) in dimethyl formamide (3.00 mL) was added NH₄HCO₃ (6.10 mg, 76.9 umol, 6.30 uL, 2.00 eq), 2-bromo-1-ethyl-pyridin-1-ium; tetrafluoroborate (12.6 mg, 46.2 umol, 1.20 eq) and diisopropylethylamine (12.4 mg, 96.2 umol, 16.8 uL, 2.50 eq). The mixture was stirred at 25° C. for 12 h. LC-MS showed starting material was completely consumed and one main peak with desired m/z was detected. The reaction mixture was filtered, and the filtrate was purified by prep-HPLC (HCl condition. Column: Welch Xtimate C18 150*25 mm*5 um; mobile phase: [water (0.04% HCl)-ACN]; (B %: 20%-35%, 10 min) to afford 5-amino-9-chloro-2-(pyridin-2-yl)-7-(2-(4-(pyrimidin-2-yl)piperazin-1-yl)ethyl)-7H-pyrrolo[3,2-e][1,2,4]triazolo[1,5-c]pyrimidine-8-carboxamide (6.60 mg, 11.8 umol, HCl) as a yellow solid. ¹H NMR (400 MHz, METHANOL-d₄) δ=8.97 (s, 2H), 8.82 (s, 1H), 8.48 (s, 2H), 8.23 (s, 1H), 6.84 (br d, J=2.4 Hz, 1H), 4.89 (br s, 4H), 3.99-3.85 (m, 2H), 3.80 (br s, 2H), 3.48 (br d, J=1.5 Hz, 2H), 3.29-3.21 (m, 2H). LCMS (ESI) m/z 519.1, Rt: 1.696 min.

### Example 71: Synthesis of 5-amino-9-chloro-2-(pyridin-2-yl)-7-(2-(4-(pyrimidin-2-yl)piperazin-1-yl)ethyl)-7H-pyrrolo[3,2-e][1,2,4]triazolo[1,5-c]pyrimidine-8-carboxamide

### Step 1: Synthesis of methyl 5-amino-9-chloro-7-(2-(4-(6-fluoro-3-methylbenzo[d]isoxazol-5-yl)-1,4-diazepan-1-yl)ethyl)-2-(pyridin-2-yl)-7H-pyrrolo[3,2-e][1,2,4]triazolo[1,5-c]pyrimidine-8-carboxylate

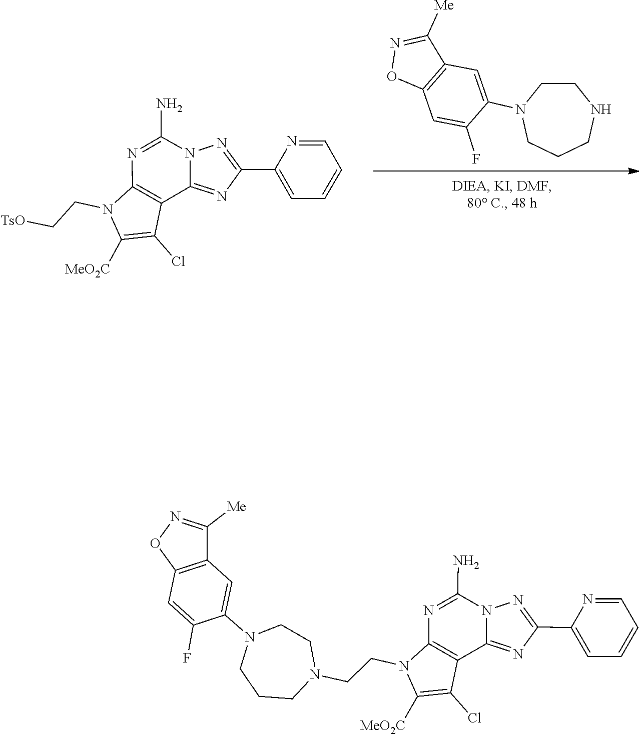 [SMILES: COC(=O)c1c(Cl)c2c(nc(N)n3nc(-c4ccccn4)nc23)n1CCOS(=O)(=O)c1ccc(C)cc1 ; Cc1noc2cc(F)c(N3CCCNCC3)cc12 ; COC(=O)c1c(Cl)c2c(nc(N)n3nc(-c4ccccn4)nc23)n1CCN1CCCN(c2cc3c(C)noc3cc2F)CC1]

To a solution of methyl 5-amino-9-chloro-7-[2-(p-tolylsulfonyloxy)ethyl]-2-(2-pyridyl)-[1,2,4]triazolo[BLAH]pyrrolo[BLAH]pyrimidine-8-carboxylate (150 mg, 276 umol, 1.00 eq) and 5-(1,4-diazepan-1-yl)-6-fluoro-3-methyl-1,2-benzoxazole (137 mg, 553 umol, 2.00 eq) in dimethyl formamide (4.50 mL) was added diisopropylethylamine (143 mg, 1.11 mmol, 192 uL, 4.00 eq) and KI (36.7 mg, 221 umol, 0.80 eq). The mixture was stirred at 80° C. for 48 h. LC-MS showed that starting material was completely consumed and one main peak with the desired m/z was detected. The reaction mixture was concentrated under vacuum and the residue was purified by prep-HPLC (TFA condition) to afford methyl 5-amino-9-chloro-7-(2-(4-(6-fluoro-3-methylbenzo[d]isoxazol-5-yl)-1,4-diazepan-1-yl)ethyl)-2-(pyridin-2-yl)-7H-pyrrolo[3,2-e][1,2,4]triazolo[1,5-c]pyrimidine-8-carboxylate (65.0 mg, 105 umol) as a yellow solid. LCMS (ESI) m/z 619.4, Rt: 1.741 min.

### Step 2: Synthesis of 5-amino-9-chloro-7-(2-(4-(6-fluoro-3-methylbenzo[d]isoxazol-5-yl)-1,4-diazepan-1-yl)ethyl)-2-(pyridin-2-yl)-7H-pyrrolo[3,2-e][1,2,4]triazolo[1,5-c]pyrimidine-8-carboxylic acid

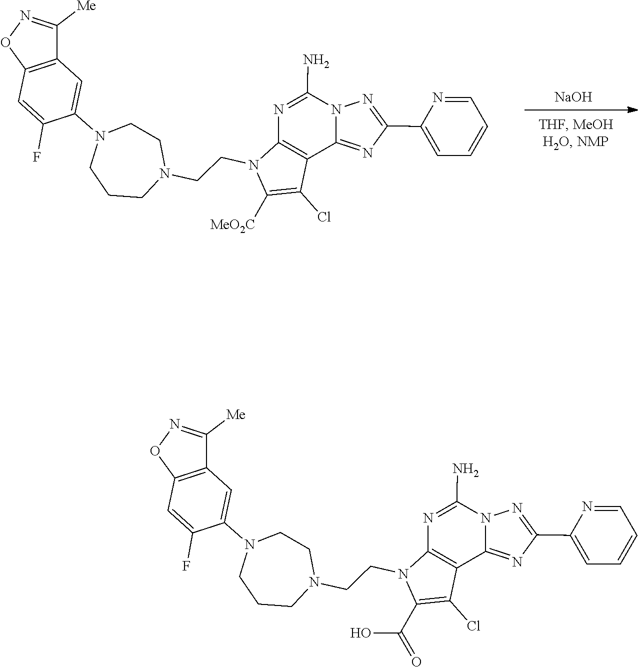 [SMILES: COC(=O)c1c(Cl)c2c(nc(N)n3nc(-c4ccccn4)nc23)n1CCN1CCCN(c2cc3c(C)noc3cc2F)CC1 ; "NaOH" ; Cc1noc2cc(F)c(N3CCCN(CCn4c(C(=O)O)c(Cl)c5c4nc(N)n4nc(-c6ccccn6)nc54)CC3)cc12]

To a solution of methyl 5-amino-9-chloro-7-[2-[4-(6-fluoro-3-methyl-1,2-benzoxazol-5-yl)-1,4-diazepan-1-yl]ethyl]-2-(2-pyridyl)-[1,2,4]triazolo[BLAH]pyrrolo[BLAH]pyrimidine-8-carboxylate (28.0 mg, 38.2 umol, 1.00 eq, TFA) in tetrahydrofuran (0.70 mL), N-methyl pyrrolidone (0.70 mL), methanol (0.70 mL) and water (0.70 mL) was added NaOH (10.7 mg, 267 umol, 7 eq). The mixture was stirred at 100° C. for 0.5 h. LC-MS showed starting material was completely consumed and one main peak with the desired m/z was detected. One additional vial was set up as described above and the two reaction mixtures were combined. The reaction mixture was filtered, and the filtrate was purified by prep-HPLC (HCl condition) to afford 5-amino-9-chloro-7-(2-(4-(6-fluoro-3-methylbenzo[d]isoxazol-5-yl)-1,4-diazepan-1-yl)ethyl)-2-(pyridin-2-yl)-7H-pyrrolo[3,2-e][1,2,4]triazolo[1,5-c]pyrimidine-8-carboxylic acid (30.0 mg, 46.7 umol, HCl) as a yellow solid. LCMS (ESI) m/z 561.2, Rt: 1.935 min. ¹H NMR (400 MHz, METHANOL-d₄) δ=8.87 (d, J=5.4 Hz, 1H), 8.67 (d, J=7.8 Hz, 1H), 8.45 (t, J=7.8 Hz, 1H), 7.95-7.90 (m, 1H), 7.30-7.26 (m, 2H), 5.06 (s, 2H), 4.00 (br s, 1H), 3.89 (br d, J=4.4 Hz, 4H), 3.51 (br s, 4H), 3.36 (br d, J=1.5 Hz, 1H), 2.51 (s, 3H), 2.48-2.43 (m, 1H), 2.26 (br s, 1H).

### Example 72: Synthesis of 5-amino-9-chloro-7-(2-(4-(6-fluoro-3-methylbenzo[d]isoxazol-5-yl)-1,4-diazepan-1-yl)ethyl)-2-(pyridin-2-yl)-7H-pyrrolo[3,2-e][1,2,4]triazolo[1,5-c]pyrimidine-8-carboxamide

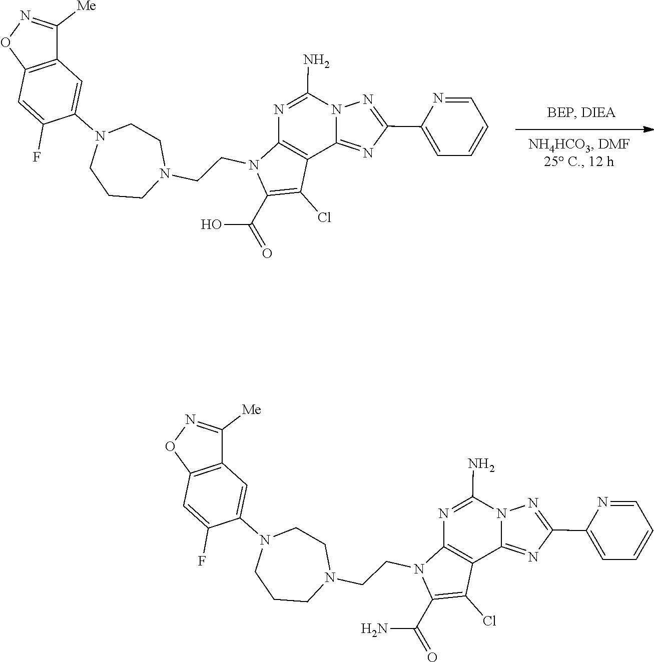 [SMILES: Cc1noc2cc(F)c(N3CCCN(CCn4c(C(=O)O)c(Cl)c5c4nc(N)n4nc(-c6ccccn6)nc54)CC3)cc12 ; Cc1noc2cc(F)c(N3CCCN(CCn4c(C(N)=O)c(Cl)c5c4nc(N)n4nc(-c6ccccn6)nc54)CC3)cc12]

To a solution of 5-amino-9-chloro-7-[2-[4-(6-fluoro-3-methyl-1,2-benzoxazol-5-yl)-1,4-diazepan-1-yl]ethyl]-2-(2-pyridyl)-[1,2,4]triazolo[BLAH]pyrrolo[BLAH]pyrimidine-8-carboxylic acid (14.0 mg, 21.8 umol, 1.00 eq, HCl) in dimethyl formamide (2.00 mL) was added NH₄HCO₃ (3.45 mg, 43.6 umol, 3.59 uL, 2.00 eq), 2-bromo-1-ethyl-pyridin-1-ium; tetrafluoroborate (7.17 mg, 26.1 umol, 1.20 eq) and diisopropylethylamine (7.05 mg, 54.5 umol, 9.50 uL, 2.50 eq). The mixture was stirred at 25° C. for 12 h. LC-MS showed starting material was completely consumed and one main peak with the desired m/z was detected. The reaction mixture was filtered and the filtrate was purified by prep-HPLC (HCl condition) to afford 5-amino-9-chloro-7-(2-(4-(6-fluoro-3-methylbenzo[d]isoxazol-5-yl)-1,4-diazepan-1-yl)ethyl)-2-(pyridin-2-yl)-7H-pyrrolo[3,2-e][1,2,4]triazolo[1,5-c]pyrimidine-8-carboxamide (6.5 mg, 10.2 umol, HCl) as a yellow solid. LCMS (ESI) m/z 604.3, Rt: 1.882 min. ¹H NMR (400 MHz, METHANOL-d₄) δ=8.78 (dd, J=0.9, 4.6 Hz, 1H), 8.50 (d, J=7.9 Hz, 1H), 8.17-8.11 (m, 1H), 7.66 (dd, J=5.2, 6.5 Hz, 1H), 7.37-7.31 (m, 2H), 4.90 (br s, 2H), 3.94-3.82 (m, 3H), 3.81-3.65 (m, 2H), 3.65-3.38 (m, 5H), 2.52 (s, 3H), 2.46-2.39 (m, 1H), 2.35-2.26 (m, 1H).

### Example 73: Synthesis of 5-amino-9-chloro-7-(2-(4-(6-fluoro-3-methylbenzo[d]isoxazol-5-yl)-1,4-diazepan-1-yl)ethyl)-2-(pyridin-2-yl)-7H-pyrrolo[3,2-e][1,2,4]triazolo[1,5-c]pyrimidine-8-carboxamide

### Step 1: Synthesis of ethyl 5-amino-9-chloro-7-(2-(3-methyl-7,8-dihydro-1,6-naphthyridin-6(5H)-yl)ethyl)-2-(pyridin-2-yl)-7H-pyrrolo[3,2-e][1,2,4]triazolo[1,5-c]pyrimidine-8-carboxylate

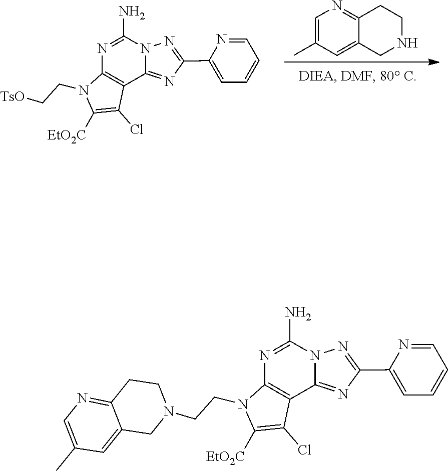

To a solution of ethyl 5-amino-9-chloro-7-[2-(p-tolylsulfonyloxy)ethyl]-2-(2-pyridyl)-[1,2,4]triazolo[BLAH]pyrrolo[BLAH]pyrimidine-8-carboxylate (300 mg, 539.58 umol, 1 eq) in DMF (6 mL) was added 3-methyl-5,6,7,8-tetrahydro-1,6-naphthyridine (95.96 mg, 647.49 umol, 1.2 eq), DIEA (209.21 mg, 1.62 mmol, 281.95 uL, 3 eq) and KI (179.14 mg, 1.08 mmol, 2 eq) under N₂. The mixture was stirred at 80° C. for 12 h. LCMS showed that the starting material was consumed, and the desired product peak was detected. The mixture was filtered, and the solid was dried and triturated in petroleum ether (10 mL). The solid was filtered to afford ethyl 5-amino-9-chloro-7-(2-(3-methyl-7,8-dihydro-1,6-naphthyridin-6(5H)-yl)ethyl)-2-(pyridin-2-yl)-7H-pyrrolo[3,2-e][1,2,4]triazolo[1,5-c]pyrimidine-8-carboxylate (65 mg, 122.18 umol) as a yellow solid. (used in the next step without further purification). LCMS for product (ESI+): m/z 532.2 [M+H]⁺, Rt: 1.091 min. ¹H NMR (400 MHz, DMSO-d₆) δ=8.76 (br s, 1H), 8.26-8.16 (m, 2H), 8.13 (s, 1H), 8.02 (br d, J=3.4 Hz, 1H), 7.61-7.52 (m, 2H), 7.27 (s, 1H), 4.76-4.66 (m, 2H), 4.30-4.20 (m, 2H), 3.61 (s, 2H), 2.84-2.72 (m, 6H), 2.21 (s, 3H), 1.30 (t, J=7.0 Hz, 3H).

### Step 2: Synthesis of 5-amino-9-chloro-7-(2-(3-methyl-7,8-dihydro-1,6-naphthyridin-6(5H)-yl)ethyl)-2-(pyridin-2-yl)-7H-pyrrolo[3,2-e][1,2,4]triazolo[1,5-c]pyrimidine-8-carboxylic acid

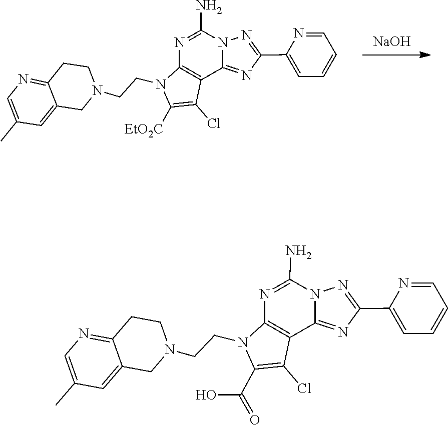

To a solution of ethyl 5-amino-9-chloro-7-[2-(3-methyl-7,8-dihydro-5H-1,6-naphthyridin-6-yl)ethyl]-2-(2-pyridyl)-[1,2,4]triazolo[BLAH]pyrrolo[BLAH]pyrimidine-8-carboxylate (60 mg, 112.78 umol, 1 eq) in MeOH (1 mL) and THE (1 mL) and water (0.5 mL) was added NaOH (31.58 mg, 789.48 umol, 7 eq). The mixture was stirred at 40° C. for 12 h. LCMS showed that the starting material was consumed, and the desired product was detected. The mixture was acidified to pH 2 by dropwise addition of 2 N HCl at 0° C. The mixture was concentrated to afford 5-amino-9-chloro-7-(2-(3-methyl-7,8-dihydro-1,6-naphthyridin-6(5H)-yl)ethyl)-2-(pyridin-2-yl)-7H-pyrrolo[3,2-e][1,2,4]triazolo[1,5-c]pyrimidine-8-carboxylic acid (30 mg, 59.53 umol) as a yellow solid (used without further purification). LCMS for product (ESI+): m/z 504.3 [M+H]⁺, Rt: 0.749 min.

### Step 3: Synthesis of 5-amino-9-chloro-7-(2-(3-methyl-7,8-dihydro-1,6-naphthyridin-6(5H)-yl)ethyl)-2-(pyridin-2-yl)-7H-pyrrolo[3,2-e][1,2,4]triazolo[1,5-c]pyrimidine-8-carboxamide

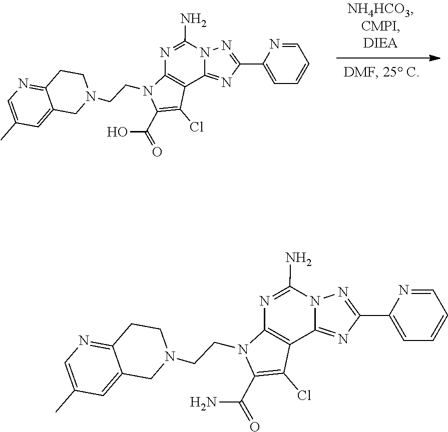 [SMILES: Cc1cnc2c(c1)CN(CCn1c(C(=O)O)c(Cl)c3c1nc(N)n1nc(-c4ccccn4)nc31)CC2 ; Cc1cnc2c(c1)CN(CCn1c(C(N)=O)c(Cl)c3c1nc(N)n1nc(-c4ccccn4)nc31)CC2]

To a solution of 5-amino-9-chloro-7-[2-(3-methyl-7,8-dihydro-5H-1,6-naphthyridin-6-yl)ethyl]-2-(2-pyridyl)-[1,2,4]triazolo[BLAH]pyrrolo[BLAH]pyrimidine-8-carboxylic acid (30 mg, 59.53 umol, 1 eq) in DMF (1 mL) was added DIEA (19.23 mg, 148.83 umol, 25.92 uL, 2.5 eq), NH₄HCO₃ (9.41 mg, 119.06 umol, 9.80 uL, 2 eq) and 2-bromo-1-ethyl-pyridin-1-ium; tetrafluoroborate (24.45 mg, 89.30 umol, 1.5 eq). The mixture was stirred at 25° C. for 2 h. LCMS showed 18% starting material was remaining, 28% product was detected. The mixture was poured into water (10 mL), extracted with ethyl acetate (3×5 mL), and the aqueous phase was concentrated. The residue was purified by prep-HPLC to afford 5-amino-9-chloro-7-(2-(3-methyl-7,8-dihydro-1,6-naphthyridin-6(5H)-yl)ethyl)-2-(pyridin-2-yl)-7H-pyrrolo[3,2-e][1,2,4]triazolo[1,5-c]pyrimidine-8-carboxamide (1.2 mg, 2.39 umol) as a yellow solid. LCMS for product (ESI+): m/z 503.1 [M+H]⁺, Rt: 1.62 min. ¹H NMR (400 MHz, DMSO-d₆) δ=8.76 (br d, J=4.6 Hz, 1H), 8.50 (br s, 1H), 8.37-8.19 (m, 3H), 8.10-7.94 (m, 2H), 7.87 (br s, 1H), 7.63 (br d, J=1.0 Hz, 1H), 7.60-7.55 (m, 1H), 4.89-4.55 (m, 4H), 3.75 (br dd, J=4.2, 4.8 Hz, 2H), 3.29 (br s, 4H), 2.35 (s, 3H).

### Example 74: Synthesis of 9-chloro-7-(2-(3-methyl-7,8-dihydro-1,6-naphthyridin-6(5H)-yl)ethyl)-2-(pyridin-2-yl)-7H-pyrazolo[4,3-e][1,2,4]triazolo[1,5-c]pyrimidin-5-amine

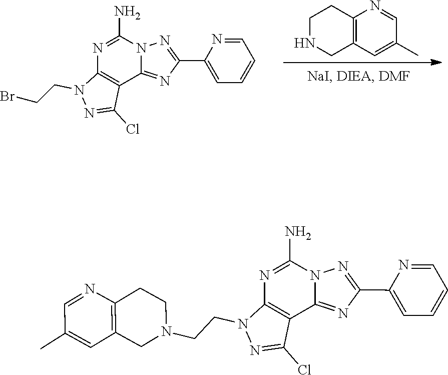 [SMILES: Nc1nc2c(c(Cl)nn2CCBr)c2nc(-c3ccccn3)nn12 ; Cc1cnc2c(c1)CNCC2 ; Cc1cnc2c(c1)CN(CCn1nc(Cl)c3c1nc(N)n1nc(-c4ccccn4)nc31)CC2]

To a mixture of 7-(2-bromoethyl)-9-chloro-2-(2-pyridyl)-[1,2,4]triazolo[BLAH]pyrazolo[BLAH]pyrimidin-5-amine (76.74 mg, 194.95 umol, 1.2 eq) and 3-methyl-5,6,7,8-tetrahydro-1,6-naphthyridine (30 mg, 162.46 umol, 1 eq, HCl) in DMF (1 mL) was added NaI (24.35 mg, 162.46 umol, 1 eq) and DIEA (41.99 mg, 324.91 umol, 56.59 uL, 2 eq) under N₂. The mixture was stirred at 80° C. for 2 h. LCMS showed starting material was consumed and one main peak with the desired m/z. The mixture was filtered, and the filtrate was purified by prep-HPLC to afford 9-chloro-7-(2-(3-methyl-7,8-dihydro-1,6-naphthyridin-6(5H)-yl)ethyl)-2-(pyridin-2-yl)-7H-pyrazolo[4,3-e][1,2,4]triazolo[1,5-c]pyrimidin-5-amine (8 mg, 17.13 umol) as a white solid. LCMS for product (ESI+): m/z 461.2 [M+H]⁺, Rt: 1.673 min. ¹H NMR (400 MHz, CDCl₃) δ=8.84 (d, J=4.5 Hz, 1H), 8.48 (d, J=8.0 Hz, 1H), 8.21 (s, 1H), 7.91 (t, J=7.8 Hz, 1H), 7.44 (dd, J=5.3, 7.3 Hz, 1H), 7.12 (s, 1H), 6.16 (br s, 2H), 4.54 (t, J=6.8 Hz, 2H), 3.71 (s, 2H), 3.10 (t, J=6.8 Hz, 2H), 2.97 (s, 4H), 2.27 (s, 3H).

### PHARMACEUTICAL COMPOSITIONS

### Example A-1: Parenteral Pharmaceutical Composition

To prepare a parenteral pharmaceutical composition suitable for administration by injection (subcutaneous, intravenous), 1-1000 mg of a water-soluble salt of a compound described herein, or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### Example A-2: Oral Solution

To prepare a pharmaceutical composition for oral delivery, a sufficient amount of a compound described herein, or a pharmaceutically acceptable salt thereof, is added to water (with optional solubilizer(s), optional buffer(s) and taste masking excipients) to provide a 20 mg/mL solution.

### Example A-3: Oral Tablet

A tablet is prepared by mixing 20-50% by weight of a compound described herein, or a pharmaceutically acceptable salt thereof, 20-50% by weight of microcrystalline cellulose, and 1-10% by weight of magnesium stearate or other appropriate excipients. Tablets are prepared by direct compression. The total weight of the compressed tablets is maintained at 100-500 mg.

### Example A-4: Oral Capsule

To prepare a pharmaceutical composition for oral delivery, 1-1000 mg of a compound described herein, or a pharmaceutically acceptable salt thereof, is mixed with starch or other suitable powder blend. The mixture is incorporated into an oral dosage unit such as a hard gelatin capsule, which is suitable for oral administration.

In another embodiment, 1-1000 mg of a compound described herein, or a pharmaceutically acceptable salt thereof, is placed into Size 4 capsule, or size 1 capsule (hypromellose or hard gelatin) and the capsule is closed.

### BIOLOGICAL EXAMPLES

### Example B-1: Adenosine Receptor Binding Assay

Membranes prepared from CHO-K1 cells stably expressing Human AiAdoR (ES-010-M400UA), HEK-293 cells stably expressing human A_{2A} AdoR (RBHA2AM400UA), HEK-293 cells stably expressing human A_{2B} AdoR (ES-013-M400UA) and CHO-K1 cells stably expressing Human A₃ AdoR (ES-012-M400UA) were purchased from PerkinElmer (Waltham, Mass., USA) and stored at −80° C. until use. Binding assays are performed using the radioligands. The final concentrations are as follows: [3H]-DPCPX (PerkinElmer, NET974001MC) at 1 nM for A₁AdoR; [³H]-CGS-21680 (PerkinElmer, NET1021250UC) at 6 nM for A_{2A}AdoR, [3H]-DPCPX (PerkinElmer, NET974001MC) at 8 nM for A_{2B}AdoR; [³H]-HEMADO (ARC, Cat: ART1456) at 1 nM for A₃AdoR. Testing compounds are diluted with DMSO to make 8-point 4-fold serial dilution. Nonspecific binding (Low control: LC) and total binding (High control: HC) are determined in the presence or absence of saturated cold ligand. Specific binding is calculated by subtracting nonspecific binding from total binding. All assays are performed in a final volume of 200 μl containing 1 μl of the test compound in DMSO, 100 μl of membrane preparation, and 99 μl of the radioligand in assay buffer (A₁AdoR: 25 mM HEPES pH 7.4, 5 mM MgCl2, 1 mM CaCl2), 100 mM NaCl; A_{2A}AdoR: 50 mM Tris HCl pH 7.4, 10 mM MgCl2, 1 mM EDTA; A_{2B}AdoR: 50 mM HEPES pH 7.0, 5 mM MgCl2, 1 mM EDTA; A₃AdoR: 25 HEPES pH 7.4, 10 mM MgCl2, 1 mM CaCl₂), 0.5% BSA). The incubation is performed at room temperature with shaking at 300 rpm for 1 hour for A₁AdoR, A_{2B}AdoR and A₃AdoR and 2 hours for A_{2A}AdoR. After the incubation, the assay mixture is filtered through 96 GF/C filter plates (Perkin Elmer #6005174) using Perkin Elmer Filtermate Harvester, and then washed four times with ice-cold washing buffer (A₁AdoR: 25 mM HEPES pH 7.4, 5 mM MgCl2, 1 mM CaCl2), 100 mM NaCl; A_{2A}AdoR: 50 mM Tris HCl pH 7.4, 154 mM NaCl; A_{2B}AdoR: 50 mM HEPES pH 6.5, 5 mM MgCl2, 1 mM EDTA, 0.2% BSA; A₃AdoR: 50 mM Tris HCl pH 7.4). The filters are dried for 1 hour at 50° C. and [³H] trapped on filter counted for radioactivity in Perkin Elmer Microscint 20 cocktail (#6013329) using Perkin Elmer MicroBeta2 Reader. The results are expressed as a percent inhibition of the control radioligand specific binding calculated using the following equation: % Inhibition=(1−(Assay well-Average_LC)/(Average_HC−Average_LC))×100%. Data are analyzed and IC50 is calculated using GraphPad Prism 5 and the model “log(inhibitor) vs. response—Variable slope”. The binding affinity of the compounds is determined by using the Cheng and Prusoff equation Ki=IC50/(1+[radioligand]/Kd).

### Example B-2: Human Whole Blood Phospho-CREB (PCREB) Assay

Fresh human blood samples are derived from healthy volunteers and processed in heparin tubes. 67.5 ul of whole blood is aliquoted to each well in a 96-well plate and incubated at 37° C. for 30 min. Testing compounds are diluted with DMSO to make 8-point 3-fold serial dilution and 3.5 ul (20×) is added to each well. Cells are incubated with the compounds at 37° C. for 30 min. NECA (5 uM) is then added to each well and cells are incubated for 30 min at 37° C. Following the stimulation, cells are transferred to a 96-well deep well plates and fixed with 1 ml/well of lyse/fix buffer (1×) (BD Biosciences #558049) with vigorous shake at 37° C. for 10 min. Cells are centrifuged at 600 g for 6 min and washed twice with PBS, followed by addition of 800 ul/well Perm buffer III (BD Biosciences #558050). Cells are then pelleted at 600 g for 6 min, washed with FACS buffer (PBS+0.2% BSA+1 mM EDTA) and stained for 40 min at room temperature in the dark with an antibody cocktail containing PE mouse anti-human CD3 (BD Biosciences #555333, 1 ul/well), FITC mouse anti-human CD4 (BD Biosciences #5555346, 1 ul/well), PerCP-Cy™5.5 mouse anti-human CD8 (BD Biosciences #565310, 1 ul/well) and phospho-CREB (Ser133) (87G3) Rabbit mAb (Alexa Fluor® 647 Conjugate, CST #14001, 0.5 ul/well). Cells are then washed twice with FACS buffer and acquired on a flow cytometer (Sony Cell Sorter SH800). Data were analyzed using FlowJo version 9. The level of CREB phosphorylation from unstimulated cells (Low control: LC) and NECA-stimulated cells (High control: HC) are determined as mean fluorescent intensity (MFI) in stimulated condition/mean fluorescent intensity in unstimulated condition. % Inhibition is calculated as (MFI of HC-MFI of assay well)/(MFI of HC-MFI of LC)×100%. Data are analyzed and IC50s calculated using GraphPad Prism 5 and the model “log(inhibitor) vs. response—Variable slope”.

### Example B-2: Modulation of PCREB in Human Immune Cells

The A_{2A} receptor is known to mediate CREB phosphorylation. This assay is intended to demonstrate that the compounds described herein effectively inhibit A_{2A}AdoR receptor by showing that CREB phosphorylation may be inhibited by exposing human immune cells to compounds disclosed herein.

Venous blood from healthy volunteers, all of whom signed an informed consent approved by the Ethics Committee (FOR—UIC-BV-050-01-01 ICF HBS HD Version 5.0), was obtained via ImmuneHealth (Centre Hospitalier Universitaire Tivoli, La Louviere,

15 Belgium). Peripheral blood cells were treated with A2AR agonists CGS-21680 or NECA (Sigma-Aldrich, Diegem, Belgium), and a serial dilution of compounds disclosed herein (all used stock solutions at 10 mM in DMSO). All dilutions were prepared in RPMI1640 medium (with UltraGlutamine; Lonza, Venders, Belgium), and cells were incubated with compounds in a 37° C. humidified tissue culture incubator with 5% CO₂.

After stimulation, cells were fixed and permeabilized, followed by intracellular staining using mouse anti-human pCREB antibodies (Clone J151-21; BD Biosciences) at room temperature. Data were acquired using an LSRFortessa flow cytometer (BD Biosciences) and analyzed using FlowJo software (FlowJo, LLC, Ashland, Oregon).

Representative data for compounds disclosed herein is provided in the following Table.

| | hA_{2A} | | pCREB | |
|---|---|---|---|---|
| | IC₅₀ | A₁/A_{2A} | EC₅₀ | A_{2B}/A_{2A} |
| Compound | (nM) | Selectivity | (nM) | Selectivity |
| 5-amino-7-[2-[4-(2-fluoro-5-oxazol-2-yl- | +++ | b | ++ | a |
| phenyl)piperazin-1-yl]ethyl]-2-(2-pyridyl)- | | | | |
| [1,2,4]triazolopyrrolopyrimidine-8-carboxamide | | | | |
| 9-chloro-7-[2-[4-(6-fluoro-3-methyl-1,2-benzoxazol- | +++ | b | +++ | a |
| 5-yl)piperazin-1-yl]ethyl]-2-(2-pyridyl)- | | | | |
| [1,2,4]triazolo-pyrazolo-pyrimidin-5-amine | | | | |
| 5-amino-7-[2-[4-(2-fluoro-5-oxazol-2-yl- | +++ | a | + | a |
| phenyl)piperazin-1-yl]ethyl]-2-(2-pyridyl)- | | | | |
| [1,2,4]triazolopyrrolopyrimidine-8-carbonitrile | | | | |
| 5-amino-7-(2-(4-(6-fluoro-3- | +++ | c | + | a |
| methylbenzo[d]isoxazol-5-yl)piperazin-1-yl)ethyl)-2- | | | | |
| (pyridin-2-yl)-7H-pyrrolo[3,2-e][1,2,4]triazolo[1,5- | | | | |
| c]pyrimidine-8-carbonitrile | | | | |
| 9-chloro-7-(2-(4-(2-fluoro-5-(oxazol-2- | +++ | c | ++ | a |
| yl)phenyl)piperazin-1-yl)ethyl)-2-(pyridin-2-yl)-7H- | | | | |
| pyrazolo[4,3-e][1,2,4]triazolo[1,5-c]pyrimidin-5- | | | | |
| amine | | | | |
| 5-amino-7-(2-(4-(4-fluoro-3- | +++ | a | NT | NT |
| methylbenzo[d]isoxazol-5-yl)piperazin-1-yl)ethyl)-2- | | | | |
| (prop-1-yn-1-yl)-7H-pyrrolo[3,2- | | | | |
| e][l,2,4]triazolo[1,5-c]pyrimidine-8-carbonitrile | | | | |
| 5-amino-7-(2-(4-(2-fluoro-5-(oxazol-2- | +++ | a | + | a |
| yl)phenyl)piperazin-1-yl)ethyl)-2-(prop-1-yn-1-yl)- | | | | |
| 7H-pyrrolo[3,2-e][1,2,4]triazolo[1,5-c]pyrimidine-8- | | | | |
| carbonitrile | | | | |
| 2-[4-[4-[2-[5-amino-8-cyano-2-(2-pyridyl)- | +++ | b | ++ | a |
| [l,2,4]triazolopyrrolopyrimidin-7-yl]ethyl]piperazin- | | | | |
| 1-yl]phenoxy]acetic acid | | | | |
| 2-[4-[4-[2-[5-amino-8-carbamoyl-2-(2-pyridyl)- | +++ | b | ++ | a |
| [1,2,4]triazolo[1,5-e]pyrrolo[3,2-e]pyrimidin-7- | | | | |
| yl]ethyl]piperazin-1-yl]phenoxy]acetic acid | | | | |
| 5-amino-7-(2-(4-(4-(2- | +++ | b | + | a |
| methoxyethoxy)phenyl)piperazin-1-yl)ethyl)-2- | | | | |
| (pyridin-2-yl)-7H-pyrrolo[3,2-e][1,2,4]triazolo[1,5- | | | | |
| e]pyrimidine-8-carbonitrile | | | | |
| 5-amino-9-chloro-7-[2-[4-(6-fluoro-3-methyl-1,2- | +++ | a | ++ | a |
| benzoxazol-5-yl)piperazin-1-yl]ethyl]-2-(2-pyridyl)- | | | | |
| [1,2,4]triazolopyrrolopyrimidine-8-carbonitrile | | | | |
| 5-amino-9-chloro-7-(2-(4-(2-fluoro-5-(oxazol-2- | +++ | NT | ++ | |
| yl)phenyl)piperazin-1-yl)ethyl)-2-(pyridin-2-yl)-7H- | | | | |
| pyrrolo[3,2-e][1,2,4]triazolo[1,5-c]pyrimidine-8- | | | | |
| carbonitrile | | | | |
| 5-amino-7-(2-(4-(4-(2- | +++ | a | + | a |
| methoxyethoxy)phenyl)piperazin-1-yl)ethyl)-2- | | | | |
| (prop-1-yn-1-yl)-7H-pyrrolo[3,2- | | | | |
| e][l,2,4]triazolo[1,5-c]pyrimidine-8-carbonitrile | | | | |
| 5-amino-7-(2-(4-(4-(2- | +++ | c | + | a |
| methoxyethoxy)phenyl)piperazin-1-yl)ethyl)-2- | | | | |
| (pyridin-2-yl)-7H-pyrrolo[3,2-e][1,2,4]triazolo[1,5- | | | | |
| c]pyrimidine-8-carboxamide | | | | |
| 7-(2-(4-(2-fluoro-5-(oxazol-2-yl)phenyl)piperazin-1- | +++ | c | ++ | |
| yl)ethyl)-9-methyl-2-(pyridin-2-yl)-7H-pyrrolo[3,2- | | | | |
| e][1,2,4]triazolo[1,5-c]pyrimidin-5-amine | | | | |
| 3-[2-[4-(2-fluoro-5-oxazol-2-yl-phenyl)piperazin-1- | +++ | a | ++ | |
| yl]ethyl]-8-prop-1-ynyl-[1,2,4]triazolo[5,1-f]purin-5- | | | | |
| amine | | | | |
| methyl 5-amino-9-chloro-7-(2-(4-(2-fluoro-5- | +++ | a | | |
| (oxazol-2-yl)phenyl)piperazin-1-yl)ethyl)-2-(pyridin- | | | | |
| 2-yl)-7H-pyrrolo[3,2-e][1,2,4]triazolo[1,5- | | | | |
| c]pyrimidine-8-carboxylate | | | | |
| 2-(4-(4-(2-(5-amino-9-chloro-8-cyano-2-(pyridin-2- | +++ | b | ++ | |
| yl)-7H-pyrrolo[3,2-e][l,2,4]triazolo[1,5-c]pyrimidin- | | | | |
| 7-yl)ethyl)piperazin-1-yl)phenoxy)- | | | | |
| N(methylsulfonyl)acetamide | | | | |
| methyl 5-amino-7-(2-(4-(2-fluoro-5-(oxazol-2- | +++ | a | + | |
| yl)phenyl)piperazin-1-yl)ethyl)-9-methyl-2-(pyridin- | | | | |
| 2-yl)-7H-pyrrolo[3,2-e][1,2,4]triazolo[1,5- | | | | |
| clpyrimidine-8-carboxylate | | | | |
| 5-amino-7-[2-[4-(2-fluoro-5-oxazol-2-yl- | +++ | a | +++ | a |
| phenyl)piperazin-1-yl]ethyl]-9-methyl-2-(2-pyridyl)- | | | | |
| [l,2,4]triazolopyrrolopyrimidine-8-carboxylic acid | | | | |
| 5-amino-9-chloro-7-[2-[4-(6-fluoro-3-methyl-1,2- | +++ | a | +++ | a |
| benzoxazol-5-yl)piperazin-1-yl]ethyl]-2-(2-pyridyl)- | | | | |
| [1,2,4]triazolo[1,5-c]pyrrolo[3,2-e]pyrimidine-8- | | | | |
| carboxamide | | | | |
| methyl 5-amino-7-(2-(4-(2-fluoro-5-(oxazol-2- | +++ | a | | |
| yl)phenyl)piperazin-1-yl)ethyl)-2-(pyridin-2-yl)-7H- | | | | |
| pyrrolo[3,2-e][1,2,4]triazolo[1,5-c]pyrimidine-8- | | | | |
| carboxylate | | | | |
| 5-amino-7-(2-(4-(2-fluoro-5-(oxazol-2- | +++ | b | + | a |
| yl)phenyl)piperazin-1-yl)ethyl)-2-(pyridin-2-yl)-7H- | | | | |
| pyrrolo[3,2-e][1,2,4]triazolo[1,5-c]pyrimidine-8- | | | | |
| carboxylic acid | | | | |
| 5-amino-7-(2-(4-(2-fluoro-5-(oxazol-2- | +++ | c | + | |
| yl)phenyl)piperazin-1-yl)ethyl)-N-methyl-2-(pyridin- | | | | |
| 2-yl)-7H-pyrrolo[3,2-e][1,2,4]triazolo[1,5- | | | | |
| c]pyrimidine-8-carboxamide | | | | |
| 5-amino-7-[2-[4-(2-fluoro-5-oxazol-2-yl- | +++ | a | + | a |
| phenyl)piperazin-1-yl]ethyl]-N-cyclopropyl-9- | | | | |
| methyl-2-(2-pyridyl)- | | | | |
| [1,2,4]triazolopyrrolopyrimidine-8-carboxamide | | | | |
| 5-amino-7-[2-[4-(2-fluoro-5-oxazol-2-yl- | +++ | a | +++ | |
| phenyl)piperazin-1-yl]ethyl]-N,9-dimethyl-2-(2- | | | | |
| pyridyl)-[1,2,4]triazolopyrrolopyrimidine-8- | | | | |
| carboxamide | | | | |
| 5-amino-9-chloro-7-(2-(4-(2-fluoro-5-(oxazol-2- | +++ | b | | |
| yl)phenyl)piperazin-1-yl)ethyl)-2-(pyridin-2-yl)-7H- | | | | |
| pyrrolo[3,2-e][1,2,4]triazolo[1,5-c]pyrimidine-8- | | | | |
| carboxylic acid | | | | |
| 5-amino-7-(2-(4-(2-fluoro-5-(oxazol-2- | +++ | a | +++ | |
| yl)phenyl)piperazin-1-yl)ethyl)9-methyl-2-(pyridin- | | | | |
| 2-yl)-7H-pyrrolo[3,2-e][1,2,4]triazolo[1,5- | | | | |
| c]pyrimidine-8-carboxamide | | | | |
| 5-amino-7-[2-[4-(2-fluoro-5-oxazol-2-yl- | +++ | b | ++ | |
| phenyl)piperazin-1-yl]ethyl]-9-methyl-2-(pyridin-2- | | | | |
| yl)-7H-pyrrolo[3,2-e][l,2,4]triazolo[1,5-c]pyrimidin- | | | | |
| 8-yl)(azetidin-1-yl)methanone | | | | |
| 5-amino-9-chloro-N-cyclopropyl-7-(2-(4-(2-fluoro-5- | +++ | a | | |
| (oxazol-2-yl)phenyl)piperazin-1-yl)ethyl)-2-(pyridin- | | | | |
| 2-yl)-7H-pyrrolo[3,2-e][1,2,4]triazolo[1,5- | | | | |
| clpyrimidine-8-carboxamide | | | | |
| 5-amino-9-chloro-7-(2-(4-(2-fluoro-5-(oxazol-2- | +++ | a | | |
| yl)phenyl)piperazin-1-yl)ethyl)-2-(pyridin-2-yl)-7H- | | | | |
| pyrrolo[3,2-e][1,2,4]triazolo[1,5-c]pyrimidine-8- | | | | |
| carboxamide | | | | |
| 5-amino-9-chloro-7-(2-(4-(2-fluoro-5-(oxazol-2- | +++ | a | | |
| yl)phenyl)piperazin-1-yl)ethyl)-N-methyl-2-(pyridin- | | | | |
| 2-yl)-7H-pyrrolo[3,2-e][1,2,4]triazolo[1,5- | | | | |
| clpyrimidine-8-carboxamide | | | | |
| 2-(4-(4-(2-(5-amino-8-cyano-2-(pyridin-2-yl)-7H- | +++ | b | + | |
| pyrrolo[3,2-e][1,2,4]triazolo[1,5-c]pyrimidin-7- | | | | |
| yl)ethyl)piperazin-1-yl)phenoxy)-N- | | | | |
| (methylsulfonyl)acetamide | | | | |
| 2-(4-(4-(2-(5-amino-8-carbamoyl-9-chloro-2- | +++ | b | | |
| (pyridin-2-yl)-7H-pyrrolo[3,2-e][1,2,4]triazolo[1,5- | | | | |
| c]pyrimidin-7-yl)ethyl)piperazin-1-yl)phenoxy)acetic | | | | |
| acid | | | | |
| 5-amino-9-chloro-7-(2-(4-(6-fluoro-3- | +++ | a | | |
| methylbenzo[d]isoxazol-5-yl)piperazin-1-yl)ethyl)-2- | | | | |
| (pyridin-2-yl)-7H-pyrrolo[3,2-e][1,2,4]triazolo[1,5- | | | | |
| c]pyrimidin-8-yl formate | | | | |
| 2-(4-(4-(2-(5-amino-8-(methoxycarbonyl)-9-methyl- | +++ | b | | |
| 2-(pyridin-2-yl)-7H-pyrrolo[3,2-e][1,2,4]triazolo[1,5- | | | | |
| c]pyrimidin-7-yl)ethyl)piperazin-1-yl)phenoxy)acetic | | | | |
| acid | | | | |
| 3-(2-(4-(6-fluoro-3-methylbenzo[d]isoxazol-5- | +++ | a | | |
| yl)piperazin-1-yl)ethyl)-8-(prop-1-yn-1-yl)-3H- | | | | |
| [1,2,4]triazolo[5,1-i]purin-5-amine | | | | |
| 5-amino-9-chloro-7-(2-(4-(2,4- | +++ | b | | |
| difluorophenyl)piperazin-1-yl)ethyl)-2-(pyridin-2- | | | | |
| yl)-7H-pyrrolo[3,2-e][1,2,4]triazolo[1,5- | | | | |
| c]pyrimidine-8-carboxylic acid | | | | |
| 5-amino-9-chloro-2-(pyridin-2-yl)-7-(2-(4-(pyridin- | ++ | b | | |
| 4-yl)piperazin-1-yl)ethyl)-7H-pyrrolo[3,2- | | | | |
| e][1,2,4]triazolo[1,5-c]pyrimidine-8-carboxylic acid | | | | |
| 5-amino-9-chloro-7-(2-(4-(4-cyano-2- | +++ | b | | |
| fluorophenyl)piperazin-1-yl)ethyl)-2-(pyridin-2-yl)- | | | | |
| 7H-pyrrolo[3,2-e][1,2,4]triazolo[1,5-c]pyrimidine-8- | | | | |
| carboxylic acid | | | | |
| 5-amino-9-chloro-7-(2-(4-(2,4- | +++ | c | | |
| difluorophenyl)piperazin-1-yl)ethyl)-2-(pyridin-2- | | | | |
| yl)-7H-pyrrolo[3,2-e][1,2,4]triazolo[1,5- | | | | |
| clpyrimidine-8-carboxamide | | | | |
| 5-amino-9-chloro-2-(pyridin-2-yl)-7-(2-(4-(pyridin- | +++ | c | | |
| 4-yl)piperazin-1-yl)ethyl)-7H-pyrrolo[3,2- | | | | |
| e][1,2,4]triazolo[1,5-c]pyrimidine-8-carboxamide | | | | |
| 5-amino-9-chloro-7-(2-(4-(4-cyano-2- | +++ | a | | |
| fluorophenyl)piperazin-1-yl)ethyl)-2-(pyridin-2-yl)- | | | | |
| 7H-pyrrolo[3,2-e][1,2,4]triazolo[1,5-c]pyrimidine-8- | | | | |
| carboxamide | | | | |
| 5-amino-7-(2-(4-(6-fluoro-3- | +++ | b | | |
| methylbenzo[d]isoxazol-5-yl)piperazin-1-yl)ethyl)-9- | | | | |
| methyl-2-(pyridin-2-yl)-7H-pyrrolo[3,2- | | | | |
| e][l,2,4]triazolo[1,5-c]pyrimidine-8-carboxylic acid | | | | |
| 5-amino-7-(2-(4-(2-fluoro-5-(oxazol-2-yl)phenyl)- | ++ | c | | |
| 1,4-diazepan-1-yl)ethyl)-9-methyl-2-(pyridin-2-yl)- | | | | |
| 7H-pyrrolo[3,2-e][1,2,4]triazolo[1,5-c]pyrimidine-8- | | | | |
| carboxylic acid | | | | |
| 7-(2-(4-(2-fluoro-5-(oxazol-2-yl)phenyl)-1,4- | +++ | c | | |
| diazepan-1-yl)ethyl)-9-methyl-2-(pyridin-2-yl)-7H- | | | | |
| pyrrolo[3,2-e][1,2,4]triazolo[1,5-c]pyrimidin-5- | | | | |
| amine | | | | |
| 5-amino-7-(2-(4-(2-fluoro-5-(oxazol-2-yl)phenyl)- | +++ | b | | |
| 1,4-diazepan-1-yl)ethyl)-N,9-dimethyl-2-(pyridin-2- | | | | |
| yl)-7H-pyrrolo[3,2-e][1,2,4]triazolo[1,5- | | | | |
| c]pyrimidine-8-carboxamide | | | | |
| 3-(2-(4-(2-fluoro-5-(oxazol-2-yl)phenyl)piperazin-1- | +++ | b | | |
| yl)ethyl)-8-(pyridin-2-yl)-3H-[1,2,4]triazolo[5,1- | | | | |
| i]purin-5-amine | | | | |
| 3-(2-(4-(6-fluoro-3-methylbenzo[d]isoxazol-5- | +++ | b | + | |
| yl)piperazin-1-yl)ethyl)-8-(pyridin-2-yl)-3H- | | | | |
| [1,2,4]triazolo[5,1-i]purin-5-amine | | | | |
| 5-amino-7-(2-(4-(6-fluoro-3- | +++ | a | | |
| methylbenzo[d]isoxazol-5-yl)piperazin-1-yl)ethyl)-9- | | | | |
| methyl-2-(pyridin-2-yl)-7H-pyrrolo[3,2- | | | | |
| e][1,2,4]triazolo[1,5-c]pyrimidine-8-carboxamide | | | | |
| 5-amino-7-(2-(4-(2,4-difluorophenyl)piperazin-1- | +++ | b | | |
| yl)ethyl)-9-methyl-2-(pyridin-2-yl)-7H-pyrrolo[3,2- | | | | |
| e][1,2,4]triazolo[1,5-c]pyrimidine-8-carboxamide | | | | |
| 5-amino-7-(2-(4-(2,4-difluorophenyl)piperazin-1- | +++ | b | | |
| yl)ethyl)-9-methyl-2-(pyridin-2-yl)-7H-pyrrolo[3,2- | | | | |
| e][1,2,4]triazolo[1,5-c]pyrimidine-8-carboxylic acid | | | | |
| 5-amino-9-methyl-2-(pyridin-2-yl)-7-(2-(4-(pyridin- | +++ | c | | |
| 4-yl)piperazin-1-yl)ethyl)-7H-pyrrolo[3,2- | | | | |
| e][1,2,4]triazolo[1,5-c]pyrimidine-8-carboxamide | | | | |
| 5-amino-9-chloro-2-(pyridin-2-yl)-7-(2-(4- | +++ | c | | |
| (pyrimidin-2-yl)piperazin-1-yl)ethyl)-7H- | | | | |
| pyrrolo[3,2-e][1,2,4]triazolo[1,5-c]pyrimidine-8- | | | | |
| carboxamide | | | | |
| 5-amino-7-(2-(4-(4-cyano-2-fluorophenyl)piperazin- | +++ | b | | |
| 1-yl)ethyl)-9-methyl-2-(pyridin-2-yl)-7H- | | | | |
| pyrrolo[3,2-e][1,2,4]triazolo[1,5-c]pyrimidine-8- | | | | |
| carboxamide | | | | |
| 5-amino-9-methyl-2-(pyridin-2-yl)-7-(2-(4- | +++ | b | | |
| (pyrimidin-2-yl)piperazin-1-yl)ethyl)-7H- | | | | |
| pyrrolo[3,2-e][1,2,4]triazolo[1,5-c]pyrimidine-8- | | | | |
| carboxamide | | | | |
| 5-amino-9-methyl-2-(pyridin-2-yl)-7-(2-(4-(pyridin- | ++ | c | | |
| 4-yl)piperazin-1-yl)ethyl)-7H-pyrrolo[3,2- | | | | |
| e][l,2,4]triazolo[1,5-c]pyrimidine-8-carboxylic acid | | | | |
| 5-amino-9-chloro-2-(pyridin-2-yl)-7-(2-(4- | ++ | c | | |
| (pyrimidin-2-yl)piperazin-1-yl)ethyl)-7H- | | | | |
| pyrrolo[3,2-e][1,2,4]triazolo[1,5-c]pyrimidine-8- | | | | |
| carboxylic acid | | | | |
| 5-amino-7-(2-(4-(4-cyano-2-fluorophenyl)piperazin- | +++ | b | | |
| 1-yl)ethyl)-9-methyl-2-(pyridin-2-yl)-7H- | | | | |
| pyrrolo[3,2-e][1,2,4]triazolo[1,5-c]pyrimidine-8- | | | | |
| carboxylic acid | | | | |
| 5-amino-9-methyl-2-(pyridin-2-yl)-7-(2-(4- | +++ | b | | |
| (pyrimidin-2-yl)piperazin-1-yl)ethyl)-7H- | | | | |
| pyrrolo[3,2-e][1,2,4]triazolo[1,5-c]pyrimidine-8- | | | | |
| carboxylic acid | | | | |
| methyl 5-amino-7-(2-(4-(6-fluoro-3- | +++ | b | | |
| methylbenzo[d]isoxazol-5-yl)-1,4-diazepan-1- | | | | |
| yl)ethyl)-9-methyl-2-(pyridin-2-yl)-7H-pyrrolo[3,2- | | | | |
| e][1,2,4]triazolo[1,5-c]pyrimidine-8-carboxylate | | | | |
| 5-amino-7-(2-(4-(5-fluoro-2-methylpyridin-4- | +++ | c | | |
| yl)piperazin-1-yl)ethyl)-9-methyl-2-(pyridin-2-yl)- | | | | |
| 7H-pyrrolo[3,2-e][1,2,4]triazolo[1,5-c]pyrimidine-8- | | | | |
| carboxamide | | | | |
| 5-amino-9-chloro-7-(2-(4-(6-fluoro-3- | +++ | b | | |
| methylbenzo[d]isoxazol-5-yl)-l,4-diazepan-1- | | | | |
| yl)ethyl)-2-(pyridin-2-yl)-7H-pyrrolo[3,2- | | | | |
| e][1,2,4]triazolo[1,5-c]pyrimidine-8-carboxamide | | | | |
| 5-amino-9-chloro-7-(2-(4-(6-fluoro-3- | + | c | | |
| methylbenzo[d]isoxazol-5-yl)-1,4-diazepan-1- | | | | |
| yl)ethyl)-2-(pyridin-2-yl)-7H-pyrrolo[3,2- | | | | |
| e][1,2,4]triazolo[1,5-c]pyrimidine-8-carboxylic acid | | | | |
| methyl 5-amino-7-(2-(4-(5-fluoro-2-methylpyridin-4- | +++ | a | | |
| yl)piperazin-1-yl)ethyl)-9-methyl-2-(pyridin-2-yl)- | | | | |
| 7H-pyrrolo[3,2-e][1,2,4]triazolo[1,5-c]pyrimidine-8- | | | | |
| carboxylate | | | | |
| 5-amino-9-chloro-7-(2-(4-(5-fluoro-2-methylpyridin- | +++ | c | | |
| 4-yl)piperazin-1-yl)ethyl)-2-(pyridin-2-yl)-7H- | | | | |
| pyrrolo[3,2-e][1,2,4]triazolo[1,5-c]pyrimidine-8- | | | | |
| carboxamide | | | | |
| 5-amino-9-chloro-7-(2-(4-(5-fluoro-2-methylpyridin- | +++ | a | | |
| 4-yl)piperazin-1-yl)ethyl)-2-(pyridin-2-yl)-7H- | | | | |
| pyrrolo[3,2-e][1,2,4]triazolo[1,5-c]pyrimidine-8- | | | | |
| carboxylic acid | | | | |
| 2-(4-(4-(2-(5-amino-8-(pyridin-2-yl)-3H- | +++ | b | | |
| [1,2,4]triazolo[5,1-i]purin-3-yl)ethyl)piperazin-1-yl)- | | | | |
| 3-fluorophenoxy)-2-methylpropanoic acid | | | | |
| methyl 2-(4-(4-(2-(5-amino-8-(prop-l-yn-1-yl)-3H- | +++ | a | | |
| [1,2,4]triazolo[5,1-i]purin-3-yl)ethyl)piperazin-1-yl)- | | | | |
| 3-fluorophenoxy)-2-methylpropanoate | | | | |
| 8-(cyclopropylethynyl)-3-(2-(4-(2-fluoro-5-(oxazol- | +++ | b | | |
| 2-yl)phenyl)piperazin-1-yl)ethyl)-3H- | | | | |
| [1,2,4]triazolo[5,1-i]purin-5-amine | | | | |
| 5-amino-9-chloro-7-(2-(3-methyl-7,8-dihydro-1,6- | +++ | c | | |
| naphthyridin-6(5H)-yl)ethyl)-2-(pyridin-2-yl)-7H- | | | | |
| pyrrolo[3,2-e][1,2,4]triazolo[1,5-c]pyrimidine-8- | | | | |
| carboxamide | | | | |
| 3-(2-(3-(oxetan-3-yl)-7,8-dihydro-1,6-naphthyridin- | ++ | c | | |
| 6(5H)-yl)ethyl)-8-(pyridin-2-yl)-3H- | | | | |
| [1,2,4]triazolo[5,1-i]purin-5-amine | | | | |
| 2-(4-(4-(2-(5-amino-8-(prop-1-yn-1-yl)-3H- | ++ | b | | |
| [1,2,4]triazolo[5,1-i]purin-3-yl)ethyl)piperazin-1- | | | | |
| yl)phenoxy)-2-methylpropanoic acid | | | | |
| 9-chloro-7-(2-(3-methyl-7,8-dihydro-1,6- | +++ | c | | |
| naphthyridin-6(5H)-yl)ethyl)-2-(pyridin-2-yl)-7H- | | | | |
| pyrazolo[4,3-e][1,2,4]triazolo[1,5-c]pyrimidin-5- | | | | |
| amine | | | | |
| 8-(prop-l-yn-1-yl)-3-(2-(3-(thiazol-2-yl)-7,8- | +++ | c | | |
| dihydro-1,6-naphthyridin-6(5H)-yl)ethyl)-3H- | | | | |
| [1,2,4]triazolo[5,1-i]purin-5-amine | | | | |
| 3-(2-(3-(oxetan-3-yl)-7,8-dihydro-l,6-naphthyridin- | ++ | b | | |
| 6(5H)-yl)ethyl)-8-(prop-1-yn-1-yl)-3H- | | | | |
| [1,2,4]triazolo[5,1-i]purin-5-amine | | | | |
| 8-(pyridin-2-yl)-3-(2-(3-(thiazol-2-yl)-7,8-dihydro- | +++ | c | | |
| l,6-naphthyridin-6(5H)-yl)ethyl)-3H- | | | | |
| [1,2,4]triazolo[5,1-i]purin-5-amine | | | | |
| 3-(2-(4-(2-fluoro-5-(IH-pyrazol-1- | +++ | a | | |
| yl)phenyl)piperazin-1-yl)ethyl)-8-(prop-1-yn-1-yl)- | | | | |
| 3H-[1,2,4]triazolo[5,1-i]purin-5-amine | | | | |
| 3-(2-(4-(2-fluoro-5-(1H-pyrazol-1- | +++ | b | | |
| yl)phenyl)piperazin-1-yl)ethyl)-8-(pyridin-2-yl)-3H- | | | | |
| [1,2,4]triazolo[5,1-i]purin-5-amine | | | | |

+++ = less than or equal to 20 nM;
++ = greater than 20 nM but less than or equal to 100 nM;
+ = active but more than 100 nM;
a = greater than 1000;
b = greater than 100 but less than or equal to 1000;
c = greater than 0 but less than or equal to 100.

The examples and embodiments described herein are for illustrative purposes only and various modifications or changes suggested to persons skilled in the art are to be included within the spirit and purview of this application and scope of the appended claims.

1. A compound of Formula (X), or a pharmaceutically acceptable salt or solvate thereof: 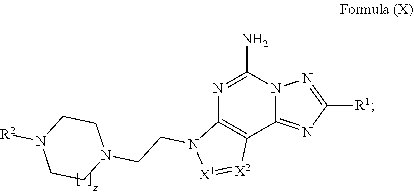
wherein:
X¹═X² is —C(R³)═N—, —N═C(R⁴)—, —C(R⁵)═C(R⁶)—, or —N═N—;
R¹ is 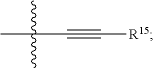
or
R¹ is a 6-membered heteroaryl ring optionally substituted with m R^{7a} groups;
m is 0, 1, 2, 3, or 4;
R² is phenyl or a monocyclic or bicyclic heteroaryl ring, wherein the phenyl or monocyclic or bicyclic heteroaryl ring is optionally substituted with n R^{7b};
n is 0, 1, 2, 3, 4, or 5;
R³ is H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, or —C(═O)N(R⁹)S(═O)₂R¹⁰;
R⁴ is H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, or —C(═O)N(R⁹)S(═O)₂R¹⁰;
R⁵ and R⁶ are each independently selected from H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, and
—C(═O)N(R⁹)S(═O)₂R¹⁰; wherein at least one of R⁵ and R⁶ is not hydrogen;
each R^{7a} is independently selected from hydrogen, halogen, —CN, C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁹, —SR⁹, —N(R⁹)₂, —C(O)OR⁹, —C(O)N(R⁹)₂, —OC(O)N(R⁹)₂,
—N(R¹¹)C(O)N(R⁹)₂, —N(R¹¹)C(O)OR¹⁰, —N(R¹¹)C(O)R¹⁰, —N(R¹¹)S(O)₂R¹⁰, —C(O)R¹⁰, —S(O)R¹⁰, —S(O)₂R¹⁰, —S(O)₂N(R⁹)₂, and —OC(O)R¹⁰, wherein C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R⁸;
each R^{7b} is independently selected from hydrogen, halogen, —CN, C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁹, —SR⁹, —N(R⁹)₂, —C(O)OR⁹, —C(O)N(R⁹)₂, —OC(O)N(R⁹)₂, —N(R¹¹)C(O)N(R⁹)₂, —N(R¹¹)C(O)OR¹⁰, —N(R¹¹)C(O)R¹⁰, —N(R¹¹)S(O)₂R¹⁰, —C(O)R¹⁰, —S(O)R¹⁰, —S(O)₂R¹⁰, —S(O)₂N(R⁹)₂, and —OC(O)R¹⁰, wherein C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R⁸;
or two R^{7b} on adjacent atoms of R² are joined together with the intervening atoms connecting the adjacent R^{7b} groups to form a phenyl, a 5-membered heteroaryl or a 6-membered heteroaryl, wherein the phenyl, the 5-membered heteroaryl or the 6-membered heteroaryl are optionally substituted with one, two, or three R⁸;
each R⁸ is independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀-aryl, —CH₂—C₆₋₁₀aryl, C₁₋₉heteroaryl, —OR¹², —SR¹², —N(R¹²)₂, —C(O)OR¹², —C(O)N(R¹²)₂, —C(O)C(O)N(R¹²)₂, —OC(O)N(R¹²)₂, —N(R¹⁴)C(O)N(R¹)₂, —N(R¹⁴)C(O)OR¹³, —N(R¹⁴)C(O)R¹³, —N(R¹⁴)S(O)₂R¹³, —C(O)R¹³, —S(O)₂R¹³, —S(O)₂N(R¹²)₂, and —OC(O)R¹³, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹², —SR¹², —N(R¹²)₂, —C(O)OR¹², —C(O)N(R¹²)₂, —C(O)C(O)N(R¹²)₂, —OC(O)N(R¹²)₂, —N(R¹⁴)C(O)N(R¹)₂, —N(R¹⁴)C(O)OR¹³, —N(R¹⁴)C(O)R¹³, —N(R¹⁴)S(O)₂R¹³, —C(O)R¹³, —S(O)₂R¹³, —S(O)₂N(R¹²)₂, and —OC(O)R¹³;
each R⁹ is independently selected from H, C₁₋₆alkyl, and C₃₋₆cycloalkyl;
or two R⁹ attached to the same N atom are taken together with the N atom to which they are attached to form an optionally substituted C₂₋₆heterocycloalkyl
each R¹⁰ is independently selected from H, C₁₋₆alkyl and C₃₋₆cycloalkyl;
each R¹¹ is independently selected from H and C₁₋₆alkyl;
each R¹² is independently selected from H, C₁₋₆alkyl, and C₃₋₆cycloalkyl;
each R¹³ is independently selected from H, C₁₋₆alkyl and C₃₋₆cycloalkyl;
each R¹⁴ is independently selected from H and C₁₋₆alkyl;
R¹⁵ is H, C₁-C₆alkyl, or C₃₋₆cycloalkyl; and
z is 1 or 2.

2. The compound of claim 1, wherein the compound has the structure of Formula (I), or a pharmaceutically acceptable salt or solvate thereof: 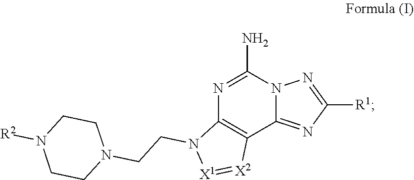
wherein:
X¹═X² is —C(R³)═N—, —N═C(R⁴)—, —C(R⁵)═C(R⁶)—, or —N═N—;
R¹ is 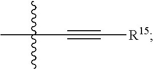
or
R¹ is a 6-membered heteroaryl ring optionally substituted with m R^{7a} groups;
m is 0, 1, 2, 3, or 4;
R² is phenyl or a monocyclic or bicyclic heteroaryl ring, wherein the phenyl or monocyclic or bicyclic heteroaryl ring is optionally substituted with n R^{7b};
n is 0, 1, 2, 3, 4, or 5;
R³ is H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, or —C(═O)N(R⁹)S(═O)₂R¹⁰;
R⁴ is H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, or —C(═O)N(R⁹)S(═O)₂R¹⁰;
R⁵ and R⁶ are each independently selected from H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, and —C(═O)N(R⁹)S(═O)₂R¹⁰; wherein at least one of R⁵ and R⁶ is not hydrogen;
each R^{7a} is independently selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁹, —SR⁹, —N(R⁹)₂, —C(O)OR⁹, —C(O)N(R⁹)₂, —OC(O)N(R⁹)₂, —N(R¹¹)C(O)N(R⁹)₂, —N(R¹¹)C(O)OR¹⁰, —N(R¹¹)C(O)R¹⁰, —N(R¹¹)S(O)₂R¹⁰, —C(O)R¹⁰, —S(O)R¹⁰, —S(O)₂R¹⁰, —S(O)₂N(R⁹)₂, and —OC(O)R¹⁰, wherein C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R⁸;
each R^{7b} is independently selected from hydrogen, halogen, —CN, C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁹, —SR⁹, —N(R⁹)₂, —C(O)OR⁹, —C(O)N(R⁹)₂, —OC(O)N(R⁹)₂, —N(R¹¹)C(O)N(R⁹)₂, —N(R¹¹)C(O)OR¹⁰, —N(R¹¹)C(O)R¹⁰, —N(R¹¹)S(O)₂R¹⁰, —C(O)R¹⁰, —S(O)R¹⁰, —S(O)₂R¹⁰, —S(O)₂N(R⁹)₂, and —OC(O)R¹⁰, wherein C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R⁸;
or two R^{7b} on adjacent atoms of R² are joined together with the intervening atoms connecting the adjacent R^{7b} groups to form a phenyl, a 5-membered heteroaryl or a 6-membered heteroaryl, wherein the phenyl, the 5-membered heteroaryl or the 6-membered heteroaryl are optionally substituted with one, two, or three R⁸;
each R⁸ is independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀-aryl, —CH₂—C₆₋₁₀aryl, C₁₋₉heteroaryl, —OR¹², —SR¹², —N(R¹²)₂, —C(O)OR¹², —C(O)N(R¹²)₂, —C(O)C(O)N(R¹²)₂, —OC(O)N(R¹²)₂, —N(R¹⁴)C(O)N(R¹)₂, —N(R¹⁴)C(O)OR¹³, —N(R¹⁴)C(O)R¹³, —N(R¹⁴)S(O)₂R¹³, —C(O)R¹³, —S(O)₂R¹³, —S(O)₂N(R¹²)₂, and —OC(O)R¹³, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹², —SR¹², —N(R¹²)₂, —C(O)OR¹², —C(O)N(R¹²)₂, —C(O)C(O)N(R¹²)₂, —OC(O)N(R¹²)₂, —N(R¹⁴)C(O)N(R¹²)₂, —N(R¹⁴)C(O)OR¹³, —N(R¹⁴)C(O)R¹³, —N(R¹⁴)S(O)₂R¹³, —C(O)R¹³, —S(O)₂R¹³, —S(O)₂N(R¹²)₂, and —OC(O)R¹³;
each R⁹ is independently selected from H, C₁₋₆alkyl, and C₃₋₆cycloalkyl;
or two R⁹ attached to the same N atom are taken together with the N atom to which they are attached to form an optionally substituted C₂₋₆heterocycloalkyl;
each R¹⁰ is independently selected from H, C₁₋₆alkyl and C₃₋₆cycloalkyl;
each R¹¹ is independently selected from H and C₁₋₆alkyl;
each R¹² is independently selected from H, C₁₋₆alkyl, and C₃₋₆cycloalkyl;
each R¹³ is independently selected from H, C₁₋₆alkyl and C₃₋₆cycloalkyl;
each R¹⁴ is independently selected from H and C₁₋₆alkyl; and
R¹⁵ is H, C₁-C₆alkyl, or C₃₋₆cycloalkyl.

3. The compound of claim 1, or a pharmaceutically acceptable salt or solvate thereof, wherein:
R¹ is

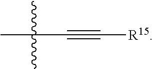

4. The compound of claim 1, or a pharmaceutically acceptable salt or solvate thereof, wherein the compound has the structure of Formula (Ib), or a pharmaceutically acceptable salt or solvate thereof:

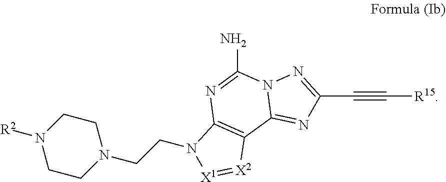

5. The compound of claim 1, or a pharmaceutically acceptable salt or solvate thereof, wherein:
R¹⁵ is C₁-C₆alkyl or C₃₋₆cycloalkyl.

6. The compound of claim 5, or a pharmaceutically acceptable salt or solvate thereof, wherein:
R¹⁵ is —CH₃ or cyclopropyl.

7. The compound of claim 1, or a pharmaceutically acceptable salt or solvate thereof, wherein:
R¹ is a 6-membered heteroaryl ring optionally substituted with m R^{7a}.

8. The compound of claim 1, or a pharmaceutically acceptable salt or solvate thereof, wherein:
R¹ is a pyridinyl optionally substituted with m R^{7a}, pyrimidinyl optionally substituted with m R^{7a}, pyrazinyl optionally substituted with m R^{7a}, pyridazinyl optionally substituted with m R^{7a}, or triazinyl optionally substituted with m R^{7a}.

9. The compound of claim 1, or a pharmaceutically acceptable salt or solvate thereof, wherein:
R¹ is a pyridinyl optionally substituted with m R^{7a}.

10. The compound of claim 1, or a pharmaceutically acceptable salt or solvate thereof, wherein:
R¹ is

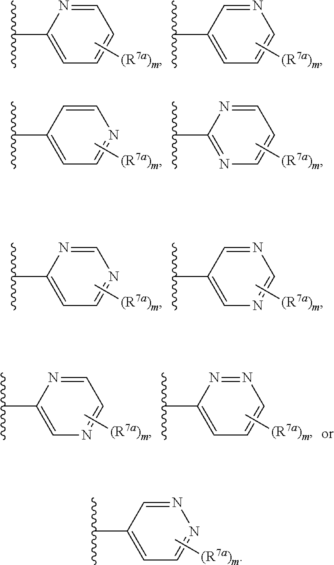

11. The compound of claim 1, or a pharmaceutically acceptable salt or solvate thereof, wherein the compound has the structure of Formula (Ia), or a pharmaceutically acceptable salt or solvate thereof: 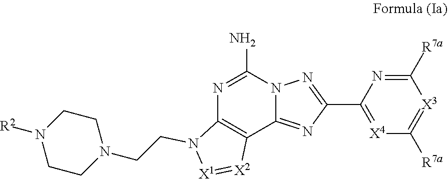
wherein,
X³ is CR^{7a} or N;
X⁴ is CR^{7a} or N.

12. The compound of claim 1, or a pharmaceutically acceptable salt or solvate thereof, wherein m is 0.

13. The compound of claim 1, or a pharmaceutically acceptable salt or solvate thereof, wherein:
X¹═X² is —C(R³)═N—.

14. The compound of claim 13, or a pharmaceutically acceptable salt or solvate thereof, wherein the compound has the structure of Formula (IIa), or a pharmaceutically acceptable salt or solvate thereof:

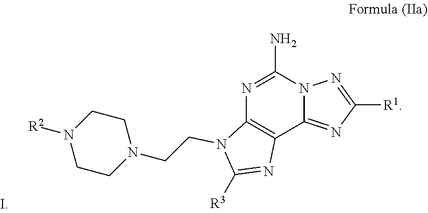

15. The compound of claim 1, or a pharmaceutically acceptable salt or solvate thereof, wherein:
R³ is H, halogen, C₁-C₆alkyl, C₃₋₆cycloalkyl, —CN, —C(═O)N(R⁹)₂, or —C(═O)N(R⁹)S(═O)₂R¹⁰.

16. The compound of claim 15, or a pharmaceutically acceptable salt or solvate thereof, wherein:
R³ is H, C₁-C₆alkyl, C₃₋₆cycloalkyl, —CN, —C(═O)N(R⁹)₂, or —C(═O)N(R⁹)S(═O)₂R¹⁰.

17. The compound of claim 1, or a pharmaceutically acceptable salt or solvate thereof, wherein:
X¹═X² is —N═C(R⁴)—.

18. The compound of claim 17, or a pharmaceutically acceptable salt or solvate thereof, wherein the compound has the structure of Formula (IIb), or a pharmaceutically acceptable salt or solvate thereof:

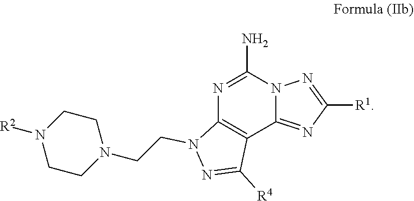

19. The compound of claim 17, or a pharmaceutically acceptable salt or solvate thereof, wherein:
R⁴ is halogen, C₁-C₆alkyl, C₃₋₆cycloalkyl, —CN, —CO₂R⁹, —C(═O)N(R⁹)₂, or —C(═O)N(R⁹)S(═O)₂R¹⁰.

20. The compound of claim 17, or a pharmaceutically acceptable salt or solvate thereof, wherein:
R⁴ is halogen, C₁-C₆alkyl, or C₃₋₆cycloalkyl.

21. The compound of claim 1, or a pharmaceutically acceptable salt or solvate thereof, wherein:
X¹═X² is —C(R⁵)═C(R⁶)—.

22. The compound of claim 21, or a pharmaceutically acceptable salt or solvate thereof, wherein the compound has the structure of Formula (IIc), or a pharmaceutically acceptable salt or solvate thereof:

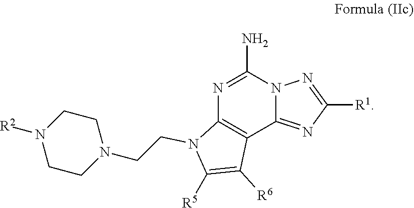

23. The compound of claim 21, or a pharmaceutically acceptable salt or solvate thereof, wherein:
R⁵ and R⁶ are each independently selected from H, halogen, C₁-C₆alkyl, C₃₋₆cycloalkyl, —CN, —CO₂R⁹, —C(═O)N(R⁹)₂, and —C(═O)N(R⁹)S(═O)₂R¹⁰.

24. The compound of claim 1, or a pharmaceutically acceptable salt or solvate thereof, wherein:
R⁵ is halogen, C₁-C₆alkyl, C₃₋₆cycloalkyl, —CN, —CO₂R⁹, —C(═O)N(R⁹)₂, or —C(═O)N(R⁹)S(═O)₂R¹⁰.

25. The compound of claim 1, or a pharmaceutically acceptable salt or solvate thereof, wherein:
R⁵ is —CN, —CO₂H, —CO₂CH₃, or —C(═O)NH₂.

26. The compound of claim 1, or a pharmaceutically acceptable salt or solvate thereof, wherein:
R⁶ is H, C₁ or CH₃.

27. The compound of claim 1, or a pharmaceutically acceptable salt or solvate thereof, wherein:
X¹═X² is —N═N—.

28. The compound of claim 1, or a pharmaceutically acceptable salt or solvate thereof, wherein:
R² is phenyl optionally substituted with one, two, or three R^{7b}.

29. The compound of claim 1, or a pharmaceutically acceptable salt or solvate thereof, wherein:
R² is

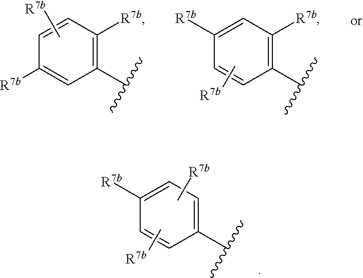

30. The compound of claim 1, or a pharmaceutically acceptable salt or solvate thereof, wherein:
R² is

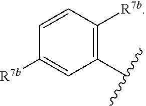

31. The compound of claim 1, or a pharmaceutically acceptable salt or solvate thereof, wherein:
R² is a monocyclic or bicyclic heteroaryl ring optionally substituted with one, two, or three R^{7b}.

32. The compound of claim 1, or a pharmaceutically acceptable salt or solvate thereof, wherein:
R² is a monocyclic heteroaryl ring selected from oxazolyl, thiazolyl, pyrazolyl, furanyl, thienyl, pyrrolyl, imidazolyl, triazolyl, tetrazolyl, isoxazolyl, isothiazolyl, oxadiazolyl, thiadiazolyl, pyridinyl, pyrimidinyl, pyrazinyl, and pyridazinyl.

33. The compound of claim 1, or a pharmaceutically acceptable salt or solvate thereof, wherein:
R² is

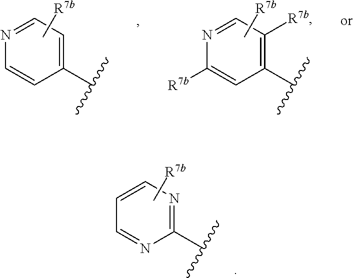

34. The compound of claim 1, or a pharmaceutically acceptable salt or solvate thereof, wherein:
R² is a bicyclic heteroaryl ring selected from indolyl, benzofuranyl, benzothienyl, benzoxazolyl, benzisoxazolyl, benzimidazolyl, imidazopyrdinyl, imidazopyridazinyl, purinyl, quinolinyl, quinazolinyl, and pyridopyrimidinyl.

35. The compound of claim 34, or a pharmaceutically acceptable salt or solvate thereof, wherein:
R² is

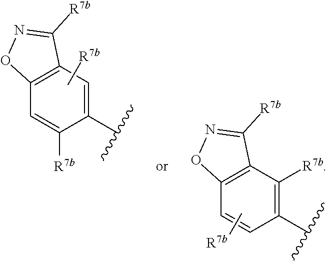

36. The compound of claim 1, or a pharmaceutically acceptable salt or solvate thereof, wherein the compound has the structure of Formula (IIIa), Formula (IIIb), or Formula (IIIc), or a pharmaceutically acceptable salt or solvate thereof: 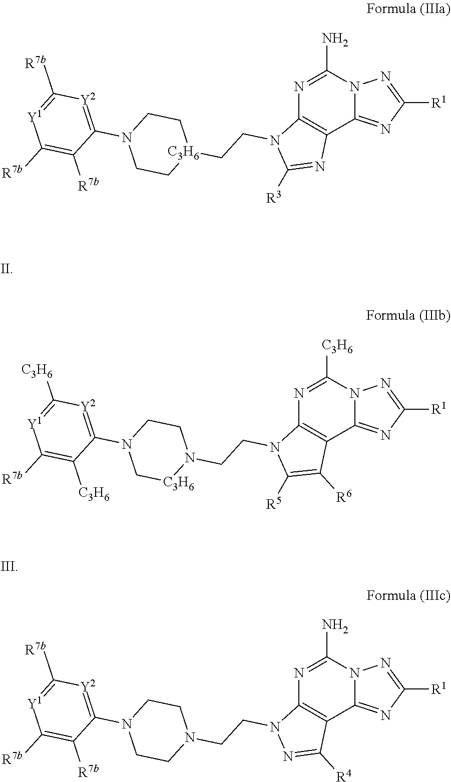
wherein:
Y¹ is CH, CR^{7b} or N; and Y² is CH, CR^{7b} or N.

37. The compound of claim 1, or a pharmaceutically acceptable salt or solvate thereof, wherein the compound has the structure of Formula (IVa), Formula (IVb), Formula (IVc), or a pharmaceutically acceptable salt or solvate thereof:

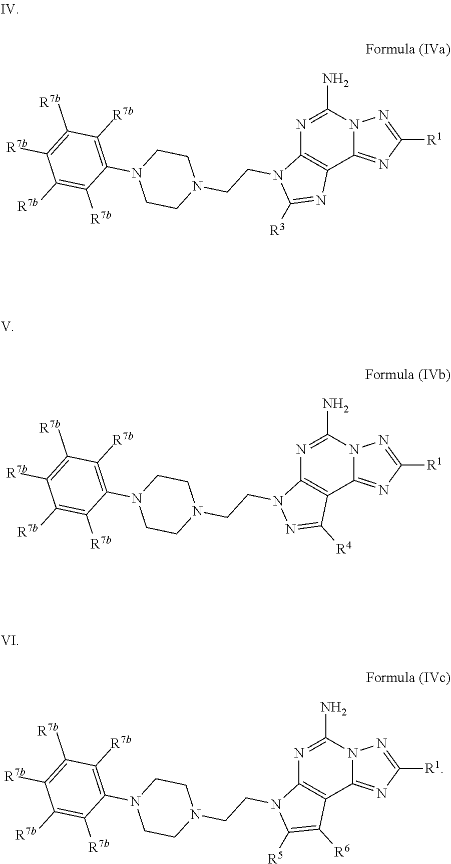

38. The compound of claim 1, or a pharmaceutically acceptable salt or solvate thereof, wherein:
each R^{7b} is independently selected from hydrogen, halogen, —CN, C₁₋₆alkyl, C₁₋₆alkoxy, phenyl, 5-membered C₁₋₄heteroaryl, 6-membered C₁₋₅heteroaryl, —OR⁹, —C(O)OR⁹, —C(O)N(R⁹)₂, —C(O)R¹⁰, and —S(O)₂N(R⁹)₂, wherein C₁₋₆alkyl, C₁₋₆alkoxy, phenyl, 5-membered C₁₋₄heteroaryl, and 6-membered C₁₋₅heteroaryl are optionally substituted with one, two, or three R⁸.

39. The compound of claim 1, or a pharmaceutically acceptable salt or solvate thereof, wherein:
each R⁸ is independently selected from halogen, —CN, C₁₋₆alkyl, —OR¹², —C(O)OR¹², and —N(R¹⁴)S(O)₂R¹³, wherein C₁₋₆alkyl is optionally substituted with one, two, or three groups independently selected from oxo, C₁₋₆alkyl, C₁₋₆alkoxy, —OR¹², —C(O)OR¹², and —N(R¹⁴)S(O)₂R¹³.

40. The compound of claim 1, or a pharmaceutically acceptable salt or solvate thereof, wherein:
R² is

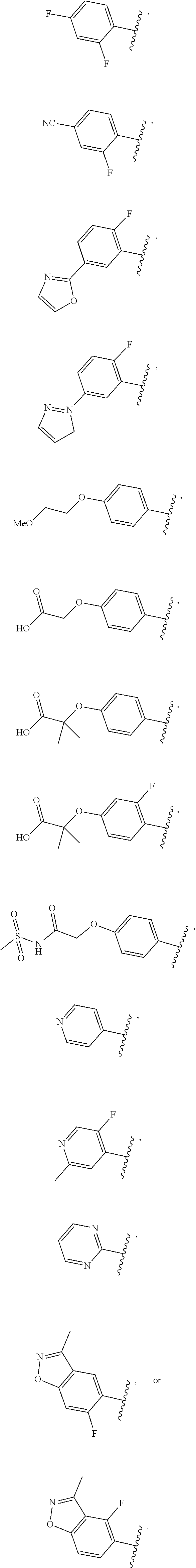

41. A compound of Formula (XI), or a pharmaceutically acceptable salt or solvate thereof: 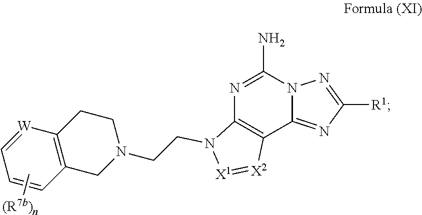
wherein:
X¹═X² is —C(R³)═N—, —N═C(R⁴)—, —C(R⁵)═C(R⁶)—, or —N═N—;
R¹ is 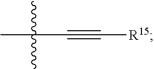
or
R¹ is a 6-membered heteroaryl ring optionally substituted with m R^{7a} groups;
m is 0, 1, 2, 3, or 4;
R³ is H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, or —C(═O)N(R⁹)S(═O)₂R¹⁰;
R⁴ is H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, or —C(═O)N(R⁹)S(═O)₂R¹⁰;
R⁵ and R⁶ are each independently selected from H, halogen, C₁-C₆alkyl, C₁-C₆haloalkyl, C₁-C₆heteroalkyl, C₃₋₆cycloalkyl, —CN, —CO₂R⁹, —C(═O)N(R⁹)₂, and —C(═O)N(R⁹)S(═O)₂R¹⁰; wherein at least one of R⁵ and R⁶ is not hydrogen;
each R^{7a} is independently selected from halogen, —CN, C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁹, —SR⁹, —N(R⁹)₂, —C(O)OR⁹, —C(O)N(R⁹)₂, —OC(O)N(R⁹)₂, —N(R¹¹)C(O)N(R⁹)₂, —N(R¹¹)C(O)OR¹⁰, —N(R¹¹)C(O)R¹⁰, —N(R¹¹)S(O)₂R¹⁰, —C(O)R¹⁰, —S(O)R¹⁰, —S(O)₂R¹⁰, —S(O)₂N(R⁹)₂, and —OC(O)R¹⁰, wherein C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R⁸;
each R^{7b} is independently selected from halogen, —CN, C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁹, —SR⁹, —N(R⁹)₂, —C(O)OR⁹, —C(O)N(R⁹)₂, —OC(O)N(R⁹)₂, —N(R¹¹)C(O)N(R⁹)₂, —N(R¹¹)C(O)OR¹⁰, —N(R¹¹)C(O)R¹⁰, —N(R¹¹)S(O)₂R¹⁰, —C(O)R¹⁰, —S(O)R¹⁰, —S(O)₂R¹⁰, —S(O)₂N(R⁹)₂, and —OC(O)R¹⁰, wherein C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R⁸;
n is 0, 1, 2, or 3;
or two R^{7b} on adjacent atoms of R² are joined together with the intervening atoms connecting the adjacent R^{7b} groups to form a phenyl, a 5-membered heteroaryl or a 6-membered heteroaryl, wherein the phenyl, the 5-membered heteroaryl or the 6-membered heteroaryl are optionally substituted with one, two, or three R⁸;
W is CR^{7c} or N;
R^{7c} is selected from hydrogen, halogen, —CN, C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁹, —SR⁹, —N(R⁹)₂, —C(O)OR⁹, —C(O)N(R⁹)₂, —OC(O)N(R⁹)₂, —N(R¹¹)C(O)N(R⁹)₂, —N(R¹¹)C(O)OR¹⁰, —N(R¹¹)C(O)R¹⁰, —N(R¹¹)S(O)₂R¹⁰, —C(O)R¹⁰, —S(O)R¹⁰, —S(O)₂R¹⁰, —S(O)₂N(R⁹)₂, and —OC(O)R¹⁰, wherein C₁₋₆alkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R⁸;
each R⁸ is independently selected from halogen, —CN, C₁₋₆alkyl, C₂₋₆alkenyl, C₂₋₆alkynyl, C₃-6cycloalkyl, —CH₂—C₃₋₆cycloalkyl, C₂₋₉heterocycloalkyl, —CH₂—C₂₋₉heterocycloalkyl, C₆₋₁₀-aryl, —CH₂—C₆₋₁₀aryl, C₁₋₉heteroaryl, —OR¹², —SR¹², —N(R¹²)₂, —C(O)OR¹², —C(O)N(R¹²)₂, —C(O)C(O)N(R¹²)₂, —OC(O)N(R¹²)₂, —N(R¹⁴)C(O)N(R¹²)₂, —N(R¹⁴)C(O)OR¹³, —N(R¹⁴)C(O)R¹³, —N(R¹⁴)S(O)₂R¹³, —C(O)R¹³, —S(O)₂R¹³, —S(O)₂N(R¹²)₂, and —OC(O)R¹³, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹², —SR¹², —N(R¹²)₂, —C(O)OR¹², —C(O)N(R¹²)₂, —C(O)C(O)N(R¹²)₂, —OC(O)N(R¹²)₂, —N(R¹⁴)C(O)N(R¹²)₂, —N(R¹⁴)C(O)OR¹³, —N(R¹⁴)C(O)R¹³, —N(R¹⁴)S(O)₂R¹³, —C(O)R¹³, —S(O)₂R¹³, —S(O)₂N(R¹²)₂, and —OC(O)R¹³;
each R⁹ is independently selected from H, C₁₋₆alkyl, and C₃₋₆cycloalkyl;
or two R⁹ attached to the same N atom are taken together with the N atom to which they are attached to form an optionally substituted C₂₋₆heterocycloalkyl
each R¹⁰ is independently selected from H, C₁₋₆alkyl and C₃₋₆cycloalkyl;
each R¹¹ is independently selected from H and C₁₋₆alkyl;
each R¹² is independently selected from H, C₁₋₆alkyl, and C₃₋₆cycloalkyl;
each R¹³ is independently selected from H, C₁₋₆alkyl and C₃₋₆cycloalkyl;
each R¹⁴ is independently selected from H and C₁₋₆alkyl; and
R¹⁵ is H, C₁-C₆alkyl, or C₃₋₆cycloalkyl.

42. The compound of claim 41, or a pharmaceutically acceptable salt or solvate thereof, wherein:
W is N.

43. The according to claim 41, or a pharmaceutically acceptable salt or solvate thereof, wherein:

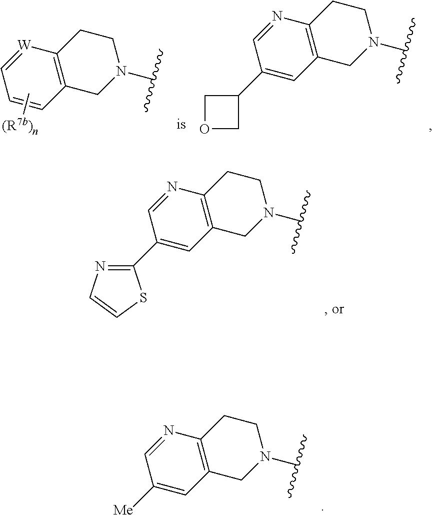

44. The compound of claim 1, or a pharmaceutically acceptable salt or solvate thereof, wherein:
R¹ is

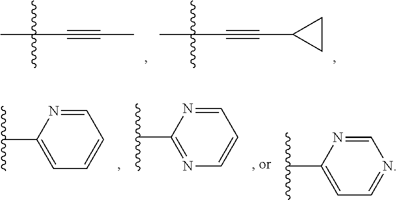

45. A compound that has one of the following structures: 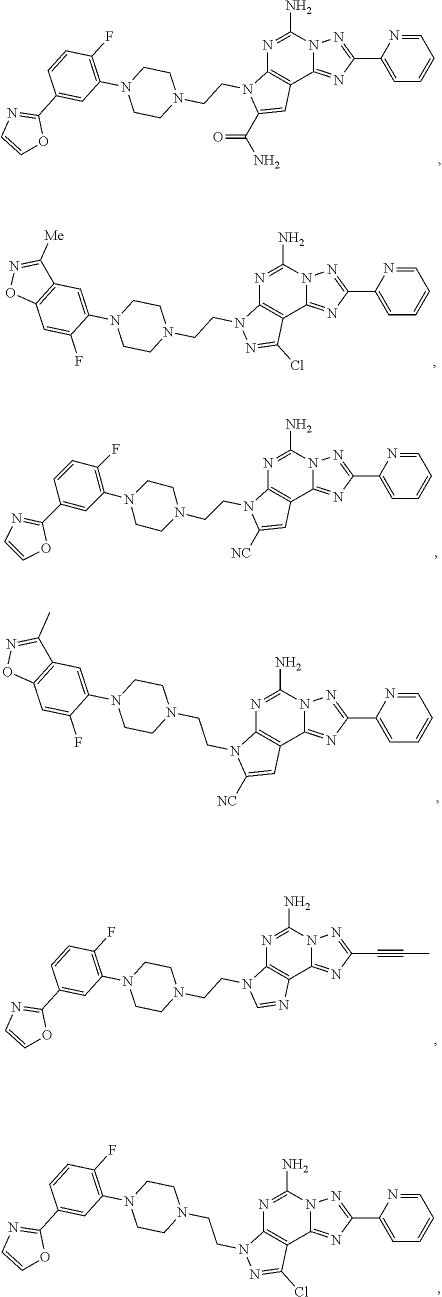 [SMILES: NC(=O)c1cc2c(nc(N)n3nc(-c4ccccn4)nc23)n1CCN1CCN(c2cc(-c3ncco3)ccc2F)CC1 ; Cc1noc2cc(F)c(N3CCN(CCn4nc(Cl)c5c4nc(N)n4nc(-c6ccccn6)nc54)CC3)cc12 ; N#Cc1cc2c(nc(N)n3nc(-c4ccccn4)nc23)n1CCN1CCN(c2cc(-c3ncco3)ccc2F)CC1 ; Cc1noc2cc(F)c(N3CCN(CCn4c(C#N)cc5c4nc(N)n4nc(-c6ccccn6)nc54)CC3)cc12 ; CC#Cc1nc2c3ncn(CCN4CCN(c5cc(-c6ncco6)ccc5F)CC4)c3nc(N)n2n1 ; Nc1nc2c(c(Cl)nn2CCN2CCN(c3cc(-c4ncco4)ccc3F)CC2)c2nc(-c3ccccn3)nn12] 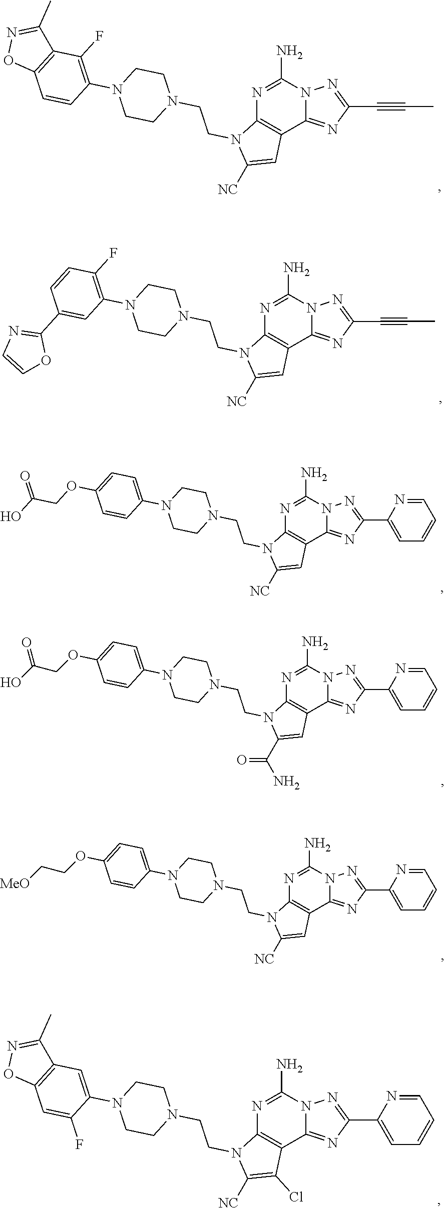 [SMILES: CC#Cc1nc2c3cc(C#N)n(CCN4CCN(c5ccc6onc(C)c6c5F)CC4)c3nc(N)n2n1 ; CC#Cc1nc2c3cc(C#N)n(CCN4CCN(c5cc(-c6ncco6)ccc5F)CC4)c3nc(N)n2n1 ; N#Cc1cc2c(nc(N)n3nc(-c4ccccn4)nc23)n1CCN1CCN(c2ccc(OCC(=O)O)cc2)CC1 ; NC(=O)c1cc2c(nc(N)n3nc(-c4ccccn4)nc23)n1CCN1CCN(c2ccc(OCC(=O)O)cc2)CC1 ; COCCOc1ccc(N2CCN(CCn3c(C#N)cc4c3nc(N)n3nc(-c5ccccn5)nc43)CC2)cc1 ; Cc1noc2cc(F)c(N3CCN(CCn4c(C#N)c(Cl)c5c4nc(N)n4nc(-c6ccccn6)nc54)CC3)cc12] 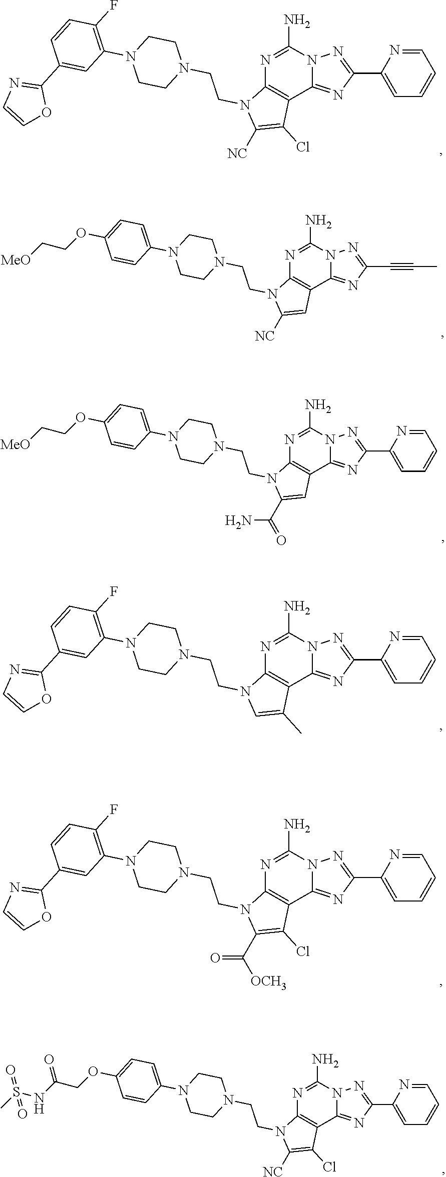 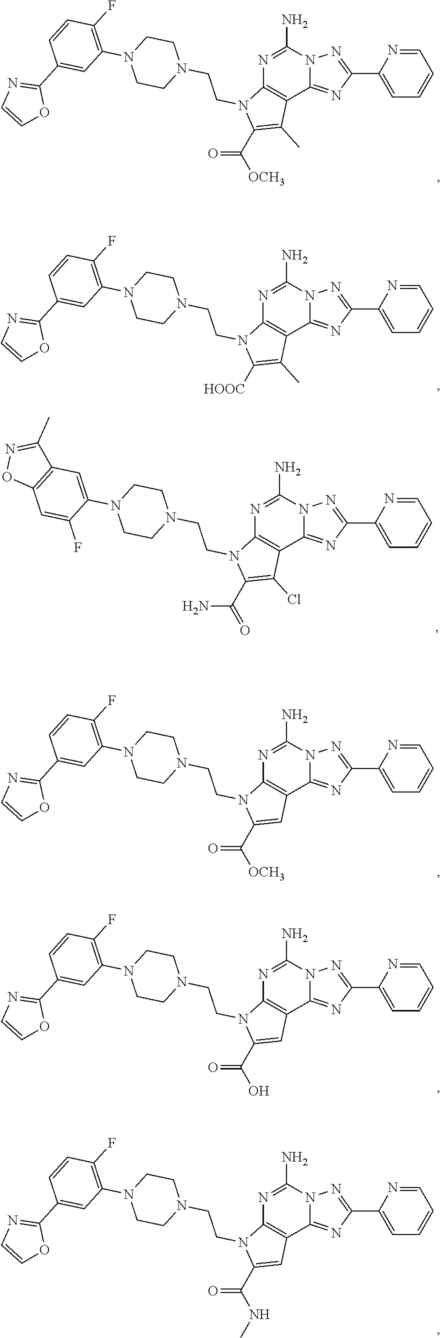 [SMILES: COC(=O)c1c(C)c2c(nc(N)n3nc(-c4ccccn4)nc23)n1CCN1CCN(c2cc(-c3ncco3)ccc2F)CC1 ; Cc1c(C(=O)O)n(CCN2CCN(c3cc(-c4ncco4)ccc3F)CC2)c2nc(N)n3nc(-c4ccccn4)nc3c12 ; Cc1noc2cc(F)c(N3CCN(CCn4c(C(N)=O)c(Cl)c5c4nc(N)n4nc(-c6ccccn6)nc54)CC3)cc12 ; COC(=O)c1cc2c(nc(N)n3nc(-c4ccccn4)nc23)n1CCN1CCN(c2cc(-c3ncco3)ccc2F)CC1 ; Nc1nc2c(cc(C(=O)O)n2CCN2CCN(c3cc(-c4ncco4)ccc3F)CC2)c2nc(-c3ccccn3)nn12 ; CNC(=O)c1cc2c(nc(N)n3nc(-c4ccccn4)nc23)n1CCN1CCN(c2cc(-c3ncco3)ccc2F)CC1] 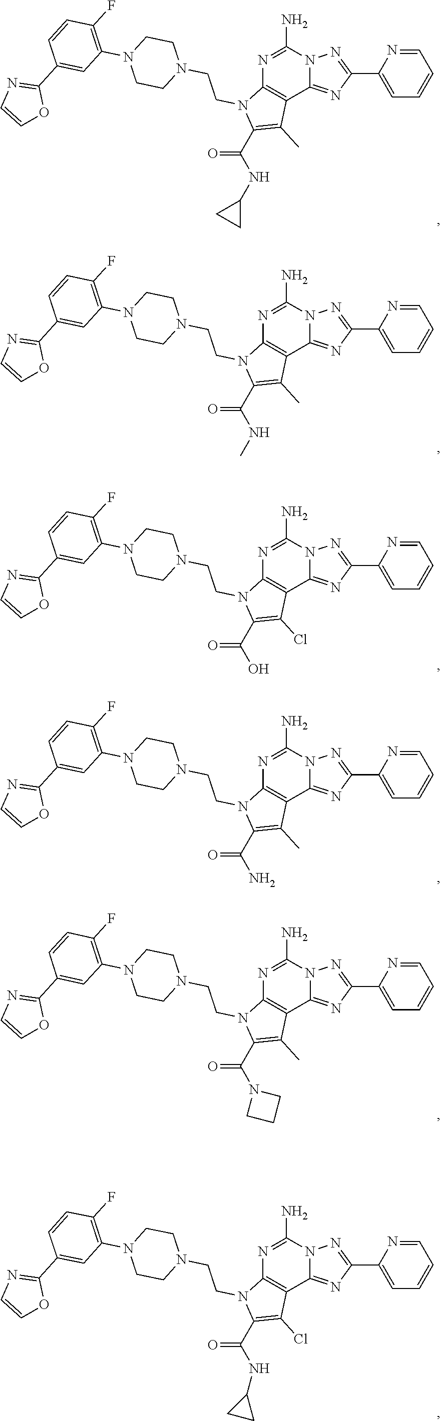 [SMILES: Cc1c(C(=O)NC2CC2)n(CCN2CCN(c3cc(-c4ncco4)ccc3F)CC2)c2nc(N)n3nc(-c4ccccn4)nc3c12 ; CNC(=O)c1c(C)c2c(nc(N)n3nc(-c4ccccn4)nc23)n1CCN1CCN(c2cc(-c3ncco3)ccc2F)CC1 ; Nc1nc2c(c(Cl)c(C(=O)O)n2CCN2CCN(c3cc(-c4ncco4)ccc3F)CC2)c2nc(-c3ccccn3)nn12 ; Cc1c(C(N)=O)n(CCN2CCN(c3cc(-c4ncco4)ccc3F)CC2)c2nc(N)n3nc(-c4ccccn4)nc3c12 ; Cc1c(C(=O)N2CCC2)n(CCN2CCN(c3cc(-c4ncco4)ccc3F)CC2)c2nc(N)n3nc(-c4ccccn4)nc3c12 ; Nc1nc2c(c(Cl)c(C(=O)NC3CC3)n2CCN2CCN(c3cc(-c4ncco4)ccc3F)CC2)c2nc(-c3ccccn3)nn12] 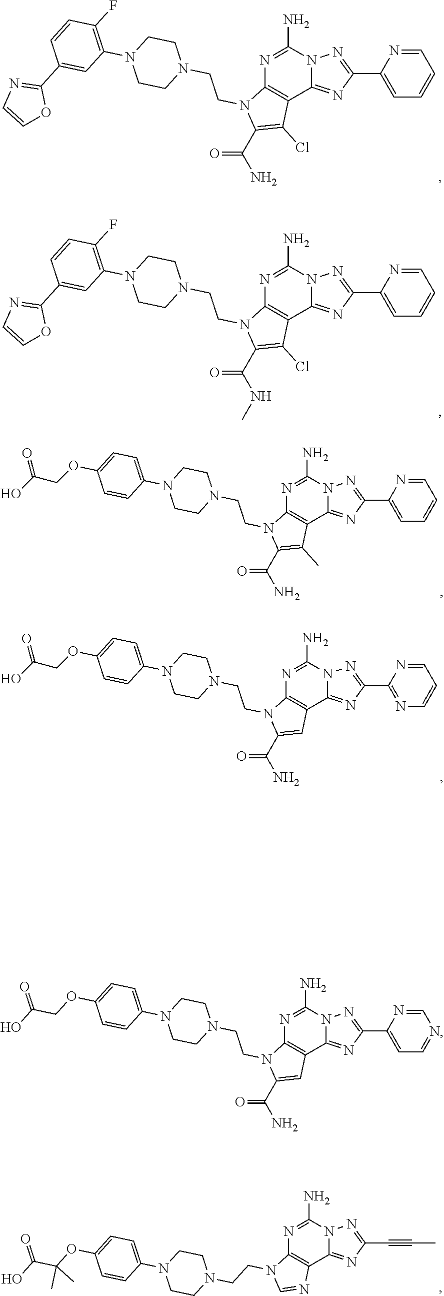 [SMILES: NC(=O)c1c(Cl)c2c(nc(N)n3nc(-c4ccccn4)nc23)n1CCN1CCN(c2cc(-c3ncco3)ccc2F)CC1 ; CNC(=O)c1c(Cl)c2c(nc(N)n3nc(-c4ccccn4)nc23)n1CCN1CCN(c2cc(-c3ncco3)ccc2F)CC1 ; Cc1c(C(N)=O)n(CCN2CCN(c3ccc(OCC(=O)O)cc3)CC2)c2nc(N)n3nc(-c4ccccn4)nc3c12 ; NC(=O)c1cc2c(nc(N)n3nc(-c4ncccn4)nc23)n1CCN1CCN(c2ccc(OCC(=O)O)cc2)CC1 ; NC(=O)c1cc2c(nc(N)n3nc(-c4ccncn4)nc23)n1CCN1CCN(c2ccc(OCC(=O)O)cc2)CC1 ; CC#Cc1nc2c3ncn(CCN4CCN(c5ccc(OC(C)(C)C(=O)O)cc5)CC4)c3nc(N)n2n1] 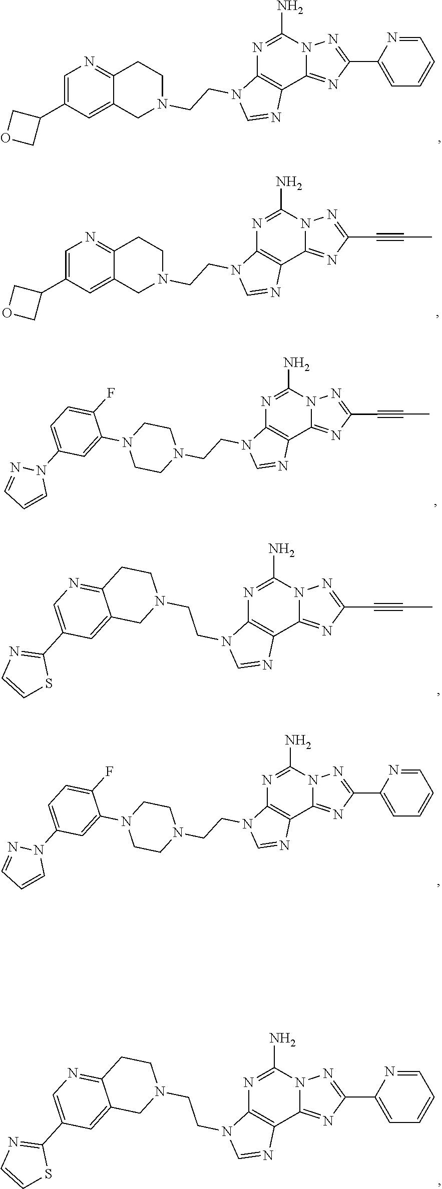 [SMILES: Nc1nc2c(ncn2CCN2CCc3ncc(C4COC4)cc3C2)c2nc(-c3ccccn3)nn12 ; CC#Cc1nc2c3ncn(CCN4CCc5ncc(C6COC6)cc5C4)c3nc(N)n2n1 ; CC#Cc1nc2c3ncn(CCN4CCN(c5cc(-n6cccn6)ccc5F)CC4)c3nc(N)n2n1 ; CC#Cc1nc2c3ncn(CCN4CCc5ncc(-c6nccs6)cc5C4)c3nc(N)n2n1 ; Nc1nc2c(ncn2CCN2CCN(c3cc(-n4cccn4)ccc3F)CC2)c2nc(-c3ccccn3)nn12 ; Nc1nc2c(ncn2CCN2CCc3ncc(-c4nccs4)cc3C2)c2nc(-c3ccccn3)nn12] 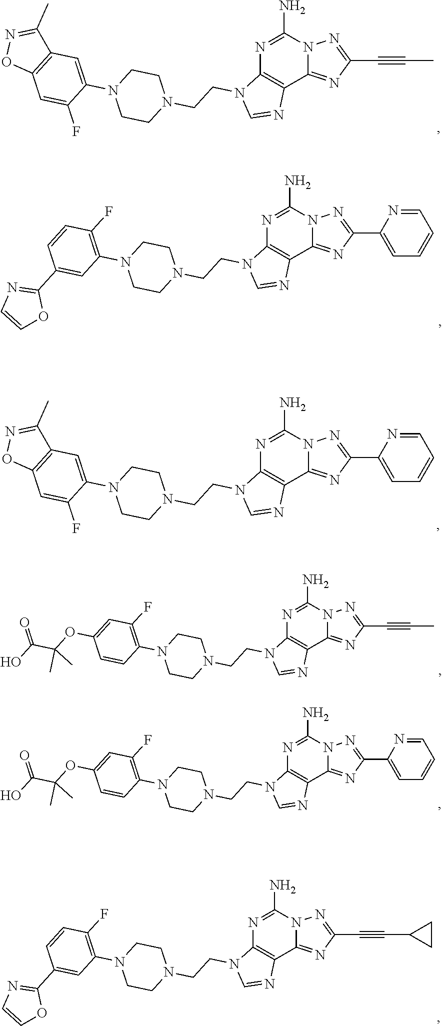 [SMILES: CC#Cc1nc2c3ncn(CCN4CCN(c5cc6c(C)noc6cc5F)CC4)c3nc(N)n2n1 ; Nc1nc2c(ncn2CCN2CCN(c3cc(-c4ncco4)ccc3F)CC2)c2nc(-c3ccccn3)nn12 ; Cc1noc2cc(F)c(N3CCN(CCn4cnc5c4nc(N)n4nc(-c6ccccn6)nc54)CC3)cc12 ; CC#Cc1nc2c3ncn(CCN4CCN(c5ccc(OC(C)(C)C(=O)O)cc5F)CC4)c3nc(N)n2n1 ; CC(C)(Oc1ccc(N2CCN(CCn3cnc4c3nc(N)n3nc(-c5ccccn5)nc43)CC2)c(F)c1)C(=O)O ; Nc1nc2c(ncn2CCN2CCN(c3cc(-c4ncco4)ccc3F)CC2)c2nc(C#CC3CC3)nn12] 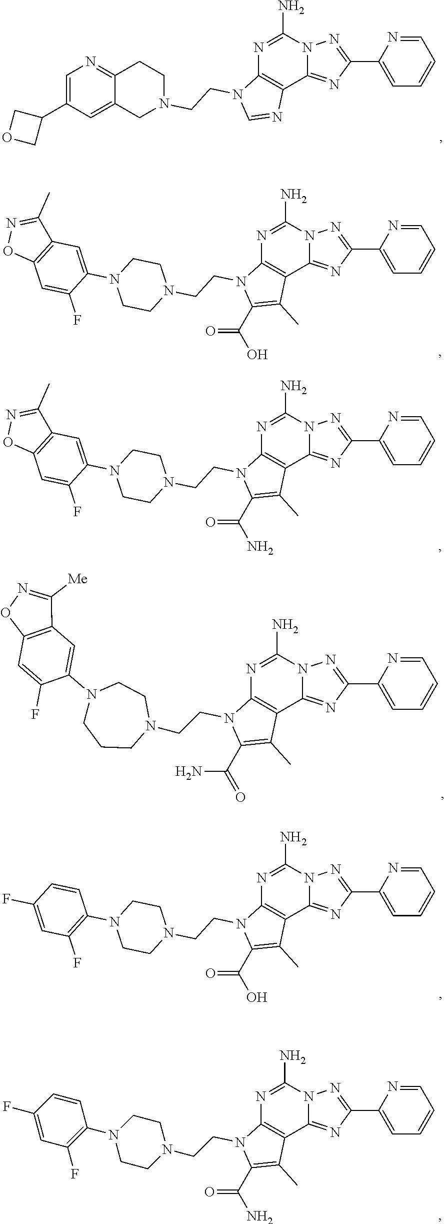 [SMILES: Nc1nc2c(ncn2CCN2CCc3ncc(C4COC4)cc3C2)c2nc(-c3ccccn3)nn12 ; Cc1noc2cc(F)c(N3CCN(CCn4c(C(=O)O)c(C)c5c4nc(N)n4nc(-c6ccccn6)nc54)CC3)cc12 ; Cc1noc2cc(F)c(N3CCN(CCn4c(C(N)=O)c(C)c5c4nc(N)n4nc(-c6ccccn6)nc54)CC3)cc12 ; Cc1noc2cc(F)c(N3CCCN(CCn4c(C(N)=O)c(C)c5c4nc(N)n4nc(-c6ccccn6)nc54)CC3)cc12 ; Cc1c(C(=O)O)n(CCN2CCN(c3ccc(F)cc3F)CC2)c2nc(N)n3nc(-c4ccccn4)nc3c12 ; Cc1c(C(N)=O)n(CCN2CCN(c3ccc(F)cc3F)CC2)c2nc(N)n3nc(-c4ccccn4)nc3c12] 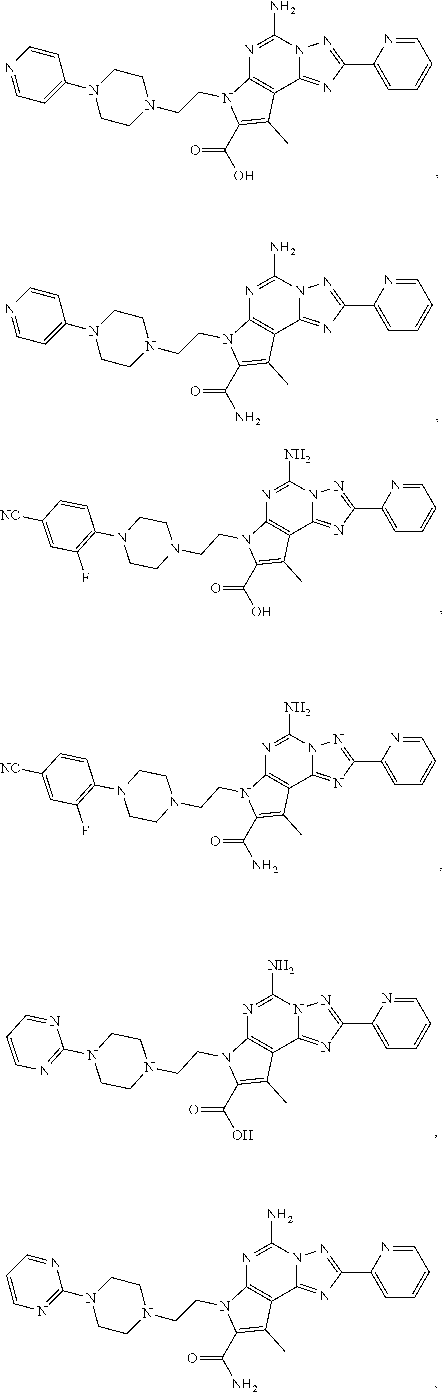 [SMILES: Cc1c(C(=O)O)n(CCN2CCN(c3ccncc3)CC2)c2nc(N)n3nc(-c4ccccn4)nc3c12 ; Cc1c(C(N)=O)n(CCN2CCN(c3ccncc3)CC2)c2nc(N)n3nc(-c4ccccn4)nc3c12 ; Cc1c(C(=O)O)n(CCN2CCN(c3ccc(C#N)cc3F)CC2)c2nc(N)n3nc(-c4ccccn4)nc3c12 ; Cc1c(C(N)=O)n(CCN2CCN(c3ccc(C#N)cc3F)CC2)c2nc(N)n3nc(-c4ccccn4)nc3c12 ; Cc1c(C(=O)O)n(CCN2CCN(c3ncccn3)CC2)c2nc(N)n3nc(-c4ccccn4)nc3c12 ; Cc1c(C(N)=O)n(CCN2CCN(c3ncccn3)CC2)c2nc(N)n3nc(-c4ccccn4)nc3c12] 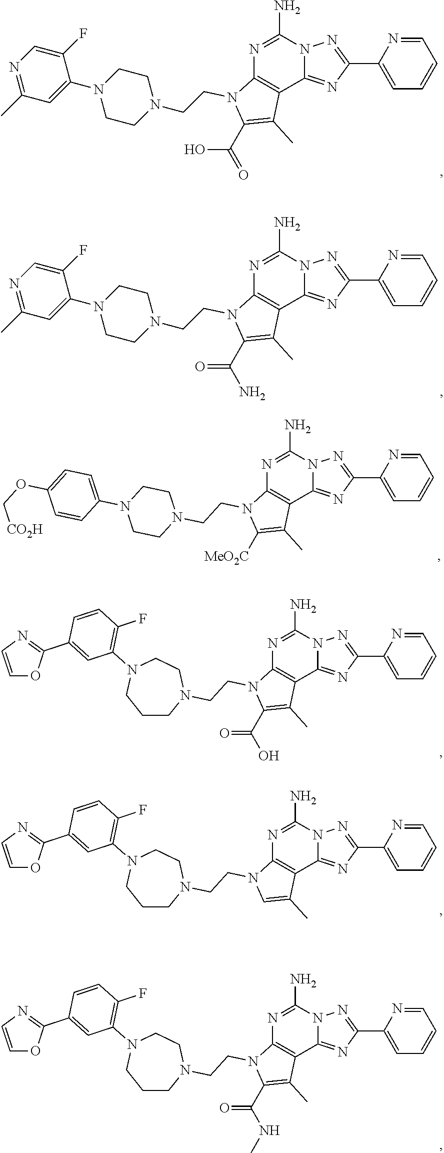 [SMILES: Cc1cc(N2CCN(CCn3c(C(=O)O)c(C)c4c3nc(N)n3nc(-c5ccccn5)nc43)CC2)c(F)cn1 ; Cc1cc(N2CCN(CCn3c(C(N)=O)c(C)c4c3nc(N)n3nc(-c5ccccn5)nc43)CC2)c(F)cn1 ; COC(=O)c1c(C)c2c(nc(N)n3nc(-c4ccccn4)nc23)n1CCN1CCN(c2ccc(OCC(=O)O)cc2)CC1 ; Cc1c(C(=O)O)n(CCN2CCCN(c3cc(-c4ncco4)ccc3F)CC2)c2nc(N)n3nc(-c4ccccn4)nc3c12 ; Cc1cn(CCN2CCCN(c3cc(-c4ncco4)ccc3F)CC2)c2nc(N)n3nc(-c4ccccn4)nc3c12 ; CNC(=O)c1c(C)c2c(nc(N)n3nc(-c4ccccn4)nc23)n1CCN1CCCN(c2cc(-c3ncco3)ccc2F)CC1] 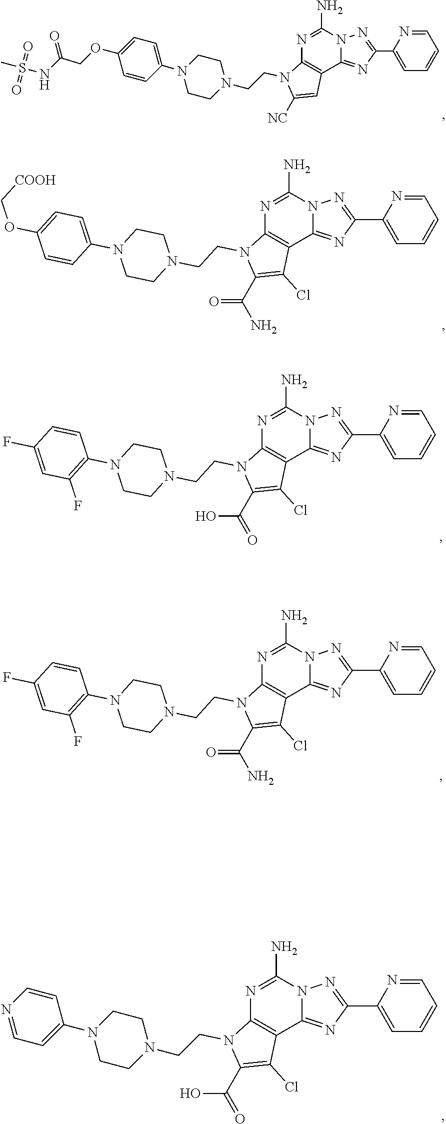 [SMILES: CS(=O)(=O)NC(=O)COc1ccc(N2CCN(CCn3c(C#N)cc4c3nc(N)n3nc(-c5ccccn5)nc43)CC2)cc1 ; NC(=O)c1c(Cl)c2c(nc(N)n3nc(-c4ccccn4)nc23)n1CCN1CCN(c2ccc(OCC(=O)O)cc2)CC1 ; Nc1nc2c(c(Cl)c(C(=O)O)n2CCN2CCN(c3ccc(F)cc3F)CC2)c2nc(-c3ccccn3)nn12 ; NC(=O)c1c(Cl)c2c(nc(N)n3nc(-c4ccccn4)nc23)n1CCN1CCN(c2ccc(F)cc2F)CC1 ; Nc1nc2c(c(Cl)c(C(=O)O)n2CCN2CCN(c3ccncc3)CC2)c2nc(-c3ccccn3)nn12] 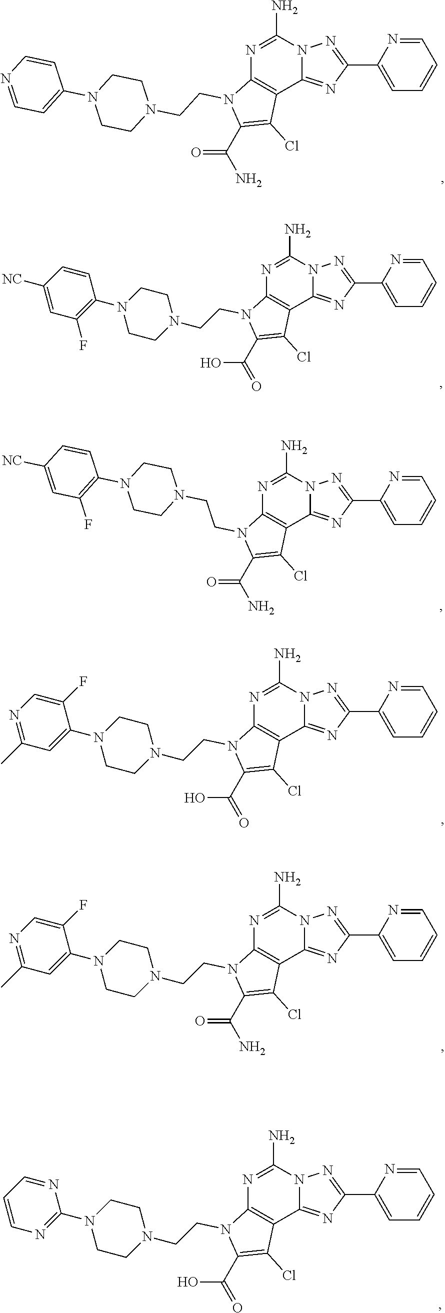 [SMILES: NC(=O)c1c(Cl)c2c(nc(N)n3nc(-c4ccccn4)nc23)n1CCN1CCN(c2ccncc2)CC1 ; N#Cc1ccc(N2CCN(CCn3c(C(=O)O)c(Cl)c4c3nc(N)n3nc(-c5ccccn5)nc43)CC2)c(F)c1 ; N#Cc1ccc(N2CCN(CCn3c(C(N)=O)c(Cl)c4c3nc(N)n3nc(-c5ccccn5)nc43)CC2)c(F)c1 ; Cc1cc(N2CCN(CCn3c(C(=O)O)c(Cl)c4c3nc(N)n3nc(-c5ccccn5)nc43)CC2)c(F)cn1 ; Cc1cc(N2CCN(CCn3c(C(N)=O)c(Cl)c4c3nc(N)n3nc(-c5ccccn5)nc43)CC2)c(F)cn1 ; Nc1nc2c(c(Cl)c(C(=O)O)n2CCN2CCN(c3ncccn3)CC2)c2nc(-c3ccccn3)nn12] 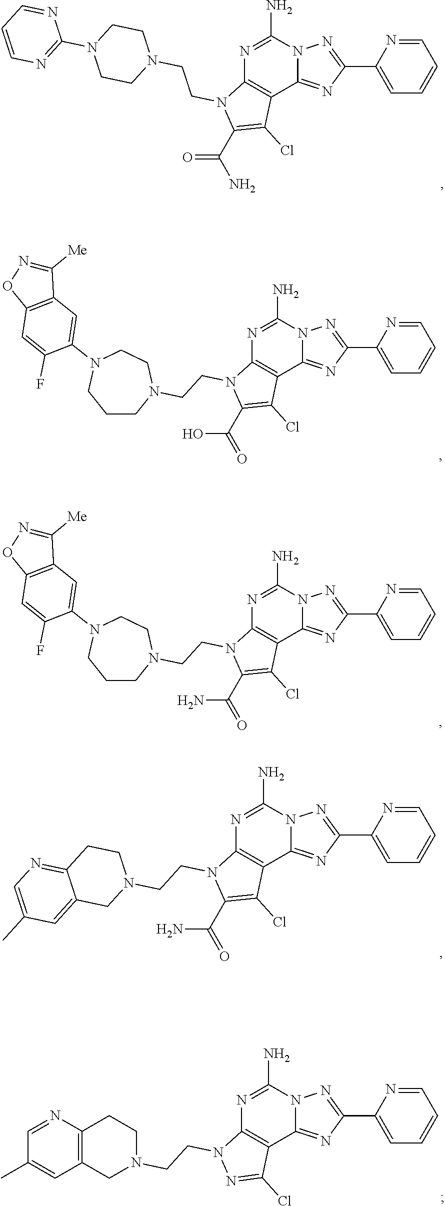 [SMILES: NC(=O)c1c(Cl)c2c(nc(N)n3nc(-c4ccccn4)nc23)n1CCN1CCN(c2ncccn2)CC1 ; Cc1noc2cc(F)c(N3CCCN(CCn4c(C(=O)O)c(Cl)c5c4nc(N)n4nc(-c6ccccn6)nc54)CC3)cc12 ; Cc1noc2cc(F)c(N3CCCN(CCn4c(C(N)=O)c(Cl)c5c4nc(N)n4nc(-c6ccccn6)nc54)CC3)cc12 ; Cc1cnc2c(c1)CN(CCn1c(C(N)=O)c(Cl)c3c1nc(N)n1nc(-c4ccccn4)nc31)CC2 ; Cc1cnc2c(c1)CN(CCn1nc(Cl)c3c1nc(N)n1nc(-c4ccccn4)nc31)CC2]
or a pharmaceutically acceptable salt or solvate thereof.

46. A pharmaceutical composition comprising a compound of claim 1, or a pharmaceutically acceptable salt or solvate thereof, and at least one pharmaceutically acceptable excipient.

47. The pharmaceutical composition of claim 46, wherein the pharmaceutical composition is formulated for administration to a mammal by oral administration, intravenous administration, or subcutaneous administration.

48. The pharmaceutical composition of claim 46, wherein the pharmaceutical composition is in the form of a tablet, a pill, a capsule, a liquid, a suspension, a dispersion, a solution, or an emulsion.

49. A method of modulating the A_{2A} adenosine receptor in a mammal comprising administering to the mammal a compound of claim 1, or any pharmaceutically acceptable salt or solvate thereof.

50. A method of treating a disease or disorder that is mediated by the A_{2A} adenosine receptor in a mammal comprising administering to the mammal in need thereof a therapeutically effective amount of a compound of claim 1, or a pharmaceutically acceptable salt or solvate thereof.

51. The method of claim 50, wherein the disease or disorder is selected from the group consisting of cardiovascular diseases, fibrosis, neurological disorders, type I hypersensitivity disorders, chronic and acute liver diseases, lung diseases, renal diseases, diabetes, obesity, and cancer.

52. The method of claim 50, wherein the disease or disorder is cancer.

53. A method for treating cancer in a mammal, the method comprising administering to the mammal a compound of claim 1, or any pharmaceutically acceptable salt or solvate thereof.

54. The method of claim 53, wherein the cancer is a solid tumor.

55. The method of claim 53, wherein the cancer is bladder cancer, colon cancer, brain cancer, breast cancer, endometrial cancer, heart cancer, kidney cancer, lung cancer, liver cancer, uterine cancer, blood and lymphatic cancer, ovarian cancer, pancreatic cancer, prostate cancer, thyroid cancer, or skin cancer.

56. The method of claim 53, wherein the cancer is prostate cancer, breast cancer, colon cancer, or lung cancer.

57. The method of claim 53, wherein the cancer is a sarcoma, carcinoma, or lymphoma.

58. The method of claim 49, further comprising administering at least one additional therapy to the mammal.

59. The method of claim 49, wherein the mammal is a human.

60. (canceled)

61. (canceled)
